(19) United States
(12) Patent Application Publication
(10) Pub. No.: US 2017/0298056 A1
(43) Pub. Date: Oct. 19, 2017

(54) SMALL MOLECULE INHIBITORS OF HIV-1 ENTRY AND METHODS OF USE THEREOF

(71) Applicants: Dana-Farber Cancer Institute, Inc., Boston, MA (US); The Trustees of the University of Pennsylvania, Philadelphia, PA (US)

(72) Inventors: Joseph Sodroski, Medford, MA (US); Alon Herschhorn, Brookline, MA (US); Christopher Gu, Brookline, MA (US); Joel R. Courter, Philadelphia, PA (US); Mark Farrell, Philadelphia, PA (US); Amos B. Smith, III, Merion, PA (US)

(21) Appl. No.: 15/309,087

(22) PCT Filed: May 8, 2015
(86) PCT No.: PCT/US15/29846

§ 371 Date: Nov. 4, 2016

Related U.S. Application Data
(60) Provisional application No. 61/990,297, filed on May 8, 2014.

(51) Int. Cl. C07D 417/12; C07D 285/14

(52) U.S. Cl. CPC C07D 417/12; C07D 285/14

(57) ABSTRACT

Described herein are small-molecule compounds that specifically inhibit a wide range of HIV-1 isolates without interfering with CD4 or CCR5 binding. Methods of using die compounds for treating or preventing HIV infection are also described.

### RELATED APPLICATIONS

This application claims the benefit of priority to U.S. Provisional Patent Application Ser. No. 61/990,297, filed May 8, 2014, the contents of which are hereby incorporated by reference.

### GOVERNMENT SUPPORT

This invention was made with government support under GM56550 and DA0346 awarded by the National Institutes of Health. The government has certain rights in the invention. This statement is included solely to comply with 37 C.F.R. §401.14(a)(f)(4) and should not be taken as an assertion or admission that the application discloses and/or claims only one invention.

### BACKGROUND OF THE INVENTION

In the absence of antiviral therapy, infection by human immunodeficiency virus type 1 (HIV-1) typically leads to acquired immunodeficiency syndrome (AIDS) and death. Entry of HIV-1 into target cells is mediated by the interaction of the viral envelope glycoproteins (Envs) with the CD4 receptor and either the CCR5 or CXCR4 coreceptor. HIV-1 Envs on the surface of virions are trimers consisting of three gp120 exterior glycoproteins non-covalently associated with three gp41 transmembrane glycoproteins. Binding of gp120 to the CD4 receptor initiates the entry process, leading to Env structural rearrangements that: i) reposition the gp120 V1/V2 and V3 regions; ii) expose the coreceptor-binding site of gp120; and iii) form and/or expose the heptad repeat 1 (HR1) coiled coil of gp41. Subsequent interaction of gp120 with the coreceptor is thought to trigger the insertion of the hydrophobic gp41 fusion peptide into the target cell membrane and the refolding of the gp41 ectodomain into a very stable six-helix bundle. This ordered sequence of events channels the energy difference between the metastable unliganded state of Env and the stable six-helix bundle into the fusion of the viral and cell membranes.

The complex HIV-1 entry process is vulnerable to inhibition by small molecules. Some gp120-directed inhibitors have been used as leads for drug development as well as probes to investigate different Env conformations. NBD-556, a small molecule that targets the CD4-binding site of gp120, was used to demonstrate that the CD4-bound conformation is rarely sampled spontaneously on primary HIV-1 isolates. Studies of BMS-806, a potent entry inhibitor, highlighted the importance of CD4-induced formation/exposure of the gp41 HR1 coiled coil in virus entry. Several derivatives of both compounds with improved breadth and potency have been developed for potential clinical application.

There exists a need for small molecules that inhibit HIV-1 Env function. Such inhibitors lead to novel antiretroviral drugs with high potency and breadth.

### SUMMARY OF THE INVENTION

In certain embodiments, the invention relates to a compound of Formula I

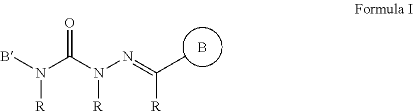

or a pharmaceutically acceptable salt or solvate thereof,
wherein, independently for each occurrence,

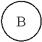

is optionally substituted aryl, optionally substituted heteroaryl, optionally substituted alkenyl, or optionally substituted cycloalkenyl;

R is hydrogen or alkyl;

B′ is optionally substituted aryl, optionally substituted heteroaryl, optionally substituted cycloalkyl, or B′, when taken together with either instance of —NR—, forms a substituted or unsubstituted heterocycloalkyl ring,

provided the compound is not

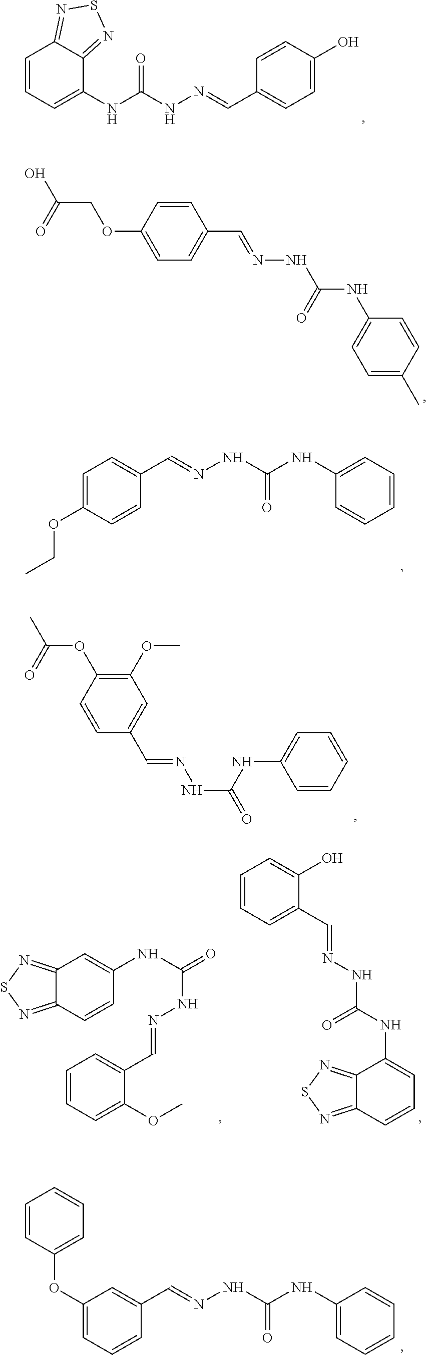

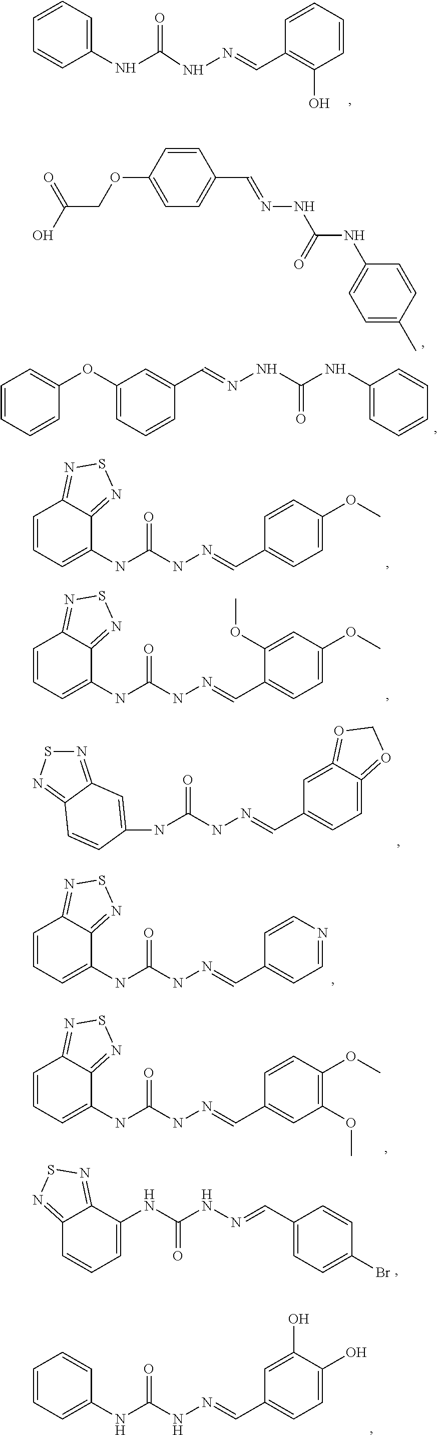

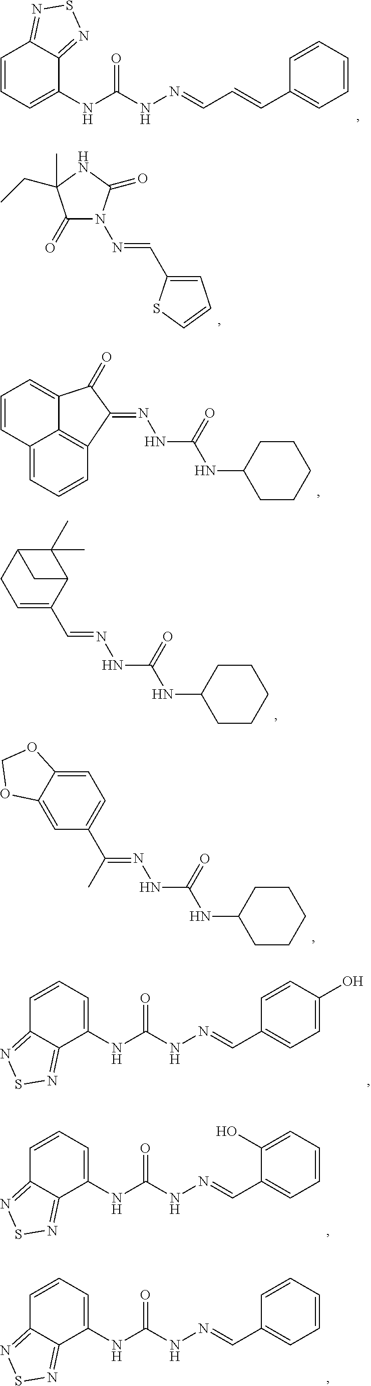

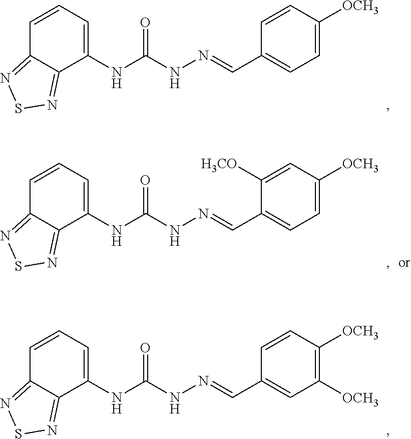 [SMILES: COc1ccc(/C=N/NC(=O)Nc2cccc3nsnc23)cc1 ; COc1ccc(/C=N/NC(=O)Nc2cccc3nsnc23)c(OC)c1 ; COc1ccc(/C=N/NC(=O)Nc2cccc3nsnc23)cc1OC]

wherein any atoms with an incomplete valence are covalently bonded to one or more hydrogen atoms to complete their valence.

In certain embodiments, the invention relates to a compound of Formula II

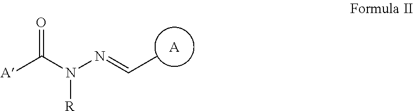

or a pharmaceutically acceptable salt or solvate thereof,
wherein, independently for each occurrence,

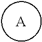

is optionally substituted aryl or optionally substituted heteroaryl; and

A′ is optionally substituted alkyl, optionally substituted cycloalkyl, optionally substituted aryl, or optionally substituted heteroaryl,

provided the compound is not

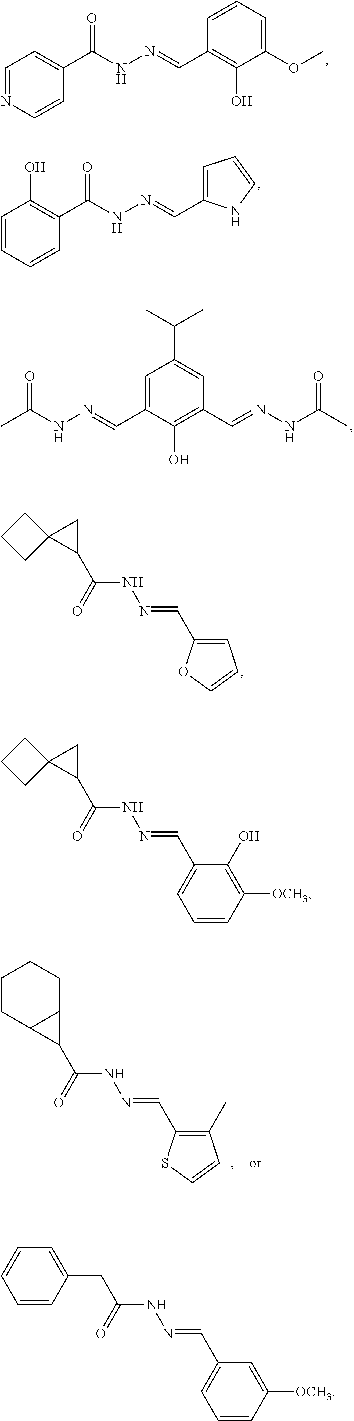

In certain embodiments, the invention relates to a compound of Formula III

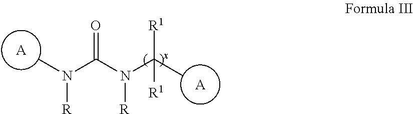

or a pharmaceutically acceptable salt or solvate thereof,
wherein, independently for each occurrence,

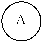

is optionally substituted aryl or optionally substituted heteroaryl;

R is hydrogen or alkyl;

R¹ is hydrogen, hydroxy, alkoxy, or alkyl; and

x is 0, 1, 2, or 3,

provided the compound is not

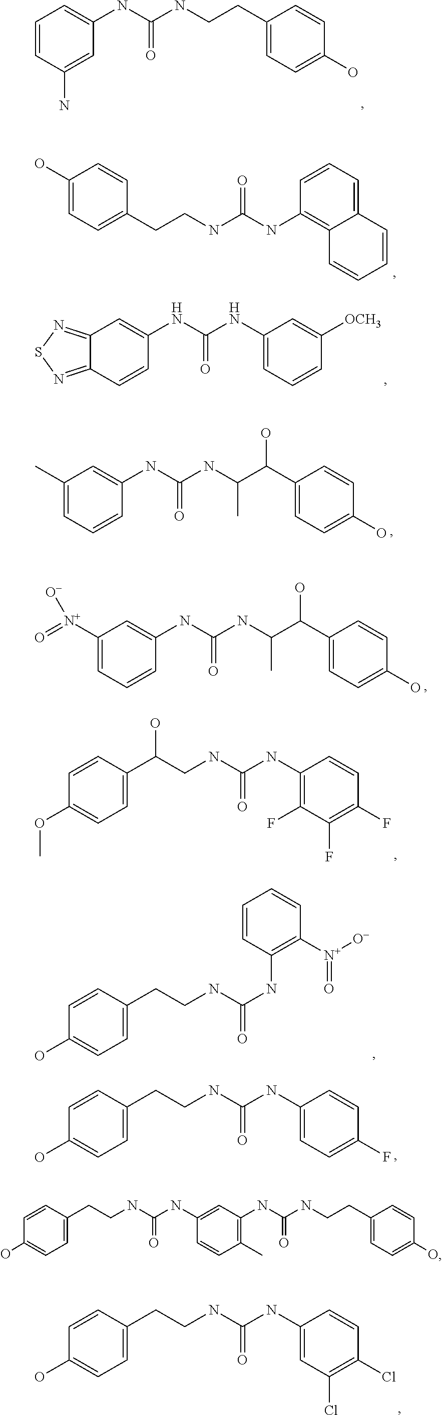

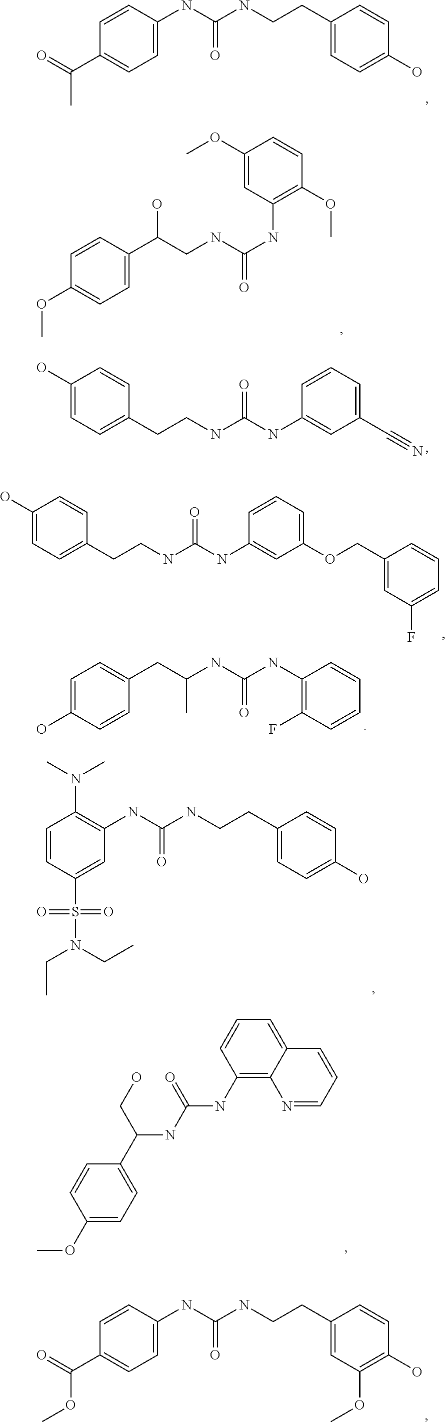

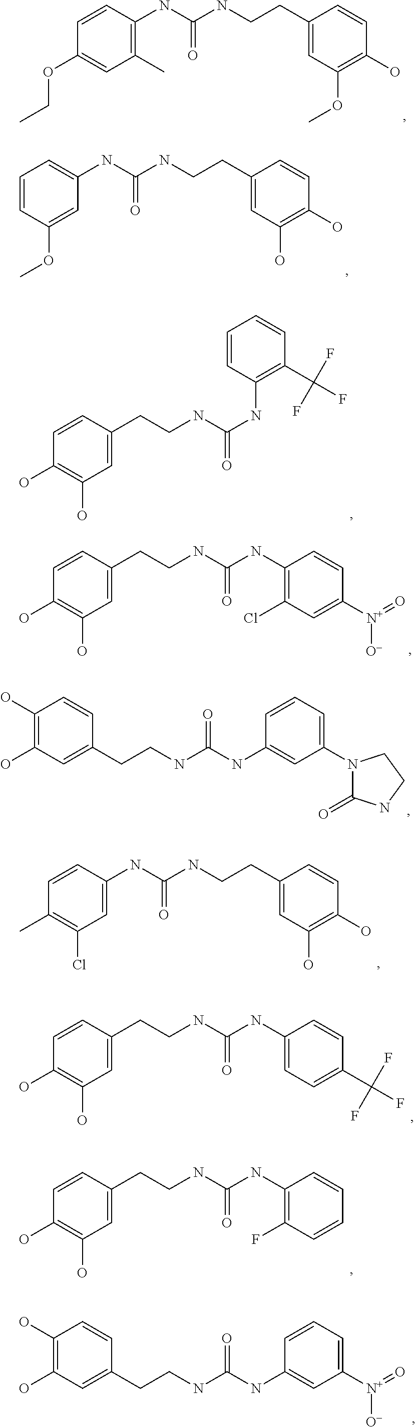

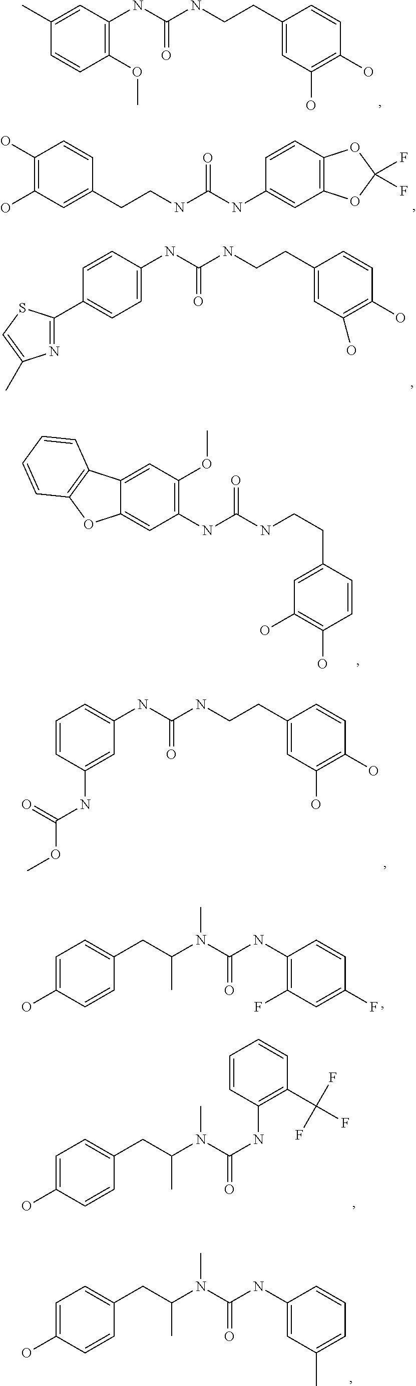

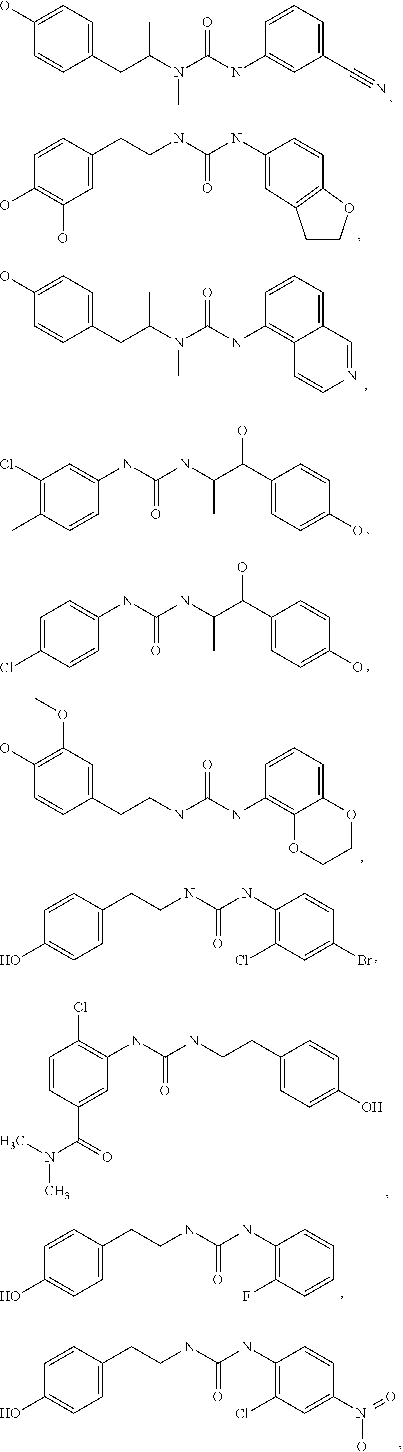

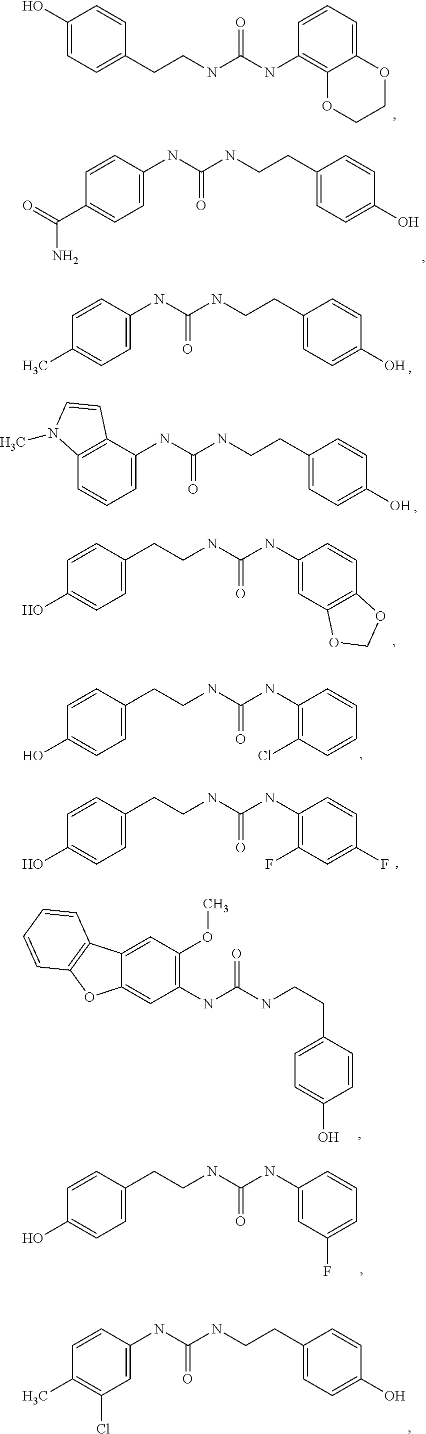

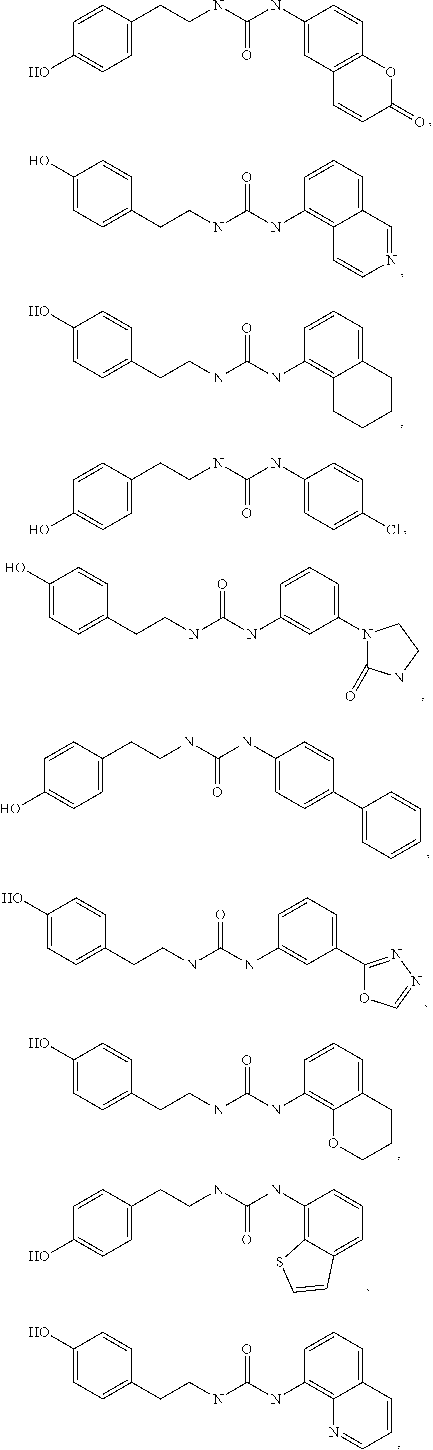

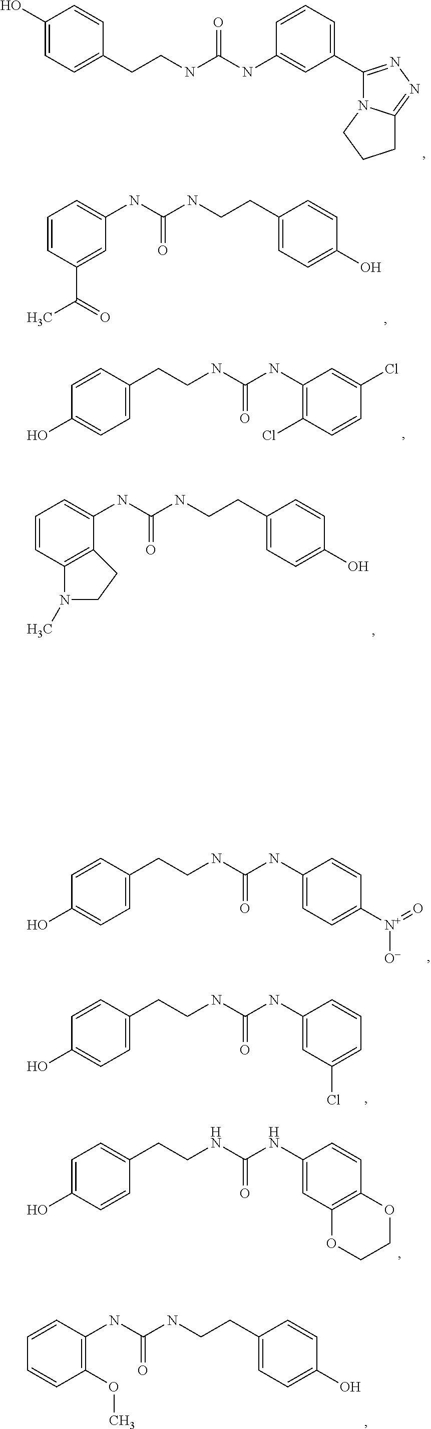

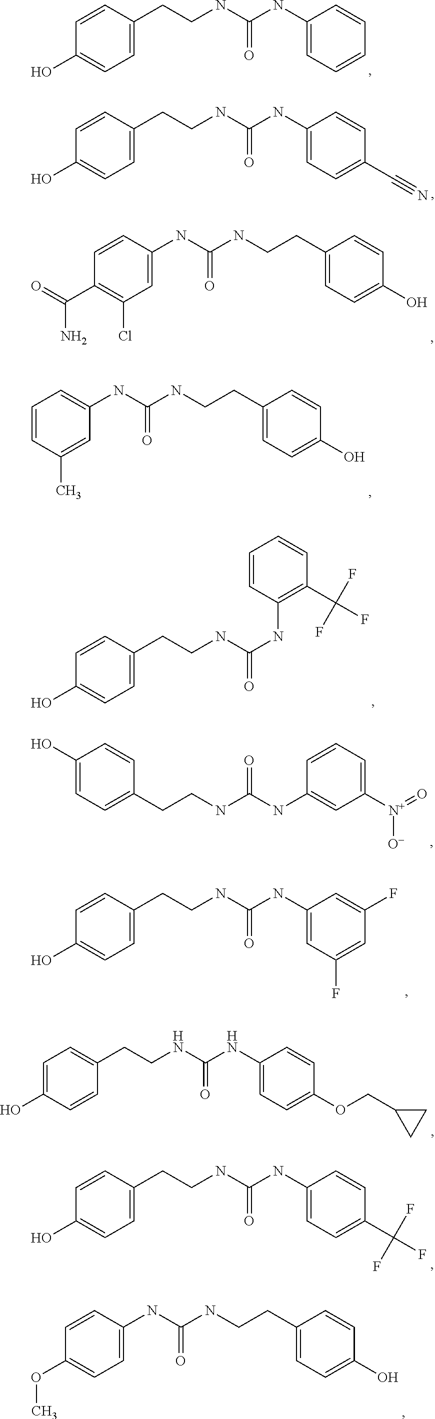

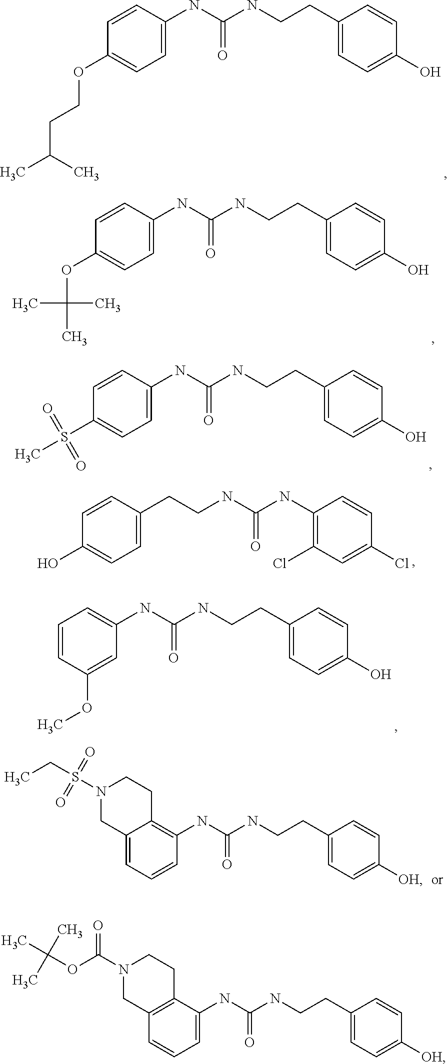

wherein any atoms with an incomplete valence are covalently bonded to one or more hydrogen atoms to complete their valence.

In certain embodiments, the invention relates to a compound of Formula IV

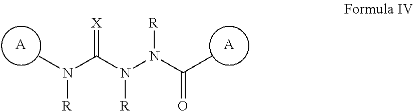

or a pharmaceutically acceptable salt or solvate thereof,
wherein, independently for each occurrence,

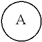

is optionally substituted aryl or optionally substituted heteroaryl;

R is hydrogen or alkyl; and

X is O or S,

provided the compound is not

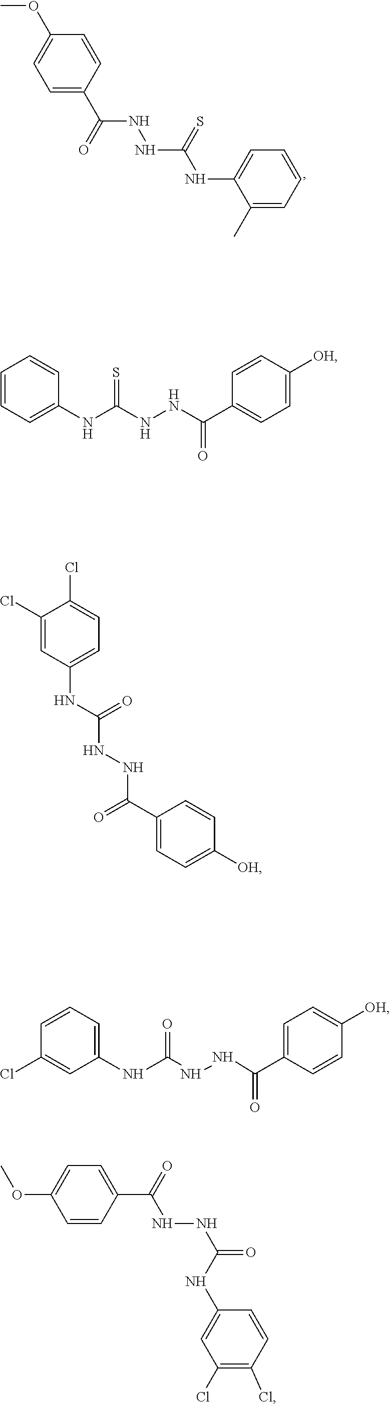 [SMILES: COc1ccc(C(=O)NNC(=S)Nc2ccccc2C)cc1 ; O=C(NNC(=S)Nc1ccccc1)c1ccc(O)cc1 ; O=C(NNC(=O)c1ccc(O)cc1)Nc1ccc(Cl)c(Cl)c1 ; O=C(NNC(=O)c1ccc(O)cc1)Nc1cccc(Cl)c1 ; COc1ccc(C(=O)NNC(=O)Nc2ccc(Cl)c(Cl)c2)cc1]

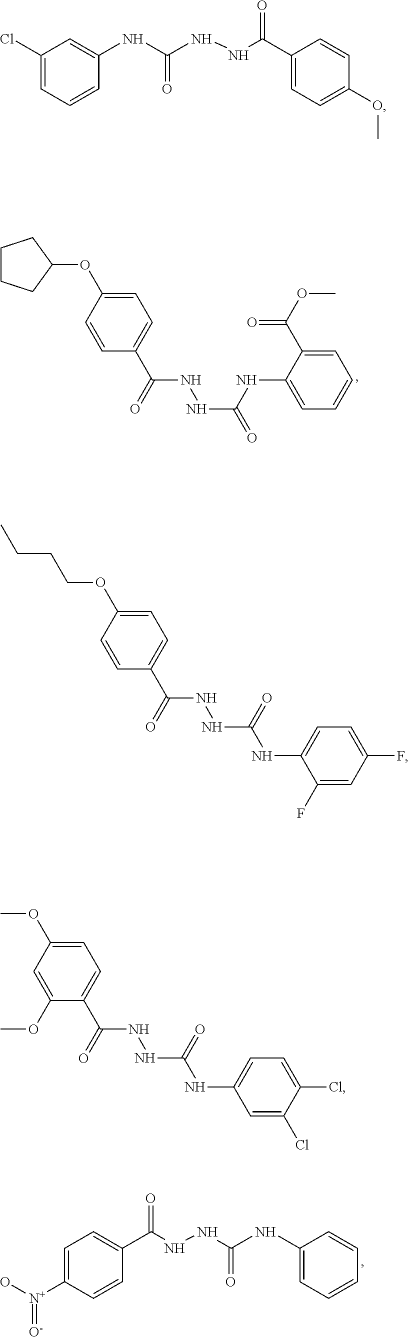

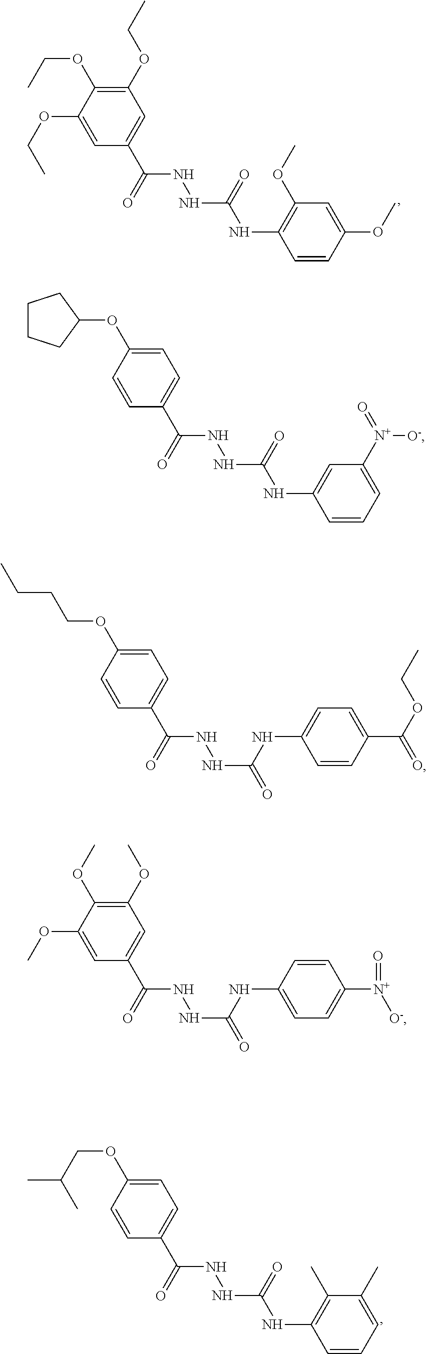

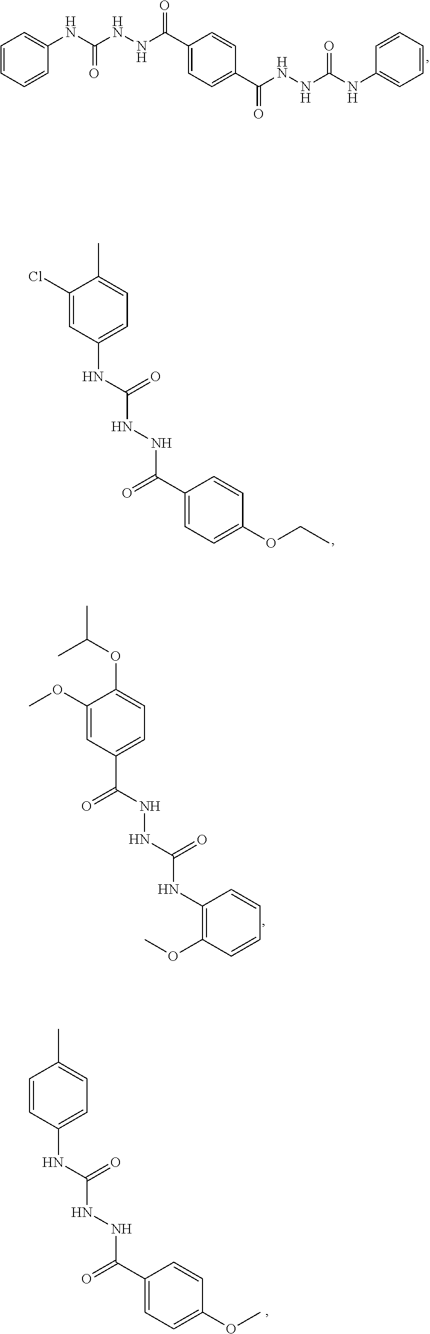

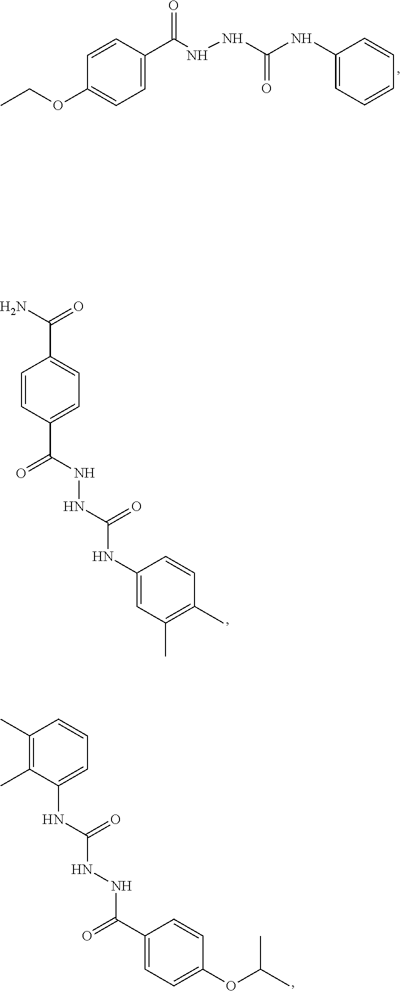 [SMILES: CCOc1ccc(C(=O)NNC(=O)Nc2ccccc2)cc1 ; Cc1ccc(NC(=O)NNC(=O)c2ccc(C(N)=O)cc2)cc1C ; Cc1cccc(NC(=O)NNC(=O)c2ccc(OC(C)C)cc2)c1C]

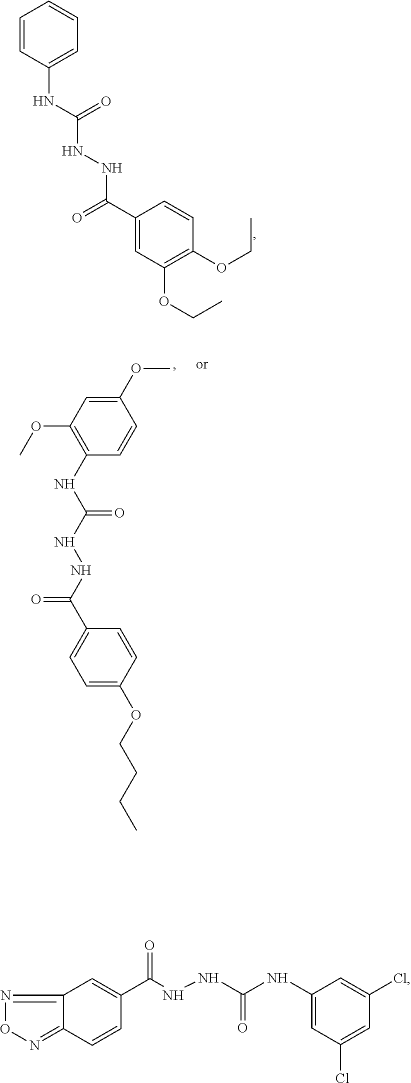

wherein any atoms with an incomplete valence are covalently bonded to one or more hydrogen atoms to complete their valence.

In certain embodiments, the invention relates to a compound of Formula V

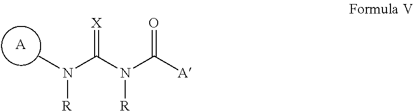

or a pharmaceutically acceptable salt or solvate thereof,
wherein, independently for each occurrence,

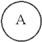

is optionally substituted aryl or optionally substituted heteroaryl;

R is hydrogen or alkyl;

A′ is optionally substituted alkyl, optionally substituted cycloalkyl, optionally substituted aryl, or optionally substituted heteroaryl; and

X is O or S,

provided the compound is not

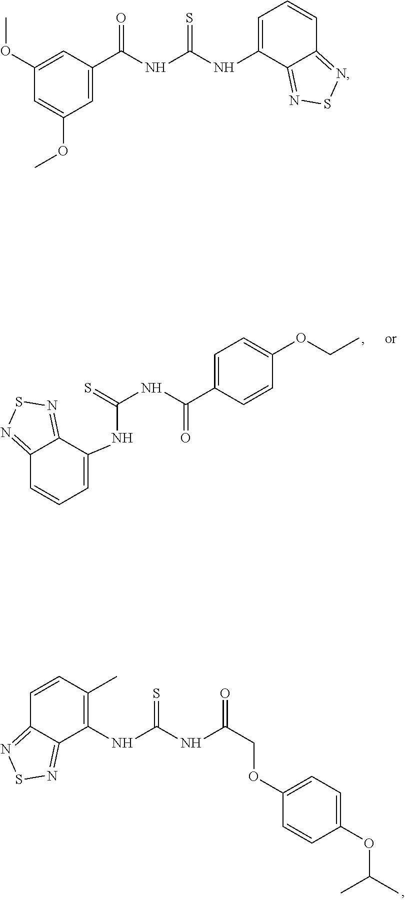 [SMILES: COc1cc(OC)cc(C(=O)NC(=S)Nc2cccc3nsnc23)c1 ; CCOc1ccc(C(=O)NC(=S)Nc2cccc3nsnc23)cc1 ; Cc1ccc2nsnc2c1NC(=S)NC(=O)COc1ccc(OC(C)C)cc1]

wherein any atoms with an incomplete valence are covalently bonded to one or more hydrogen atoms to complete their valence.

In certain embodiments, the invention relates to a compound of Formula VI

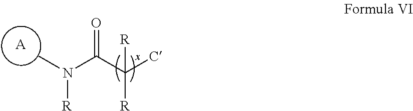

or a pharmaceutically acceptable salt or solvate thereof,
wherein, independently for each occurrence,

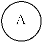

is optionally substituted aryl or optionally substituted heteroaryl;

R is hydrogen or alkyl;

x is 0, 1, 2, or 3; and

C′ is optionally substituted aryl, optionally substituted heteroaryl, optionally substituted cycloalkyl, optionally substituted heterocyclyl, optionally substituted aryloxy, optionally substituted heteroaryloxy optionally substituted arylthio, or optionally substituted heteroarylthio;

provided the compound is not

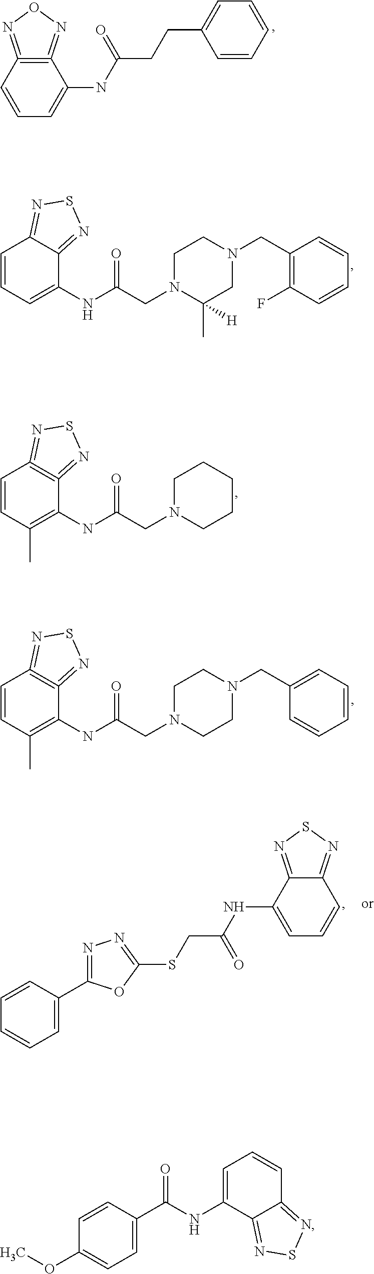

wherein any atoms with an incomplete valence are covalently bonded to one or more hydrogen atoms to complete their valence.

In certain embodiments, the invention relates to a compound of Formula VII

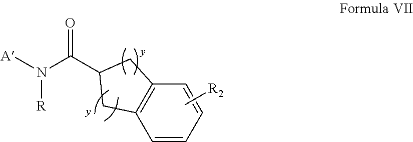

or a pharmaceutically acceptable salt or solvate thereof,
wherein, independently for each occurrence,

A′ is optionally substituted alkyl, optionally substituted cycloalkyl, optionally substituted aryl, or optionally substituted heteroaryl,

R is hydrogen or alkyl;

y is 1 or 2; and

R² is halo, hydroxy, alkoxy, alkylthio, or amino,

provided the compound is not

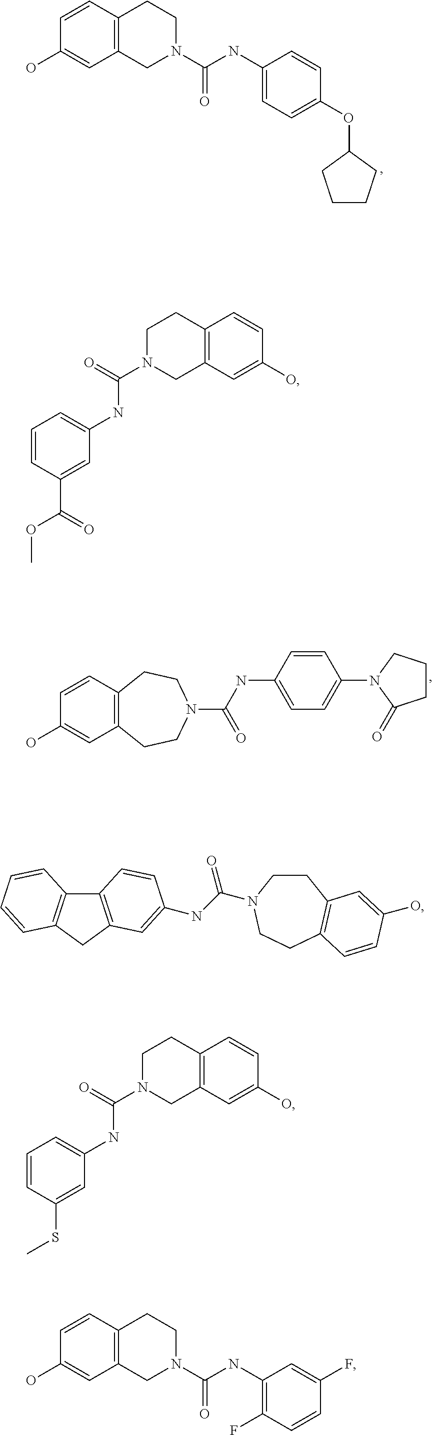

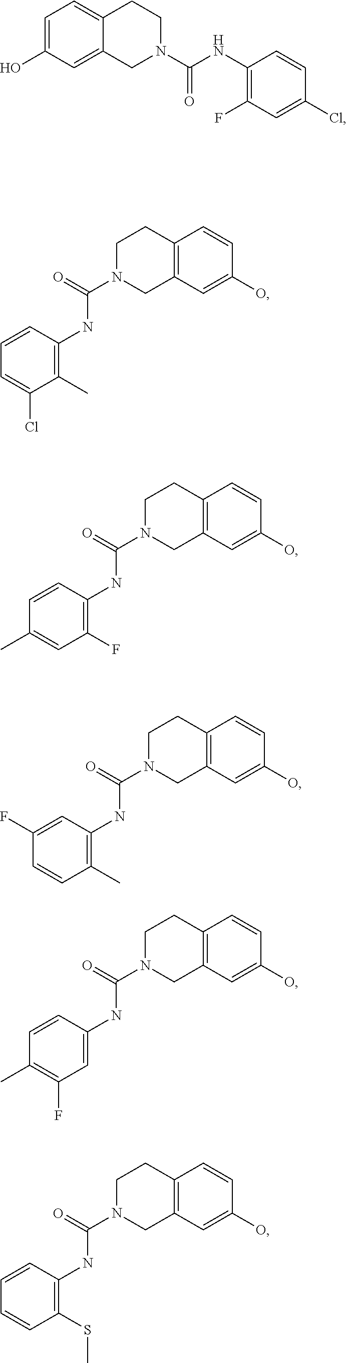

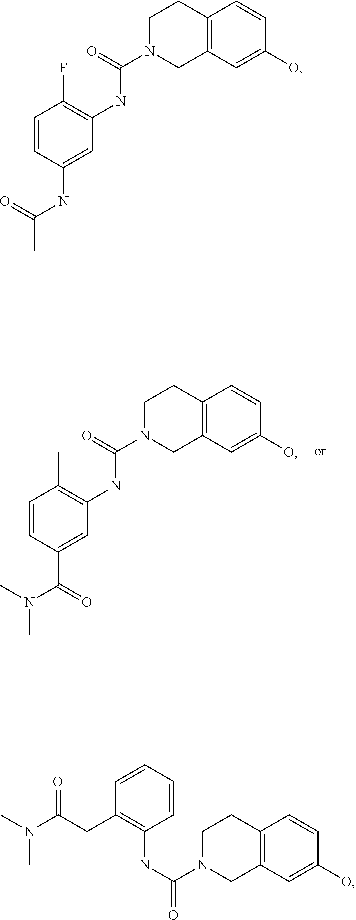

wherein any atoms with an incomplete valence are covalently bonded to one or more hydrogen atoms to complete their valence.

In certain embodiments, the invention relates to a method of inhibiting HIV exterior envelope glycoprotein gp120 comprising the step of: contacting HIV with an effective amount of a compound according to any one of Formulae I-VII.

In certain embodiments, the invention relates to a method of inhibiting transmission of HIV to a cell comprising the step of: contacting HIV with an effective amount of a compound according to any one of one of Formulae I-VII, thereby inhibiting transmission of HIV to said cell.

In certain embodiments, the invention relates to a method of inhibiting the progression of HIV infection in a human host comprising the step of: contacting HIV with an effective amount of a compound according to any one of Formulae I-VII, thereby inhibiting progression of HIV in the human host.

In certain embodiments, the invention relates to a method of

(a) inhibiting HIV exterior envelope glycoprotein gp120 comprising the step of: contacting HIV with an effective amount of a compound;

(b) inhibiting transmission of HIV to a cell comprising the step of: contacting HIV with an effective amount of a compound, thereby inhibiting transmission of HIV to said cell; or

(c) inhibiting the progression of HIV infection in a human host comprising the step of: contacting HIV with an effective amount of a compound, thereby inhibiting progression of HIV in the human host,

wherein the compound is selected from the group consisting of:

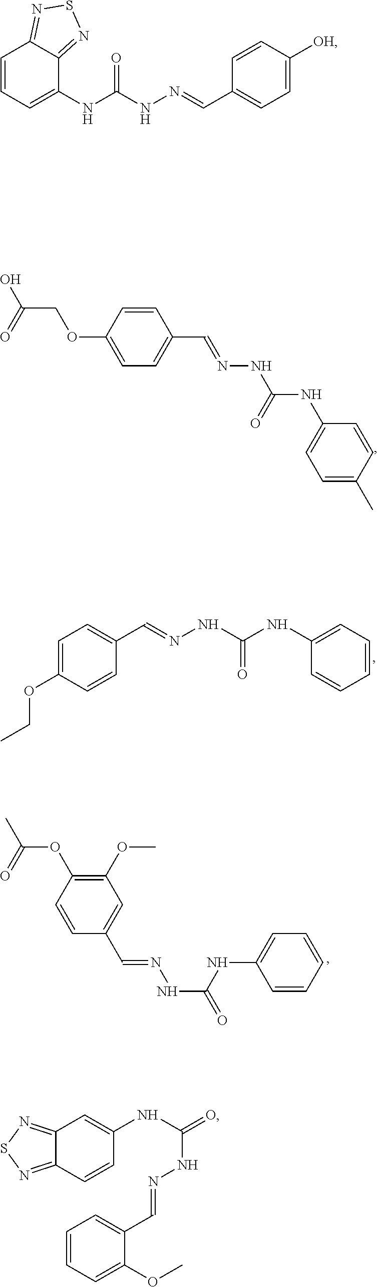

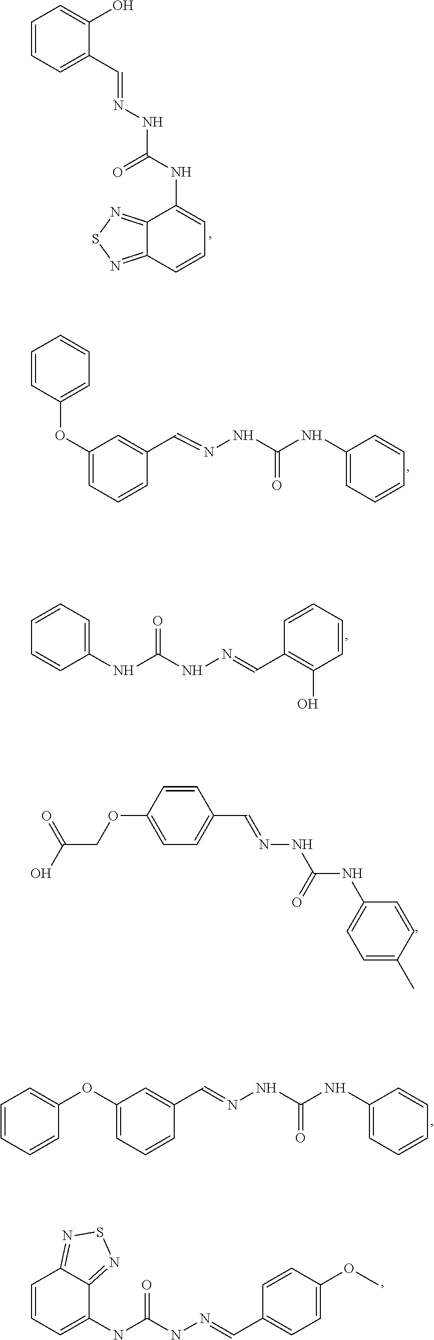 [SMILES: O=C(N/N=C/c1ccccc1O)Nc1cccc2nsnc12 ; O=C(N/N=C/c1cccc(Oc2ccccc2)c1)Nc1ccccc1 ; O=C(N/N=C/c1ccccc1O)Nc1ccccc1 ; Cc1ccc(NC(=O)N/N=C/c2ccc(OCC(=O)O)cc2)cc1 ; O=C(N/N=C/c1cccc(Oc2ccccc2)c1)Nc1ccccc1 ; COc1ccc(/C=N/NC(=O)Nc2cccc3nsnc23)cc1]

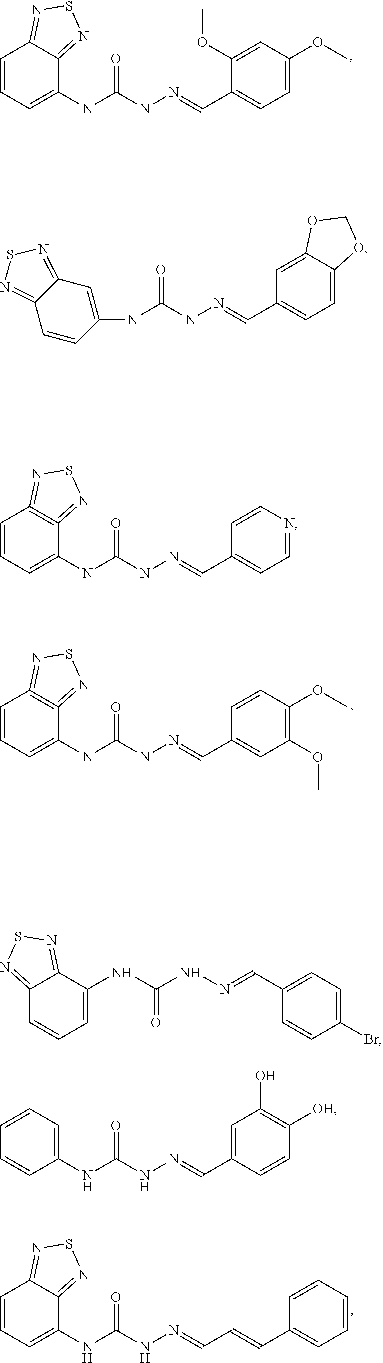

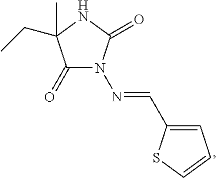 [SMILES: CCC1(C)NC(=O)N(/N=C/c2cccs2)C1=O]

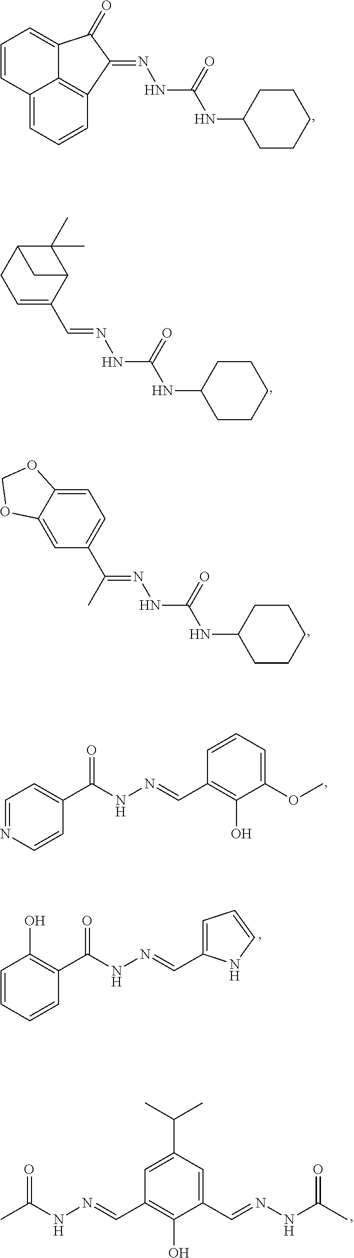

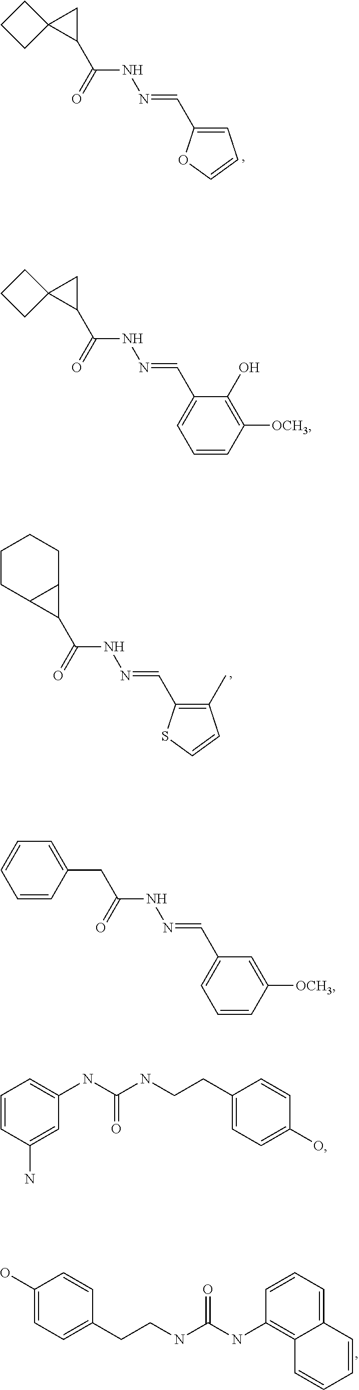

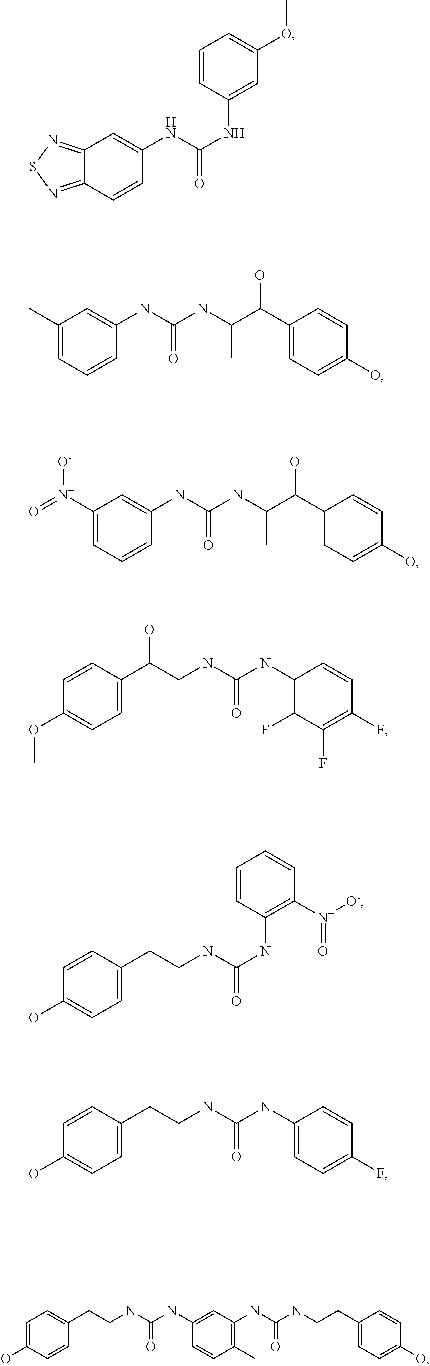

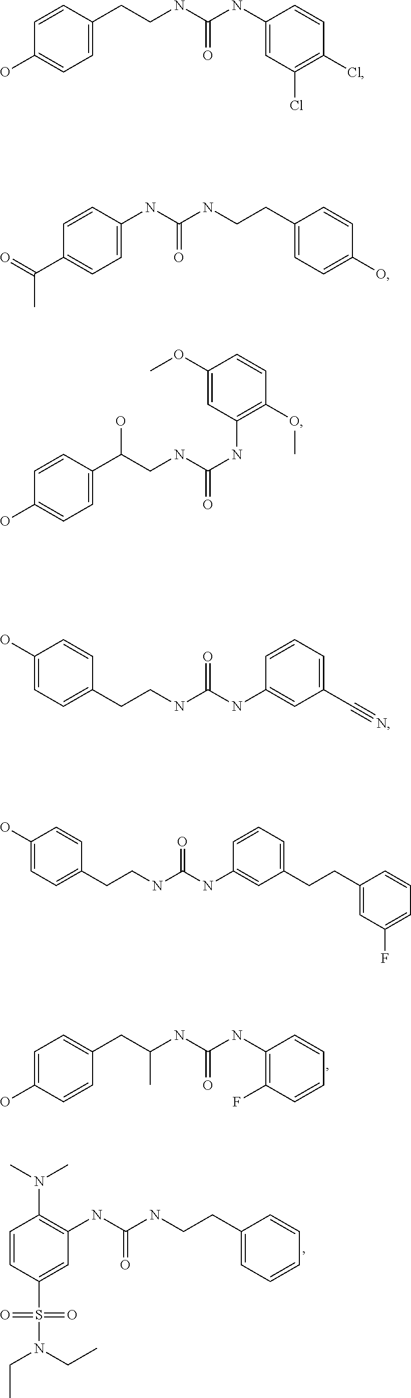

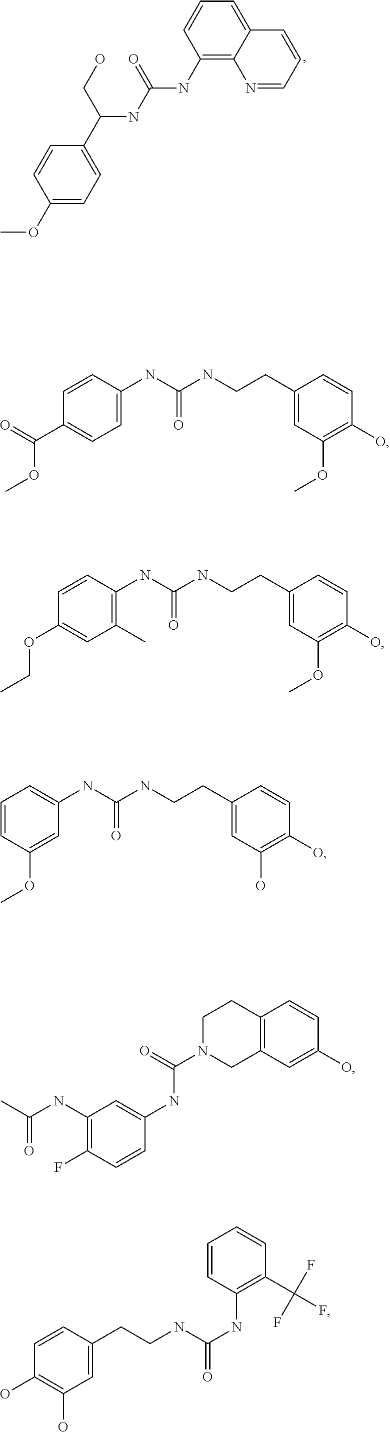

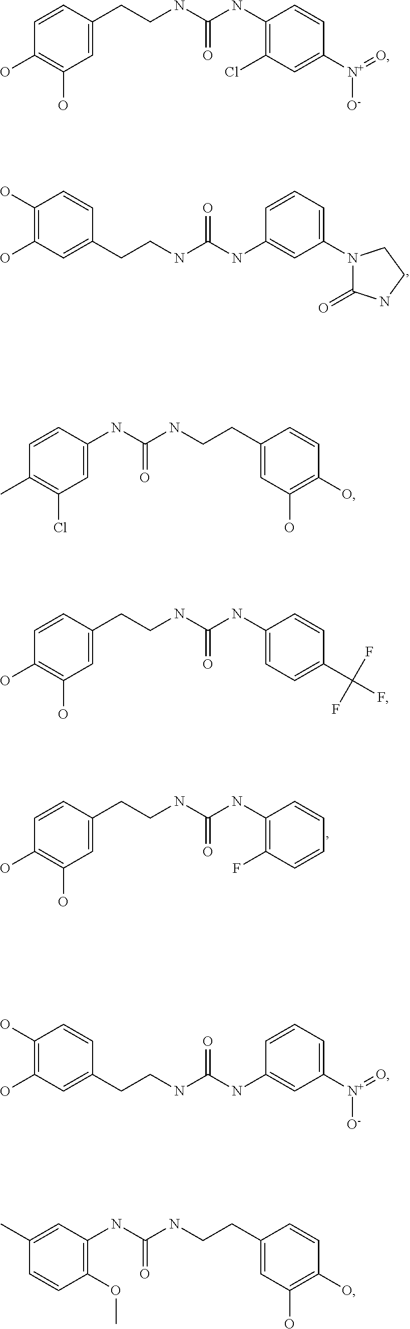

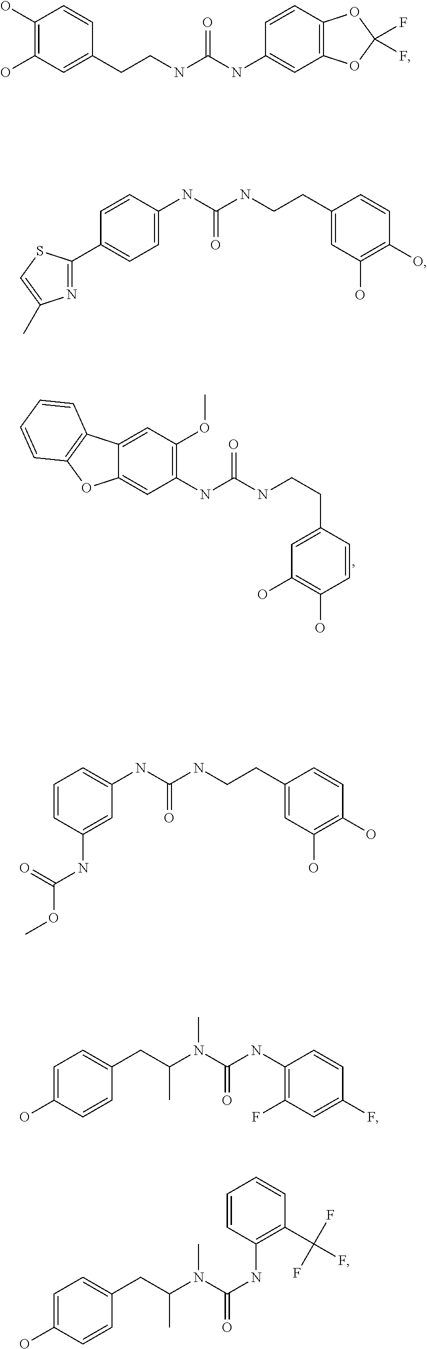

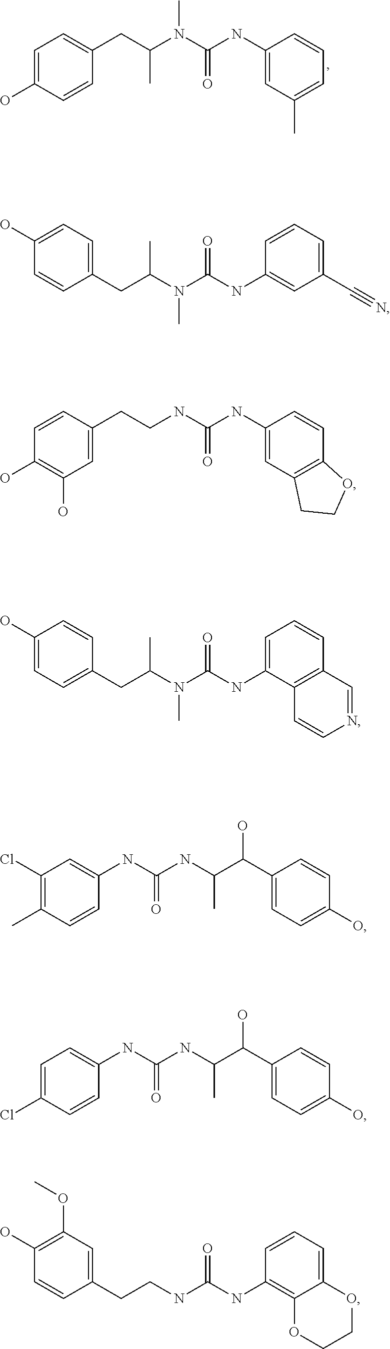

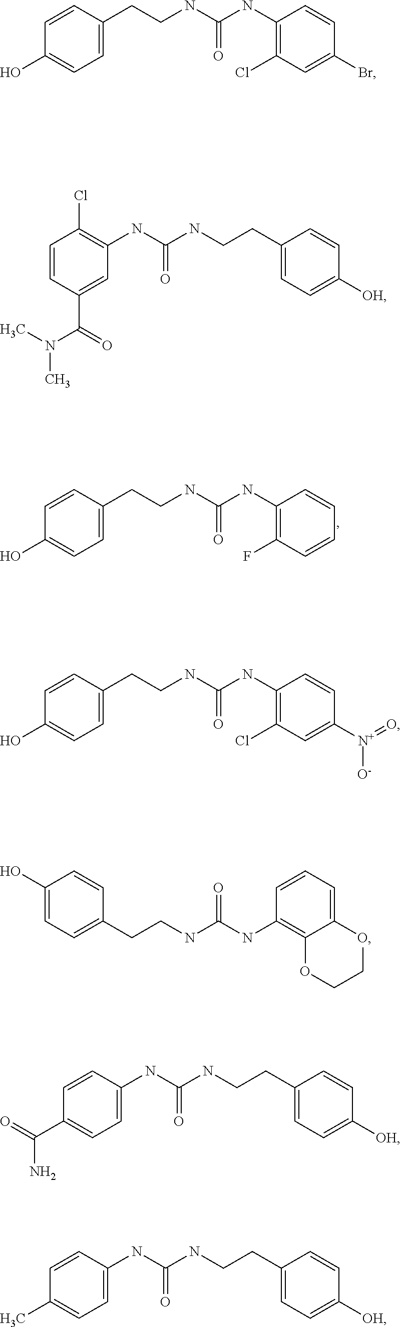

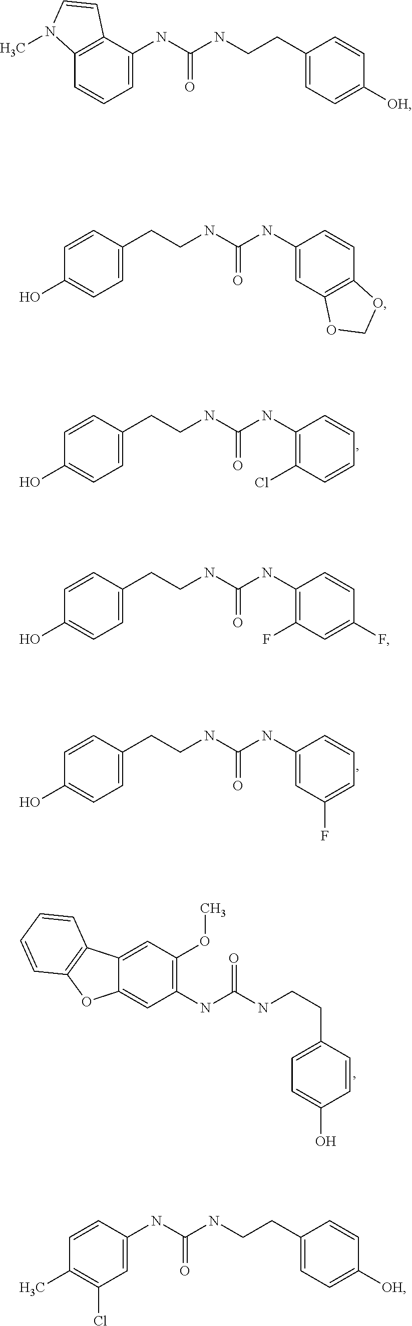

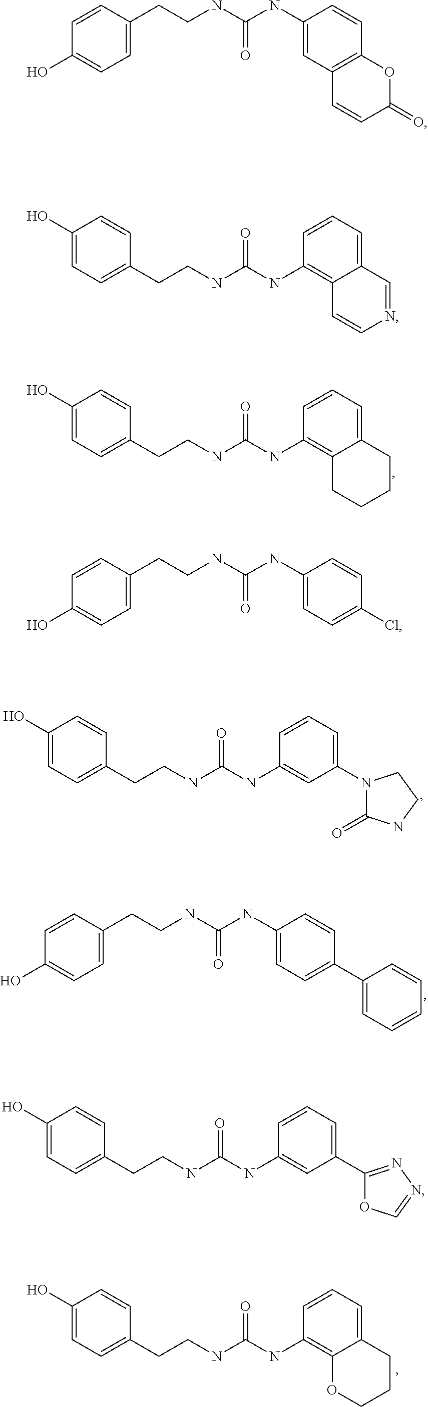

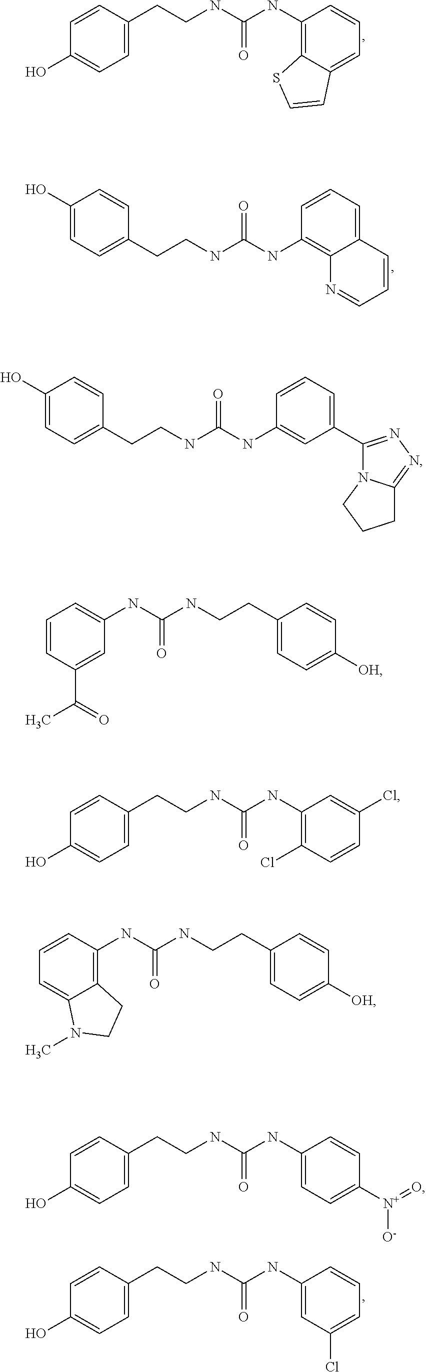

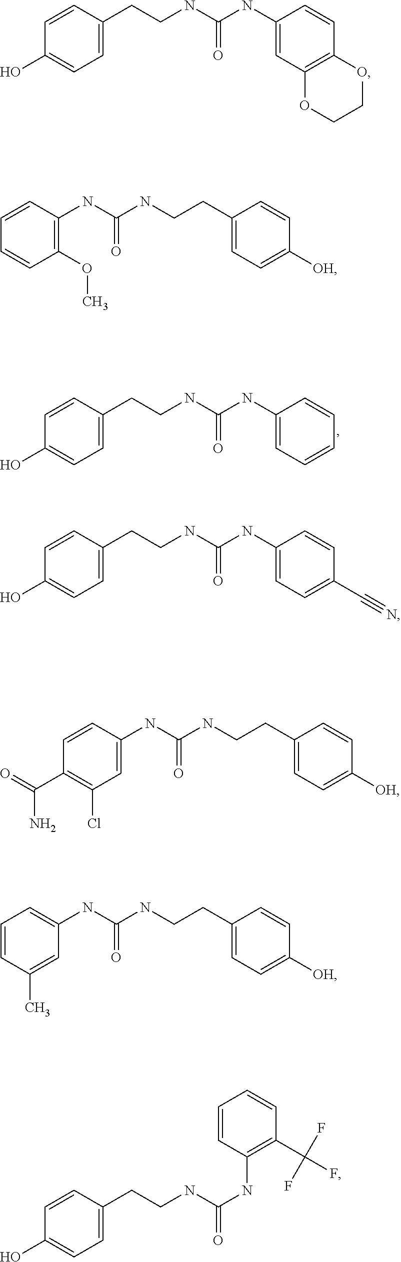

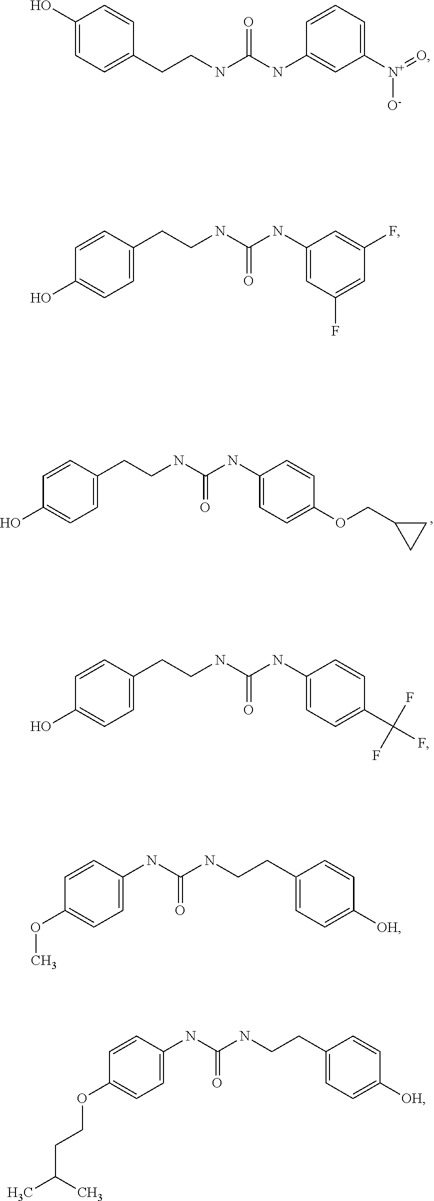 [SMILES: O=C(NCCc1ccc(O)cc1)Nc1cccc([N+](=O)[O-])c1 ; O=C(NCCc1ccc(O)cc1)Nc1cc(F)cc(F)c1 ; O=C(NCCc1ccc(O)cc1)Nc1ccc(OCC2CC2)cc1 ; O=C(NCCc1ccc(O)cc1)Nc1ccc(C(F)(F)F)cc1 ; COc1ccc(NC(=O)NCCc2ccc(O)cc2)cc1 ; CC(C)CCOc1ccc(NC(=O)NCCc2ccc(O)cc2)cc1]

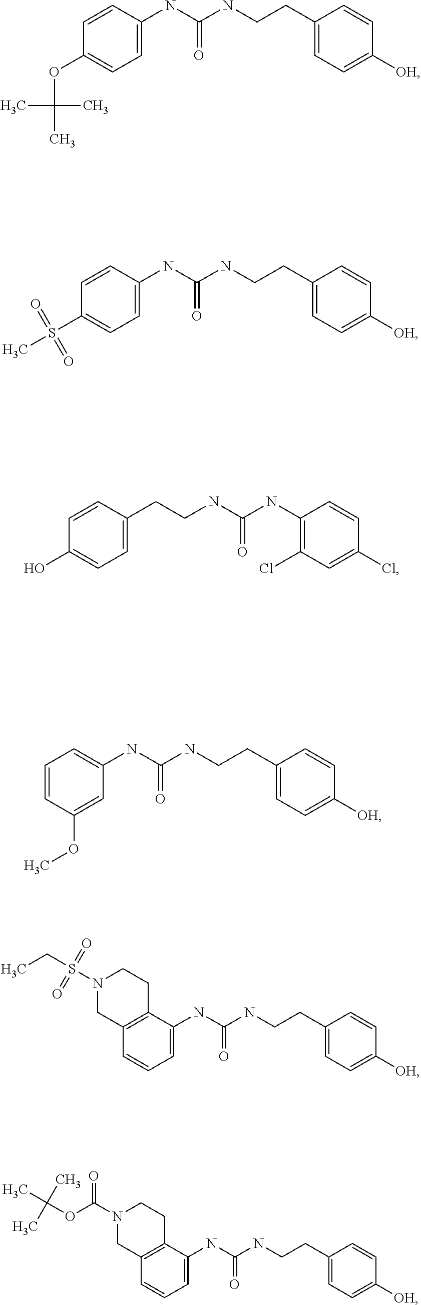 [SMILES: CC(C)(C)Oc1ccc(NC(=O)NCCc2ccc(O)cc2)cc1 ; CS(=O)(=O)c1ccc(NC(=O)NCCc2ccc(O)cc2)cc1 ; O=C(NCCc1ccc(O)cc1)Nc1ccc(Cl)cc1Cl ; COc1cccc(NC(=O)NCCc2ccc(O)cc2)c1 ; CCS(=O)(=O)N1CCc2c(cccc2NC(=O)NCCc2ccc(O)cc2)C1 ; CC(C)(C)OC(=O)N1CCc2c(cccc2NC(=O)NCCc2ccc(O)cc2)C1]

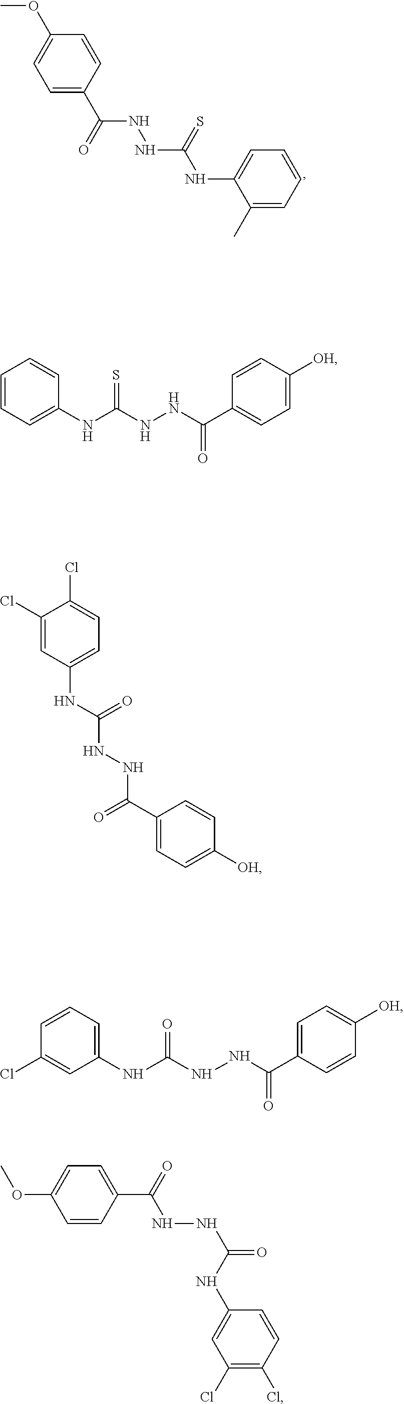 [SMILES: COc1ccc(C(=O)NNC(=S)Nc2ccccc2C)cc1 ; O=C(NNC(=S)Nc1ccccc1)c1ccc(O)cc1 ; O=C(NNC(=O)c1ccc(O)cc1)Nc1ccc(Cl)c(Cl)c1 ; O=C(NNC(=O)c1ccc(O)cc1)Nc1cccc(Cl)c1 ; COc1ccc(C(=O)NNC(=O)Nc2ccc(Cl)c(Cl)c2)cc1]

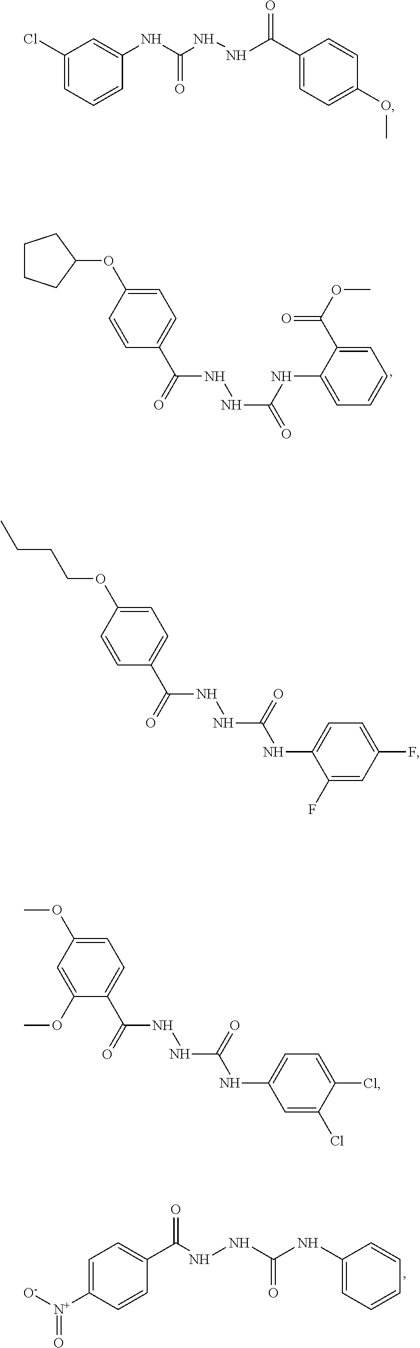

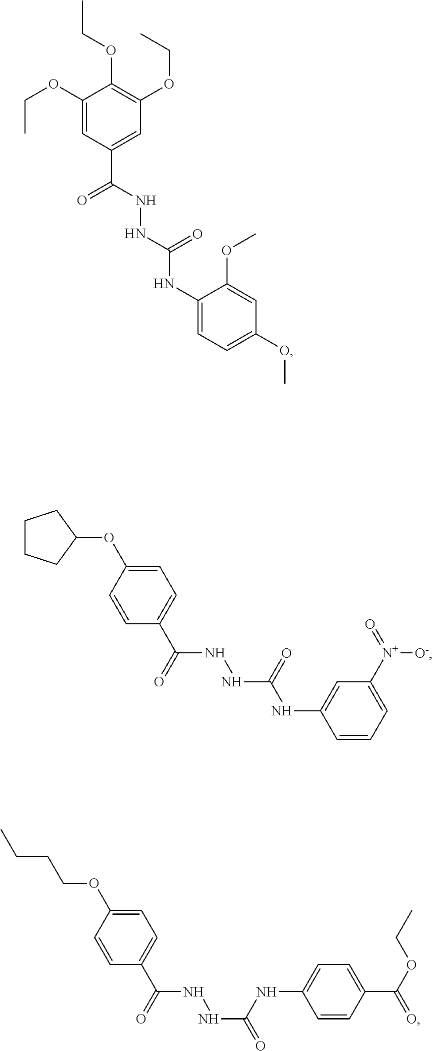

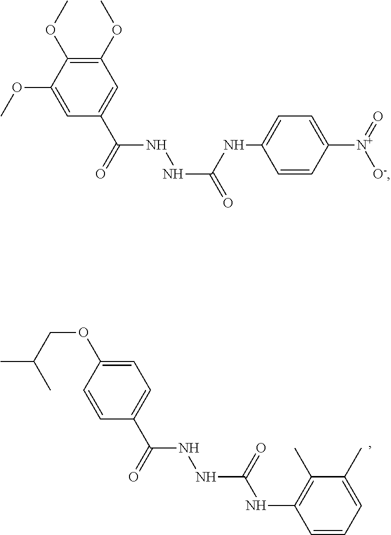

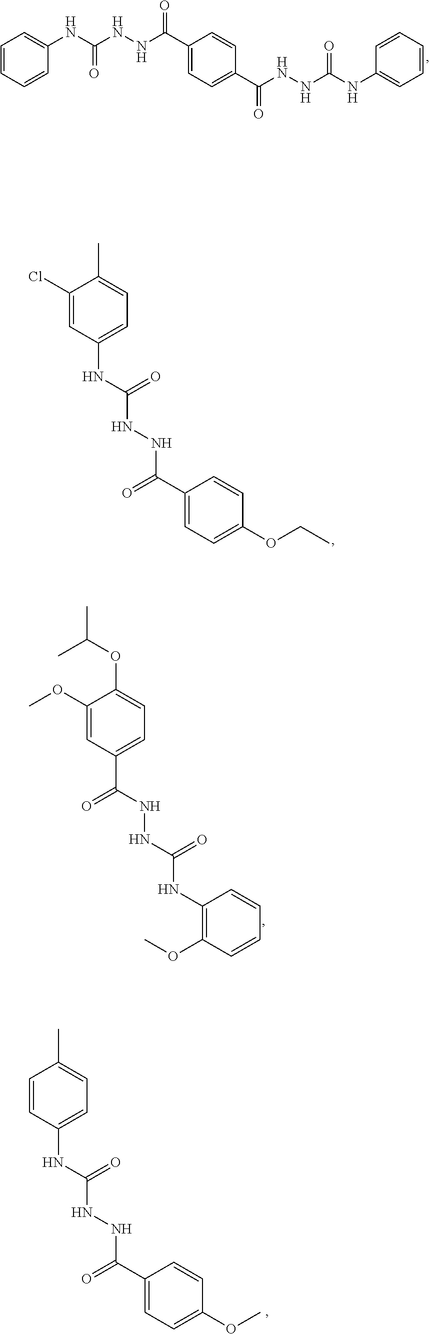

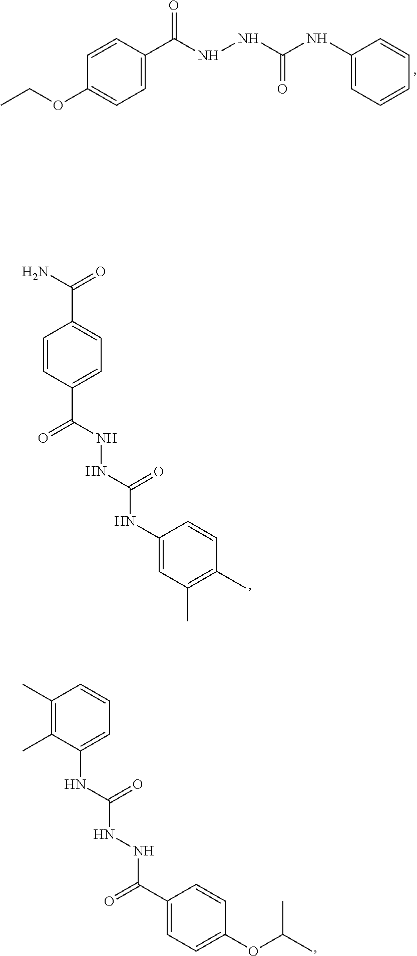 [SMILES: CCOc1ccc(C(=O)NNC(=O)Nc2ccccc2)cc1 ; Cc1ccc(NC(=O)NNC(=O)c2ccc(C(N)=O)cc2)cc1C ; Cc1cccc(NC(=O)NNC(=O)c2ccc(OC(C)C)cc2)c1C]

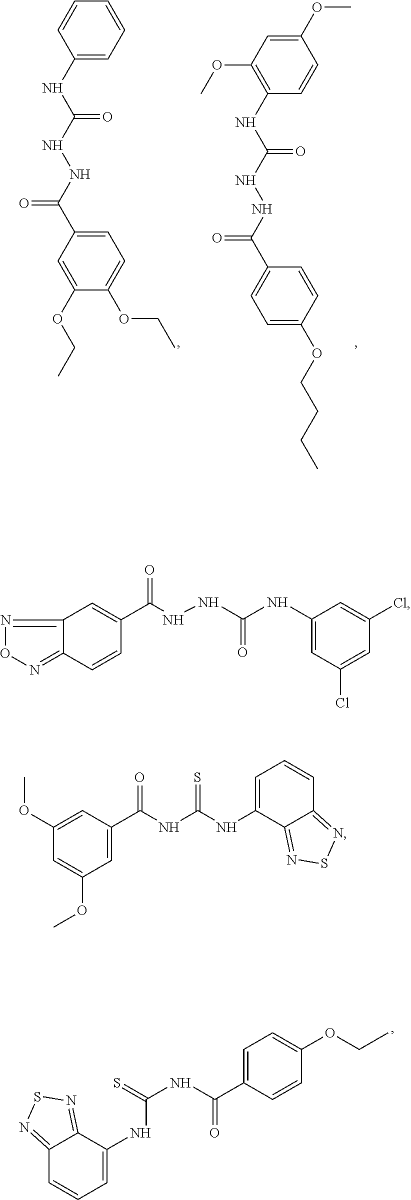

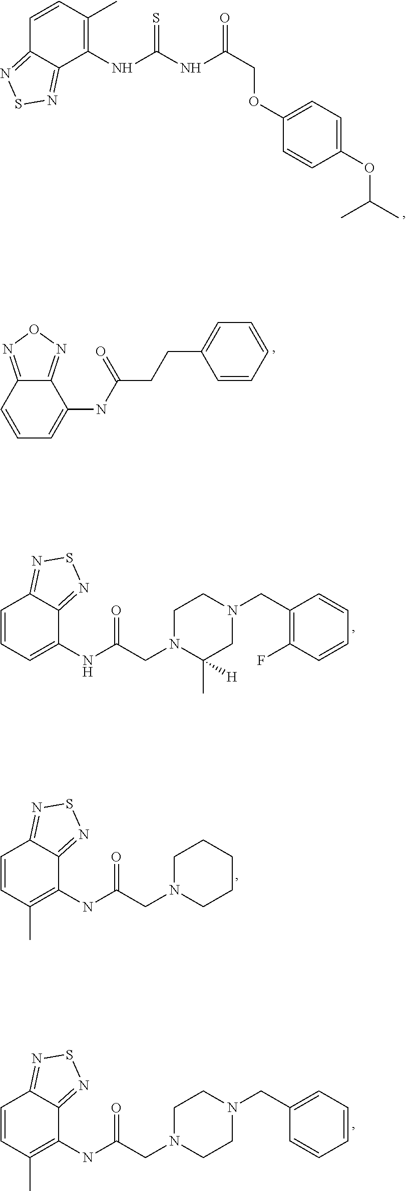 [SMILES: Cc1ccc2nsnc2c1NC(=S)NC(=O)COc1ccc(OC(C)C)cc1 ; O=C(CCc1ccccc1)Nc1cccc2nonc12 ; C[C@@H]1CN(Cc2ccccc2F)CCN1CC(=O)Nc1cccc2nsnc12 ; Cc1ccc2nsnc2c1NC(=O)CN1CCCCC1 ; Cc1ccc2nsnc2c1NC(=O)CN1CCN(Cc2ccccc2)CC1]

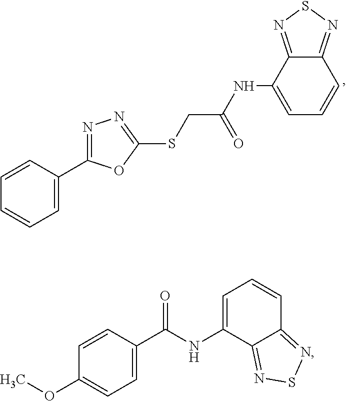 [SMILES: O=C(CSc1nnc(-c2ccccc2)o1)Nc1cccc2nsnc12 ; COc1ccc(C(=O)Nc2cccc3nsnc23)cc1]

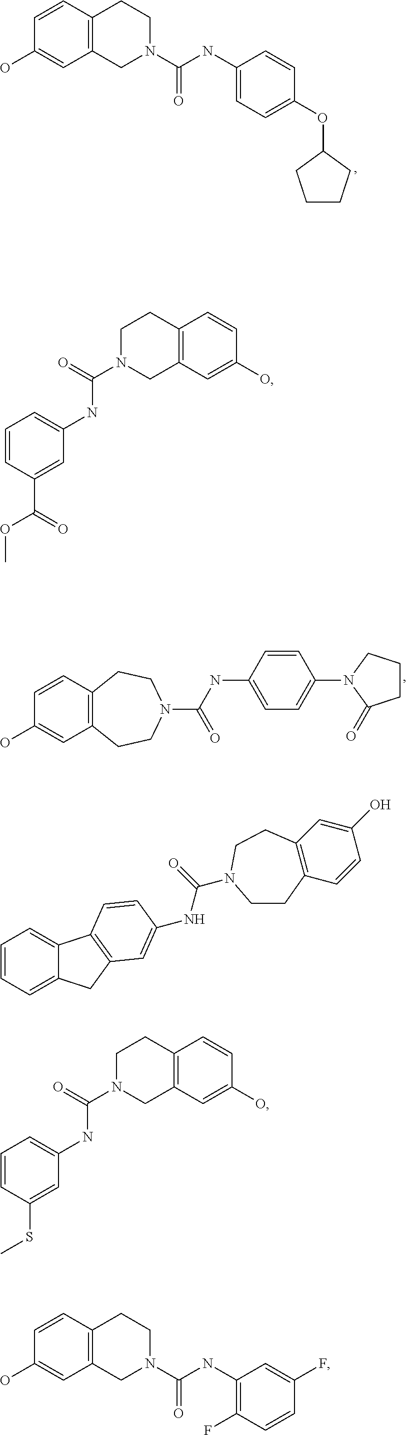

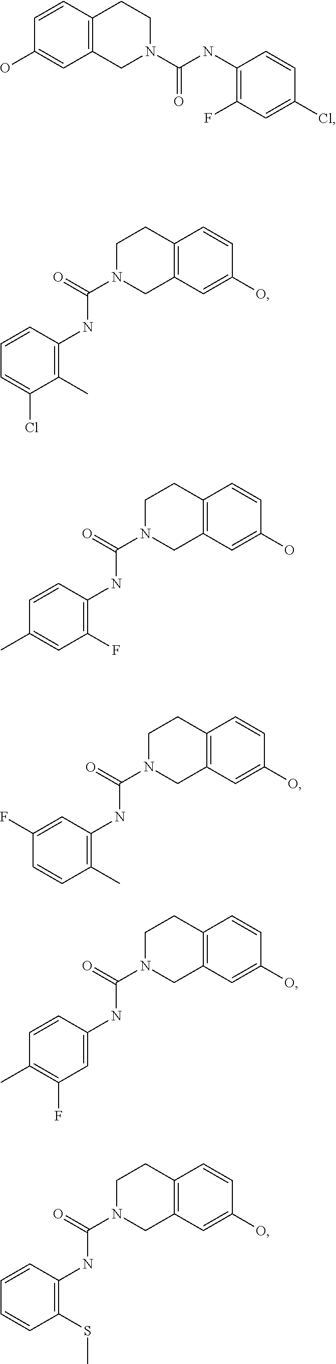

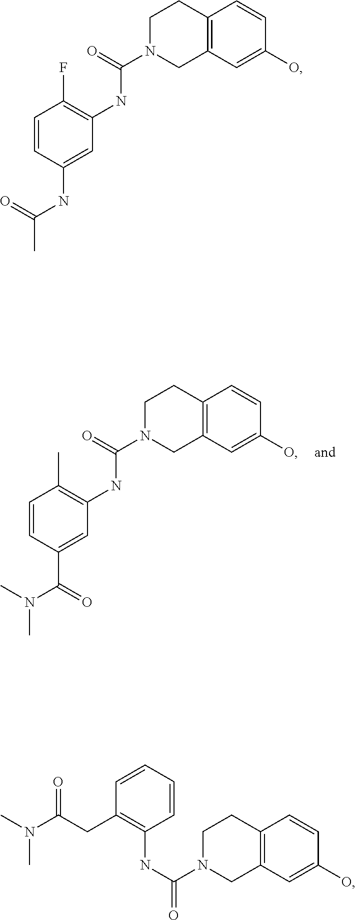

wherein any atoms with an incomplete valence are covalently bonded to one or more hydrogen atoms to complete their valence.

### BRIEF DESCRIPTION OF THE FIGURES

FIG. 1 has four panes (a-d) depicting a schematic of the screening assay and an analysis of the data from the assay. Panel (a) shows cell-cell fusion and specificity control assays. In the cell-cell fusion assay (left), Env-mediated membrane fusion enables diffusion of a tetracycline-regulated transactivator (tTA) that activates firefly luciferase (F-luc) expression in the target cells. In the specificity control assay (right) used as a counterscreen, F-luc is induced to allow measurement of any off-target effects. Panel (b) depicts that the two assays were validated with known HIV-1 entry inhibitors (Maraviroc, T20) and cytotoxic/off-target compounds. Dox, doxcycline (a tTA inhibitor), CHX, cycloheximide. Panel (c) depicts data from primary screen. The readout of each test compound was normalized to the assay readout without a compound. The effect of each compound on the cell-cell fusion assay versus its effect on the specificity control assay was plotted. A filter for inhibition (vertical dashed line) and a specificity threshold (diagonal dotted line, high ratio of normalized residual specificity to normalized residual inhibition) were applied to all compounds. Hits are identified at the top left portion of the plot. Analysis of ˜8000 out of the 212,285 screened compounds is shown. Panel (d) depicts data from secondary screen. Identified hits were retested in the cell-cell fusion and specificity assays and, in addition, were tested for any effect on target cell viability. The results are plotted as in panel c, but the size of each circle indicates the effect of each compound on target cell viability. Confirmed inhibitors that showed high selectivity and were not cytotoxic to the target cells are shown. 18A is shown.

FIG. 2 has six panels (a-f) depicting the effects of 18A on infection of R5- and X4-tropic viruses. Panel (a) shows the structure of 18A. Panel (b) shows the effect of 18A on infection of Cf2Th-CD4/CCR5 cells by R5 HIV-1. The viruses associated with the symbols in panel (b) and panel (c) are listed in panel (e). Panel (c) shows the same data as panel (b), but using CXCR4-tropic viruses and Cf2Th-CD4/CXCR4 cells. Infection of the control A-MLV was enhanced at low concentrations and steeply decreased at higher concentrations; the data could not be fit to a standard inhibition curve, but in a separate experiment, the measured CC50 of 18A for these cells was 63 μM. Panel (d) depicts specific inhibition of HIV-1JR-FL infection of human PBMC by 18A. Panel (e) tabulates the inhibitory concentrations of 18A for a large panel of viruses that includes primary, laboratory-adapted and transmitted/founder HIV-1 isolates from different phylogenetic clades (indicated in parentheses). A-MLV was used as a control. IC₅₀ values were calculated by fitting the average inhibition data from 2-5 independent experiments, most of them performed in triplicate, to a four-parameter logistic equation. Panel (f) shows the average IC₅₀ values of 18A inhibition for HIV-1 from the indicated phylogenetic clades, for all HIV-1 isolates, and for other primate immunodeficiency viruses.

FIG. 3 has five panels (a-e) depicting an investigation of the target of 18A inhibition. Panel (a) shows that chimeras between a sensitive (JR-FL) and a resistant (KB9) HIV-1 strain were tested for inhibition by 18A. Panel (b) depicts the requirement of 18A inhibition for CD4 was tested by challenging CD4/CCR5-expressing cells and CCR5-expressing cells with the CD4-independent HIV-1ADA N 197S mutant. Panel (c) depicts the dependence of 18A inhibition on complex glycans on HIV-1 Env was measured by preparing recombinant JR-FL viruses in the presence and absence of two glycosidase inhibitors and testing their sensitivity to 18A. Panel (d) shows the profile of binding of a large panel of monoclonal antibodies with known epitopes to the 18A-bound gp120 glycoprotein. Binding was normalized to the antibody binding in the absence of 18A. OD, outer domain; left bar=17 μM 18A; right bar=69 μM 18A. Panel (e) depicts an analysis of the interference of 18A with antibody binding. The monoclonal antibodies from panel (d) were grouped according to their binding site on gp120 and the effect of 18A on their binding at a concentration of 0.1 μg/mL was averaged. An ANOVA test for significant differences between the means of the groups showed P values of 0.003 and 0.015 for the 17 μM (left bar) and 69 μM (right bar) concentrations of 18A, respectively. Student's t-tests for pairwise comparison between the groups are shown on the right. P-values correspond to the 17 μM (0.003, 0.944, 0.014) and 69 μM (0.031, 0.713, 0.005) concentrations of 18A, respectively. The data shown are the means±standard errors of the means from 2-5 independent experiments, each performed with two or three replicates.

FIG. 4 has nine panels (a-i) depicting the effect of gp120 changes on HIV-1 sensitivity to 18A. Panel (a) depicts relative resistance (1154A through Y435A) or sensitivity (first four lines) of HIV-1 gp120 mutants to 18A inhibition, compared with the wild-type Env (see Table 3). J, HIV-1JR-FL, A, HIV-1ADA. Fold change, ratio of mutant to wild-type IC₅₀ values. Inhibition was calculated from data derived from 2-5 independent experiments, each performed in triplicate. Panel (b) shows amino acid residues associated with 18A resistance (M434, I424, L193, N156, Y177) or hypersensitivity (W479, I109, V430, R178) are shown on the crystal structure of the BG505 SOSIP.664 soluble gp140 (PDB 4NC0). The IKQI sequence, which is shared by the epitopes of the CD4i antibodies, is shown in cyan. V3 region is shown; V1/V2 region is shown. The Env structure was displayed using the UCSF Chimera package. Panels (c) and (d) show statistical analysis of the susceptibility of 18A-resistant (left) and 18A-sensitive (right) HIV-1 Env mutants to sCD4 inhibition (c), and cold inactivation (d) (see FIG. 12). The Mann-Whitney test was used to calculate the indicated P values; black bar, median; the boxes include the first, second and third quartiles; whiskers are extended to the interquartile range from the box; IT50, half-life on ice. Panel (e) depicts the correlation between sCD4 inhibition and cold sensitivity of 18A-resistant (squares) and 18A-sensitive (circles) mutants. Asterisks in panels (c-e) indicate wild-type HIV-1_{JR-FL} Env. Panels (f, g, and h) show the sensitivity of 18A-resistant mutants (as in panel (a)) and 18A-sensitive mutants (as in panel (a)) to neutralization by the 19b (f), 17b (g) and 2G12 (h) antibodies. Panel (i) depicts the infectivity of the recombinant virus with the HIV-1_{HXBc2} Env after preincubation on ice for the indicated times.

FIG. 5 has eight panels (a-h) depicting data showing the mechanism of 18A inhibition of HIV-1 infection. Panel (a) shows the effect of 18A on the binding of the PG9 antibody to the cell-surface HIV-1_{JR-FL} E168K+N188AΔCT Env trimer (designated WT_{KA}) in the presence or absence of sCD4, measured by two-color flow cytometry. Control, secondary antibody only. APC, allophycocyanin; FITC, fluorescein isothiocyanate. Panel (b) shows the response of PG9 binding to different doses of 18A (left graph: left bar=DMSO, second left bar=25 μM; second right bar=50 μM; right bar=100 μM) and sCD4 (right graph: left bar=DMSO, right bar=50 μM). Panel (c) shows the effect of 18A on binding of the indicated antibodies to HIV-1 Env (left bar=DMSO, second left bar=18A, second right bar=+sCD4, right bar=18A+sCD4). Panels (b) and (c) show normalized mean fluorescence intensity of binding of the indicated antibodies to cell-surface HIV-1_{JR-FL} WT_{KA} Env. Panel (d) depicts the effect of 18A on CD4-induced gp41 HR1 exposure in the cell-expressed HIV-1_{JR-FL}ΔCT Env trimer. Additional controls are shown in FIG. 14. Panels (e-h) show the mechanism of resistance to 18A. The HIV-1_{JR-FL} WT_{KA} backbone was used in all experiments. Panel (e) shows the effect of 18A on PG9 binding to WT_{KA} and 18A-resistant mutants was examined as in panel (c). Panel (f) shows the sCD4-mediated decrease of PG9 binding and the restoration of PG9 binding by 18A in the presence of sCD4 (left bar=decrease, right bar=restoration). Panel (g) depicts the effect of 18A on the sCD4-induced gp41 HR1 exposure for WT_{KA} and 18A-resistant mutants (left bar=+sCD4, right bar=18A+sCD4). Panel (h) depicts the correlation between fold resistance to 18A and the integrated ability to counteract CD4-induced V1/V2 rearrangement (V1/V2ᵣₑ=decrease−restoration of PG9 binding) and HR1 exposure (HR1frac=HR1 exposure in the presence/HR1 exposure in the absence of 18A) for the panel of 18A-resistant mutants. Data shown are representative (a, d) or average (all other panels) results from 2-4 independent experiments.

FIG. 6 has two panels (a and b) depicting schematics showing models for the inhibition of entry by 18A. Panel (a) shows the molecular mechanism of 18A inhibition. Binding to CD4 “opens” the HIV-1 Env trimer and induces V1/V2 movement and gp4 I HR1 exposure, which can be detected by a decrease in the binding of the PG9 antibody and an increase in the binding of C34-Ig, respectively (right). Interaction of 18A with the HIV-1 Env prior to CD4 engagement blocks the V1/V2 movement and gp41 HR1 exposure (left). Panel (b) shows interaction points of 18A with HIV-1 Env along the entry pathway. The postulated free energies associated with the metastable unliganded states of the wild-type and 18A-resistant Env variants are indicated. Compared with the wild-type Env, 18A-resistant mutants exhibit higher envelope reactivity and a lower activation barrier separating the unliganded states from downstream conformations. The proposed points of 18A inhibition of Env movement into the CD4-bound conformation are indicated.

FIG. 7 depicts data showing validation of the fusion assay with known entry inhibitors. The specified inhibitors were incubated with the cocultivated effector and target cells during the cell-cell fusion assay. Luminescence was read after 20 hours and the readout was normalized to that seen in the absence of compound. The results were fitted to the four-parameter logistic equation.

FIG. 8 depicts charts showing the progress of the screen. The different steps and outcomes in the screening process are shown. The 179 hits identified in the secondary screen were further tested for selective inhibition of the entry of recombinant HIV-1 into cells. Compound 18A was identified as the most selective entry inhibitor.

FIG. 9 has four panels (a-d) depicting compounds with a shared hydrazone group and associated data. Panel (a) shows the structures of compounds with a shared hydrazone group that were identified in the screen. Panel (b) depicts the effect of three compounds on the cell-cell fusion activity (left bar), specificity of inhibition (right bar in left graph) and the viability (right bar in right graph) of CEM #21 target cells in the primary and secondary screens. Panel (c) shows the activity of 18A in the secondary screen. Data in panel (b) and panel (c) represent the average and range of a duplicate measurement. All compounds were assayed at final concentration of 11 μg/ml. Panel (d) depicts dose-response inhibition by 18A of the cell-cell fusion assay using effector cells expressing the HIV-1_{AD8} (circles) or HIV-1_{JR-FL} (squares) Envs. The CC₅₀ of the CEM #21 cells was 26.6±2.1 μM and IC₅₀ of the specificity assay was 15.1±1.1 μM.

FIG. 10 has two panels (a and b) depicting reversible inhibition of HIV-1_{JR-FL} infection by 18A. Panel (a) shows that viruses were incubated with DMSO or with different concentrations of 18A at 37° C. and then pelleted by ultra centrifugation. The virions were resuspended in medium and used to infect Cf2Th-CD4/CCR5 cells. Panel (b) depicts the inhibition of HIV-1_{JR-FL} viruses with different levels of infectivity by 18A. Infectivity is measured as relative light units (RLU) produced by the luciferase reporter protein.

FIG. 11 depicts the effect of 18A on gp120 binding to CCR5 (right bar). Binding of soluble HIV-1_{JR-FL} gp120 to Cf2Th-CCR5 cells, which express human CCR5 but not CD4, was measured by flow cytometry in the absence or prescence of indicated concentrations of 18A and sCD4. All mean fluorescence values were normalized to the binding of gp120 to the Cf2Th-CCR5 cells in the absence of 18A and sCD4.

FIG. 12 depicts properties of 18A-sensitive and 18A-resistant HIV-1 mutants. The sensitivity of each mutant virus to cold and to the indicated ligands is shown. For some treatments of mutant viruses, 50% inhibition was not achieved (in these cases, the highest tested concentration of ligand or the longest incubation time on ice is marked as 200 μG/ml (IC₅₀ 17b), 12 μG/ml (IC₅₀ 19b), >100 hours (IT₅₀ cold)). IT₅₀, half life on ice.

FIG. 13 has two panels (a and b) depicting the relationship between 18A resistance and envelope reactivity. Resistance to 18A is associated with increased envelope reactivity due to localized effects. Panel (a) shows two pairs of matched viruses, in which one (J1HX pair) or three (J3-197 pair) amino acid residue changes are associated with significant alteration of envelop reactivity, were tested for sensitivity to 18A. Similar inhibition of these Env mutants by 18A demonstrates that not all Env changes that increase Env reactivity result in resistance to 18A. Panel (b) depicts a schematic representation of the relationship between 18A resistance and envelope reactivity. Specific changes in the β20-β21 and the V1/V2 region (circle) cause resistance to 18A and also increase envelope reactivity. Increased Env reactivity does not necessarily lead to 18A resistance. The L179G mutant exhibited intermediate levels of Env reactivity and 18A resistance.

FIG. 14 has four panels (a-d) depicting the effect of 18A on the binding of different ligands to cells expressing HIV-1_{JR-FL} Env variants Panel (a) shows the effect of 100 μg/mL sCD4 on PG9 binding to cells expressing the indicated HIV-1 Envs. 293T cells (left) and HOS cells (right) were tested using flow cytometry and cell-based ELISA, respectively. JR-FL_{KA}, HIV-1_{JR-FL} E168K+N188A; FL, full-length, ΔCT; cytoplasmic tail deleted (left bar=PG9 (no sCD4), right bar=sCD4+PG9). Panel (b) depicts the effect of the order of 18A and sCD4 addition on the CD4-mediated decrease of PG9 binding to HIV-1_{JR-FL} WT_{KA} (JR-FL_{KA} ΔCT) Env. Panel (c) shows the effect on CD4-induced HR1 exposure after washout of 18A. Cells expressing HIV-1_{JR-FL} WT were treated with DMSO or 18A and then sCD4. 18A was washed out and exposure of gp41 HR1 was detected with C34-Ig using flow cytometry. Binding of sCD4 was detected with an anti-CD4 antibody. Percentage of positive cells are shown in each quadrant.

### DETAILED DESCRIPTION OF THE INVENTION

### Overview

Binding to the primary receptor, CD4, triggers conformational changes in the metastable envelope glycoprotein (Env) tamer (gp1203/gp413) of human immunodeficiency virus (HIV-1) that are important for virus entry into host cells. These changes include an “opening” of the trimer, creation of a binding site for the CCR5 coreceptor, and formation/exposure of a gp41 coiled coil. In certain embodiments, the invention relates to compounds that specifically inhibit the entry of a wide range of HIV-1 isolates. In certain embodiments, the compounds of the invention do not interfere with CD4 or CCR5 binding, but inhibit the CD4-induced disruption of quaternary structures at the trimer apex and the formation/exposure of the gp41HR1 coiled coil. Analysis of HIV-1 variants exhibiting increased sensitivity or resistance to an inhibitor, such as 18A, suggests that the inhibitor can distinguish distinct conformational states of gp120 in the unliganded Env trimer.

In certain embodiment, the invention relates to small molecule compounds that exhibit broad inhibitory activity against diverse HIV-1 strains by blocking the function of Env. The HIV-1 Env trimer is a membrane-fusing molecular machine with high potential free energy; in certain embodiments, the compounds of the invention inhibit CD4-triggered conformational changes in this machine that are critical for membrane fusion and virus entry. One change involves the rearrangement of the gp120 V1/V2 region, which is located in the trimer association domain at the trimer apex. The CD4-induced “opening” of the HIV-1 Env trimer results gp120 movement/rotation away from the trimer axis. During this process, the V1/V2 region relocates to near domain 1 of the bound CD4 molecule, while the V3 region projects towards the target cell to interact with the coreceptor. In certain embodiments the compounds of the invention specifically interfere with the relocation of the V1/V2 regions, which make important contributions to the PG9 epitope, without any apparent effect on the. CD4-induced movement of the V3 region. A second CD4-induced change that is inhibited by various compounds of the invention, the formation/exposure of the gp41 HR1 coiled coil, is also blocked by BMS-806. Despite this similarity between the compounds of the invention and BMS-806, several features distinguish these compounds: i) compounds of the invention have much broader inhibitory activity, inhibiting infection of some HIV-2 and SIV isolates; ii) compounds of the invention weakly stabilize the unliganded state of HIV-1 Env; and compounds of the invention inhibit two different rearrangements involved in the transition to the CD4-bound conformation. So, in certain embodiments, the invention relates to new, dual-effect blockers that exhibit both potency and breadth.

In certain embodiments, the invention relates to compounds, such as 18A, that inhibit a wide spectrum of HIV-1 strains. The breadth of inhibition suggests that the compounds of the invention interact with a conserved site on HIV-1 Env. In all current models of the HIV-1 Env trimer, the gp120 β20-β21 strands, which critically contribute to the epitopes of all CD4i antibodies, are adjacent to the trimer apex, where the V1/V2 regions reside. While not wishing to be bound by any particular theory, a binding site in this locality could explain the observed ability of compounds such as 18A to impede the CD4-induced down-regulation of the PG9 epitope, which involves movement of the V1/V2 region. Given that the critical site of 18A interaction must be well-conserved in HIV-1 strains and that an HIV-1 mutant with a deletion of the V1/V2 region remains susceptible to 18A inhibition, a conformation-dependent gp120 target near the β20-β21 strands is consistent with the available data. According to this model, interaction of 18A with this site restrains the CD4-induced movement of the V1/V2 region.

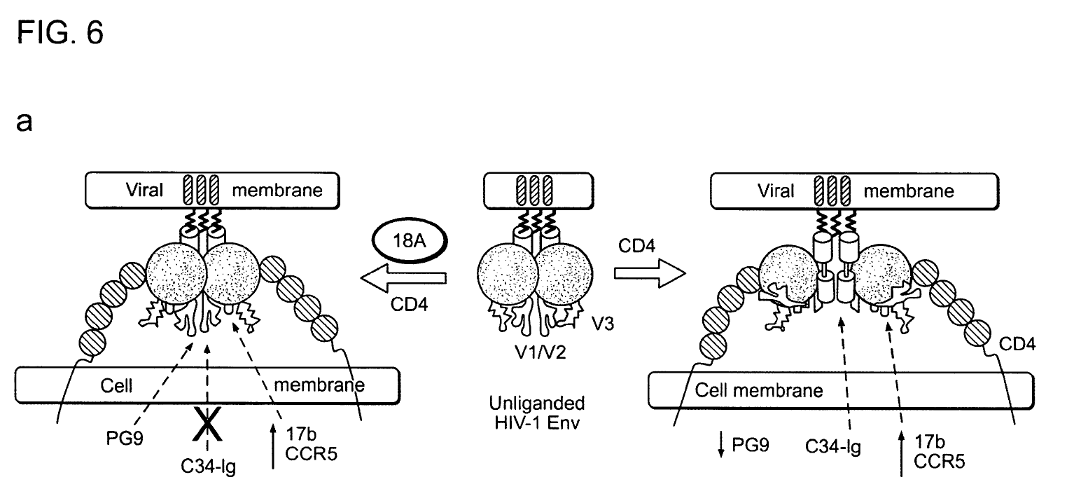

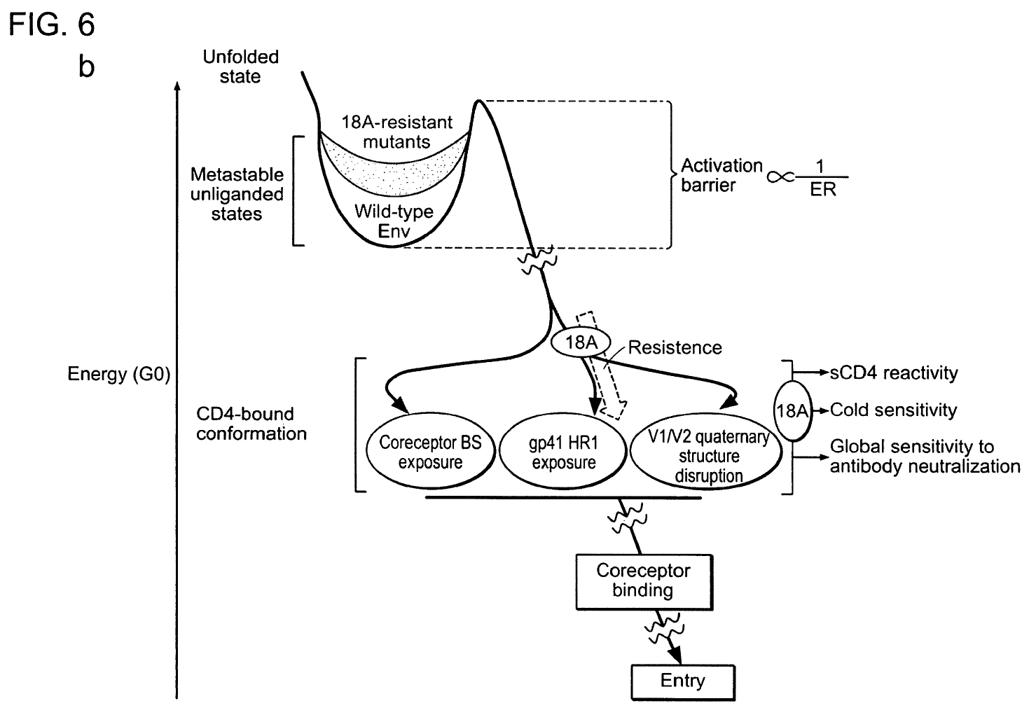

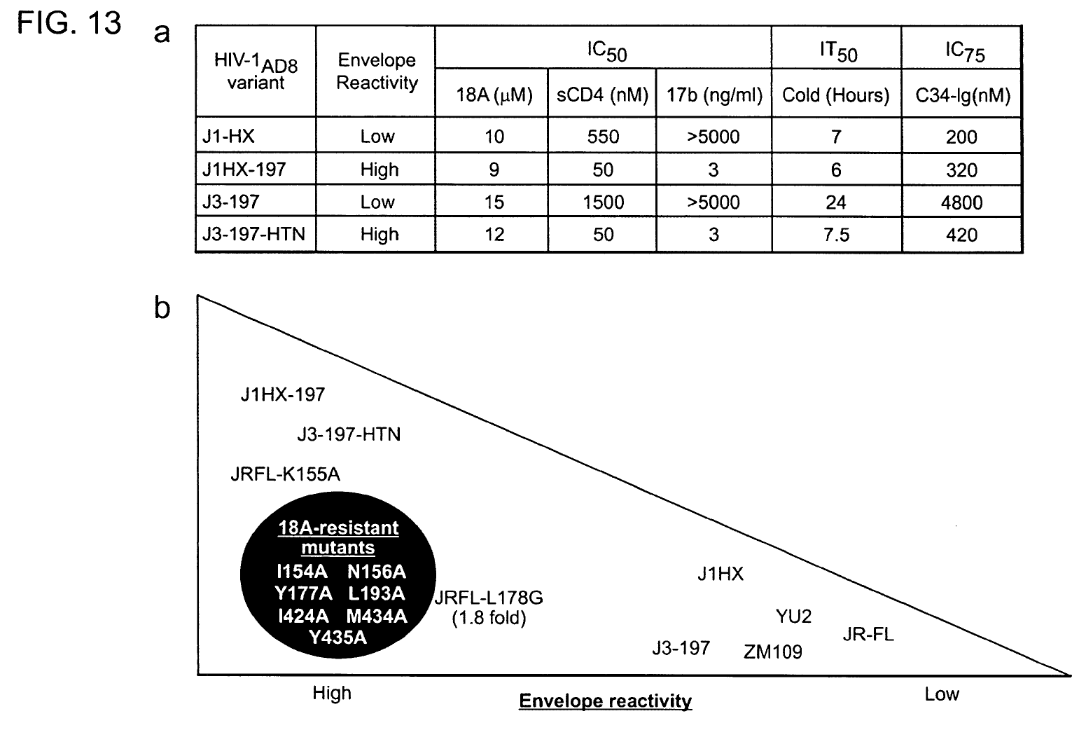

The study of resistant Env mutants revealed potential pathways to remove the block imposed by compounds such as 18A on CD4-induced V1/V2 movement and gp41 HR1 exposure. For some mutants, such as L179G, resistance appears to rely primarily on the exposure of the gp41 HR1 region in the presence of 18A. Other resistant mutants demonstrated enhanced CD4-triggered movement of the V1/V2 region with low 18A-mediated restoration of the PG9 epitope, relative to that of the wild-type Env. One mutant, M434A, combined high resistance to the 18A-mediated blockade of both V1/V2 movement and gp41 HR1 exposure. Interestingly, 18A resistance was usually accompanied by increased envelope reactivity. As Env reactivity is inversely related to the activation barriers that maintain the unliganded state of Env, the alterations that confer resistance to 18A likely involve changes in Env conformation. However, increased Env reactivity is not sufficient for decreased sensitivity to 18A (FIG. 13), suggesting that 18A interacts with regions that are specifically sensitive to alterations in the conformation of the unliganded Env trimer. Indeed, the phenotypes of the mutant Env panel suggest that multiple gp120 conformational states are able to be accommodated within functional Env trimers (FIG. 6).

In summary, the compounds and methods of the invention represent a valuable new probe to investigate different conformational states of HIV-1 Env and to define their importance to HIV-1 entry into cells. In certain embodiments, 18A inhibition demonstrated a wide coverage of diverse HIV-1 strains, and resistance was accompanied by high envelope reactivity. Enhanced sensitivity of 18A-resistant mutants to neutralization by antibodies that do not neutralize wild-type HIV-1 represents a beneficial aspect of 18A. These types of antibodies are commonly elicited during natural HIV-1 infection and may synergize with 18A to limit pathways of HIV-1 escape. The attractive attributes of 18A and related compounds make them good candidates for further development.

Definitions

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The definition of each expression, e.g., alkyl, m, n, and the like, when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., a compound which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein below. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The term “lower” when appended to any of the groups listed below indicates that the group contains less than seven carbons (i.e. six carbons or less). For example “lower alkyl” refers to an alkyl group containing 1-6 carbons, and “lower alkenyl” refers to an alkenyl group containing 2-6 carbons.

The term “saturated,” as used herein, pertains to compounds and/or groups which do not have any carbon-carbon double bonds or carbon-carbon triple bonds.

The term “unsaturated,” as used herein, pertains to compounds and/or groups which have at least one carbon-carbon double bond or carbon-carbon triple bond.

The term “aliphatic,” as used herein, pertains to compounds and/or groups which are linear or branched, but not cyclic (also known as “acyclic” or “open-chain” groups).

The term “cyclic,” as used herein, pertains to compounds and/or groups which have one ring, or two or more rings (e.g., spiro, fused, bridged).

The term “aromatic” refers to a planar or polycyclic structure characterized by a cyclically conjugated molecular moiety containing 4n+2 electrons, wherein n is the absolute value of an integer. Aromatic molecules containing fused, or joined, rings also are referred to as bicyclic aromatic rings. For example, bicyclic aromatic rings containing heteroatoms in a hydrocarbon ring structure are referred to as bicyclic heteroaryl rings.

The term “hydrocarbon” as used herein refers to an organic compound consisting entirely of hydrogen and carbon.

For purposes of this invention, the chemical elements are identified in accordance with the Periodic Table of the Elements, CAS version, Handbook of Chemistry and Physics, 67th Ed., 1986-87 inside cover.

The term “heteroatom” as used herein is art-recognized and refers to an atom of any element other than carbon or hydrogen. Illustrative heteroatoms include boron, nitrogen, oxygen, phosphorus, sulfur and selenium.

The term “alkyl” means an aliphatic or cyclic hydrocarbon radical containing from 1 to 12 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 2-methylcyclopentyl, and 1-cyclohexylethyl.

The term “substituted alkyl” means an aliphatic or cyclic hydrocarbon radical containing from 1 to 12 carbon atoms, substituted with 1, 2, 3, 4, or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, haloalkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfinyl, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, haloalkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy. haloalkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl and silyloxy.

The term “carbocyclyl” as used herein means monocyclic or multicyclic bicyclic, tricyclic, etc.) hydrocarbons containing from 3 to 12 carbon atoms that is completely saturated or has one or more unsaturated bonds, and for the avoidance of doubt, the degree of unsaturation does not result in an aromatic ring system (e.g. phenyl). Examples of carbocyclyl groups include 1-cyclopropyl, 1-cyclobutyl, 2-cyclopentyl, 1-cyclopentenyl, 3-cyclohexyl, 1-cyclohexenyl and 2-cyclopentenylmethyl.

The term “heterocyclyl”, as used herein include non-aromatic, ring systems, including, but not limited to, monocyclic, bicyclic (e.g. fused and spirocyclic) and tricyclic rings, which can be completely saturated or which can contain one or more units of unsaturation, for the avoidance of doubt, the degree of unsaturation does not result in an aromatic ring system, and have 3 to 12 atoms including at least one heteroatom, such as nitrogen, oxygen, or sulfur. For purposes of exemplification, which should not be construed as limiting the scope of this invention, the following are examples of heterocyclic rings: azepines, azetidinyl, morpholinyl, oxopiperidinyl, oxopyrrolidinyl, piperazinyl, piperidinyl, pyrrolidinyl, quinicludinyl, thiomorpholinyl, tetrahydropyranyl and tetrahydroluranyl. The heterocyclyl groups of the invention are substituted with 0, 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, haloalkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenysulfinyl, alkynylsulfinyl, alkoxysulfinyl, haloalkoxysulfinyl, fluoroalkoxysulfinyl, alkenyloxysulfinyl, alkynyloxysulfinyl, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, haloalkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, haloalkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the heterocyclyl group through an alkylene moiety (e.g. methylene).

The term “N-heterocyclyl” as used herein is a subset of heterocyclyl, as defined herein, which have at least one nitrogen atom through which the N-heterocyclyl moiety is bound to the parent moiety. Representative examples include pyrrolidin-1-yl, piperidin-1-yl, piperazin-1-yl, hexahydropyrimidin-1-yl, morpholin-1-yl, 1,3-oxazinan-3-yl and 6-azaspiro[2.5]oct-6-yl. As with the heterocyclyl groups, the N-heterocyclyl groups of the invention are substituted with 0, 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, haloalkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfinyl, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, haloalkoxysulfonyloxy, fluroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, haloalkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the N-heterocyclyl group through an alkylene moiety (e.g. methylene).

The term “aryl,” as used herein means a phenyl group, naphthyl anthracenyl group. The aryl groups of the present invention can be optionally substituted with 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, haloalkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfinyl, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, haloalkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, haloalkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the heterocyclyl group through an alkylene moiety (e.g. methylene).

The term “arylene,” is art-recognized, and as used herein pertains to a bidentate moiety obtained by removing two hydrogen atoms of an aryl ring, as defined above.

The term “arylalkyl” or “aralkyl” as used herein means an aryl group, as defined herein, appended to the parent molecular moiety through an alkyl group, as defined herein, Representative examples of aralkyl include, but are not limited to, benzyl, 2-phenylethyl, 3-phenylpropyl, and 2-naphth-2-ylethyl.

The term “heteroaryl” as used herein include aromatic ring systems including, hut not limited to, monocyclic, bicyclic and tricyclic rings, and have 3 to 12 atoms including at least one heteroatom, such as nitrogen, oxygen, or sulfur. For purposes of exemplification, which should not be construed as limiting the scope of this invention: azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrmidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl. The heteroaryl groups of the invention are substituted with 0, 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, haloalkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfiny, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, haloalkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, haloalkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the heteroaryl group through an alkylene moiety (e.g. methylene).

The term “heteroarylene,” is art-recognized, and as used herein pertains to a bidentate moiety obtained by removing two hydrogen atoms of a heteroaryl ring, as defined above.

The term “heteroarylalkyl” or “heteroaralkyl” as used herein means a heteroaryl, as defined herein, appended to the parent molecular moiety through an alkyl group, as defined herein. Representative examples of heteroarylalkyl include, but are not limited to, pyridin-3-ylmethyl and 2-(thien-2-yl)ethyl.

The term “halo” or “halogen” means —Cl, —Br, —I or —F.

The term “haloalkyl” means an alkyl group, as defined herein, wherein at least one hydrogen is replaced with a halogen, as defined herein. Representative examples of haloalkyl include, but are not limited to, chloromethyl, 2-fluoroethyl, trifluoromethyl, pentafluoroethyl, and 2-chloro-3-fluoropentyl.

The term “fluoroalkyl” means an alkyl group, as defined herein, wherein all the hydrogens are replaced with fluorines.

The term “hydroxy” as used herein means an —OH group.

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy. The terms “alkenyloxy”, “alkynyloxy”, “carbocyclyloxy”, and “heterocyclyloxy” are likewise defined.

The term “haloalkoxy” as used herein means an alkoxy group, as defined herein, wherein at least one hydrogen is replaced with a halogen, as defined herein. Representative examples of haloalkoxy include, but are not limited to, chloromethoxy, 2-fluoroethoxy, trifluoromethoxy, and pentafluoroethoxy. The term “fluoroalkyloxy” is likewise defined.

The term “aryloxy” as used herein means an aryl group, as defined herein, appended to the parent molecular moiety through an oxygen. The term “heteroaryloxy” as used herein means a heteroaryl group, as defined herein, appended to the parent molecular moiety through an oxygen. The terms “heteroaryloxy” is likewise defined.

The term “arylalkoxy” or “arylalkyloxy” as used herein means an arylalkyl group, as defined herein, appended to the parent molecular moiety through an oxygen. The term “heteroarylalkoxy” is likewise defined. Representative examples of aryloxy and heteroarylalkoxy include, but: are not limited to, 2-chlorophenylmethoxy, 3-trifluoromethyl-phenylethoxy, and 2,3-dimethylpyridinylmethoxy.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The term “oxy” refers to a —O— group,

The term “carbonyl” as used herein means a —C(═O)— group.

The term “formyl” as used herein means a —C(═O)H group,

The term “alkylcarbonyl” as used herein means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl. The terms “haloalkylcarbonyl”, “fluoroalkylcarbonyl”, “alkenylcarbonyl”, “alkynylcarbonyl”, “carbocyclylcarbonyl”, “heterocyclylcarbonyl”, “arylcarbonyl”, “aralkylcarbonyl”, “heteroarylcarbonyl”, and “heteroalkylcarbonyl” are likewise defined.

The term “carboxy” as used herein means a —CO₂H group.

The term, “alkoxycarbonyl” as used herein means an alkoxy group, as defined herein, appended to the parent molecular moiety through a carbonyl group, as defined herein. Representative examples of alkoxycarbonyl include, but are not limited to, methoxycarbonyl, ethoxycarbonyl and tert-butoxycarbonyl. The terms “haloalkoxycarbonyl”, “fluoroalkoxycarbonyl”, “alkenyloxycarbonyl”, “alkynyloxycarbonyl”, “carbocyclyloxycarbonyl”, “heterocyclyloxycarbonyl”, “aryloxycarbonyl”, “aralkyloxycarbonyl”, “heteroaryloxycarbonyl”, and “heteroaralkyloxycarbonyl” are likewise defined.

The term “alkylcarbonyloxy” as used herein means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. The terms “haloalkylcarbonyloxy”, “fluoroalkylcarbonyloxy”, “alkenylcarbonyloxy”, “alkynylcarbonyloxy”, “carbocyclylcarbonyloxy”, “heterocyclylcarbonyloxy”, “arylcarbonyloxy”, “aralkylcarbonyloxy”, “heteroarylcarbonyloxy”, and “heteroaralkylcarbonyloxy” are likewise defined.

The term “amino” as used herein refers to —NH₂ and substituted derivatives thereof wherein one or both of the hydrogens are independently replaced with substituents selected from the group consisting of alkyl, haloalkyl, fluoroalkyl, alkenyl, alkynyl, carbocyclyl, heterocyclyl, aryl, aralkyl, heteroaryl, heteroaralkyl, alkylcarbonyl, haloalkylcarbonyl, fluoroalkylcarbonyl, alkenylcarbonyl, alkynylcarbonyl, carbocyclylcarbonyl, heterocyclylcarbonyl, arylcarbonyl, aralkylcarbonyl, heteroarylcarbonyl, heteroaralkylcarbonyl and the sulfonyl and sulfinyl groups defined above; or when both hydrogens together are replaced with an alkylene group (to form a ring which contains the nitrogen). Representative examples include, but are not limited to methylamino, acetylamino, and dimethylamino.

The term “amido” as used herein means an amino group, as defined herein, appended to the parent molecular moiety through a carbonyl.

The term “cyano” as used herein means a —C≡N group.

The term “nitro” as used herein means a —NO₂ group.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

As used herein, the term “administering” means providing a pharmaceutical agent or composition to a subject, and includes, but is not limited to, administering by a medical professional and self-administering.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides and (22) other non-toxic compatible substances employed in pharmaceutical formulations.

As used herein, the phrase “pharmaceutically-acceptable salts” refers to the relatively non-toxic, inorganic and organic salts of compounds.

As used herein, the term “subject” means a human or non-human animal selected for treatment or therapy.

As used herein, the phrase “subject suspected of having” means a subject exhibiting one or more clinical indicators of a disease or condition.

As used herein, the phrase “therapeutic effect” refers to a local or systemic effect in animals, particularly mammals, and more particularly humans caused by an agent. The phrases “therapeutically-effective amount” and “effective amount” mean the amount of an agent that produces some desired effect in at least a sub-population of cells. A therapeutically effective amount includes an amount of an agent that produces some desired local or systemic effect at a reasonable benefit/risk ratio applicable to any treatment. For example, certain agents used in the methods of the present invention may be administered in a sufficient amount to produce a reasonable benefit/risk ratio applicable to such treatment.

As used herein, the term “treating” disease in a subject or “treating” a subject having or suspected of having to disease refers to subjecting the subject to a pharmaceutical treatment, e.g., the administration of an agent, such that at least one symptom of the disease is decreased or prevented from worsening.

As used herein, “HIV” refers to any virus that can infect a host cell of a subject through activation of the gp120 envelope glycoproteins (Env gps). “HIV” encompasses all strains of HIV-1 and HIV-2. Compounds of the present invention, however, are also useful to treat other immunodeficiency viruses expressing gp120 such as some strains of simian immunodeficiency virus SIV.

As used herein “gp120” refers to the gp120 envelope glycoprotein, and “Env gps” refers to the complete envelope glycoprotein complex which is a trimer of three gp120s and three gp41s.

As used herein, the term “activating” when referring to gp120 envelope glycoprotein means the association of a natural or non-natural ligand with the conserved domain of gp 120 that induces a conformational change that activates binding to the chemokine receptors CCR5 or CXCR4. Examples of natural ligands include CD4 and sCD4. Examples of non-natural ligands include NBD-556 and NBD-557.

As used herein, the term “contacting” when used in the context of compounds of the present invention and gp120, refers to the process of supplying compounds of the present invention to the HIV envelope glycoprotein either in vitro or in vivo in order to effect the selective binding of the compounds of the present invention to gp120. For the in vitro process, this can entail simply adding an amount of a stock solution of one or more compounds of the present invention to a solution preparation of gp120. For an in vivo process, “selective binding” involves making compounds of the present invention available to interact with gp120 in a host organism, wherein the compounds of the invention exhibit a selectivity for a conserved element of gp120. Making the compounds available to interact with gp120 in the host organism can be achieved by oral administration, intravenously, peritoneally, mucosally, intramuscularly, and other methods familiar to one of ordinary skill in the art.

As used herein, the term “inhibiting” when referring to transmission means reducing the rate of or blocking the process that allows fusion of the viral membrane to a host cell and introduction of the viral core into the host cell. In this regard, inhibiting transmission includes prophylactic measures to prevent viral spread from one host organism to another. When referring to progression, “inhibiting” refers to the treatment of an already infected organism and preventing further viral invasion within the same organism by blocking the process that allows fusion of the viral membrane and introduction of viral core into additional host cells of the organism.

As used herein, the term “inhibitor,” when referring to a protein, enzyme, or group of proteins, refers to compounds lowering or abolishing the activity of the protein or enzyme. For example, an inhibitor of gp120 lowers the activing of gp120, said activity being defined herein in detail. Briefly, the activity of gp120 in the context of the present invention means the capability of gp120 to bind to its receptor, i.e. the CD4-receptor or alpha4 beta7, on the surface of the target cell and thereby initiate viral entry. Methods to determine said activity of gp120 are well-known in the art. In certain embodiments, inhibition effected by an inhibitor in accordance with the invention refers to a reduction in activity of at least (for each value) 10, 20, 30, 40, 50, 60, 70, 80, 90, 95, 98, or 99%. In certain embodiments, an inhibitor reduces the activity to less than 10⁻², less than 10⁻³, less than 10⁻⁴ or less than 10⁻⁵ times as compared to the activity in the absence of the inhibitor.

### Exemplary Compounds

In certain embodiments, the invention relates to a compound of Formula I

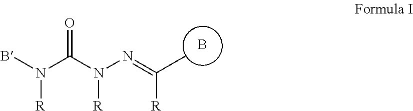

or a pharmaceutically acceptable salt or solvate thereof,
wherein, independently for each occurrence,

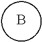

is optionally substituted aryl, optionally substituted heteroaryl, optionally substituted alkenyl, or optionally substituted cycloalkenyl;

R is hydrogen or alkyl;

B′ is optionally substituted aryl, optionally substituted heteroaryl, optionally substituted cycloalkyl, or B′, when taken together with either instance of —NR—, forms a substituted or unsubstituted heterocycloalkyl ring,

provided the compound is not

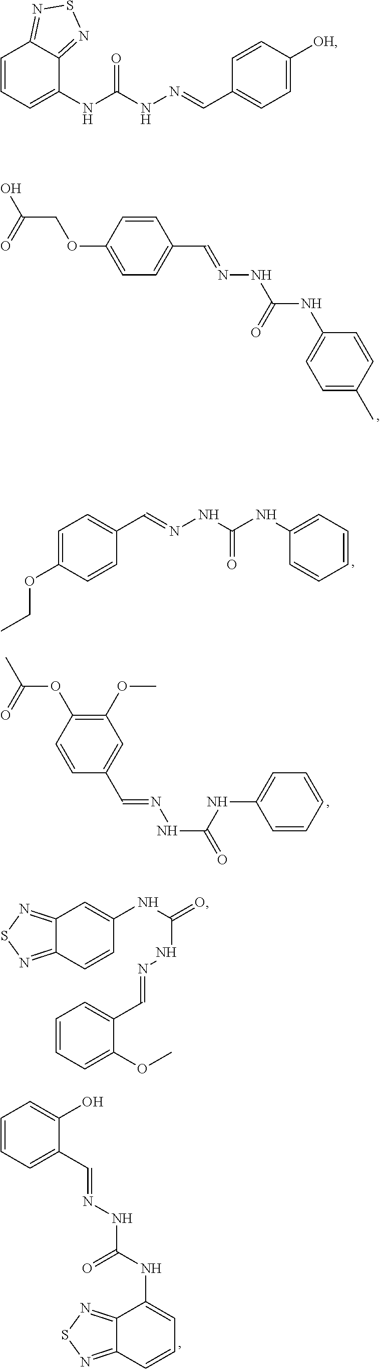

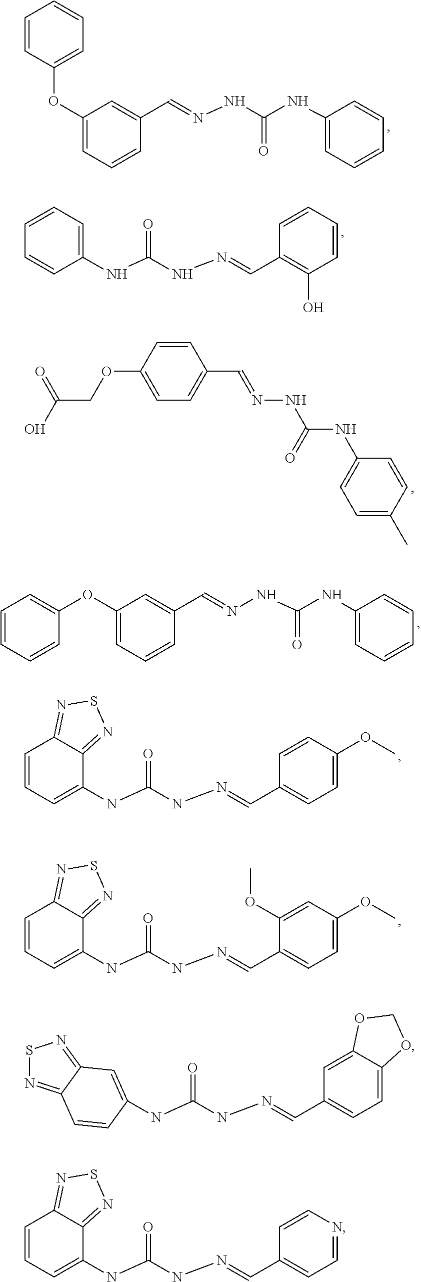

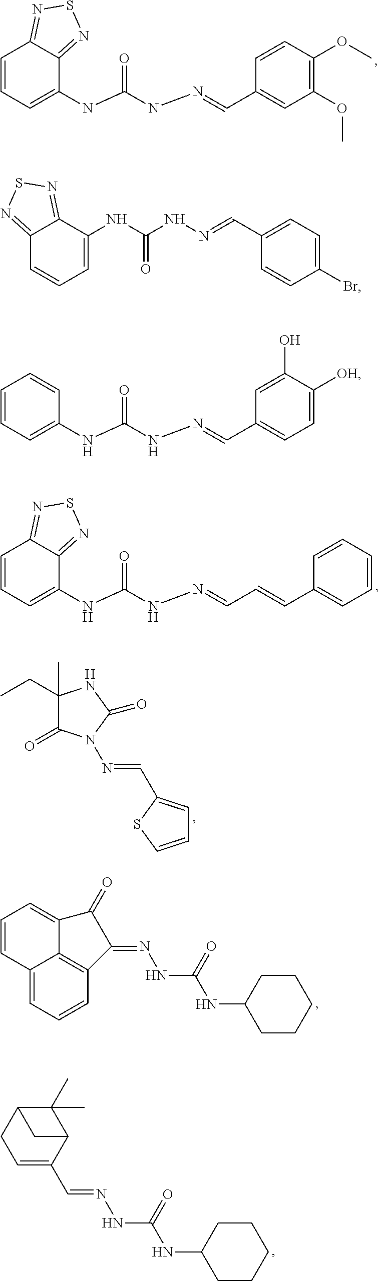

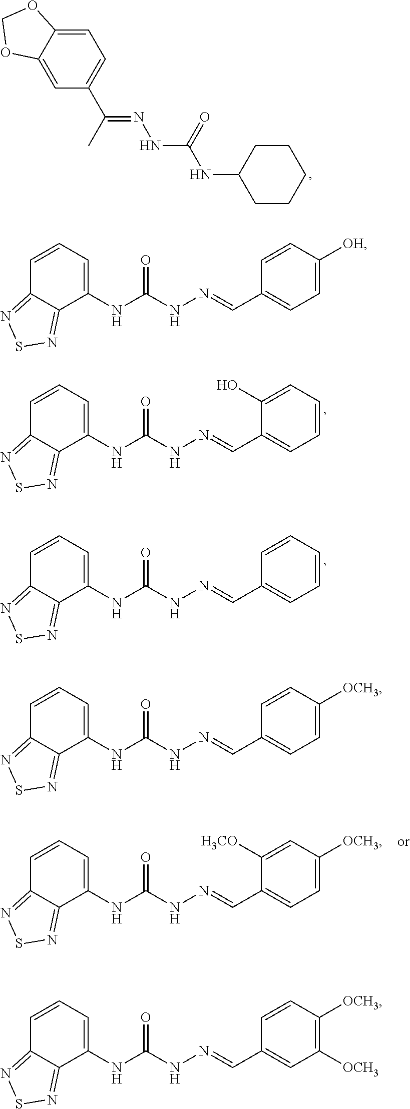

wherein any atoms with an incomplete valence are covalently bonded to one or more hydrogen atoms to complete their valence.

In certain embodiments, the invention relates to any of the compounds described herein, wherein B′ is

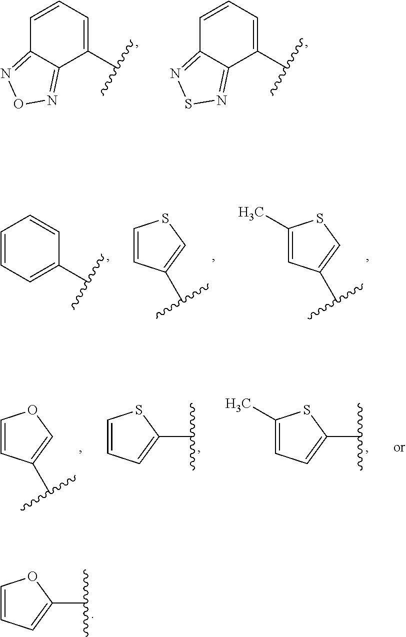

In certain embodiments, the invention relates to any of the compounds described herein, wherein

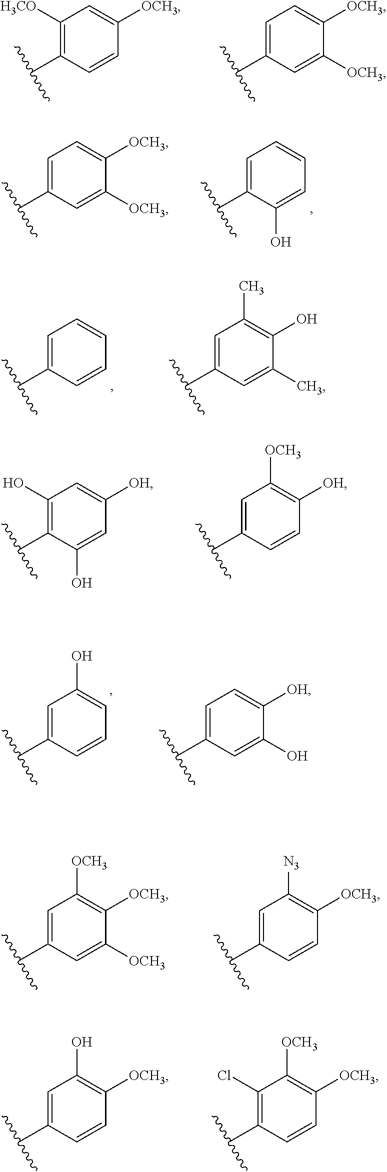

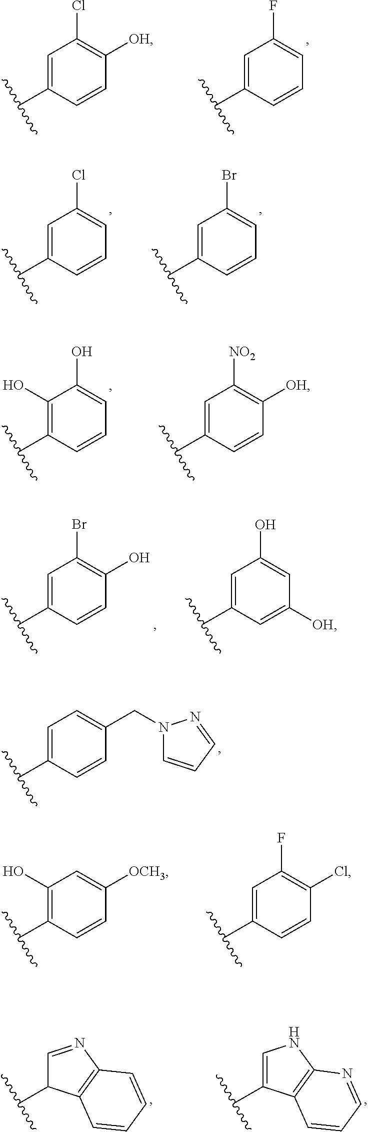

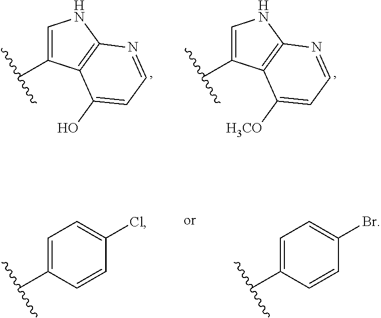 [SMILES: *c1c[nH]c2nccc(O)c12 ; *c1c[nH]c2nccc(OC)c12 ; *c1ccc(Cl)cc1 ; "or" ; *c1ccc(Br)cc1]

In certain embodiments, the invention relates to any of the compounds described herein, wherein

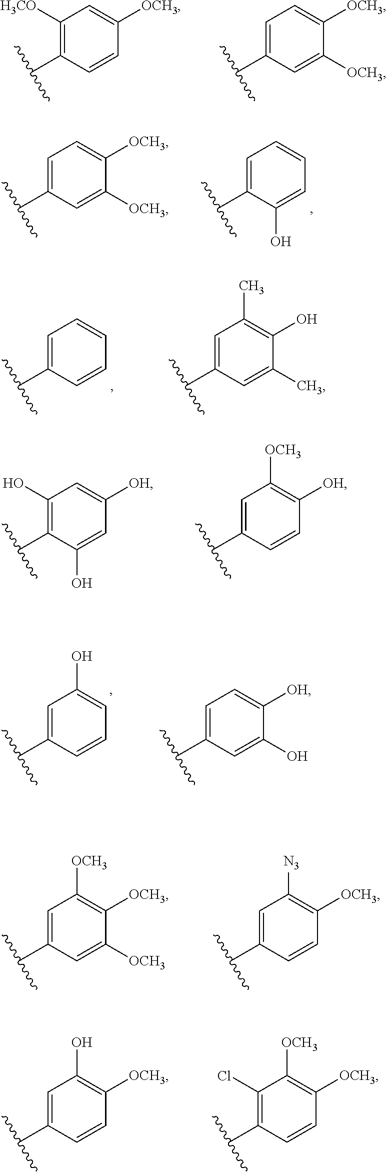

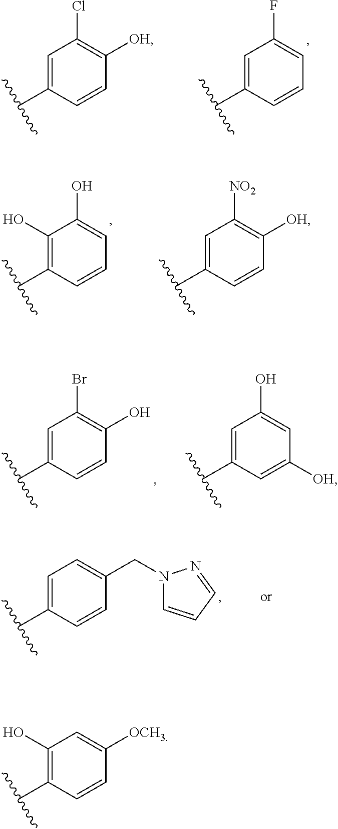

In certain embodiments, the invention relates to any of the compounds described herein, wherein the compound is

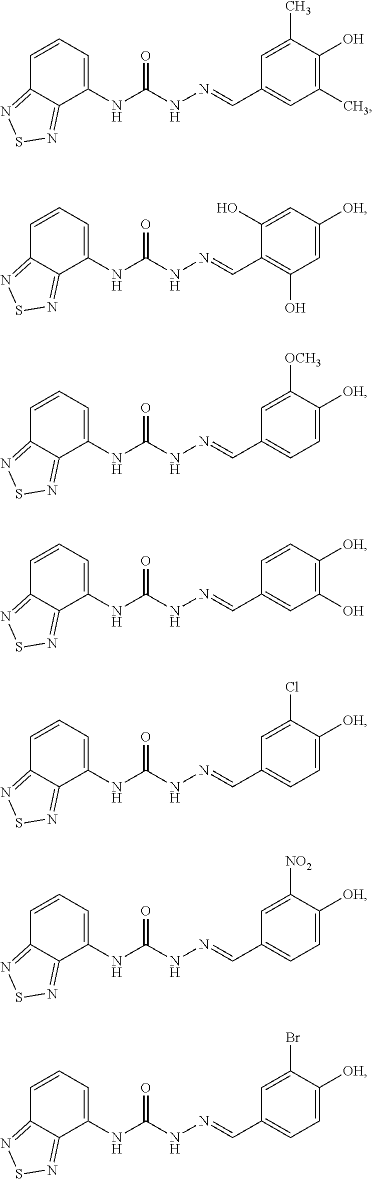

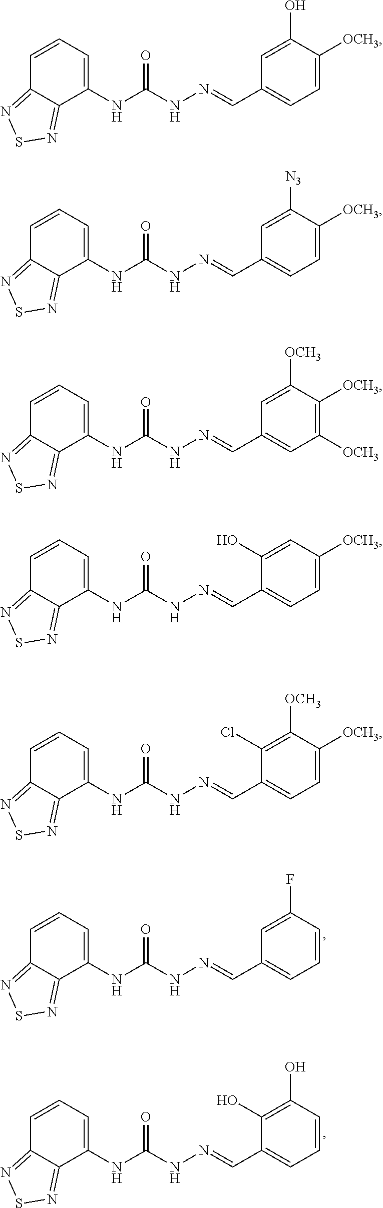

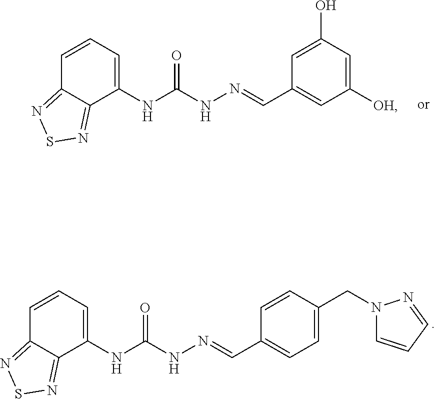 [SMILES: O=C(N/N=C/c1cc(O)cc(O)c1)Nc1cccc2nsnc12 ; "or" ; O=C(N/N=C/c1ccc(Cn2cccn2)cc1)Nc1cccc2nsnc12]

In certain embodiments, the invention relates to a compound of Formula II

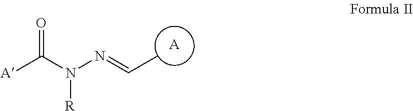

or a pharmaceutically acceptable salt or solvate thereof,
wherein, independently for each occurrence,

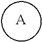

is optionally substituted aryl or optionally substituted heteroaryl; and

A′ is optionally substituted alkyl, optionally substituted cycloalkyl, optionally substituted aryl, or optionally substituted heteroaryl,

provided the compound is not

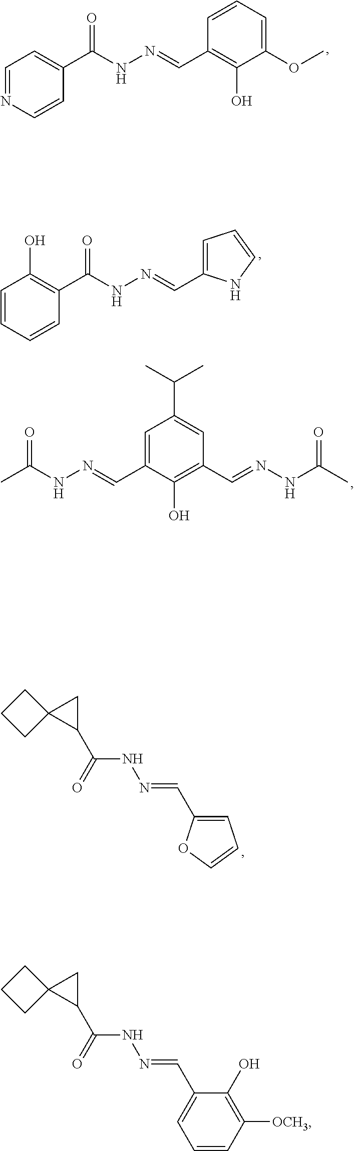

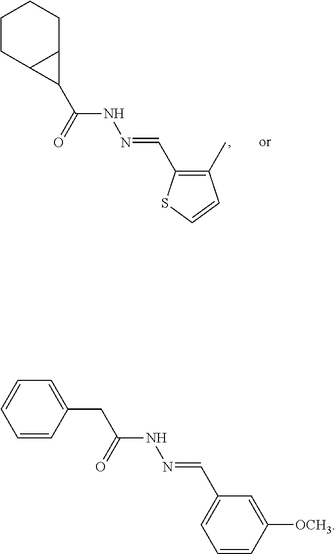 [SMILES: Cc1ccsc1/C=N/NC(=O)C1C2CCCCC21 ; COc1cccc(/C=N/NC(=O)Cc2ccccc2)c1]

In certain embodiments, the invention relate to any of the compounds described herein, wherein

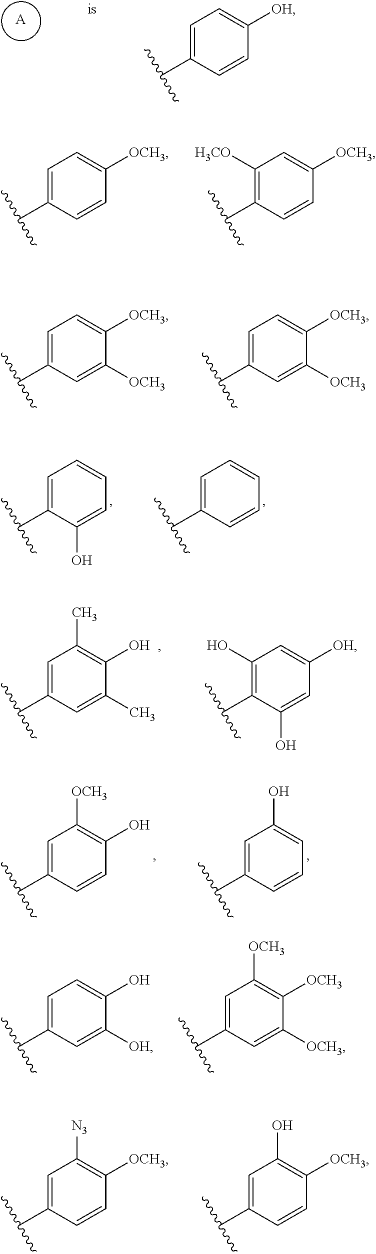

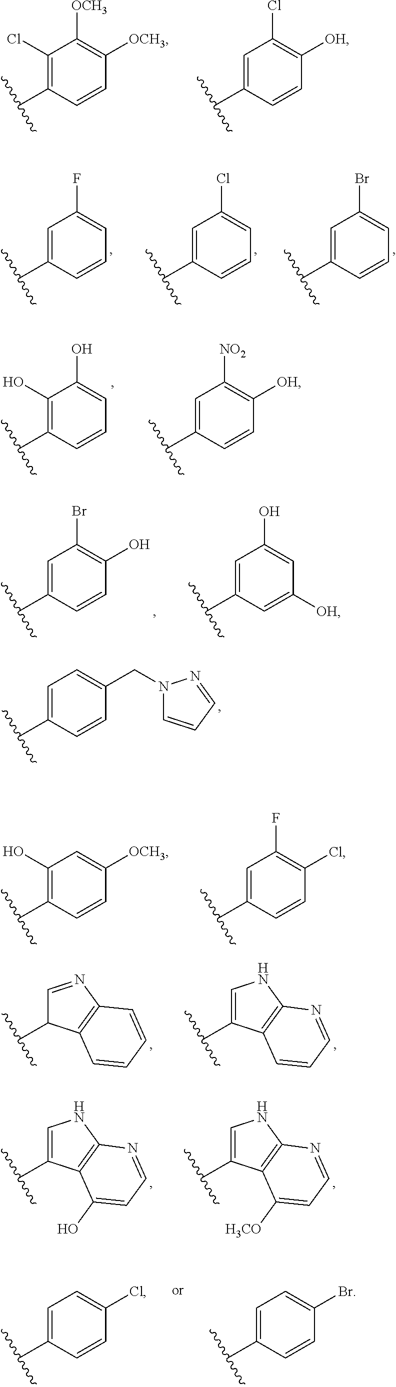

In certain embodiments, the invention relates to any of the compounds described herein, wherein A′ is

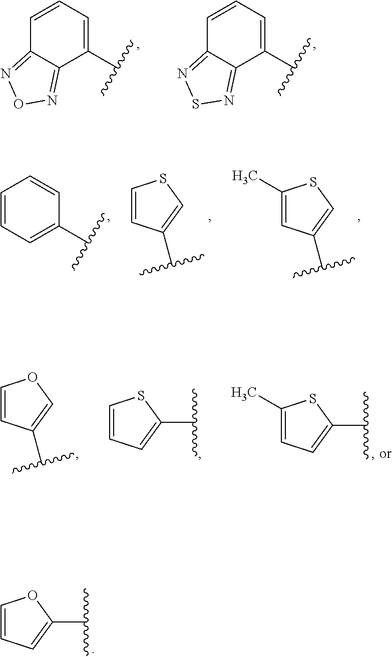

In certain embodiments, the invention relates to a compound of Formula III

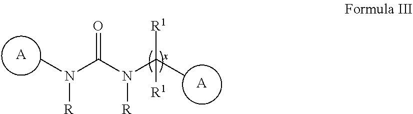

or a pharmaceutically acceptable salt or solvate thereof,
wherein, independently for each occurrence,

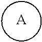

is optionally substituted aryl or optionally substituted heteroaryl;

R is hydrogen or alkyl;

R¹ is hydrogen, hydroxy, alkoxy, or alkyl; and

x is 0, 1, 2, or 3,

provided the compound is not

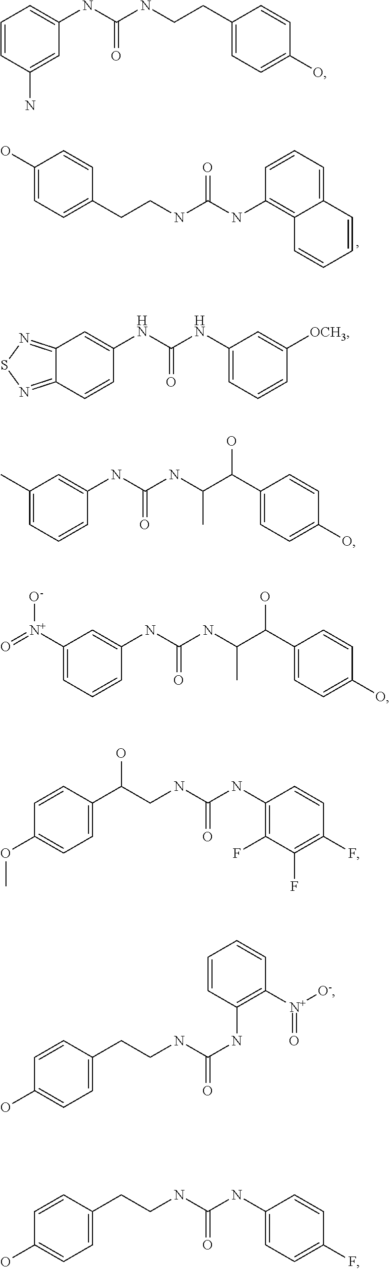

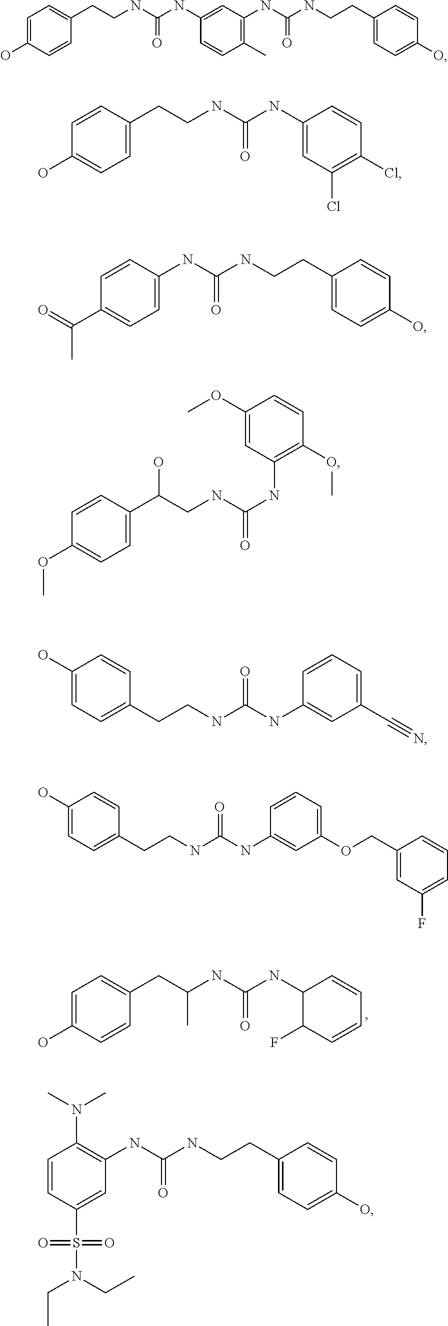

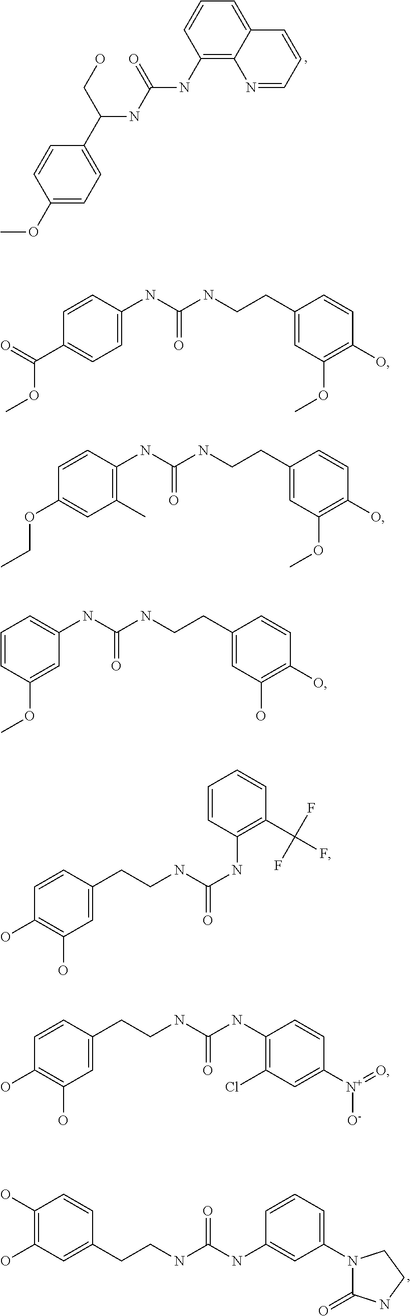

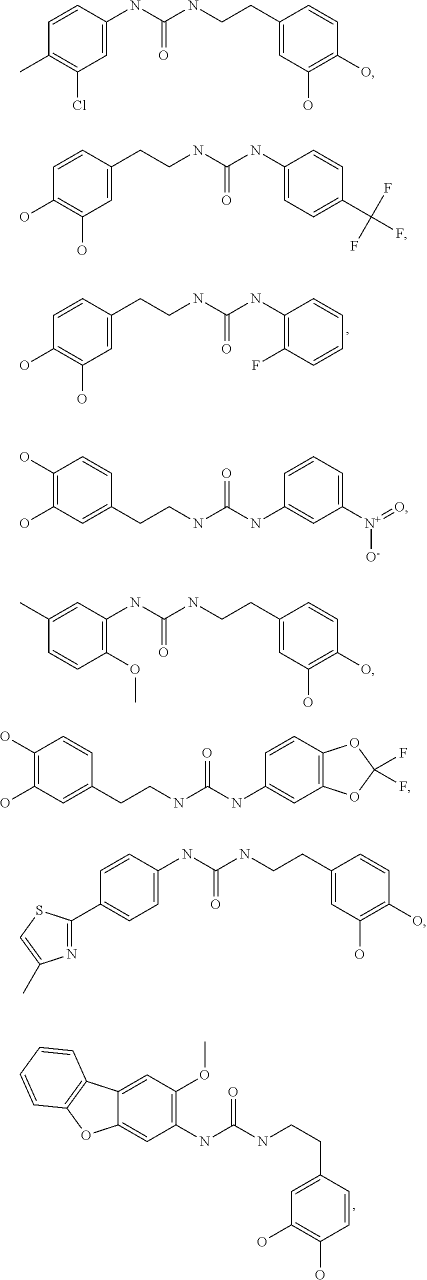

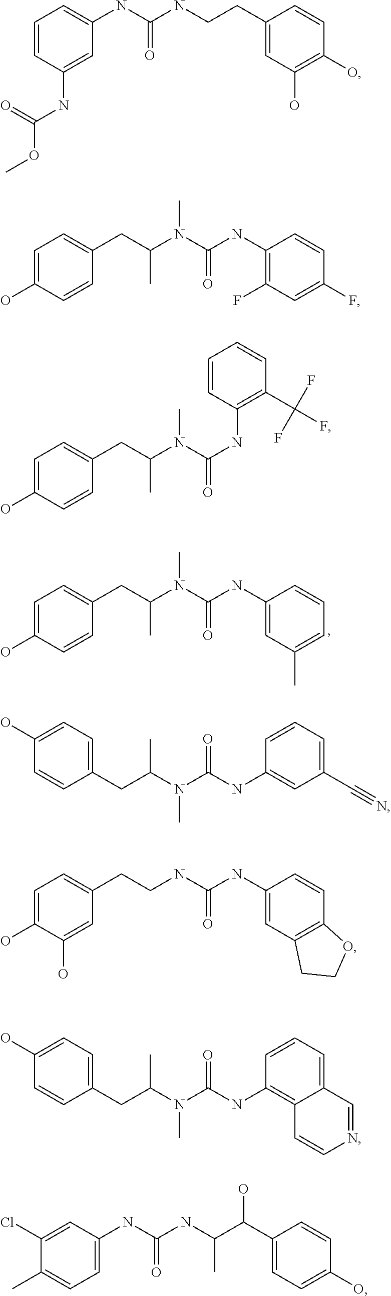

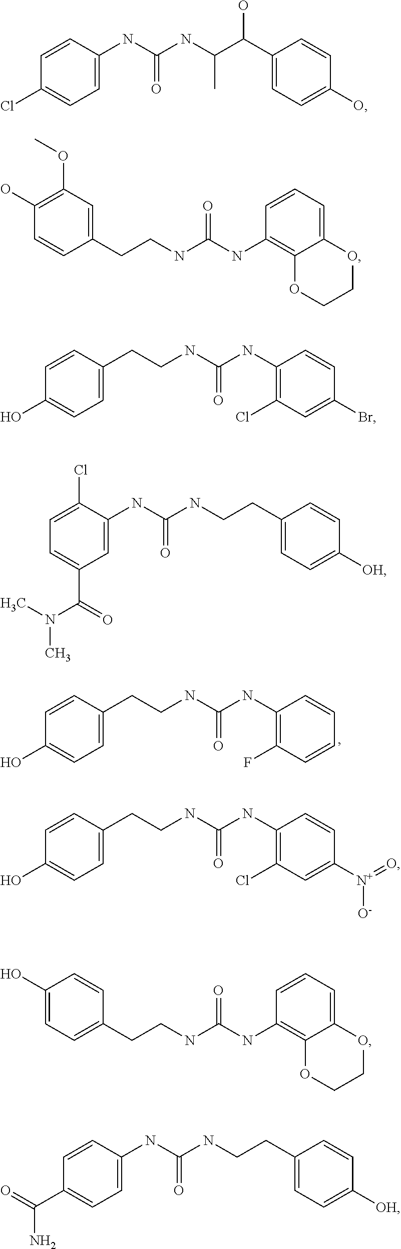

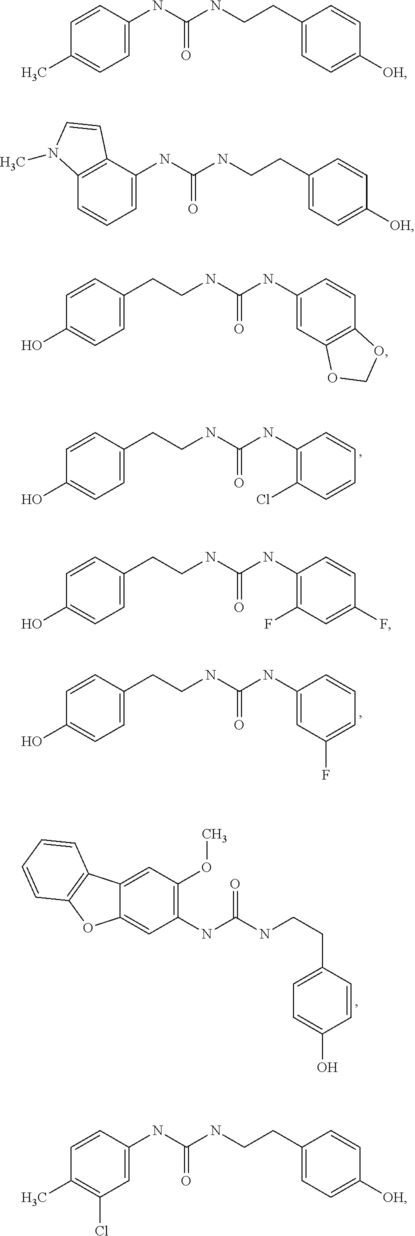

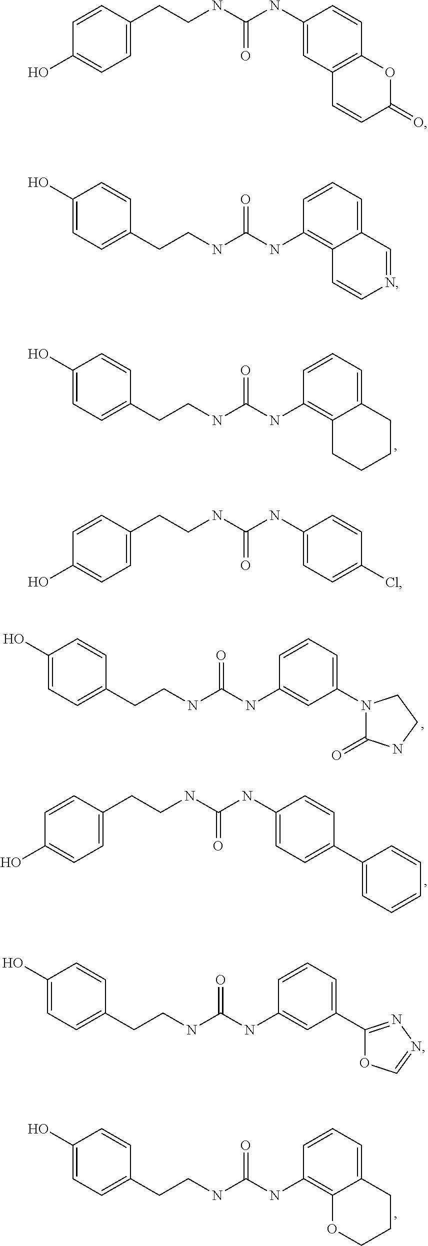

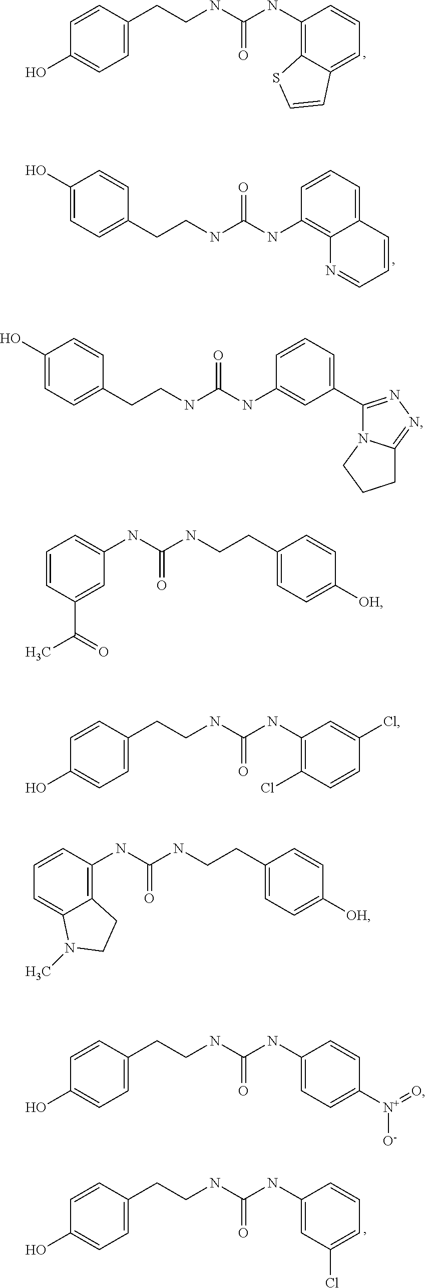

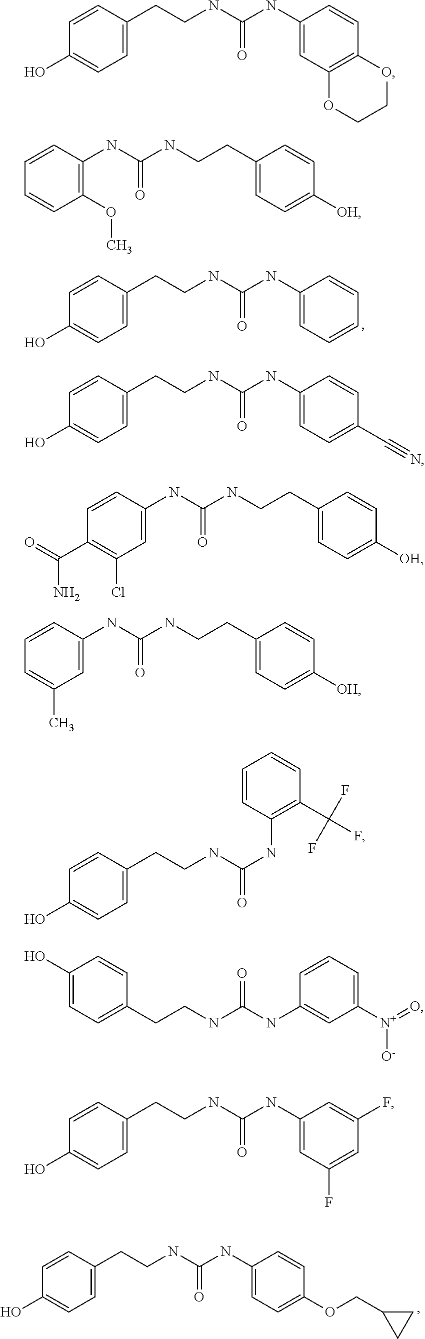

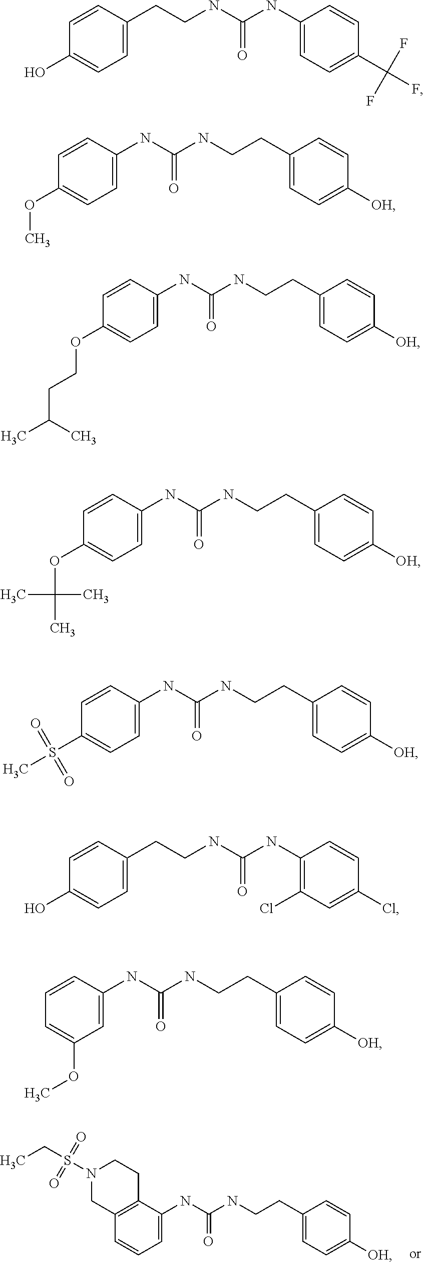

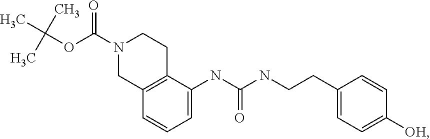 [SMILES: CC(C)(C)OC(=O)N1CCc2c(cccc2NC(=O)NCCc2ccc(O)cc2)C1]

wherein any atoms with an incomplete valence art covalently bonded to one or more hydrogen atoms to complete their valence.

In certain embodiments, the invention relates to any of the compounds described herein, wherein

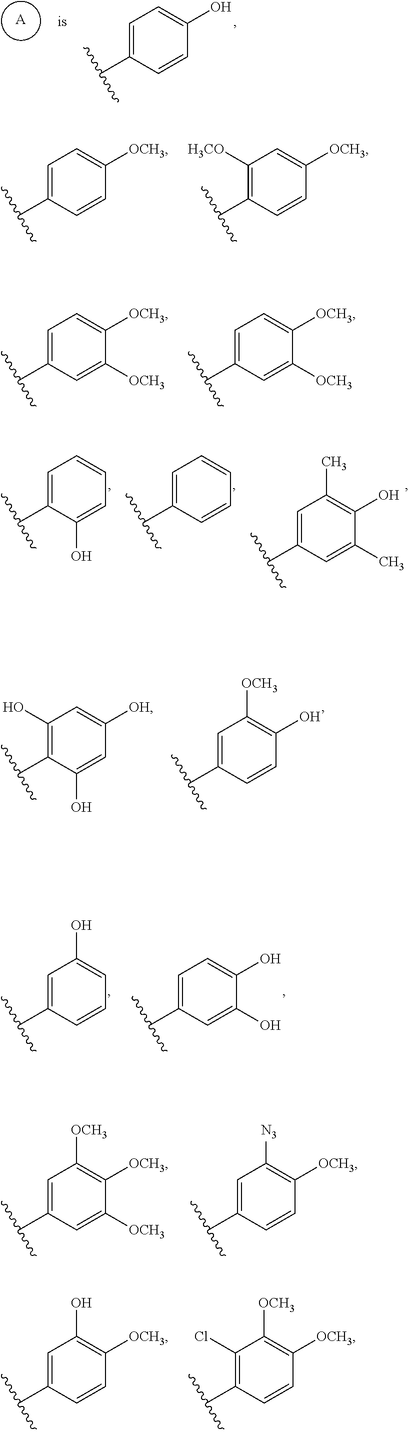

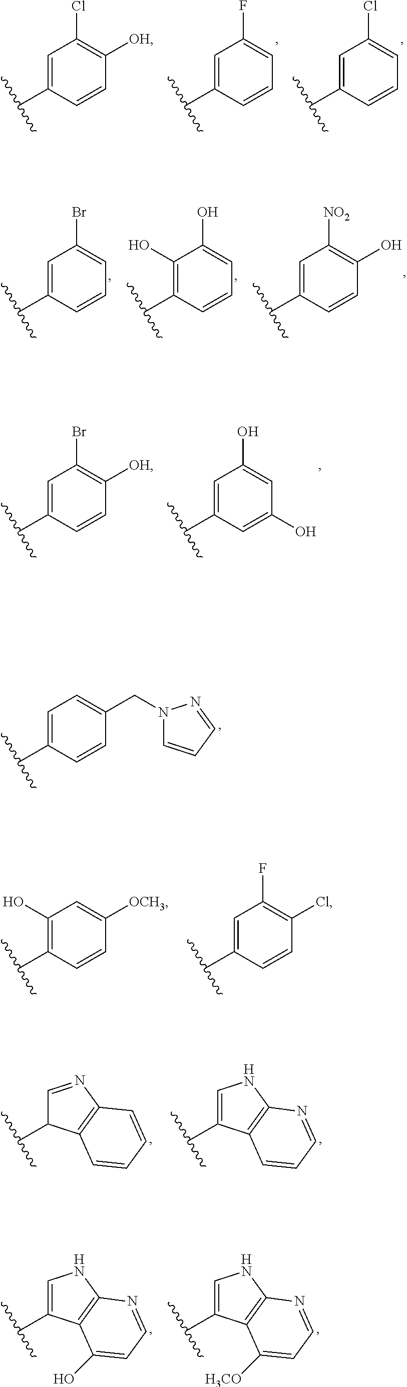

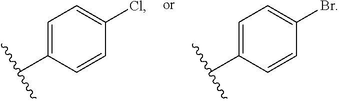 [SMILES: *c1ccc(Cl)cc1 ; "or" ; *c1ccc(Br)cc1]

In certain embodiments, the invention relates to a compound of Formula IV

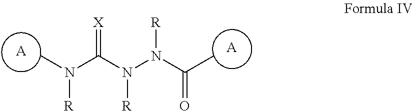

or a pharmaceutically acceptable salt or solvate thereof,
wherein, independently for each occurrence,

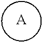

is optionally substituted aryl or optionally substituted heteroaryl;

R is hydrogen or alkyl; and

X is O or S,

provided the compound is not

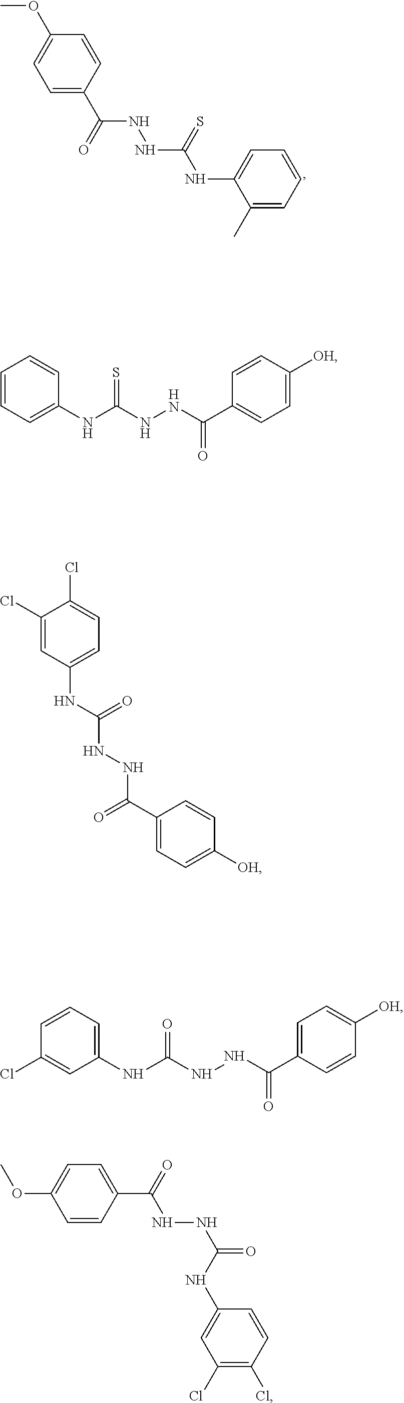 [SMILES: COc1ccc(C(=O)NNC(=S)Nc2ccccc2C)cc1 ; O=C(NNC(=S)Nc1ccccc1)c1ccc(O)cc1 ; O=C(NNC(=O)c1ccc(O)cc1)Nc1ccc(Cl)c(Cl)c1 ; O=C(NNC(=O)c1ccc(O)cc1)Nc1cccc(Cl)c1 ; COc1ccc(C(=O)NNC(=O)Nc2ccc(Cl)c(Cl)c2)cc1]

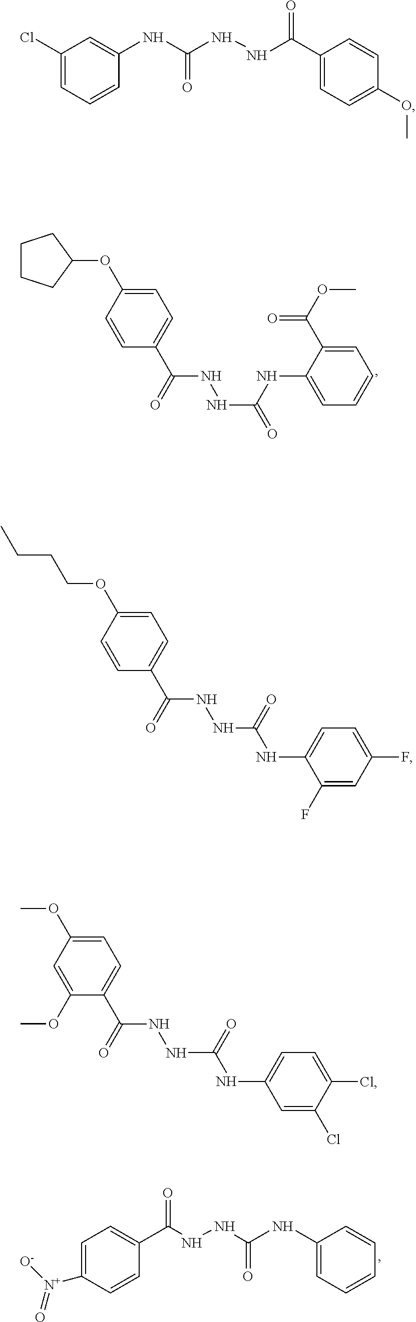

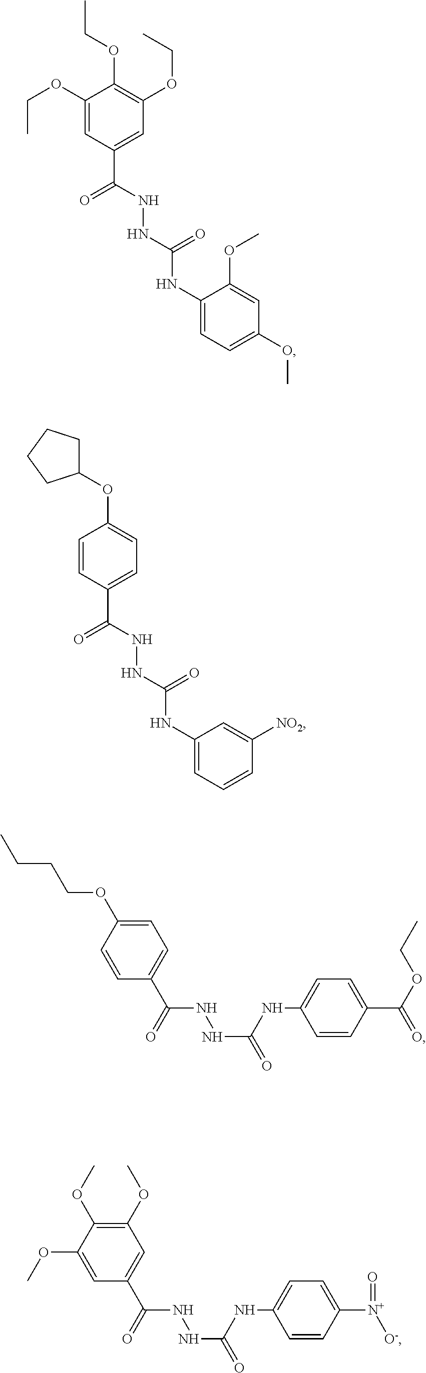

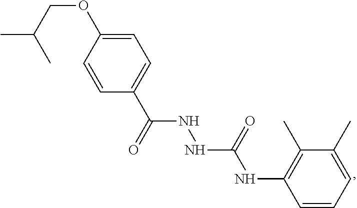 [SMILES: Cc1cccc(NC(=O)NNC(=O)c2ccc(OCC(C)C)cc2)c1C]

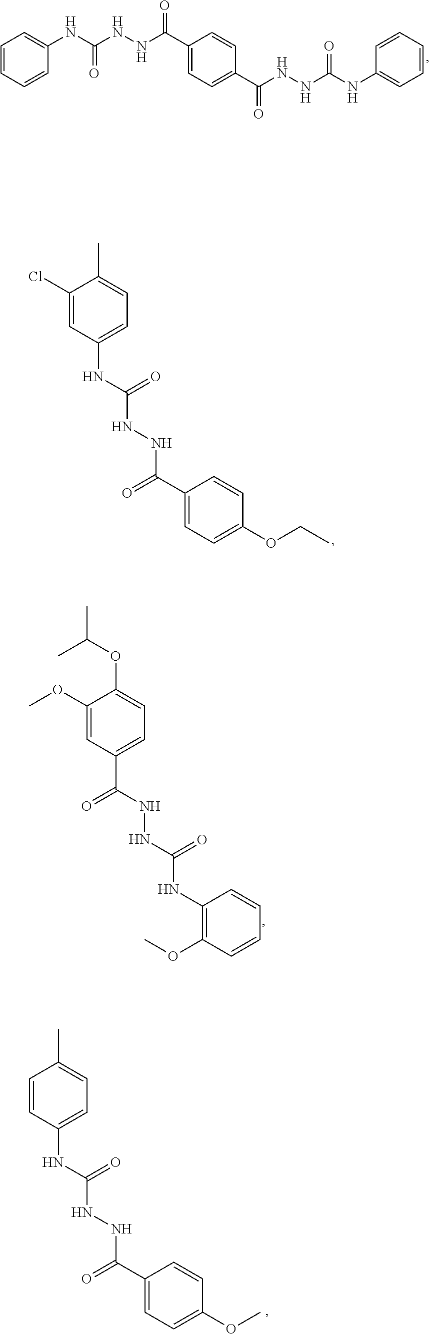

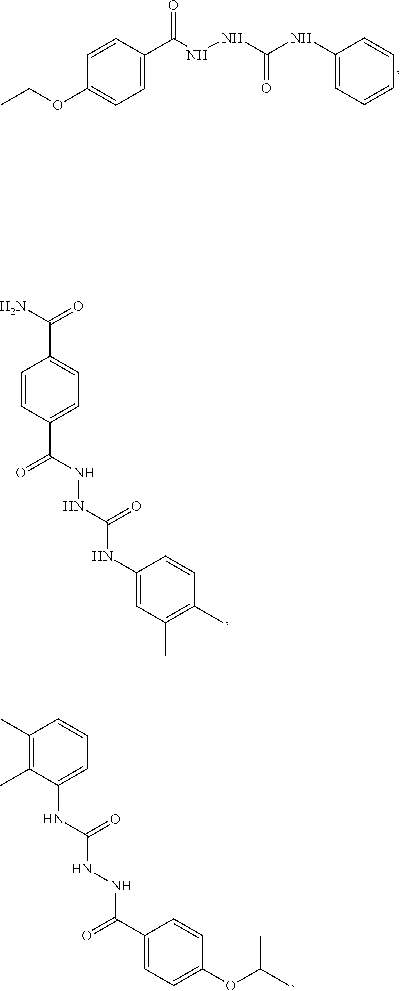 [SMILES: CCOc1ccc(C(=O)NNC(=O)Nc2ccccc2)cc1 ; Cc1ccc(NC(=O)NNC(=O)c2ccc(C(N)=O)cc2)cc1C ; Cc1cccc(NC(=O)NNC(=O)c2ccc(OC(C)C)cc2)c1C]

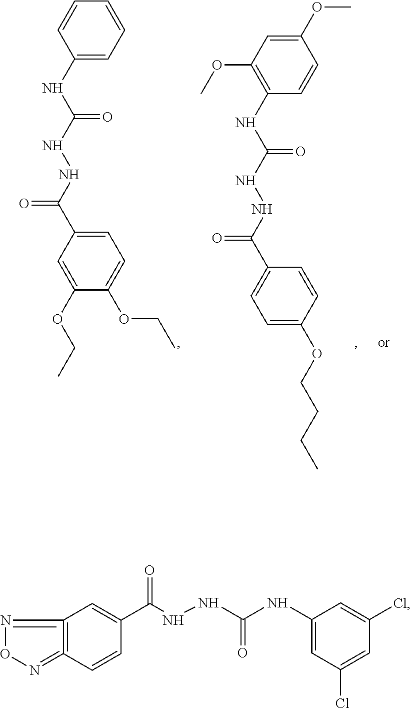

wherein any atoms with an incomplete valence are covalently bonded to one or more hydrogen atoms to complete their valence.

In certain embodiments, the invention relates to any of the compounds described herein, wherein

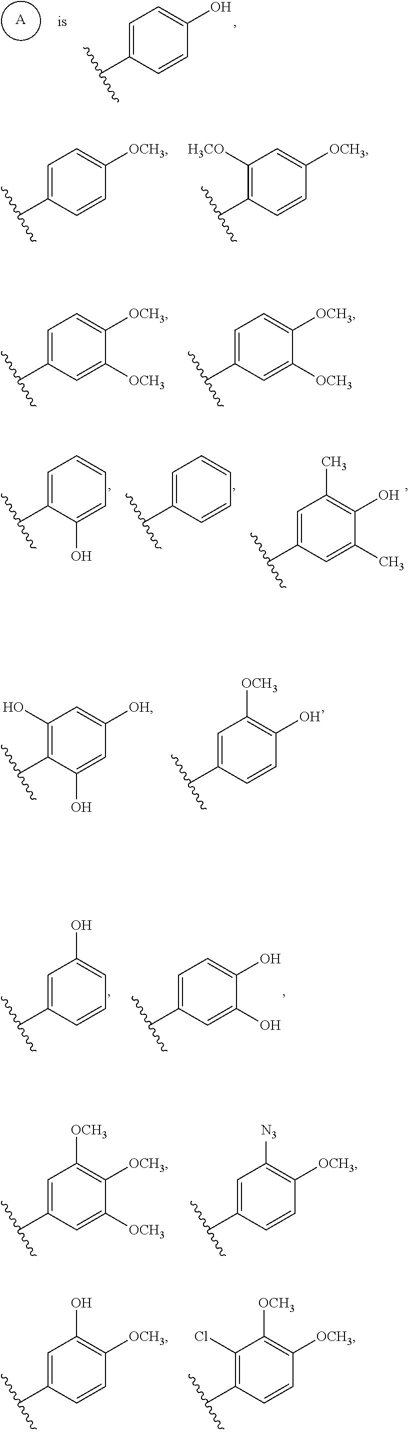

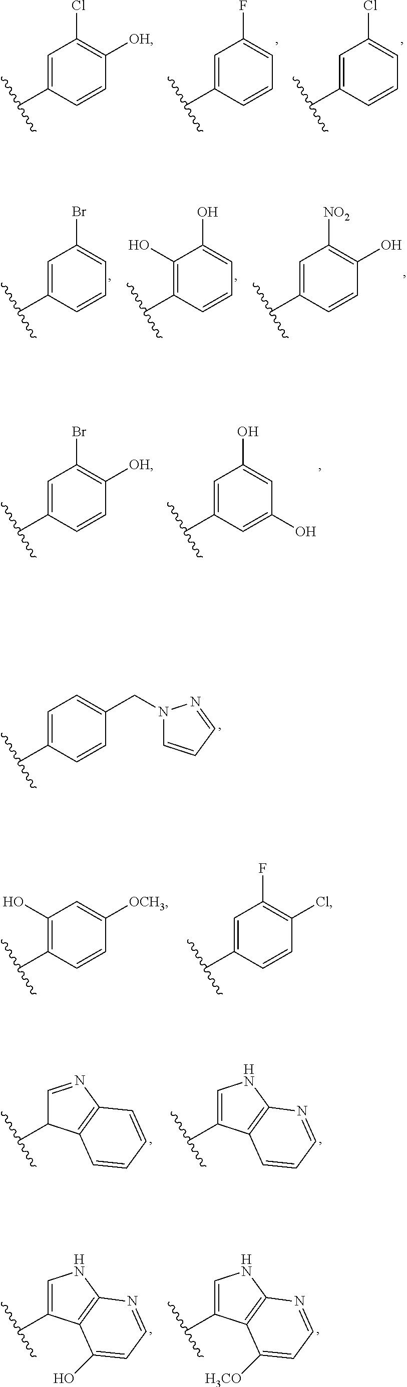

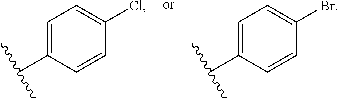 [SMILES: *c1ccc(Cl)cc1 ; "or" ; *c1ccc(Br)cc1]

In certain embodiments, the invention relates to a compound of Formula V

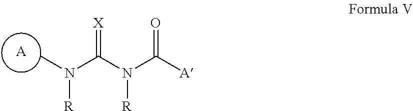

or a pharmaceutically acceptable salt or solvate thereof,
wherein, independently for each occurrence,

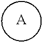

is optionally substituted aryl or optionally substituted heteroaryl;

R is hydrogen or alkyl;

A′ is optionally substituted alkyl, optionally substituted cycloalkyl, optionally substituted aryl, or optionally substituted heteroaryl; and

X is O or S,

provided the compound is not

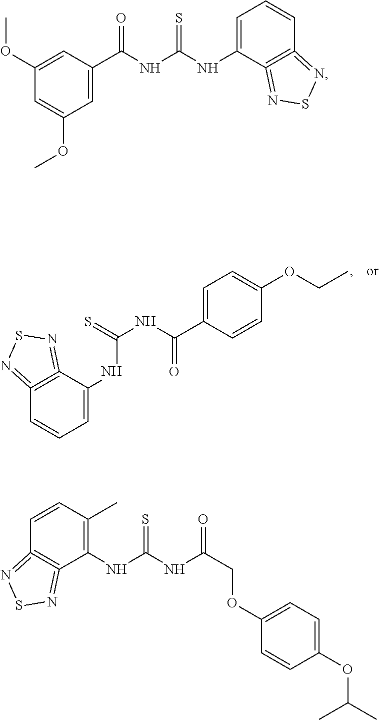

wherein any atoms with an incomplete valence are covalently bonded to one or more hydrogen atoms to complete their valence.

In certain embodiments, the invention relates to any of the compounds described herein, wherein

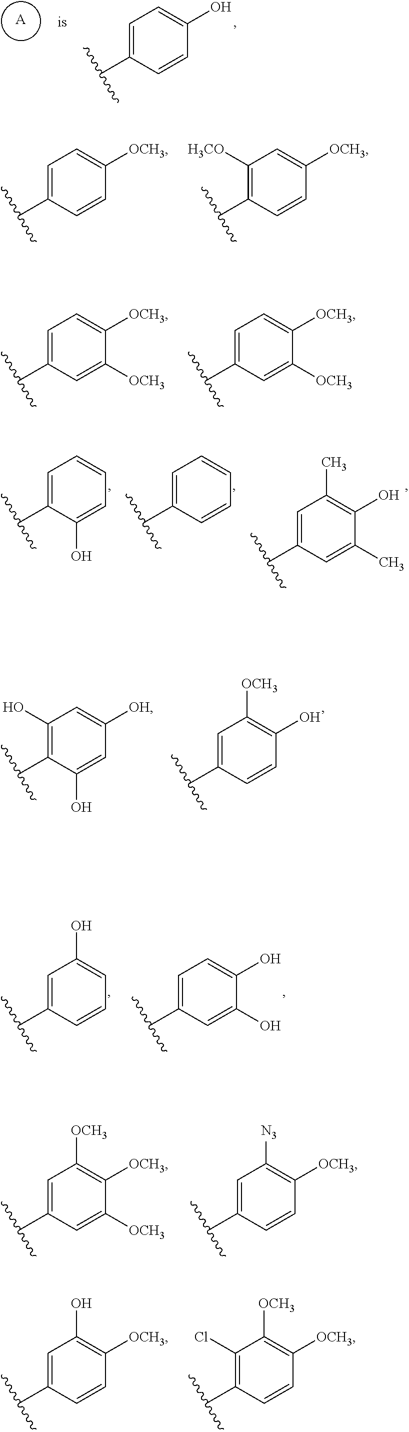

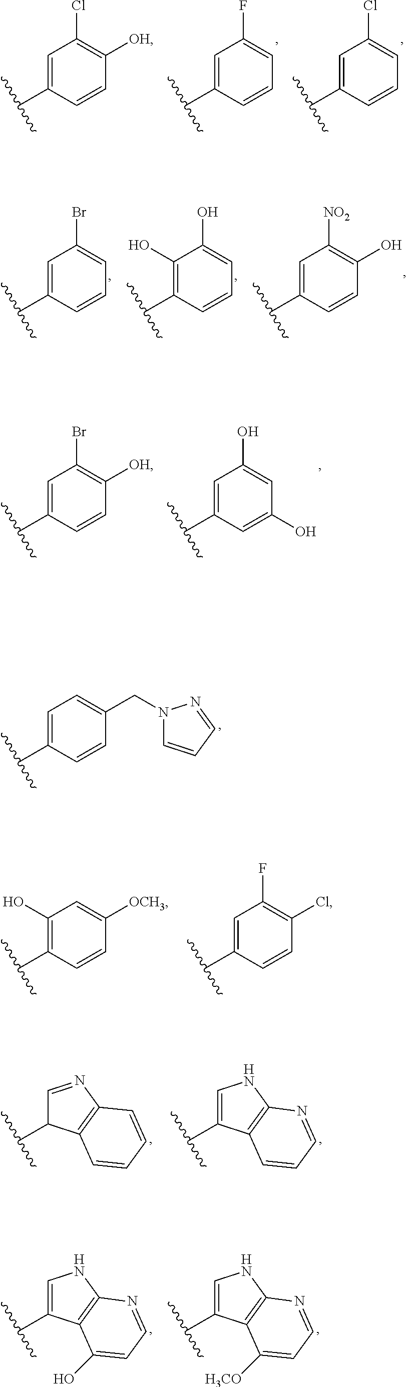

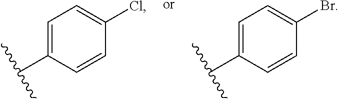 [SMILES: *c1ccc(Cl)cc1 ; "or" ; *c1ccc(Br)cc1]

In certain embodiments, the invention relates to any of the compounds described herein, wherein A′ is

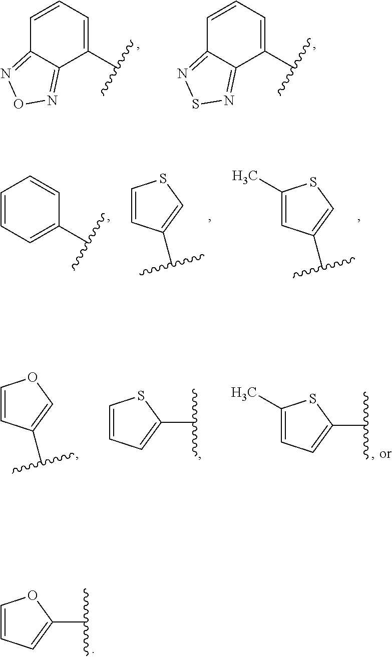

In certain embodiments, the invention relates to a compound of Formula VI

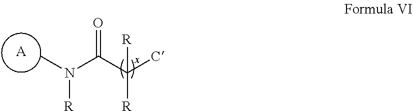

or a pharmaceutically acceptable salt or solvate thereof,
wherein, independently for each occurrence,

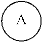

is optionally substituted aryl or optionally substituted heteroaryl;

R is hydrogen or alkyl;

x is 0, 1, 2, or 3; and

C′ is optionally substituted aryl, optionally substituted heteroaryl, optionally substituted cycloalkyl, optionally substituted heterocyclyl, optionally substituted aryloxy, optionally substituted heteroaryloxy, optionally substituted arylthio, or optionally substituted heteroarylthio;

provided the compound is not

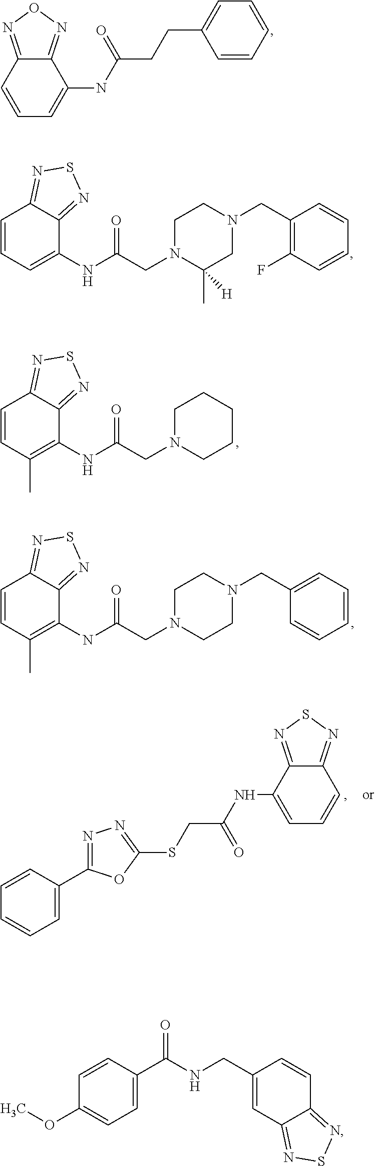 [SMILES: O=C(CCc1ccccc1)Nc1cccc2nonc12 ; C[C@@H]1CN(Cc2ccccc2F)CCN1CC(=O)Nc1cccc2nsnc12 ; Cc1ccc2nsnc2c1NC(=O)CN1CCCCC1 ; Cc1ccc2nsnc2c1NC(=O)CN1CCN(Cc2ccccc2)CC1 ; O=C(CSc1nnc(-c2ccccc2)o1)Nc1cccc2nsnc12 ; COc1ccc(C(=O)NCc2ccc3nsnc3c2)cc1]

wherein any atoms with an incomplete valence are covalently bonded to one or more hydrogen atoms to complete their valence.

In certain embodiments, the invention relates to any of the compounds described herein, wherein

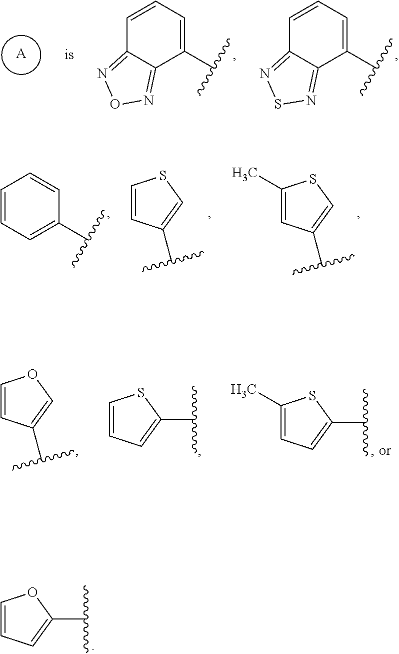

In certain embodiments, the invention relates to any of the compounds described herein, wherein C′ is

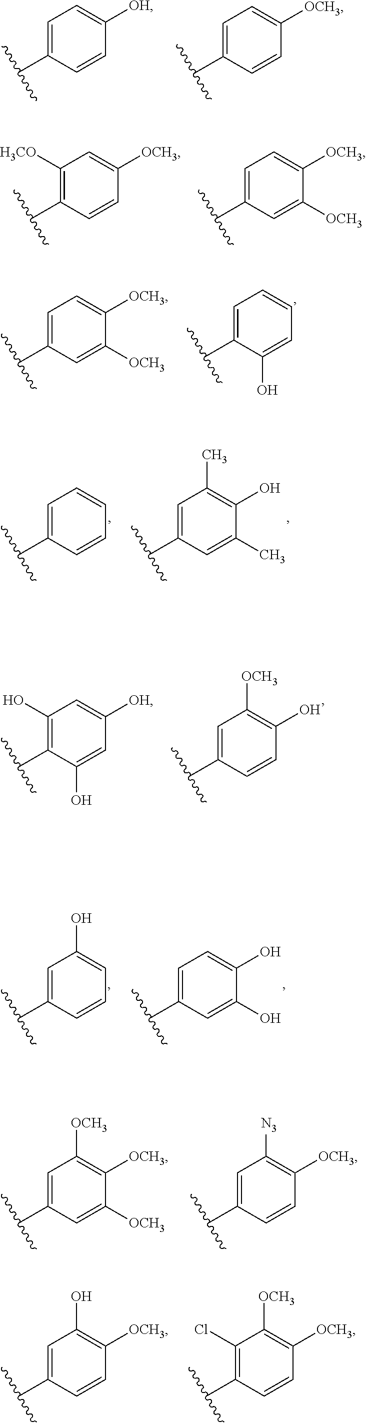

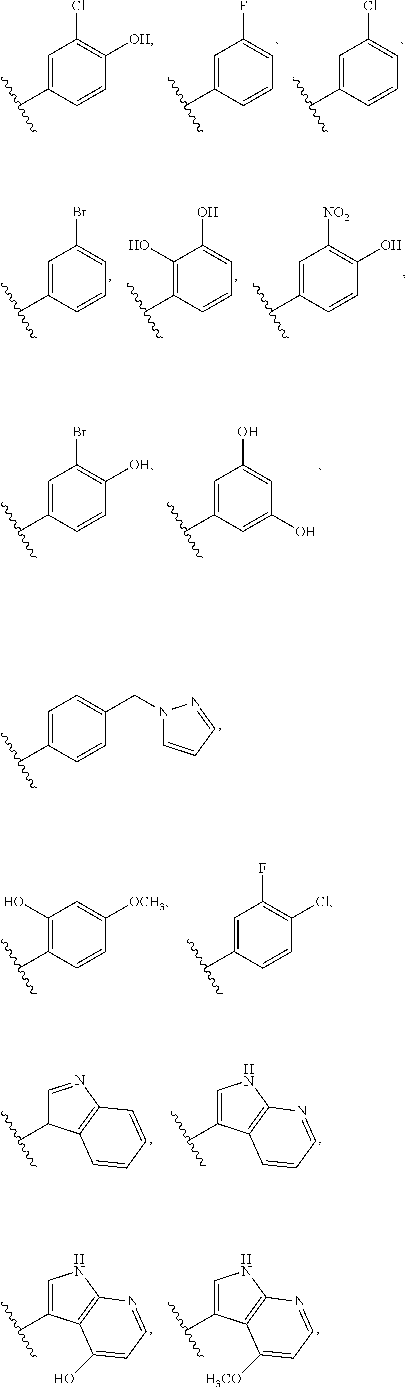

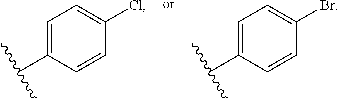 [SMILES: *c1ccc(Cl)cc1 ; "or" ; *c1ccc(Br)cc1]

In certain embodiments, the invention relates to a compound of Formula VII

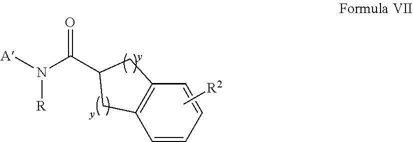

or a pharmaceutically acceptable salt or solvate thereof,
wherein, independently for each occurrence,

A′ is optionally substituted alkyl, optionally substituted cycloalkyl, optionally substituted aryl, or optionally substituted heteroaryl,

R is hydrogen or alkyl;

y is 1 or 2; and

R² is halo, hydroxy, alkoxy, alkylthio, or amino,

provided the compound is not

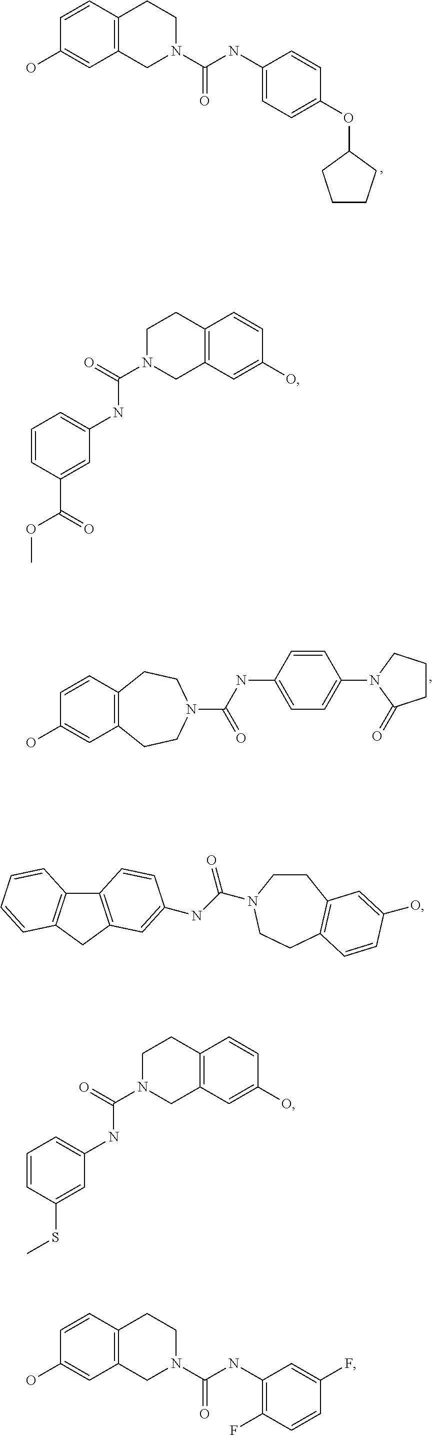

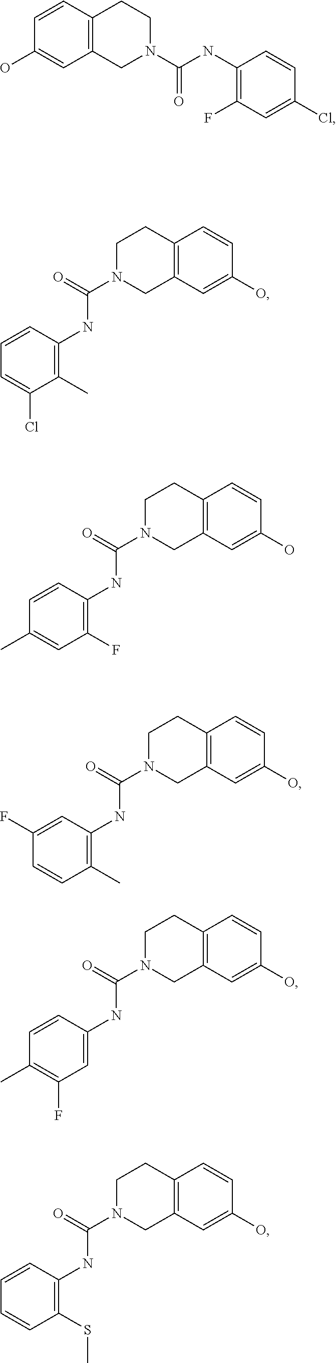

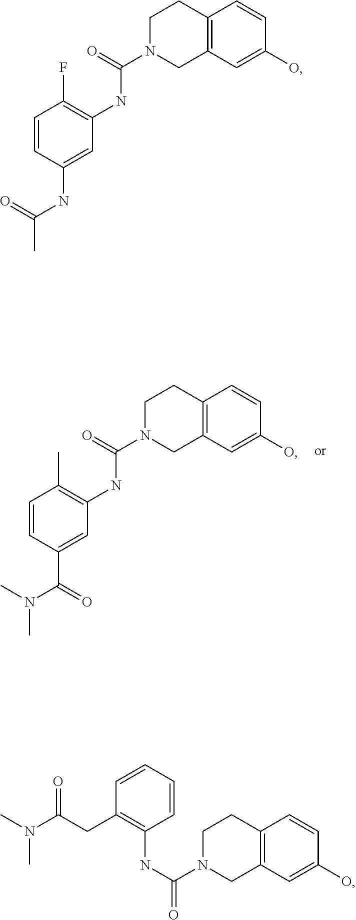

wherein any atoms with an incomplete valence are covalently bonded to one or more hydrogen atoms to complete their valence.

### Exemplary Methods

In certain embodiments, the invention relates to a method of inhibiting HIV exterior envelope glycoprotein gp120 comprising the step of: contacting HIV with an effective amount of a compound according to any one of Formulae I-VII.

In certain embodiments, the invention relates to a method of inhibiting transmission of HIV to a cell comprising the step of: contacting HIV with an effective amount of a compound according to any one of one of Formulae I-VII, thereby inhibiting transmission of HIV to said cell.

In certain embodiments, the invention relates to a method of inhibiting the progression of HIV infection in a human host comprising the step of: contacting HIV with an effective amount of a compound according to any one of Formulae I-VII, thereby inhibiting progression of HIV in the human host.

In certain embodiments, the invention relates to any one of the aforementioned methods, wherein the HIV is HIV-1 or HIV-2.

In certain embodiments, the invention relates to a method of inhibiting HIV exterior envelope glycoprotein gp120 comprising the step of: contacting HIV with an effective amount of a compound.

In certain embodiments, the invention relates to a method of inhibiting transmission of HIV to a cell comprising the step of: contacting HIV with an effective amount of a compound, thereby inhibiting transmission of HIV to said cell.

In certain embodiments, the invention relates to a method of inhibiting the progression of HIV infection in a human host comprising the step of: contacting HIV with an effective amount of a compound, thereby inhibiting progression of HIV in the human host.

In certain embodiments, the invention relates to any one of the aforementioned methods, wherein the compound is selected from the group consisting of:

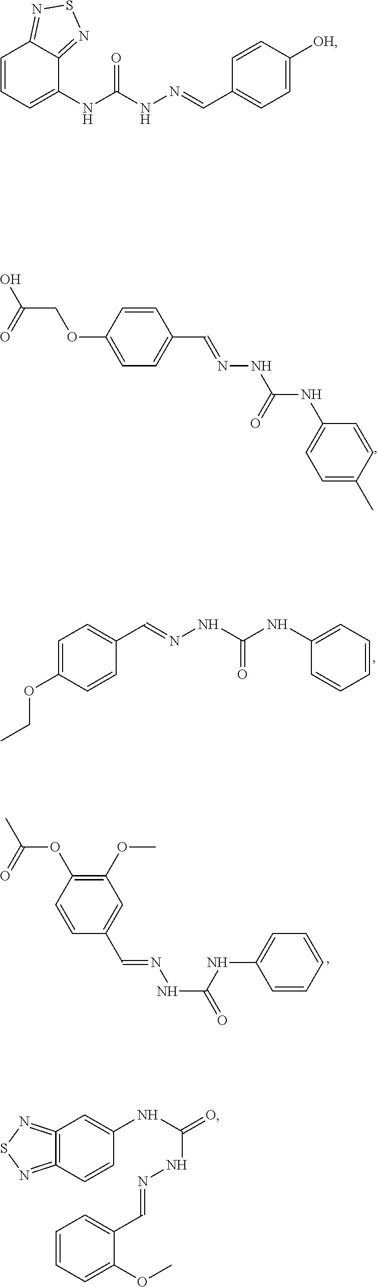

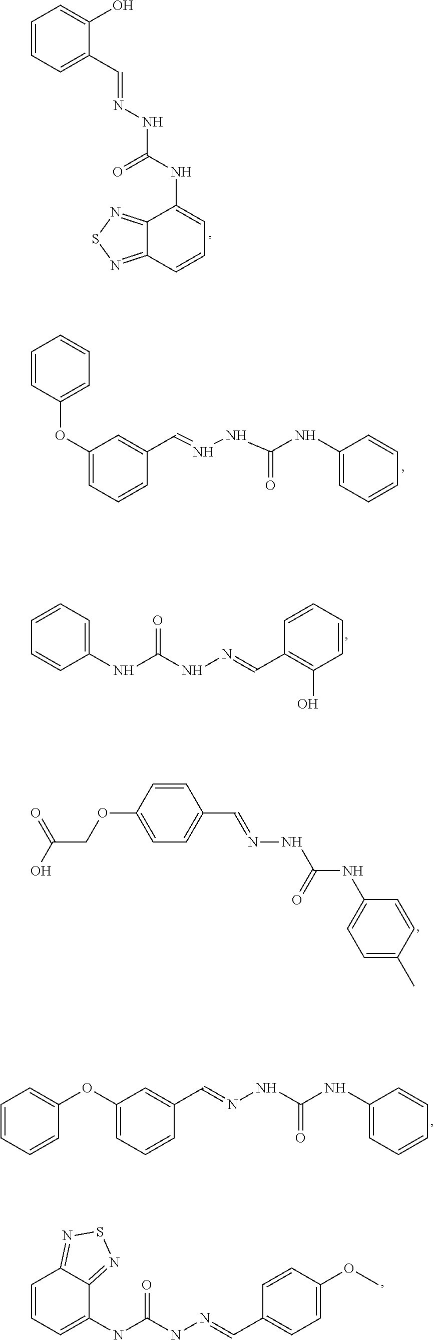 [SMILES: O=C(N/N=C/c1ccccc1O)Nc1cccc2nsnc12 ; O=C(N/N=C/c1cccc(Oc2ccccc2)c1)Nc1ccccc1 ; O=C(N/N=C/c1ccccc1O)Nc1ccccc1 ; Cc1ccc(NC(=O)N/N=C/c2ccc(OCC(=O)O)cc2)cc1 ; O=C(N/N=C/c1cccc(Oc2ccccc2)c1)Nc1ccccc1 ; COc1ccc(/C=N/NC(=O)Nc2cccc3nsnc23)cc1]

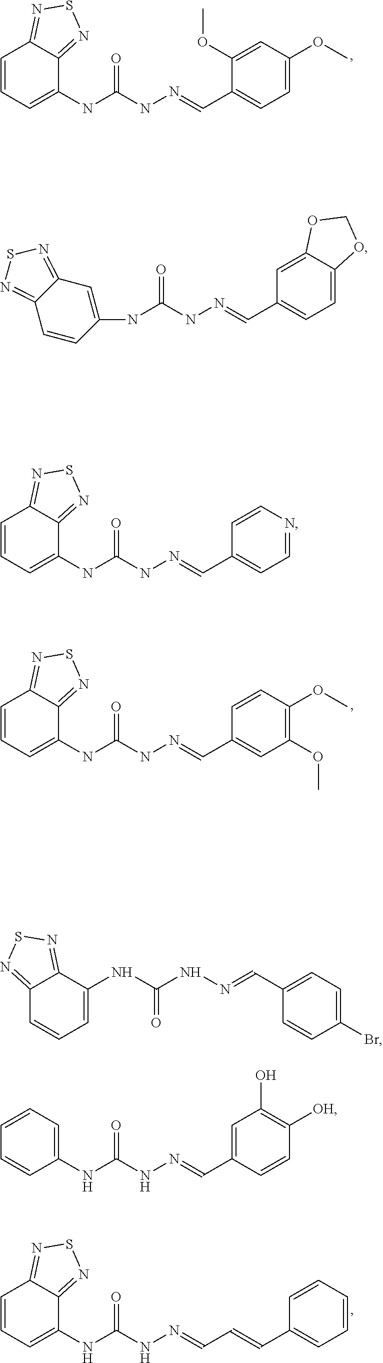

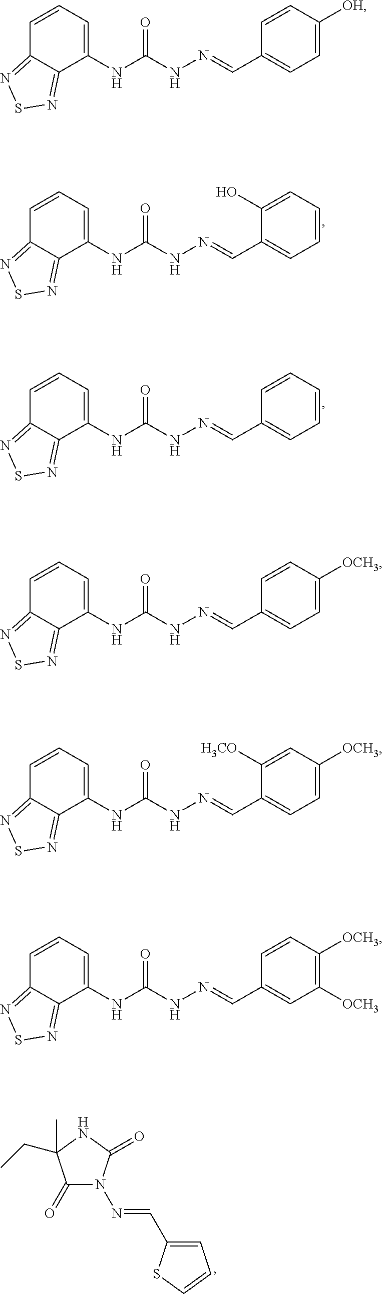

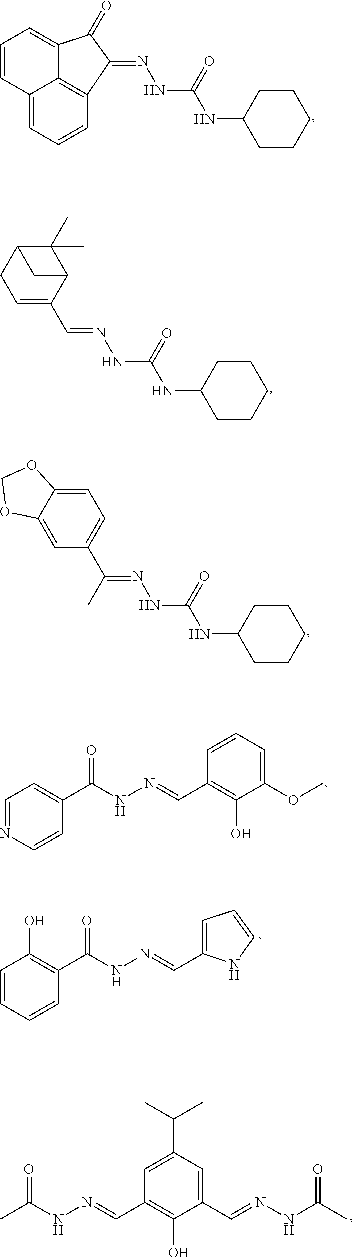

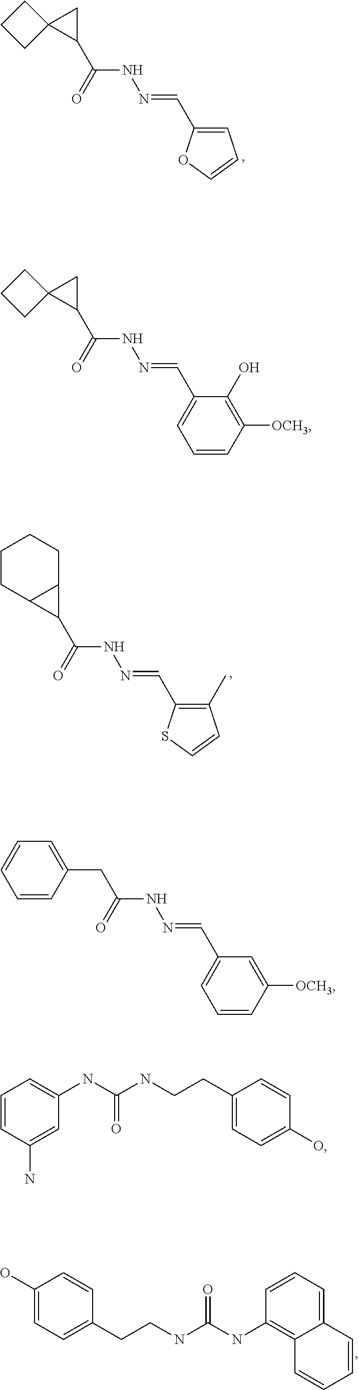

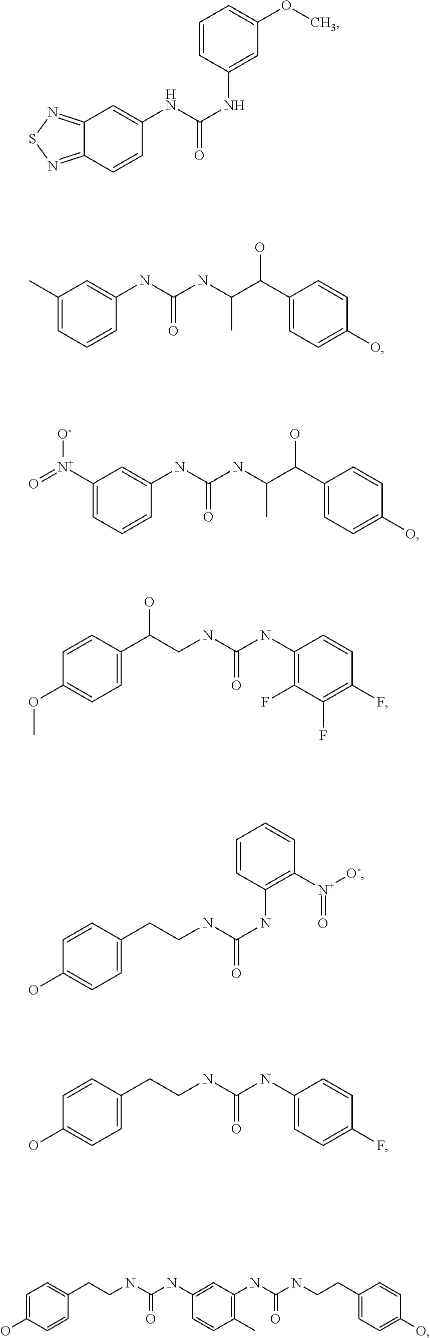

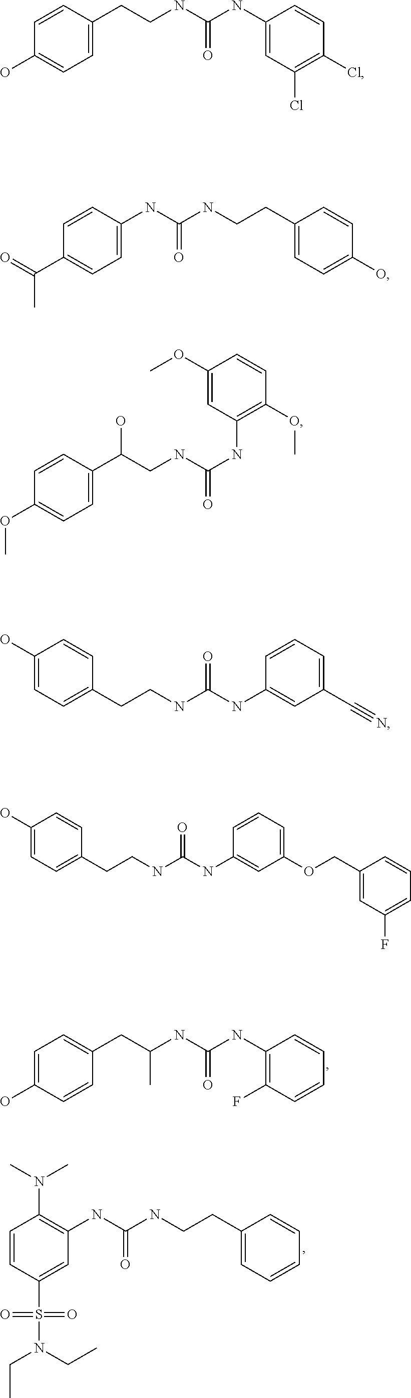

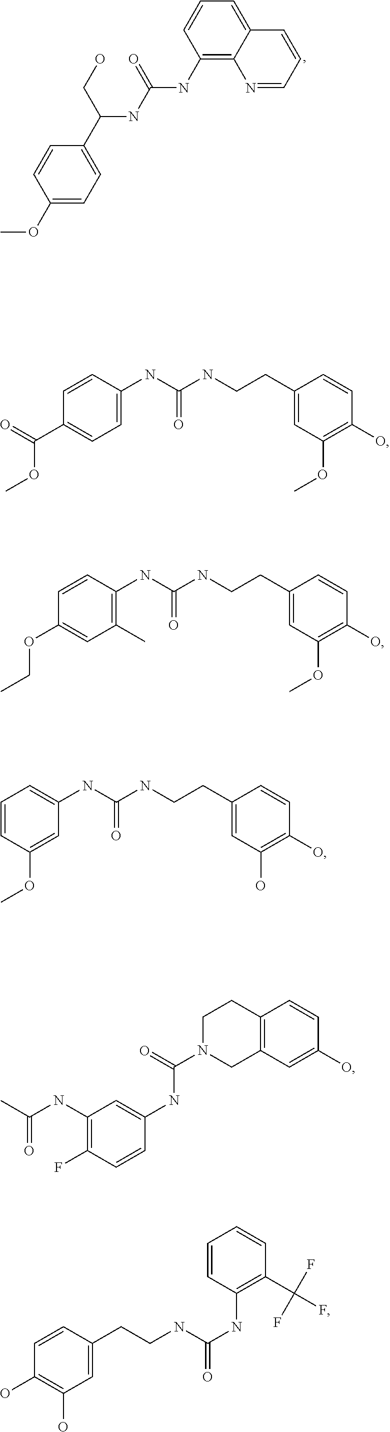

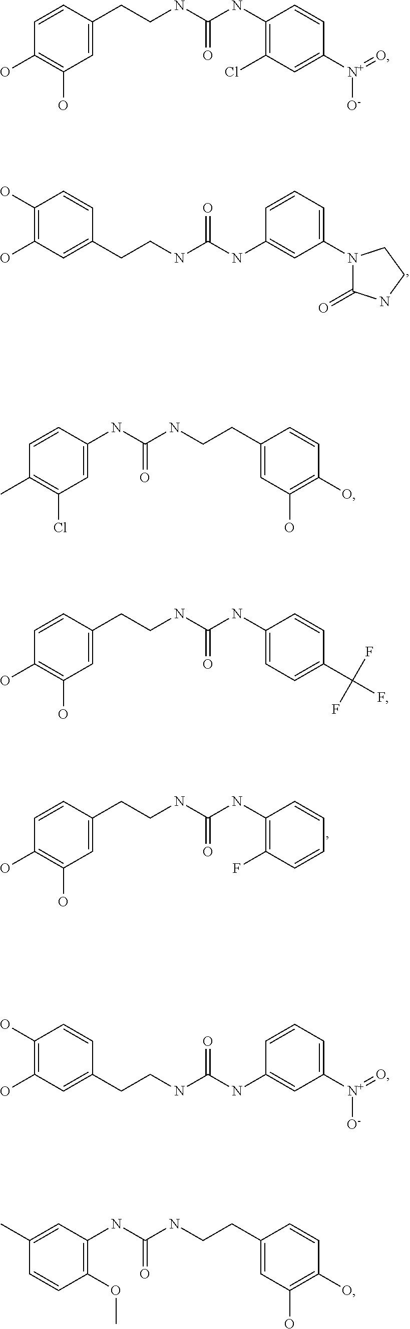

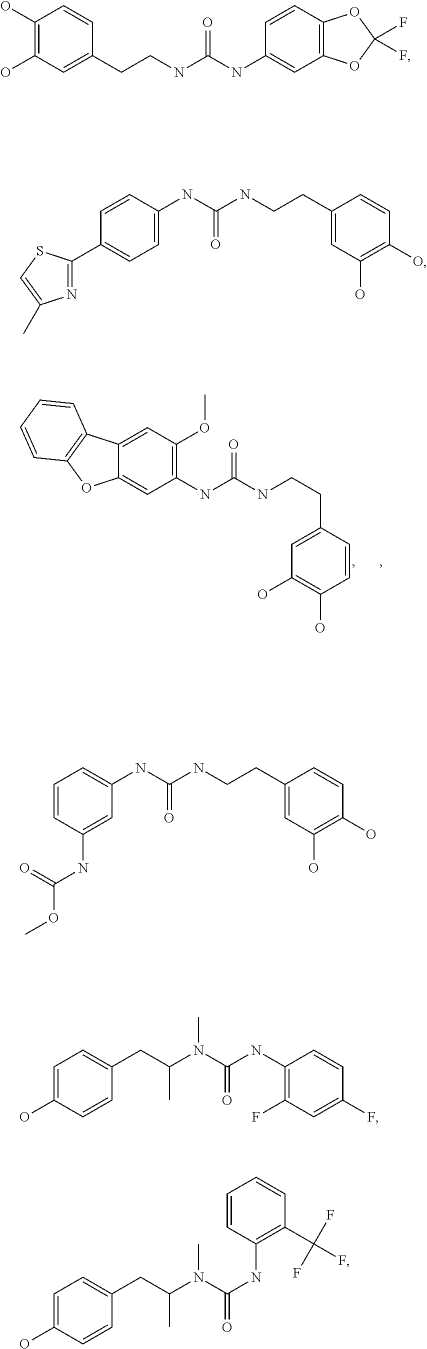

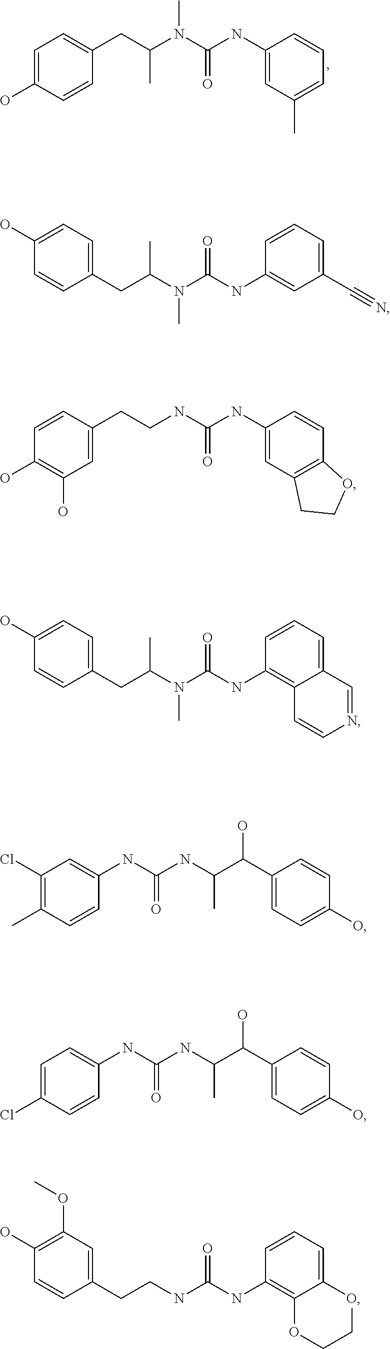

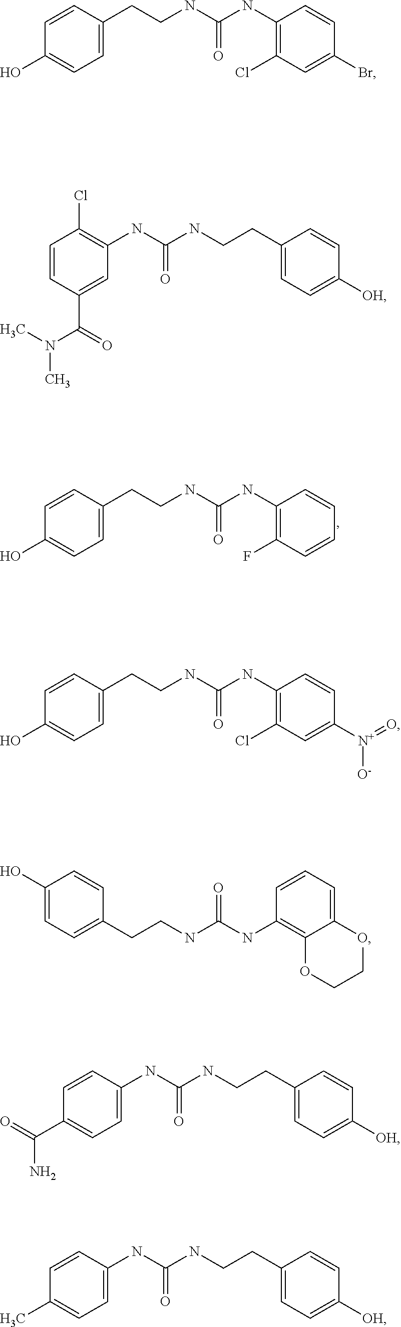

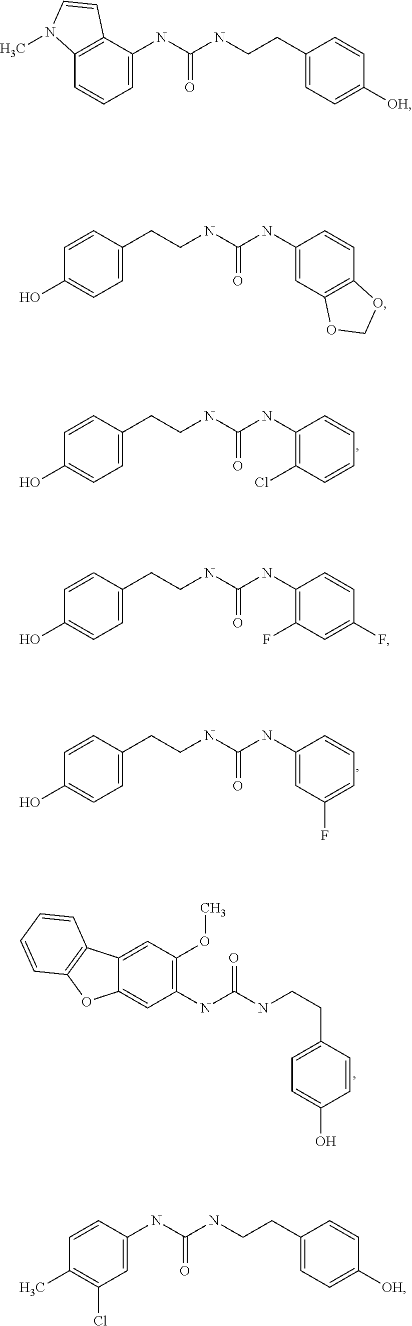

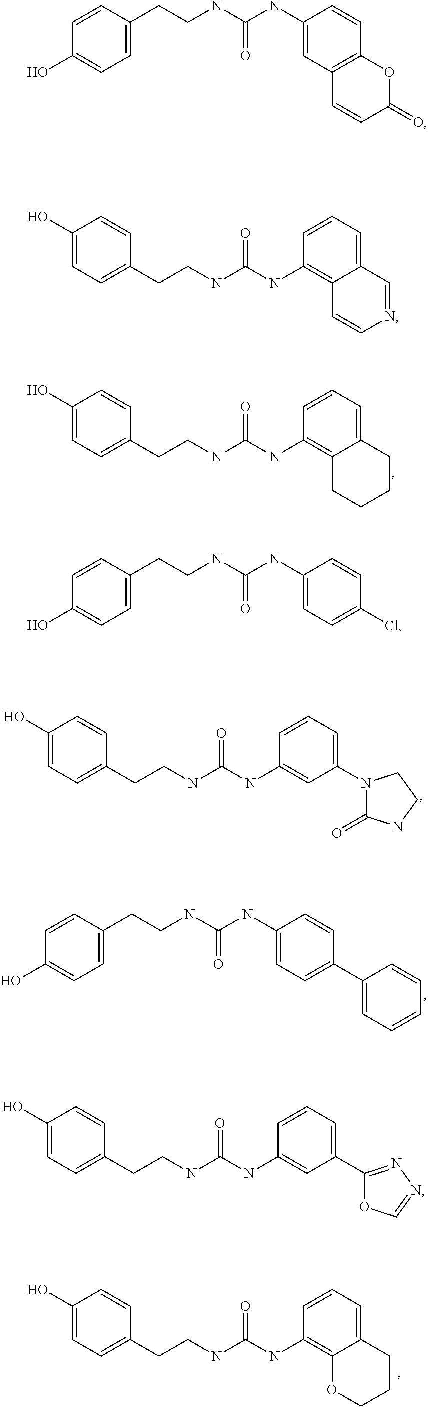

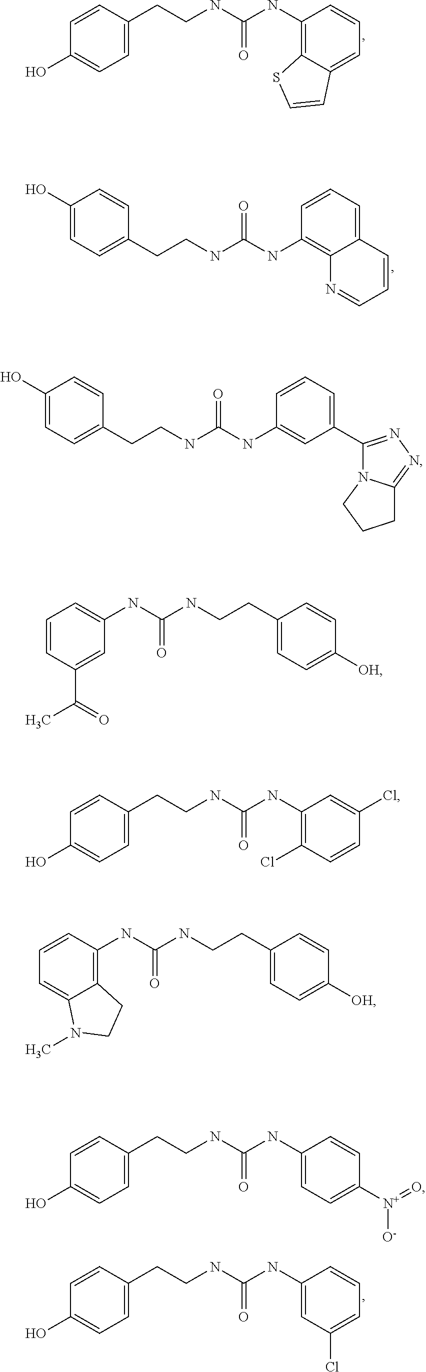

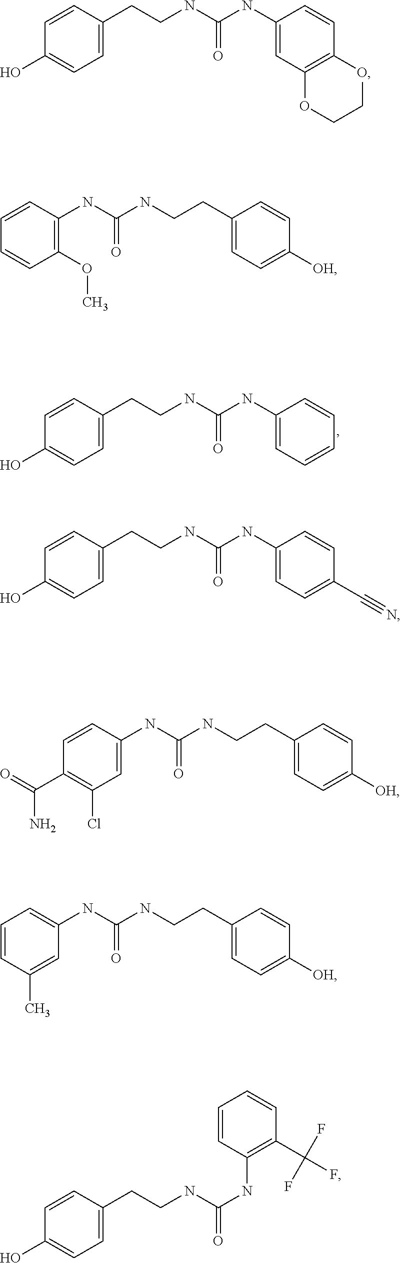

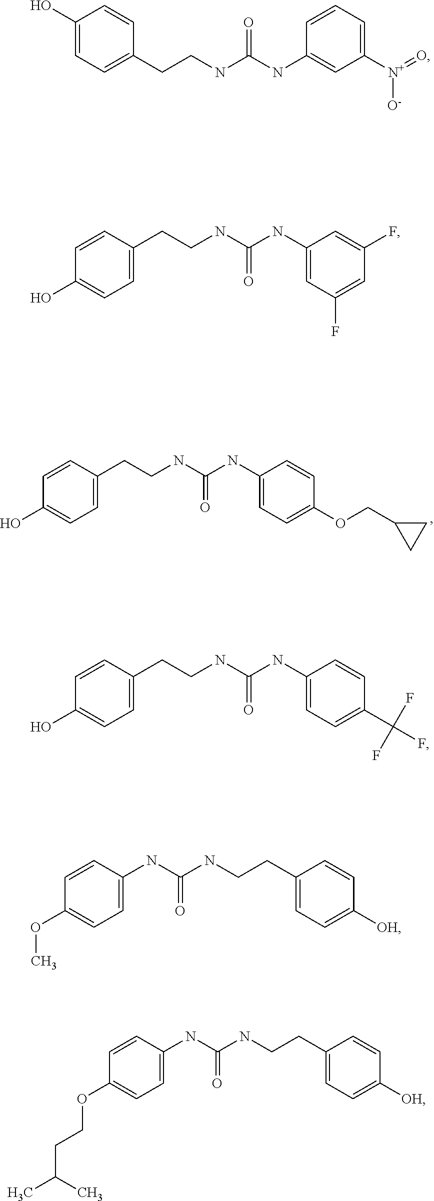 [SMILES: O=C(NCCc1ccc(O)cc1)Nc1cccc([N+](=O)[O-])c1 ; O=C(NCCc1ccc(O)cc1)Nc1cc(F)cc(F)c1 ; O=C(NCCc1ccc(O)cc1)Nc1ccc(OCC2CC2)cc1 ; O=C(NCCc1ccc(O)cc1)Nc1ccc(C(F)(F)F)cc1 ; COc1ccc(NC(=O)NCCc2ccc(O)cc2)cc1 ; CC(C)CCOc1ccc(NC(=O)NCCc2ccc(O)cc2)cc1]

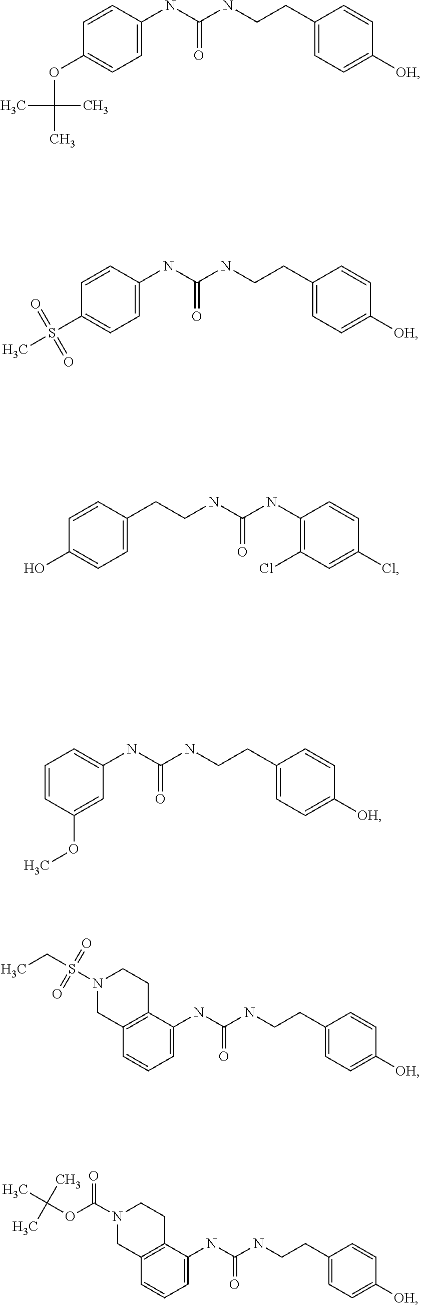 [SMILES: CC(C)(C)Oc1ccc(NC(=O)NCCc2ccc(O)cc2)cc1 ; CS(=O)(=O)c1ccc(NC(=O)NCCc2ccc(O)cc2)cc1 ; O=C(NCCc1ccc(O)cc1)Nc1ccc(Cl)cc1Cl ; COc1cccc(NC(=O)NCCc2ccc(O)cc2)c1 ; CCS(=O)(=O)N1CCc2c(cccc2NC(=O)NCCc2ccc(O)cc2)C1 ; CC(C)(C)OC(=O)N1CCc2c(cccc2NC(=O)NCCc2ccc(O)cc2)C1]

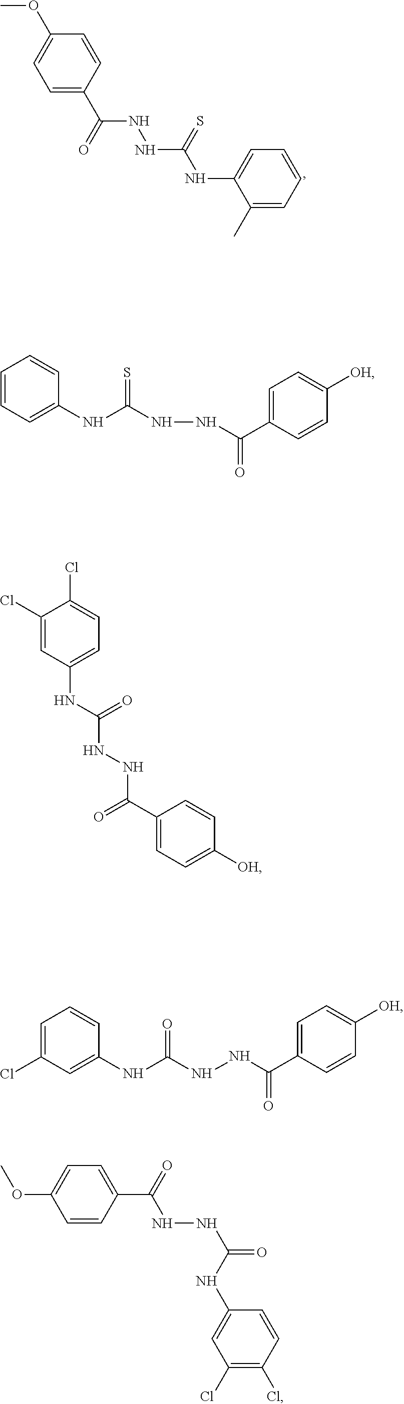 [SMILES: COc1ccc(C(=O)NNC(=S)Nc2ccccc2C)cc1 ; O=C(NNC(=S)Nc1ccccc1)c1ccc(O)cc1 ; O=C(NNC(=O)c1ccc(O)cc1)Nc1ccc(Cl)c(Cl)c1 ; O=C(NNC(=O)c1ccc(O)cc1)Nc1cccc(Cl)c1 ; COc1ccc(C(=O)NNC(=O)Nc2ccc(Cl)c(Cl)c2)cc1]

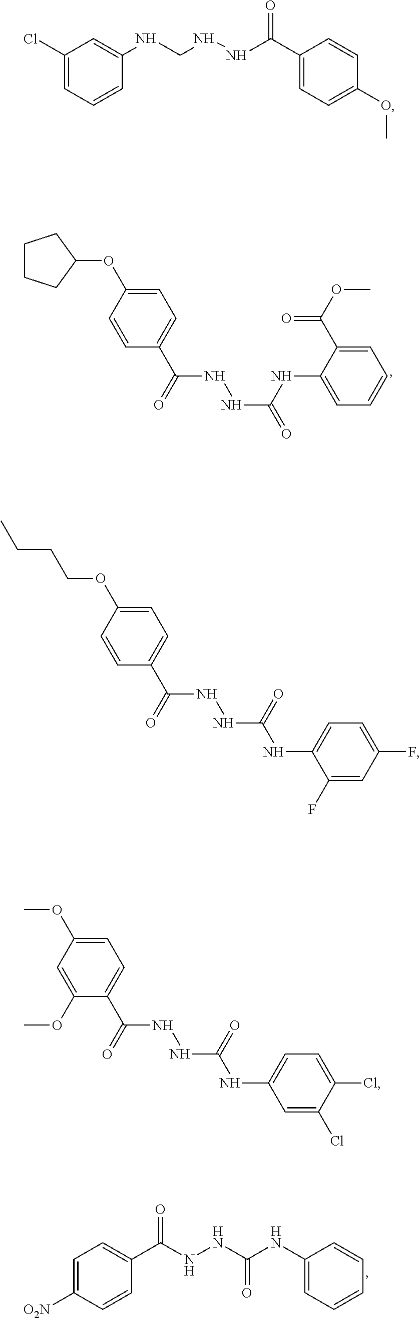

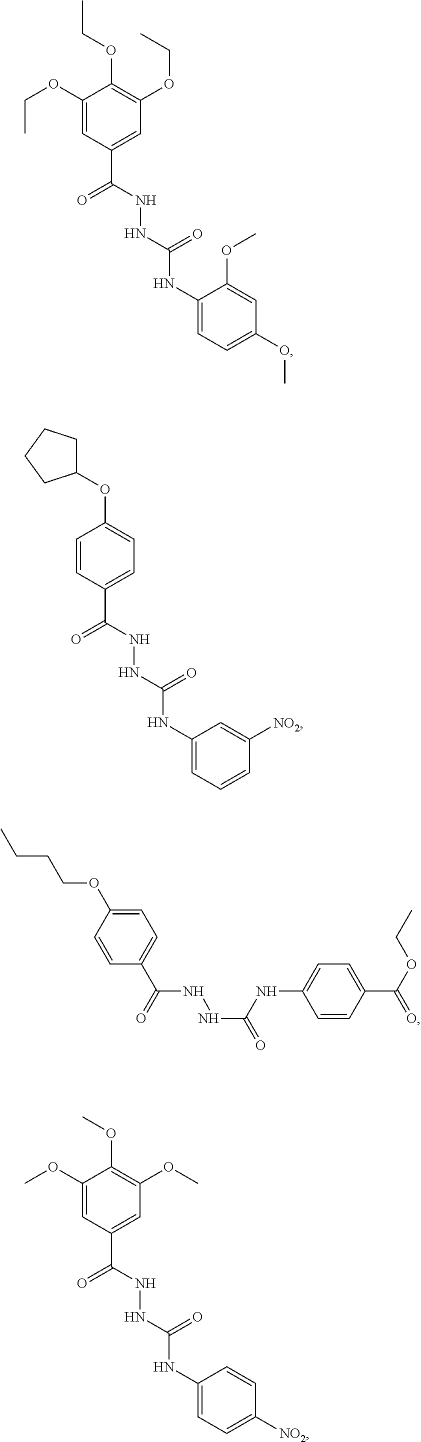

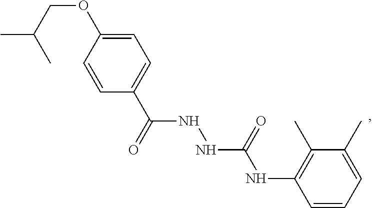 [SMILES: Cc1cccc(NC(=O)NNC(=O)c2ccc(OCC(C)C)cc2)c1C]

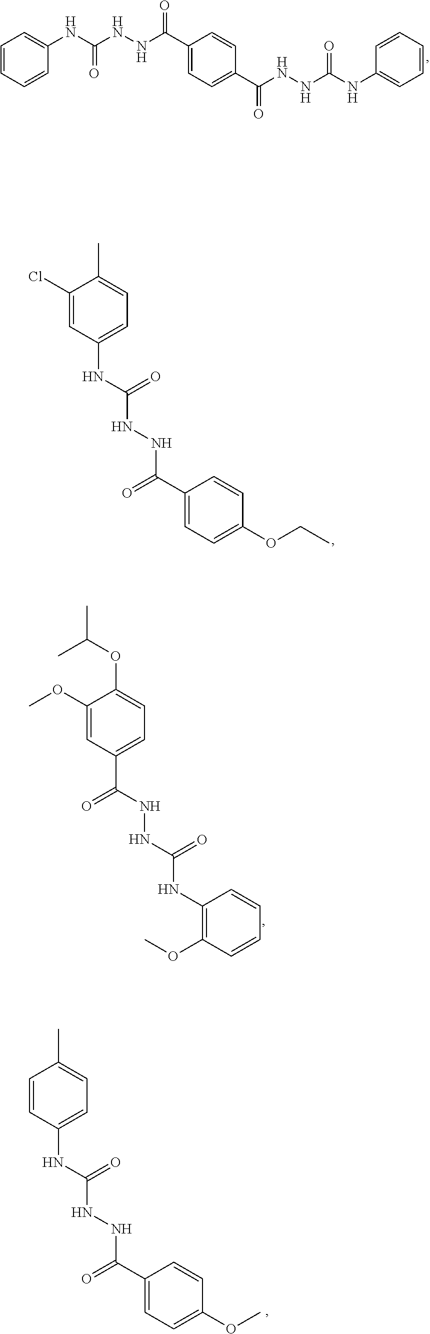

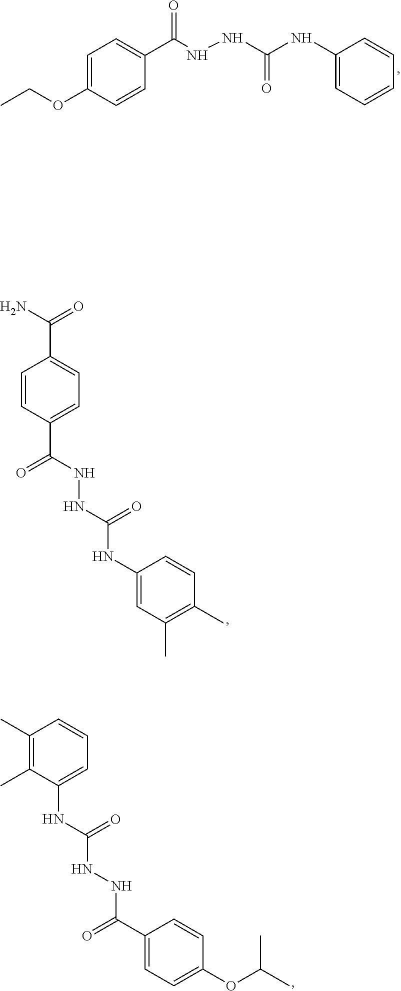 [SMILES: CCOc1ccc(C(=O)NNC(=O)Nc2ccccc2)cc1 ; Cc1ccc(NC(=O)NNC(=O)c2ccc(C(N)=O)cc2)cc1C ; Cc1cccc(NC(=O)NNC(=O)c2ccc(OC(C)C)cc2)c1C]

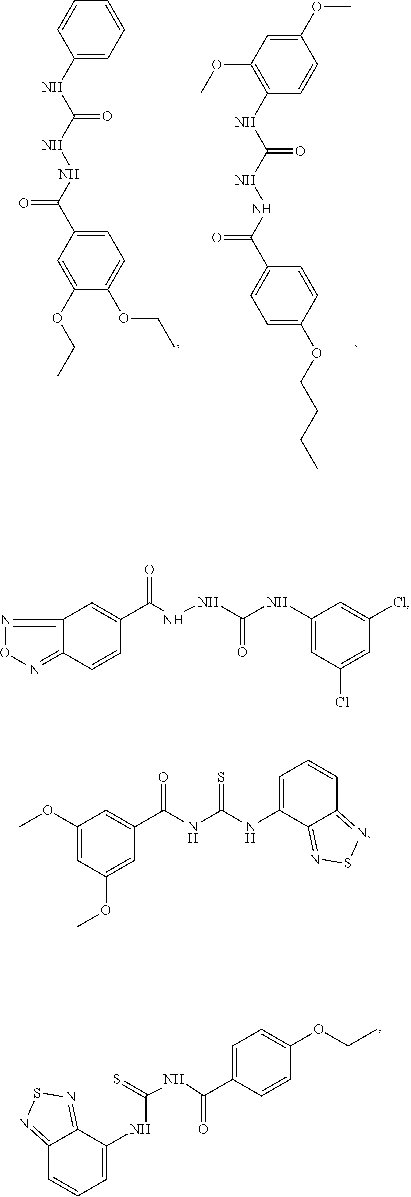

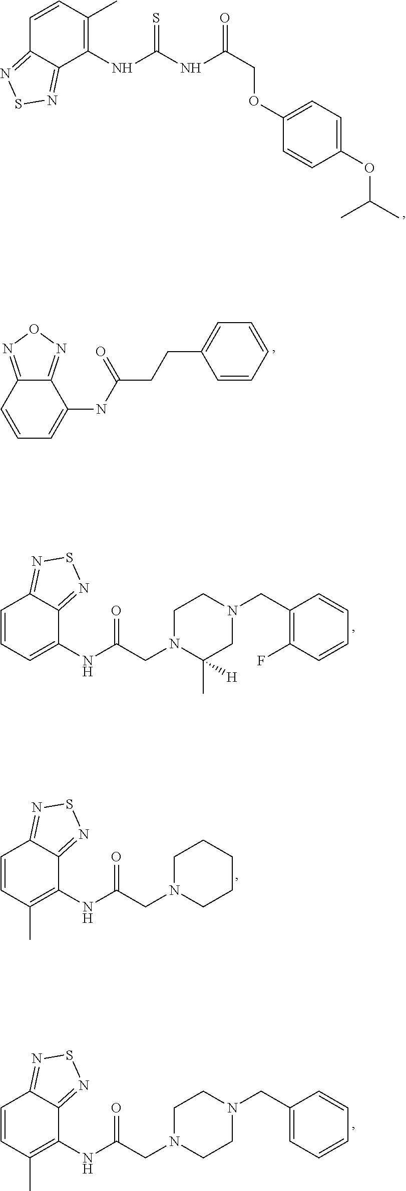 [SMILES: Cc1ccc2nsnc2c1NC(=S)NC(=O)COc1ccc(OC(C)C)cc1 ; O=C(CCc1ccccc1)Nc1cccc2nonc12 ; C[C@@H]1CN(Cc2ccccc2F)CCN1CC(=O)Nc1cccc2nsnc12 ; Cc1ccc2nsnc2c1NC(=O)CN1CCCCC1 ; Cc1ccc2nsnc2c1NC(=O)CN1CCN(Cc2ccccc2)CC1]

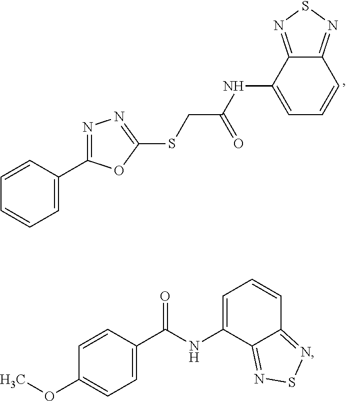 [SMILES: O=C(CSc1nnc(-c2ccccc2)o1)Nc1cccc2nsnc12 ; COc1ccc(C(=O)Nc2cccc3nsnc23)cc1]

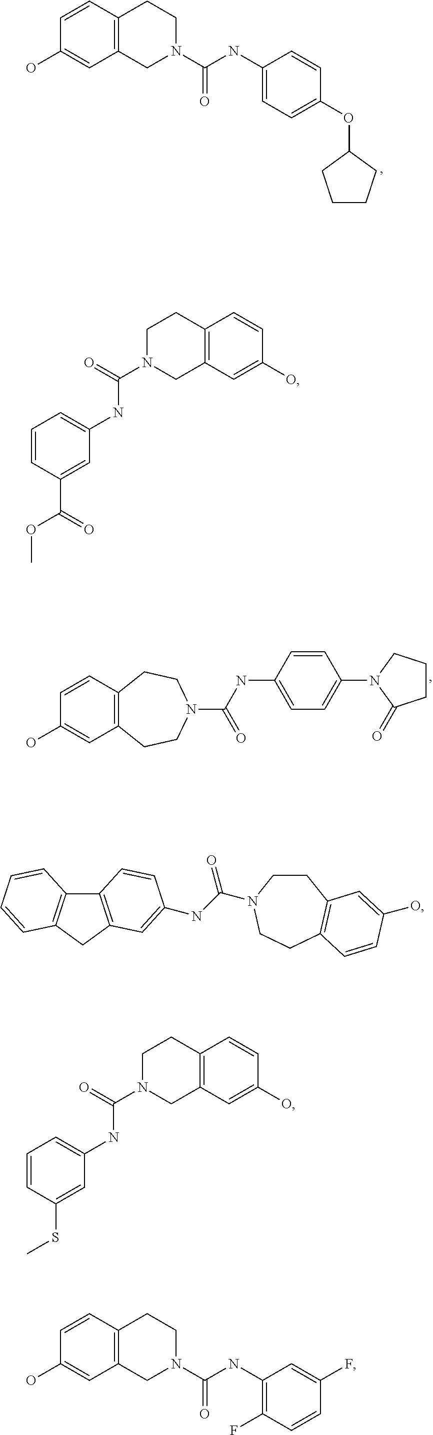

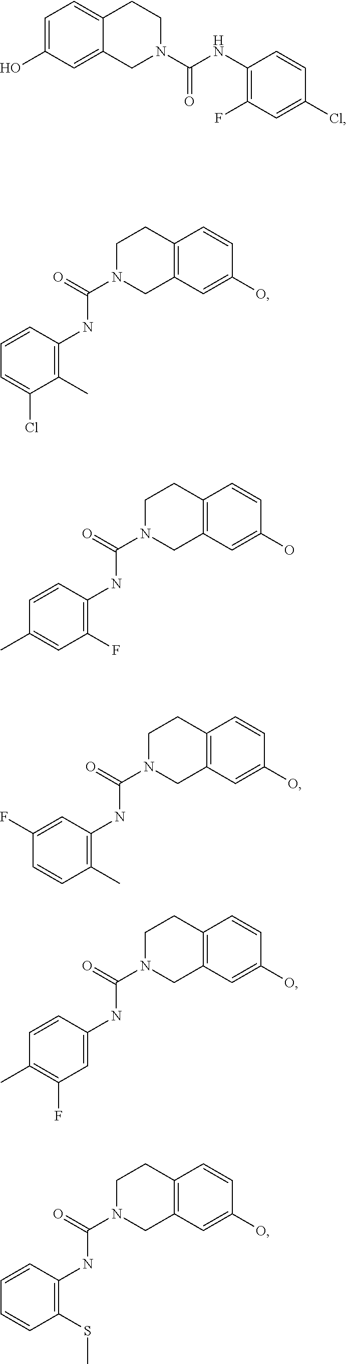

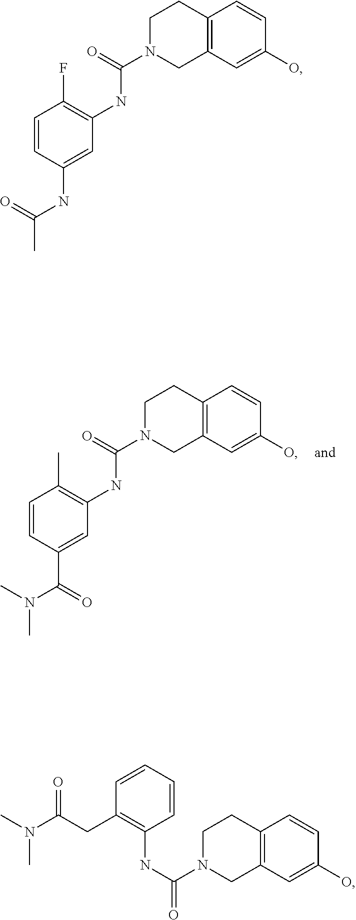

wherein any atoms with an incomplete valence are covalently bonded to one or more hydrogen atoms to complete their valence.

In certain embodiments, the invention relates to any one of the aforementioned methods, wherein the compound is

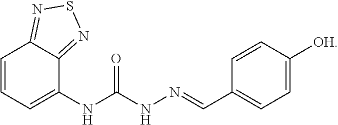 [SMILES: O=C(N/N=C/c1ccc(O)cc1)Nc1cccc2nsnc12]

In certain embodiments, the invention relates to any one of the aforementioned methods, provided the compound is not

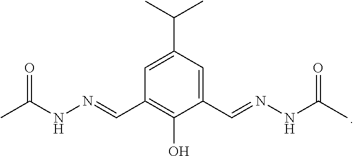 [SMILES: CC(=O)N/N=C/c1cc(C(C)C)cc(/C=N/NC(C)=O)c1O]

In certain embodiments, the invention relates to any one of the aforementioned methods, wherein the HIV is HIV-1 or HIV-2.

### Exemplary Pharmaceutical Compositions

While it is possible for compounds of the present invention to be administered as the raw chemical, it is also possible to present them as a pharmaceutical formulation. Accordingly, the present invention provides a pharmaceutical formulation comprising a compound or a pharmaceutically acceptable salt, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients can be used as suitable and as understood in the art, e.g., in Remington's Pharmaceutical Sciences. The pharmaceutical compositions of the present invention can be manufactured in a manner that is itself known, e.g., by means of conventional mixing, dissolving, granulating, dragee-making, levigating, emulsifying, encapsulating, entrapping or compression processes, for example.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route depends upon for example the condition and disorder of the recipient. The formulations can conveniently be presented in unit dosage form and can be prepared by any of the methods well known in the art. All methods include the step of bringing into association a compound of the present invention or a pharmaceutically acceptable salt, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration can be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient can also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets can be made by compression or molding, optionally with one or more accessory ingredients. Compressed tablets can be prepared by compressing in a suitable machine the active ingredient in a free-flowing form such as a powder or granules, optionally mixed with binders, inert diluents, or lubricating, surfaceactive or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. The formulations can be presented in unit-dose or multi-dose containers, for example sealed ampoules and vials, and can be stored in powder form or in a freeze-dried (lyophilized) condition requiring only the addition of the sterile liquid carrier, for example, saline or sterile pyrogen-free water, immediately prior to use. Extemporaneous injection solutions and suspensions can be prepared from sterile powders, granules and tablets of the kind previously described.

Formulations for parenteral administration include aqueous and non-aqueous (oily) sterile injection solutions of the active compounds which can contain antioxidants, buffers, bacteriostats and solutes which render the formulation isotonic with the blood of the intended recipient; and aqueous and non-aqueous sterile suspensions which can include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

In addition to the formulations described previously, the compounds of the present invention can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compounds can also be formulated in rectal compositions such as suppositories or retention enemas, e.g., containing conventional suppository bases such as cocoa butter, polyethylene glycol, or other glycerides. The compounds can also be formulated in vaginal compositions as gels, suppositories, or as dendrimers conjugates. Compounds of the present invention can be administered topically, that is by non-systemic administration. Formulations suitable for topical administration include liquid or semi-liquid preparations suitable for penetration through the skin such as gels, liniments, lotions, creams, ointments or pastes.

Gels for topical or transdermal administration of compounds of the present invention can include a mixture of volatile solvents, nonvolatile solvents, and water. The volatile solvent component of the buffered solvent system can include lower (C1-C6) alkyl alcohols, lower alkyl glycols and lower glycol polymers. In certain embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound can crystallize due to evaporation of volatile solvent, while an excess will result in a lack of bioavailability due to poor release of drug from solvent mixture. The buffer component of the buffered solvent system can be selected from any buffer commonly used in the art; in certain embodiments, water is used. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions or liniments for application to the skin can also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They can be made by mixing the active ingredient in finely-divided or powdered form, alone or in solution or suspension in an aqueous or non-aqueous fluid, with the aid of suitable machinery, with a greasy or non greasy base. The base can comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation can incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin can also be included.

### EXEMPLIFICATION

This invention is further illustrated by the following examples, which should not be construed as limiting.

### Example 1

### General Materials and Methods

High-throughput screening. The cell-cell fusion and specificity control assays were used in parallel to identify potential inhibitors of HIV-1 entry. Both assays were used to screen several libraries of chemical compounds from different sources (Table 1) at the Institute of Chemistry and Cell Biology, Harvard Medical School.

TABLE 1
Libraries of chemical compounds that were screened

| | | | |
|---|---|---|---|
| | Compounds | | |
| Library Name | screened | Concentration | Provider |

Known bioactive collections

| | | | | |
|---|---|---|---|---|
| NIH Clinical Collection 2 | 281 | 10 | mM | NIH |
| NINDS Custom Collection 2 | 1040 | 10 | mM | NINDS |
| Prestwick2 Collection | 1120 | 2 | mg/mL | Prestwick |
| Tocriscreen Mini Library | 1120 | 10 | mM | Tocris |
| BIOMOL ICCB Known Bioactives 3 | 480 | 0.1-13 | mM | Biomol |
| Library of Pharmacologically Active | 1280 | 10 | mM | Sigma-Aldrich |
| Compounds 1 | | | | |
| MicroSource Discovery 1 | 270 | 10 | mM | MicroSource |
| Microsource 1 - US Drug Collection | 1040 | 2 | mg/mL | Microsource |

Commercial libraries

| | | | | |
|---|---|---|---|---|
| ActiMolTimTec1 | 8518 | 5 | mg/mL | Biomol-TimTec |
| Asinex 1 | 12378 | 5 | mg/mL | Asinex |
| Bionet 1 | 4160 | 5 | mg/mL | Bionet |
| Bionet 2 | 1700 | 5 | mg/mL | Bionet |
| CEREP | 4800 | 5 | mg/mL | CEREP |
| ChemBridge3 | 10560 | 5 | mg/mL | ChemBridge |
| ChemDiv1 | 26048 | 5 | mg/mL | ChemDiv |
| Chemical Diversity 2 | 8560 | 5 | mg/mL | ChemDiv |
| ChemDiv3 | 16544 | 5 | mg/mL | ChemDiv |
| ChemDiv4 | 14677 | 5 | mg/mL | ChemDiv |
| ChemDiv5 | 1249 | 5 | mg/mL | ChemDiv |
| ChemDiv6 | 44000 | 10 | mM | ChemDiv |
| Enamine 1 | 6004 | 5 | mg/mL | Enamine |
| Enamine 2 | 26576 | 5 | mg/mL | Enamine |
| IFLab2 | 292 | 5 | mg/mL | IFLab |
| Life Chemicals 1 | 3893 | 5 | mg/mL | Life Chemicals |
| Maybridge3 | 7639 | 5 | mg/mL | Maybridge |
| Maybridge4 | 4576 | 5 | mg/mL | Maybridge |
| Maybridge5 | 3212 | 5 | mg/mL | Maybridge |
| Mixed commercial plate 5 | 268 | 5 | mg/mL | ChemDiv and Maybridge |

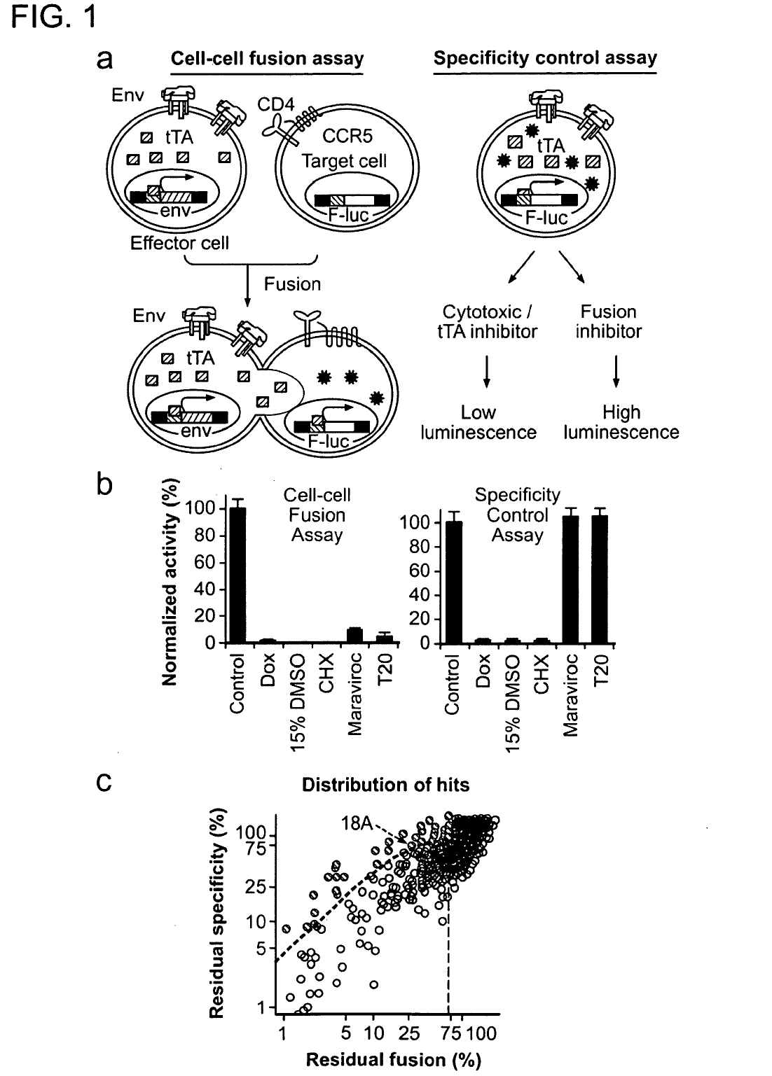

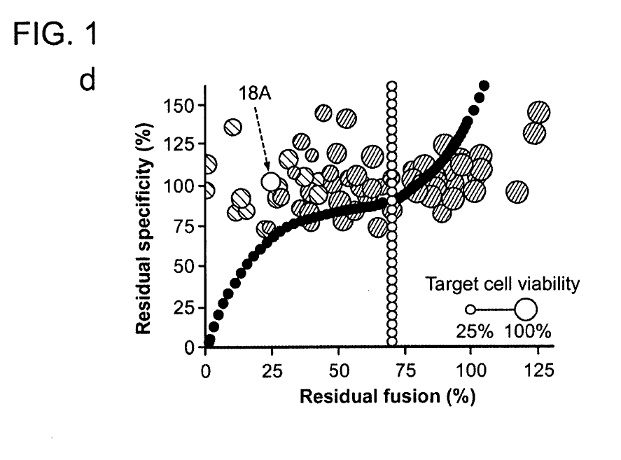

TABLE 2
Small-molecule screening data

| Category | Parameter | Description |
|---|---|---|
| Assay | Type of assay | Cell-based fusion assay |
| | Target | HIV-1 entry (see FIG. 1) |
| | Primary measurement | Luminescence |
| | Key reagents | SteadyGlo |
| | Assay protocol | See Methods |
| | Additional comments | |
| Library | Library size | See Table 1 |
| | Library composition | See Table 1 |
| | Source | See Table 1 |
| | Additional comments | |
| Screen | Format | 384-well plate |
| | Concentration(s) tested | 5 mg/ml |
| | Plate controls | Maraviroc and doxycycline |
| | Reagent/compound | Pin transfer using a Seiko robot |
| | dispensing system | |
| | Detection instrument | EnVision plate reader (Perkin Elmer) |
| | and software | |
| | Assay validation/QC | Z′ >0.7 in pilot screen and |
| | | throughout the primary screen. |
| | | Validation with known inhibitors |
| | | is shown in FIG. S1. |
| | Correction factors | None |
| | Normalization | Wells with DMSO |
| | Additional comments | |
| Post-HTS | Hit criteria | See Methods |
| analysis | Hit rate | See FIG. S2 |
| | Additional assay(s) | Viral assay |
| | Confirmation on hit | NMR of purchased compounds |
| | purity and structure | |
| | Additional comments | |

### Reagents

The following reagents were purchased from Invitrogen (Carlsbad, Calif.): Dulbecco's Modified Eagle Medium (DMEM) high glucose (cat #11965-085), Roswell Park Memorial Institute (RPMI) 1640 (cat #11875-085), DMEM high glucose without phenol red (cat #31053-028), RPMI 1640 without phenol red (cat #11835-030), Glutamax 200 mM (×100) (Cat #35050), G418 (Geneticin® Selective Antibiotic, cat #11811-031), and StemPro Accutase Cell Dissociation Reagent (cat #A11105-01). Tet System Approved PBS, US-Sourced (cat #631101) and Doxycyline (cat #631311) were purchased from Clontech (Mountain View, Calif.). Puromycin dihydrochloride from Streptomyces alboniger (cat #P8833-25MG) was purchased from Sigma-Aldrich (St. Louis, Mo.) and Steady-Glo substrate (cat #E2550) was purchased from Promega (Madison, Wis.).

### Cell Lines

H-JRFL#1336 (effector) and H-Fluc4 (control) cells were grown in DMEM containing 10% FBS, 100 μg/ml streptomycin, 100 units/ml penicillin, 200 μg/ml G418, 1 μg/ml puromycin and 2 μg/ml doxycycline. H-JRFL#13 cells carry an HIV-1_{JR-FL} env gene that is induced by growing the cells in the absence of doxycycline (Tet-Off expression system). H-Fluc4 cells, which carry a firefly luciferase gene that is induced in the absence of doxycycline, were used as specificity controls. Both cell lines were derived from HeLa cells and constitutively express the tetracycline-regulated transactivator (Tet-Off expression system). CEM#21 target cells were grown in RPMI containing 10% FBS, 100 μg/ml streptomycin, 100 units/ml penicillin and 1 μg/ml puromycin.

### Primary Screen

H-JRFL#13 or H-FLuc4 cells were washed thrice, detached with StemPro Accutase, centrifuged at 200×g for 6 minutes at 10° C. and seeded in DMEM containing 10% tetracycline-approved FBS, 100 μg/ml streptomycin, 100 units/ml penicillin, 100 μg/ml G418, 1 μg/ml puromycin and without Phenol Red. Medium was replaced after 3-6 hours to remove traces of doxycycline and cells were induced for a further 16-18 hours (40 hours for HFluc4 cells). Cells were washed and detached as above and 30 μl of 1.7×10⁵ cells/ml in RPMI_{assay} medium (RPMI containing 10% tetracycline-approved FBS, 100 μg/ml streptomycin, 100 units/ml penicillin, and without Phenol Red) were dispensed into 384-well plates. After an incubation of 2-4 hours, 100 nanoliters of the chemical compounds to be screened (the concentration of the compounds in each library is shown in Table 1) were pin-transferred to the assay plate using a Seiko robot; doxcycline (4 μg/ml) and Maraviroc (a CCR5 inhibitor) (300 nM) were added manually to control wells. CEM#21 cells were centrifuged at 150×g for 6 min and 15 μl of 8×10⁵ cells/ml in RPMI_{assay} medium were dispensed into each well of the 384-well plate. Following an incubation of 20 hours at 37° C., the plate was equilibrated to room temperature, 15 μl of Steady-Glo substrate (Promega) pre-diluted 1:1.5 in double-distilled water was added to each well, and the plate was incubated for an additional ˜30 minutes at room temperature. Firefly luciferase activity was measured using an EnVision Multilabel Plate Reader (PerkinElmer, Boston, Mass.). Cells and substrates were dispensed into the 384-well plates using a Matrix WellMate (ThermoFisher Scientific, Waltham, Mass.) and all assays were performed in duplicate.

### Secondary Screen

A confirmatory screen of selected hits was performed similarly to the primary screen, but with the following modifications: 1) test compounds were transferred using pocket tips (ThermoFisher Scientific), and 2) three assays were used in parallel: the cell-cell fusion assay, the specificity control assay, and a viability assay to measure potential cytotoxic effects of each compound on the CEM#21 cells (see viability assay below).

Analysis of screening data. For each compound, residual cell-cell fusion and residual specificity control activities measured in the primary and secondary screens were normalized using the equation:

% activity=(readout_{compound}−background)/(readout_{blank}−background)×100

In this equation, activity represents the residual activity in the cell-cell fusion assay or specificity control assay after incubation with the compound; readout_{compound}=measurement in the presence of the compound and readout_{blank}=measurement in the absence of the compound; background=measurement in the presence of doxycycline.

Readouts of duplicate measurements were used to calculate the mean and range of the % activity of each compound. Single concentration selectivity (SCS) was defined as the ratio of % Specificity:% Fusion and calculated for each compound. Compounds that resulted in: 1) % Fusion readout<(% Fusion without compound−4 standard deviations) or 72.5%; and 2) SCS<4.299−(% Fusion×0.0766)+(% Fusion²×0.0004781) were selected for the secondary screen (this equation was empirically derived to exclude highly toxic compounds and retain selective compounds, even if their inhibition activity was weak; this function is plotted in broken red symbols in FIGS. 1c and 1d). All data were processed and analyzed by a computer program written for this purpose in R.

Inhibition data were fitted to the four-parameter logistic equation using the nonlinear curve fit module in Origin 8.1 software (OriginLab, Northampton, Mass.).

Peripheral blood mononuclear cells (PBMC). Human blood was purchased from Research Blood Components, who obtained a consent firm from each donor, according to the American Association of Blood Banks guidelines, PBMC were isolated from the whole blood using a Ficoll-Paque gradient (Ficoll-Paque PLUS, Amersham Biosciences) and activated at concentration of 10⁶ live cells/ml for 3 days in RPMI-PBMC medium (RPMI-1640 supplemented with 20% FBS, 10% IL-2 (Hemagen, Columbia, Md.), 100 μg/ml primocin (InvivoGen, San Diego, Calif.)) with 4 μg/ml phytohemagglutinin (Sigma-Aldrich, St. Louis, Mo.). In some cases, cells were frozen, thawed and activated prior to the assay.

Construction of plasmids expressing JR-FL and KB9 Env chimeras. A plasmid for expression of the JR-FL gp120/KB9 gp41 Env chimera was built by digesting pCO-JRFLgp160 with XbaI and BsrGI restriction enzymes and subcloning the ˜1500-bp (˜1500-bp) JR-FL gp120-coding fragment into the same sites of pCO-KB9gp160. Similarly, an expression plasmid for the KB9 gp120/JR-FL gp41 env chimera was generated by digesting pCO-JRFLgp160 with BsrGI and AflII restriction enzymes and subcloning the ˜1100-bp JR-FL gp41-coding fragment into pCO-KB9gp160, using the same sites. This strategy results in an Env chimera in which 22 amino acids upstream of the gp120-gp41 cleavage site is derived from the gp41-donating isolate. KB9(JR-FL V1-V5) and KB9(JR-FL C1-C5) chimeras contain the variable and constant regions of JR-FL gp120 engrafted into the KB9 Env, respectively. Each gene was constructed by PCR assembly of two block gene fragments (Integrated DNA technology, Coralville, Iowa) of the corresponding sequence. An overlapping short sequence allowed assembly of the DNA fragments and the PCR product was cut with XbaI and BsrGI restriction enzymes and cloned into the same sites of pCO-KB9gp160. The constructs expressing the chimeric Envs were confirmed by restriction site and DNA sequence analysis.

HIV-1 Env mutants. Mutations were introduced into the plasmid expressing the fulllength HIV-1_{YU2} or HIV-1_{JR-FL} Envs using the QuikChange II site-directed mutagenesis protocol or the QuikChange multi site-directed mutagenesis kit (Stratagene). The presence of the desired mutations was confirmed by DNA sequencing. All HIV-1 Env residues are numbered based on alignment with the HXBc2 prototypic sequence, according to current convention.

To study CD4-independent infection, the full-length HIV-1_{ADA} N197S Env mutant was used. This Env is an ADA-HXBc2 chimera with an N197S change. The control “wild-type” ADA Env used in these experiments is also an ADA-HXBc2 chimera.

Production of recombinant HIV-1 expressing luciferase. 293T cells were cotransfected with an Env expression plasmid, a firefly luciferase-expressing lentiviral vector (pHIVcc2.luc) and an HIV-1-based packaging plasmid (psPAX2, cat #11348, NIH AIDS Research and Reference Reagent Program) at a ratio of 1:6:3 using Effectene (Qiagen, Germantown, Md.). After 48 hours, the supernatant was collected, buffered with 50 mM Hepes pH 7.4 (final concentration) and centrifuged for 5 minutes at 750×g and 4° C. The virus-containing supernatant was used directly or frozen at −80° C.

Viral infection assay. Each test compound was serially diluted in DMSO in a 96-well B&W isoplate (PerkinElmer, Boston, Mass.) using an HP D300 Digital Dispenser, to a final volume of 450 nl. DMSO was used as a control. Viruses pseudotyped with a specific Env were added to each well and incubated briefly at room temperature. Cf2Th-CD4/CCR5 cells were detached with StemPro Accutase Cell Dissociation Reagent (Invitrogen, cat #A11105-01), washed once, and 5000 cells were added to each well. After 3-4 hour incubation at 37° C. the viruses and compounds were removed, the medium was replaced and the cells were further incubated for a total of 24-30 hours (in a few cases, cells were incubated for a total of 44 hours; when the experiment was repeated with an incubation period of 30 hours, no differences in 18A inhibition were observed). Following incubation, the medium was aspirated and cells were lysed with 30 μl of Passive Lysis Buffer (Promega, cat #E1941). The activity of the firefly luciferase, which was used as a reporter protein in the system, was measured with a Centro LB 960 luminometer (Berthold Technologies, Oak Ridge, Tenn.). One hundred microliters of assay buffer (15 mM MgSO₄, 15 mM KH₂PO₄/K₂HPO₄ pH 7.8, 1 mM ATP and 1 mM DTT) was injected to each well, followed by a 50 μl injection of 1 mM Dluciferin potassium salt (BD Pharmingen, San Jose, Calif.); luminescence was measured for 2 sec. Infection of PBMC was done as above, but 20,000-40,000 cells/well and viruses concentrated by ultracentrifugation were used. After four hours of incubation with viruses, the cells were centrifuged at 200×g for 6 minutes at 4° C., resuspended in 100 μl RPMI-PBMC medium and incubated at 37° C. for an additional 36-40 hours (total 40-44 hours). Supernatant was removed after centrifugation at 400×g for 5 minutes and cells were processed to detect luciferase activity, as described above.

For washout experiments, different concentrations of 18A were incubated with the recombinant viruses at 37° C. for 20 minutes. The viruses were laid on a 20% sucrose cushion and ultracentrifuged at 30,000 RPM in an SW55 rotor for 1.5 hours at 4° C. After the supernatant was aspirated, viruses were suspended in 500-1000 μl medium and 90 μl of the virus suspension was used to infect 5000 Cf2Th-CD4/CCR5 cells (5 replicates). The cells were incubated for 48 hours and processed as described above. Sensitivity of recombinant viruses to cold inactivation was measured.

Viability Assay. As part of the secondary screen, cells were incubated for ˜20 hours in 384-well plates in a final volume of 45 μl growth medium at 37° C. The plate was equilibrated to room temperature, 15 μl of CellTiterGlo (Promega) was added to each well and luminescence was measured as described above. The viability assay with 18A was done in parallel to the viral infection assay for the same length of time. 18A was diluted using an HP D300 Digital Dispenser in a 96-well B&W isoplate and cells were added. After incubation of 26-30 hours (Cf2Th cells) or 40-44 hours (PBMC). 30 μl (Cf2Th cells) or 100 μl (PBMC) of CellTiterGlo was added and luminescence was measured as described above.

Enzyme-linked immunosorbent assay (ELISA). A white, high-binding microliter plate (Corning) was coated by incubating 0.125 μg of mouse anti-polyhistidine antibody (Catalog no. sc-53073, Santa Cruz Biotechnology) diluted to a final concentration of 1.25 μg/ml in 100 μl PBS in each well overnight. Wells were blocked with blocking buffer (5% nonfat dry milk (Bio-Rad) in PBS) for 2 hours and then washed twice with PBS. Between 0.25 and 0.5 μg of purified HIV-1_{JR-FL} gp120 in blocking buffer was added to each well; the plate was incubated for 60 minutes and washed thrice with PBS. Eighty microliters of either DMSO or 18A (at concentrations of either 21.4 or 86 μM) in blocking buffer were added to the wells and after a 30-minute incubation, 20 μl of the specified antibody in blocking buffer was added. The plate was incubated for a further 30 minutes and washed thrice with 0.05% Tween in PBS and thrice with PBS. Peroxidase-conjugated F(ab′)₂ fragment donkey anti-human IgG (1:3,600 dilution; catalog no. 706-036-098; Jackson ImmunoResearch Laboratories) in blocking buffer was added to each well. The plate was incubated for 30 minutes, washed three times with 0.05% Tween in PBS and three times with PBS, as before, and 80 μl of SuperSignal chemiluminescent substrate (Pierce) was added to each well. The relative light units in each well were measured for 2 sec with a Centro LB 960 luminometer (Berthold Technologies). All procedures were performed at room temperature. Cellbased ELISA was performed.

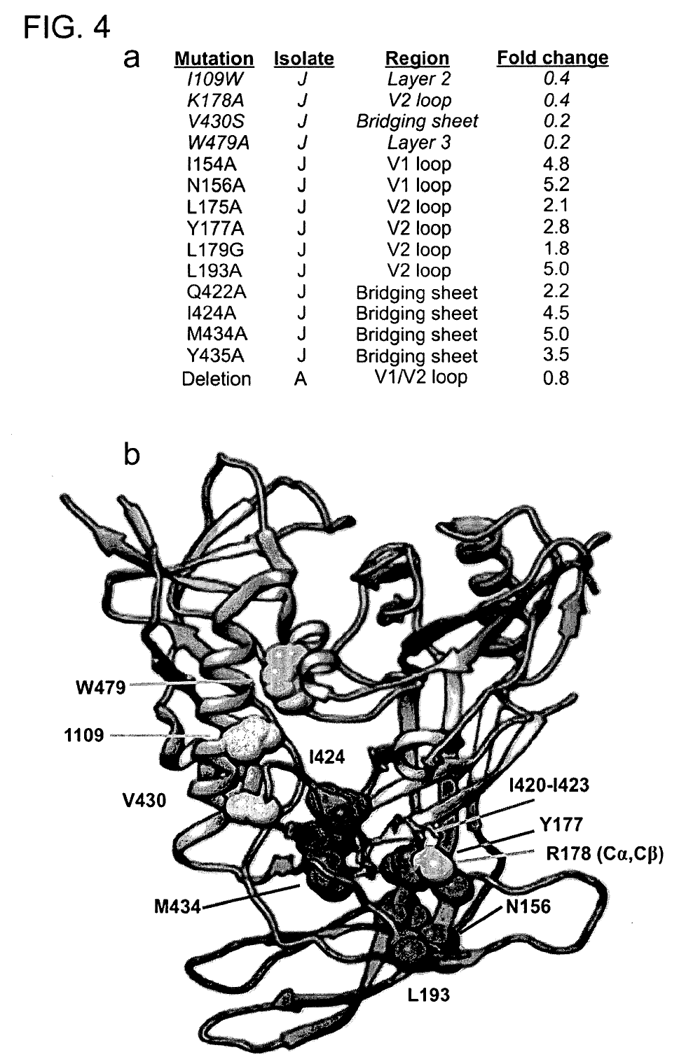

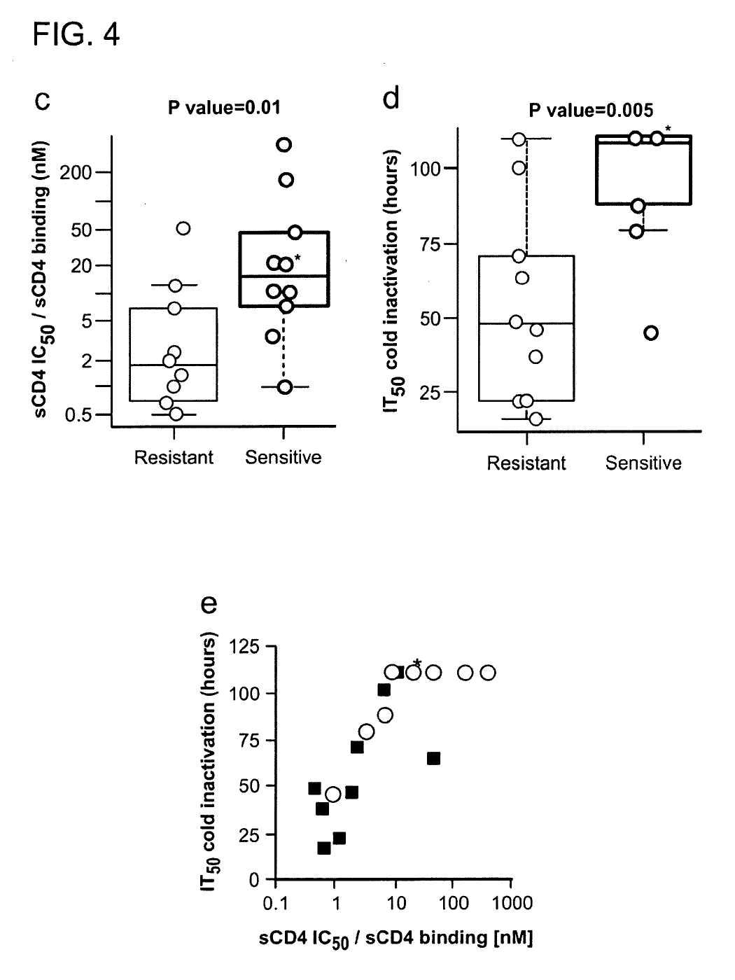

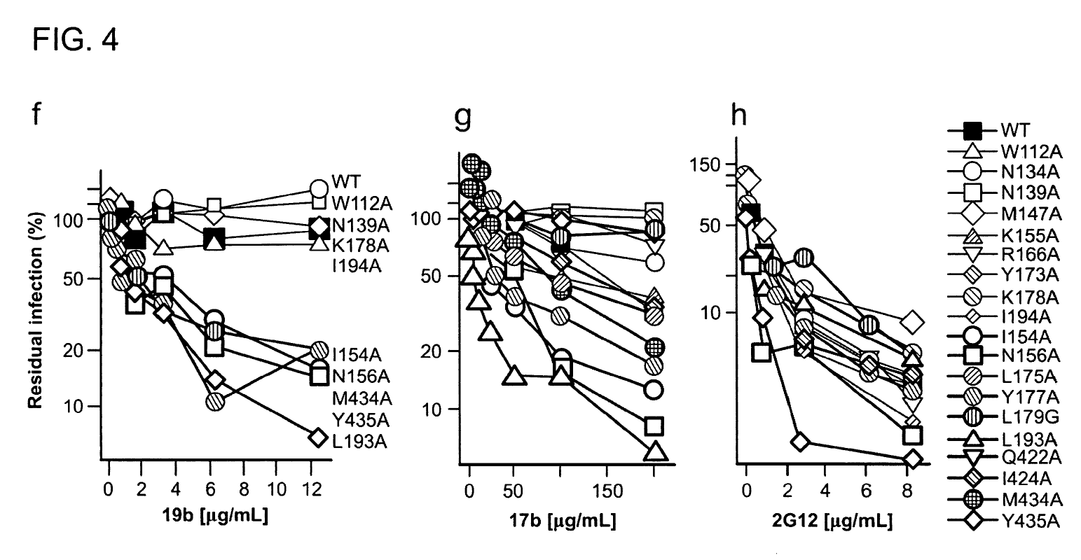

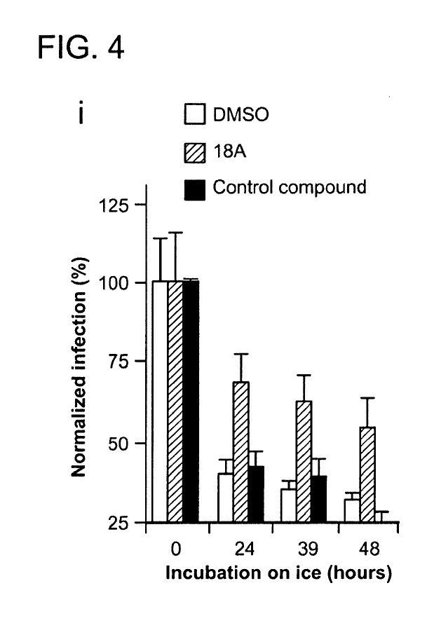

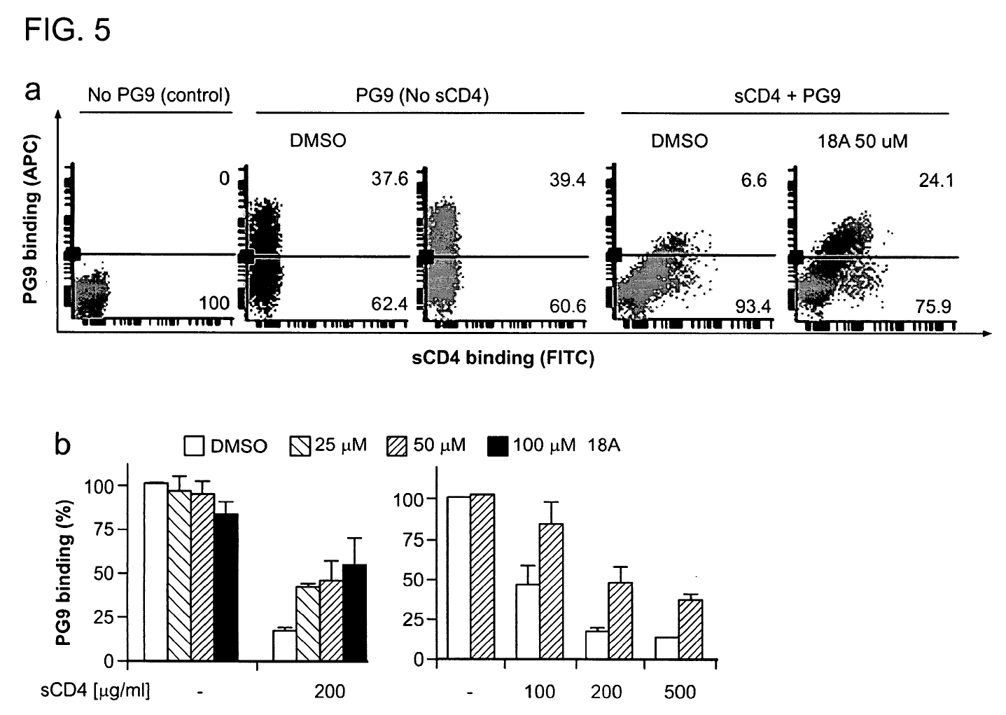

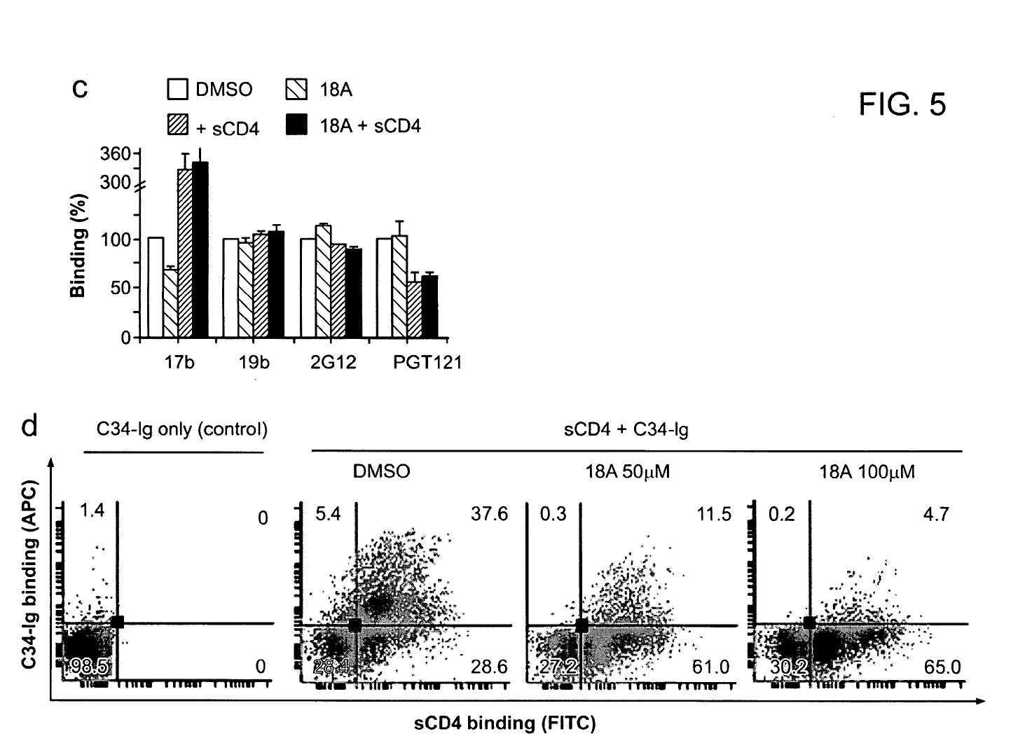

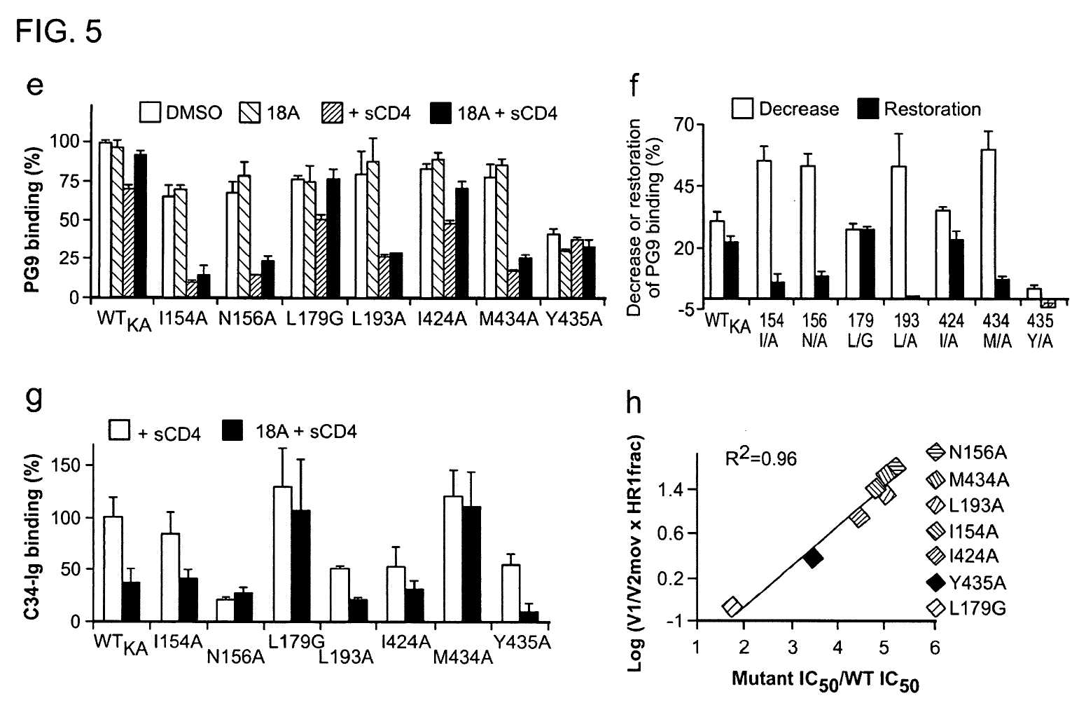

Flow cytometry. Plasmids expressing the wild-type HIV-1_{JR-FL}ΔCT Env or the double mutant HIV-1_{JR-FL} E168K+N188AΔCT Env were transfected with the Effectene transfection reagent (Qiagen) into 293T cells, according to the manufacturer's instructions. After 48-72 hours, cells were detached with 5 mM EDTA/PBS and between 0.5-1 million cells were briefly incubated with various concentrations of 18A and then with or without the indicated concentrations of sCD4. C34-Ig (at a final concentration of 20 μg/ml) or a specified antibody (at final concentration of 1 μg/ml) was added to the cells. After a 30-minute incubation, the cells were washed twice and incubated with Allophycocyanin-conjugated F(ab′)₂ fragment donkey anti-human IgG antibody (1:100 dilution; catalog no. 709-136-149; Jackson ImmunoResearch Laboratories) and Fluorescein isothiocyanate-conjugated anti-CD4 antibody (1:33 dilution, E-biosciences) for 15 minutes. Cells were washed twice and analyzed with a BD FACSCanto II flow cytometer (BD Biosciences). For analysis of resistant mutants (FIG. 5e-g), the measurements were normalized for the level of sCD4 binding of each mutant, and the response of each mutant was further normalized to that of the wild-type HIV-1_{JR-FL} E168K+N188AΔCT Env (herein designated WT_{KA}). In the absence of sCD4, the binding of C34-Ig to cells expressing the wild-type and mutant Envs was similar to that of the control cells without Env. The sCD4 IC₅₀/sCD4 binding ratio (FIGS. 4c and e) was calculated as follows. Binding of sCD4 to different envelope mutants was measured by flow cytometry and normalized first to 2G12 binding to account for potentially different Env expression levels, and then to sCD4 binding to the wild-type HIV-1_{JR-FL} Env. The IC₅₀ of sCD4 for inhibition of virus infection by each mutant Env was divided by the normalized sCD4 binding to Env-expressing cells to calculate the sCD4 IC₅₀/sCD4 binding value.

Binding of soluble JR-FL gp120 to CCR5 was tested by incubating 1 million Cf2Th-CCR5 cells with 20 μg/mL purified gp120 in the presence or absence of 20 μg/mL sCD4 for 1 hour. After two washes, the cells were incubated with the 2G12 antibody followed by Allophycocyanin-conjugated F(ab′)₂ fragment donkey anti-human IgG antibody to detect bound gp120. The cells were analyzed by FACS. All procedures were performed at room temperature.

### Example 2

### Selectivity Analysis Identifies HIV-1 Fusion Inhibitors

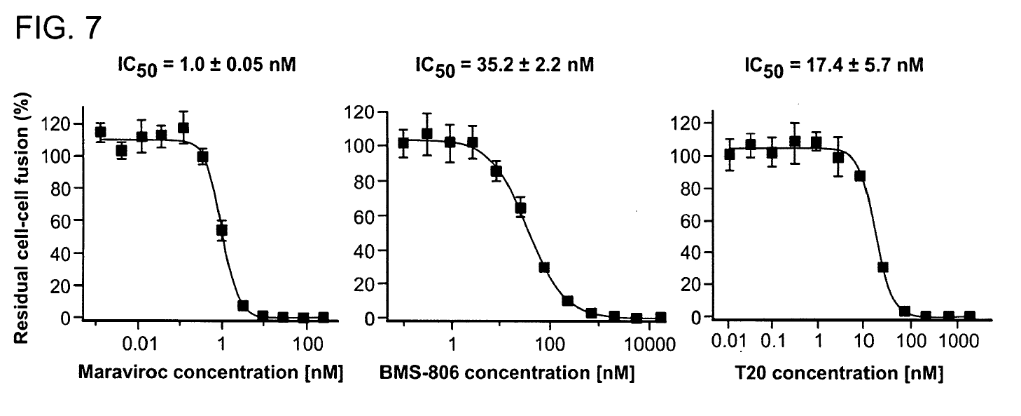

To identify new molecules that potentially affect HIV-1 entry, a cell-cell fusion assay that mimics the entry of HIV-1 into cells was established (FIG. 1a). The assay uses a firefly luciferase (F-luc) readout to measure the fusion of HeLa effector cells that express the Envs from a primary HIV-1 strain and target cells coexpressing the CD4 and CCR5 receptors. As a control assay designed to evaluate the specificity of each compound, HeLa cells were induced to express the F-luc reporter protein. The two assays were validated with known inhibitors, confirming that off-target compounds decreased the readout of both assays, whereas known HIV-1 entry inhibitors selectively inhibited the fusion assay (FIG. 1b and FIG. 7). Thus, fusion inhibitors could be distinguished from cytotoxic and non-specific compounds by combining the two assays.

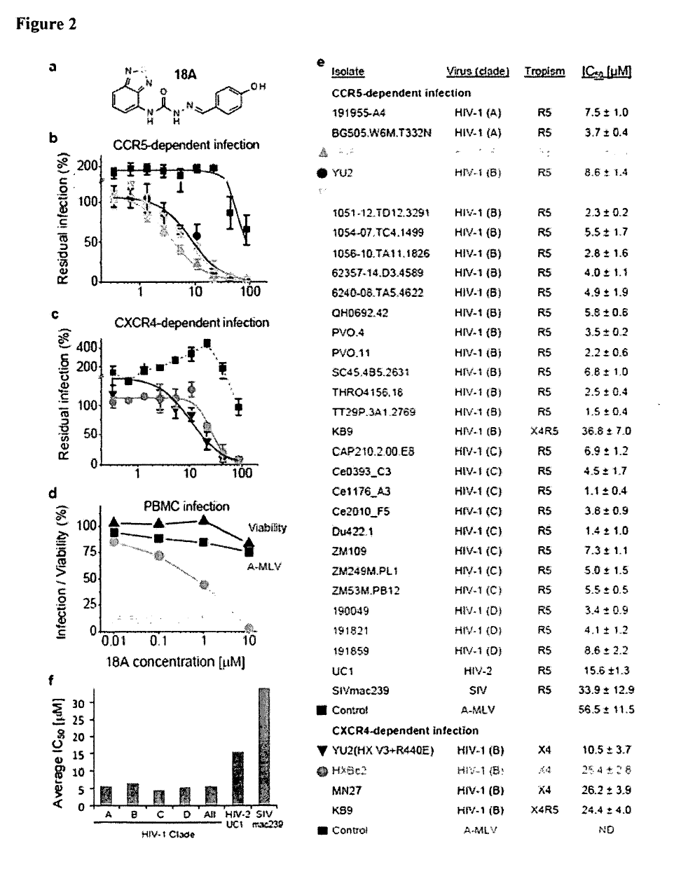

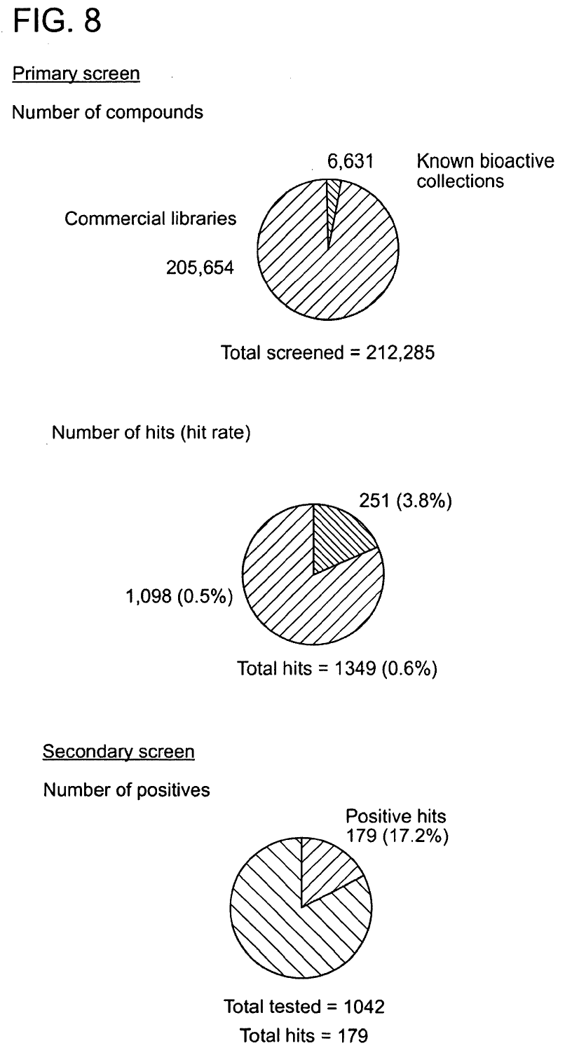

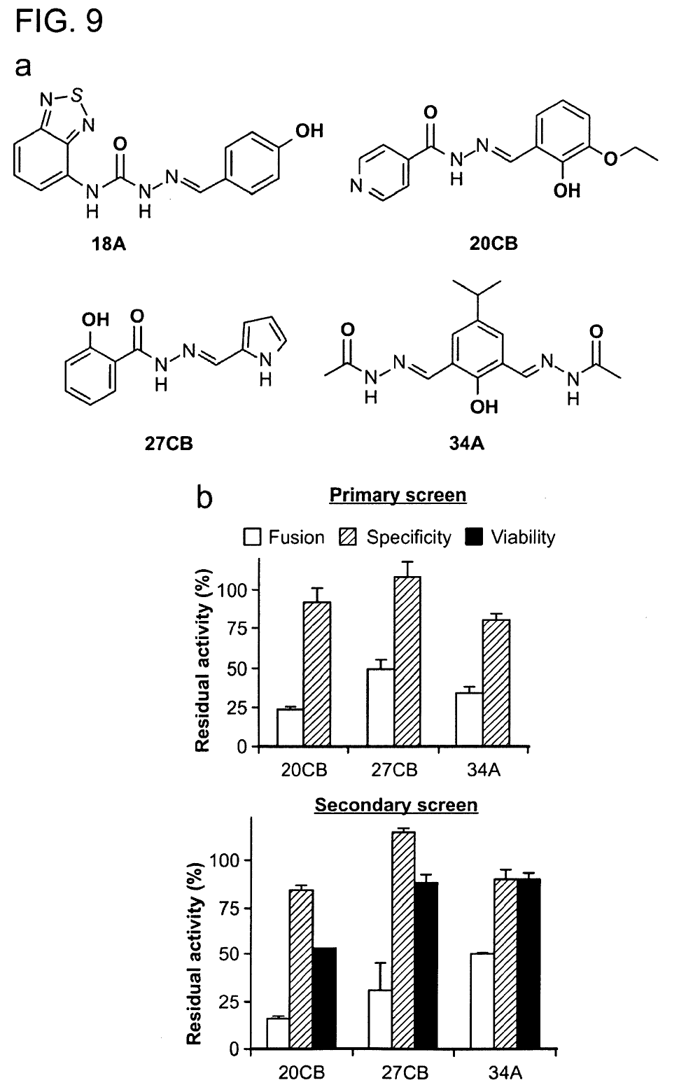

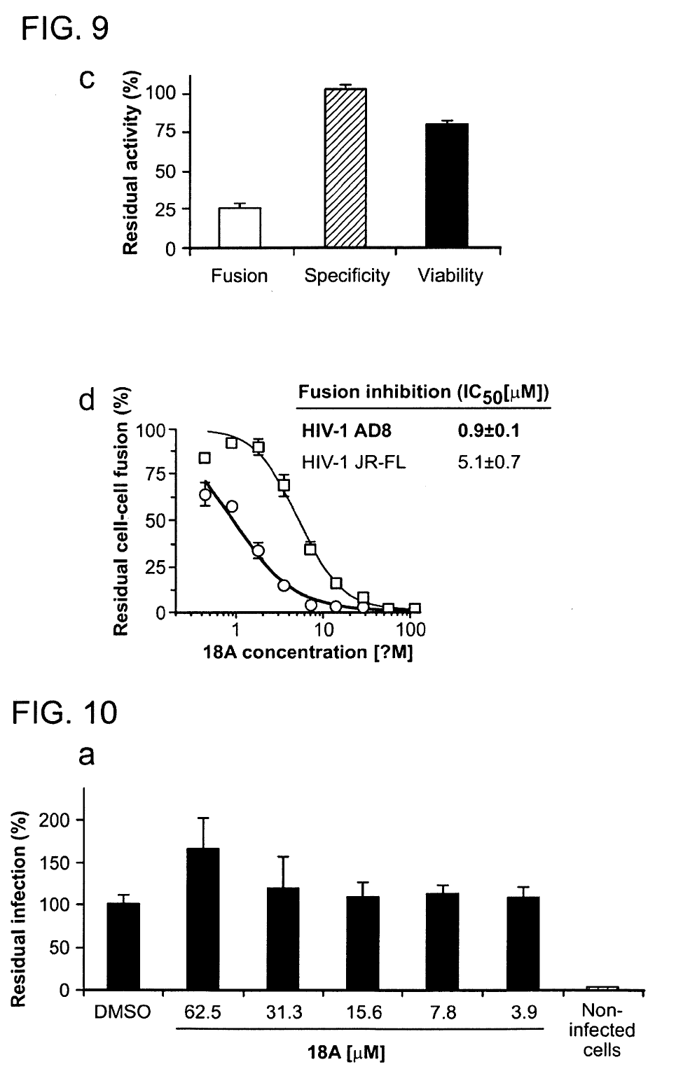

The cell-cell fusion and control assays were used in parallel to screen 212,285 compounds (Table 1 and FIG. 8), and readouts from the two assays were integrated to analyze the activity of each compound. Plotting the effect of each compound on the control readout versus its effect on the fusion readout allowed a comparison of the selective inhibition of the compounds (FIG. 1c). Fusion inhibitors that exhibited high specificity localized in the top left portion of the plot; these were identified by using an inhibitory cutoff to sort active compounds and a selectivity threshold to retain the most specific ones (FIG. 1c). Compounds satisfying both criteria were retested (FIG. 8) and a group of compounds, which share a common acyl hydrazone core and an adjacent aromatic ring (FIG. 1d and FIG. 9), was identified. The most effective of these, 18A, specifically inhibited cell-cell fusion and HIV-1 infection mediated by HIV-1_{JR-FL} and HIV-1_{ADS} Envs (FIG. 2b and FIG. 9).

### Example 3

### Compound 18A Inhibits a Wide Spectrum of CCR5- and CXCR4-Tropic HIV-1

The effect of 18A on viral infection was tested using recombinant HIV-1 pseudotyped with the envelope glycoproteins of different primate immunodeficiency viruses or the amphotropic murine leukemia virus (A-MLV) as a control. 18A efficiently inhibited infection of all CCR5-tropic (R5) HIV-1 tested, including viruses from phylogenetic clades A, B, C and D (FIGS. 2b and e). 18A also inhibited infection of viruses with HIV-2_{OC1} and SIV_{mac239} Envs, although the inhibition was significantly less potent than that of most viruses with HIV-1 Envs (FIG. 2e). HIV-1_{JR-FL}, which was used for the initial screen, was one of the most sensitive strains with a half-maximal inhibitory concentration (IC₅₀) value of 3.6 μM, whereas the dual-tropic HIV-1_{KB9} isolate was relatively resistant to inhibition (FIG. 2e). Notably, 18A effectively inhibited a wide spectrum of diverse HIV-1 strains, including transmitted/founder and primary isolates, with an average IC₅₀ of 5.7 μM for all CCR5-using HIV-1 isolates, and with the majority (75%) of these isolates showing IC₅₀ values less than 6 μM (FIG. 2f). The half-maximal cytotoxic concentration (CC₅₀) of 18A for the CD4/CCR5-expressing target cells in the assay was 44 μM, consistent with the observed IC₅₀ of 56 μM for the control A-MLV (FIGS. 2b and e).

The effect of 18A on CXCR4-tropic (X4) viruses was tested using Cf2Th target cells expressing CD4 and CXCR4. The laboratory-adapted HIV-1_{HXBc2} and HIV-1_{MN27} from clade B showed similar inhibition profiles, with IC₅₀ values of ˜25 μM (FIGS. 2c and e). Similar concentrations of 18A were required to inhibit the dual-tropic HIV-1_{KB9} isolate in CD4/CXCR4-positive target cells (FIG. 2e). The measured CC₅₀ of 18A was 63 μM, confirming that the observed 18A inhibition of HIV-1 infection of cells expressing CD4 and CXCR4 is specific. Of note, 18A inhibited infection of Cf2Th-CD4/CXCR4 cells by the chimeric YU2(HXV3+R440E) virus with an IC₅₀ similar to that of the parental R5 YU2 virus infecting Cf2Th-CD4/CCR5 cells; therefore, infection of CXCR4-expressing cells by X4 viruses is not necessarily less sensitive to 18A inhibition than infection of CCR5-expressing cells by R5 viruses. Moreover, as CCR5 and CXCR4 are structurally distinct, 18A inhibition of HIV-1 infection is unlikely to depend on binding these coreceptors.

The activity of 18A in primary CD4+ T cells (human PBMC), which express lower levels of CD4 and CCR5 on their surface compared to the Cf2Th cells and represent more physiologically-relevant target cells, was tested. Inhibition of HIV-1_{JR-FL} infection by 18A was even more potent under these conditions with an IC₅₀ of 0.4 μM (FIG. 2d). No significant effect on A-MLV infection or on the viability of the cells was observed within the range of tested concentrations (FIG. 2). In summary, 18A exhibited broad-range and specific inhibition of CCR5- and CXCR4-tropic HIV-1.

### Example 4

### Target for 18A Inhibition

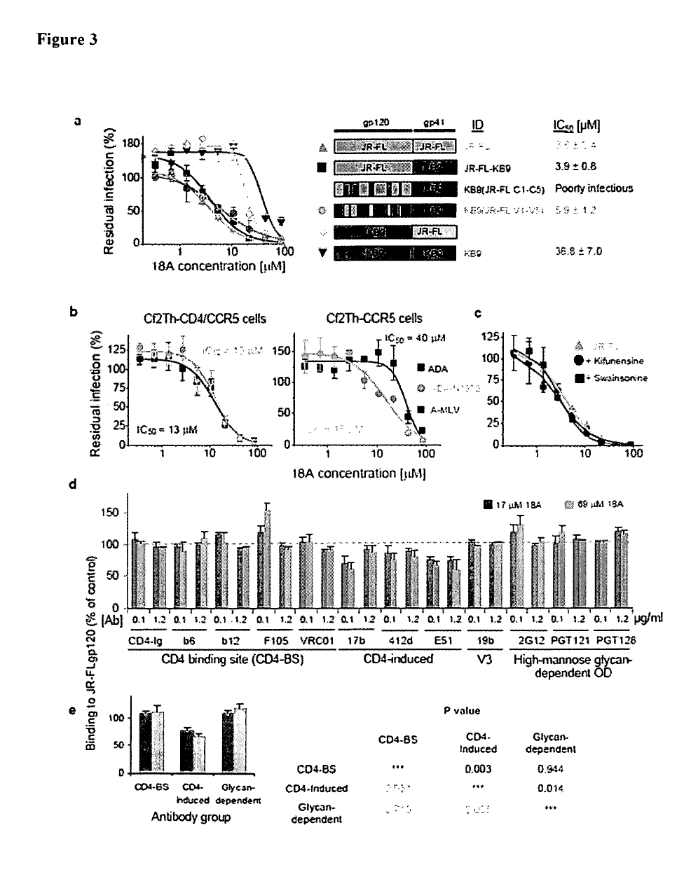

To study the target of 18A inhibition, chimeric Envs between the Env of HIV-1_{JR-FL}, one of the most sensitive strains, and that of HIV-1_{KB9}, the most resistant strain, were constructed. An Env with the JR-FL gp120 and the KB9 gp41 was nearly as sensitive as the parental JR-FL Env to inhibition by 18A (FIG. 3a). By contrast, an Env containing the KB9 gp120 and the JR-FL gp41 was about 5-fold more resistant to 18A than the JR-FL Env, and slightly more sensitive (2-fold) than the parental KB9 Env. Thus, gp120 is the major determinant of sensitivity to 18A. A chimera in which the major variable loops of JR-FL gp120 were grafted onto the KB9 Env was nearly as sensitive to 18A inhibition as JR-FL, indicating that the major variable regions of gp120 significantly contribute to 18A sensitivity (FIG. 3a).

The contribution of CD4 to inhibition was measured by testing the effect of 18A on the infection of a CD4-independent virus. Productive infection of wild-type HIV-1_{ADA} requires expression of both CD4 and CCR5 on target cells, but the HIV-1_{ADA} N197S Env mutant does not require CD4 and infects CCR5-expressing target cells. The entry of both isolates into CD4/CCR5-expressing cells was blocked by 18A with a similar profile of inhibition, indicating a comparable susceptibility of both viruses to 18A (FIG. 3b). Moreover, 18A protected CD4-negative, CCR5-expressing cells from infection by HIV-_{ADA} N197S, demonstrating that inhibition does not depend on the presence of CD4.

To test possible contacts of 18A with complex glycans HIV-1 Env, HIV-1 to virions were produced in the presence of two glycosidase inhibitors. Neither treatment had a significant effect on 18A inhibition of viruses with the JR-FL Env (FIG. 3c). Apparently, complex glycans are not required for the binding of 18A to the envelope glycoproteins or for HIV-1 inhibition by 18A.

### Example 5

### Reversible Interaction of Compound 18A with gp120

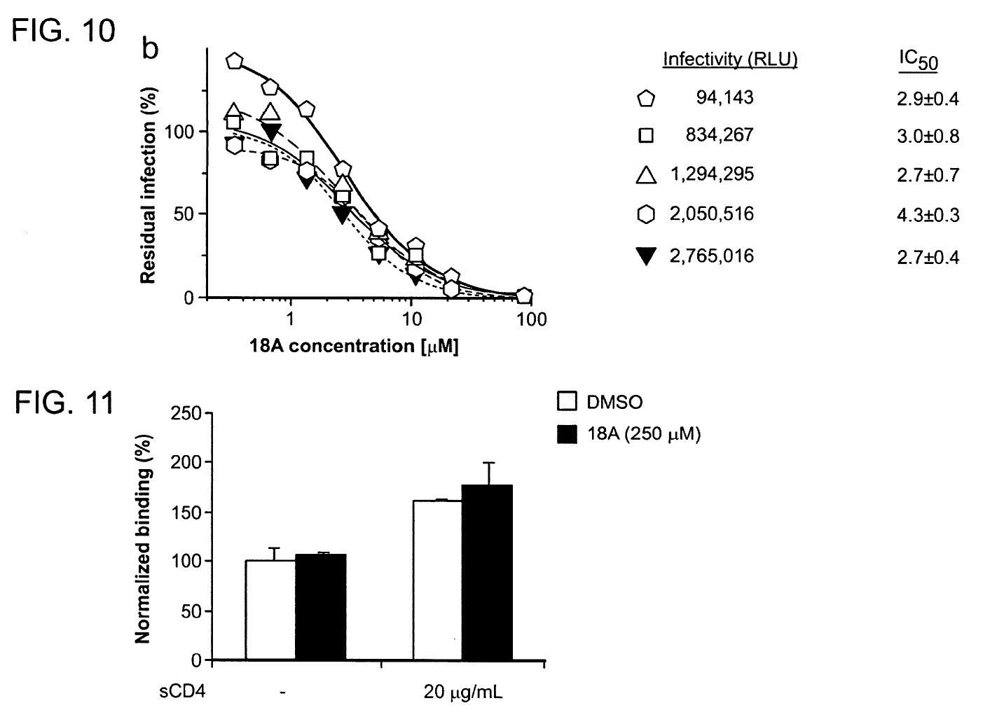

Inhibition of HIV-1 infection by 18A was reversible. Washing out 18A before infection with HIV-1 virions alleviated any blocking effect of the inhibitor (FIG. 10a). In addition, similar IC₅₀ values were measured for different levels of infection by HIV-1 (FIG. 10b). These results are consistent with a reversible mechanism of inhibition.

To study the interaction of 18A with gp120, the binding of a panel of monoclonal antibodies with known epitopes 19-22 to HIV-1 gp120 was studied in the presence or absence of 18A. The binding of most antibodies was not affected by preincubation of gp120 with 18A (FIG. 3d). A modest but reproducible decrease in the binding of the E51, 17b and 412d antibodies was detected. The E51, 412d and 17b antibodies bind discontinuous CD4-induced (CD4i) gp120 epitopes that overlap the CCR5-binding site and include the highly conserved sequence IKQI (residues 420-423) located in the β20 strand of gp120. Evaluating each group of antibodies separately showed that the of 18A on the CD4-induced antibodies was unique and statistically significant (FIG. 3e).

The effect of 18A on the binding of gp120 to the CCR5 coreceptor in the absence and presence of soluble CD4 was tested (FIG. 11). No effect of 18A on CCR5 binding was observed.

### Example 6

### Sensitivity and Resistance of HIV-1 gp120 Mutants to 18A

To investigate the interaction of 18A with HIV-1 Env, we tested the sensitivity of a large panel of HIV-1_{JR-FL} and HIV-1_{YU2} Env mutants to inhibition by 18A. Consistent with its wide spectrum of inhibition of primary HIV-1 isolates (FIG. 2), 18A inhibited ail of the mutants, including several BMS-806-resistant mutants, to some extent (Table 3).

TABLE 3
The effect of 18A on infectivity of different HIV-1 mutants

| | | Secondary | | Fold |
|---|---|---|---|---|
| | Region | structure | IC₅₀ [μM]^{b} | change^{c} |
| JR-FL single mutants^{a} | | | | |
| Wild type | | | 3.6 ± 0.4 | 1 |
| I109W | C1 | α1 | 1.6 ± 0.8 | 0.4 |
| W112A | C1 | α1 | 2.8 ± 0.7 | 0.8 |
| Q114E | C1 | α1 | 3.4 ± 0.6 | 0.9 |
| V134A | V1 | | 2.8 ± 2.1 | 0.8 |
| N136A | V1 | | 1.8 ± 0.1 | 0.5 |
| N139A | V1 | | 1.9 ± 0.2 | 0.5 |
| N141A | V1 | | 1.7 ± 0.3 | 0.5 |
| M147A | V1 | | 4.8 ± 1.2 | 1.3 |
| E153A | V1 | | 3.1 ± 0.7 | 0.9 |
| I154A | V1 | | 17.1 ± 0.9 | 4.8 |
| K155A | V1 | | 2.1 ± 0.9 | 0.6 |
| N156A | | | 18.7 ± 1.5 | 5.2 |
| R166A | V2 | | 2.9 ± 0.6 | 0.8 |
| D167A | V2 | | 5.3 ± 1.5 | 1.5 |
| Y173A | V2 | | 3.1 ± 1.2 | 0.9 |
| L175A | V2 | | 7.7 ± 1.2 | 2.1 |
| Y177A | V2 | | 10.2 ± 1.0 | 2.8 |
| K178A | V2 | | 1.6 ± 0.3 | 0.4 |
| L179G | V2 | | 6.5 ± 0.7 | 1.8 |
| N188A | V2 | | 4.2 ± 0.8 | 1.2 |
| L193A | V2 | | 18.1 ± 2.0 | 5.0 |
| I194A | V2 | | 1.8 ± 0.4 | 0.5 |
| Q422A | C4 | β20 | 7.8 ± 1.7 | 2.2 |
| I424A | C4 | β20 | 16.2 ± 2.0 | 4.5 |
| V430S | C4 | β21 | 0.7 ± 0.3 | 0.2 |
| M434A^{b} | C4 | β21 | 17.9 ± 1.2 | 5.0 |
| Y435A | C4 | β21 | 12.7 ± 3.5 | 3.5 |
| W479A | C5 | α5 | 0.8 ± 0.2 | 0.2 |
| JR-FL double mutants^{a} | | | | |
| T143A + S146A | V1 | | 2.4 ± 0.2 | 0.7 |
| T163A + S164A | V2 | | 2.7 ± 0.3 | 0.8 |
| E168K + N188A | V2 | | 2.4 ± 1.4 | 0.7 |
| N187A + N188A | V2 | | 2.5 ± 0.6 | 0.7 |
| ADA^{a} | | | | |
| Wild type | | | 12.8 ± 1.5 | 1 |
| ΔV1V2 | V1/V2 | | 10.8 ± 2.0 | 0.8 |
| YU2 single mutants^{a} | | | | |
| Wild type | | | 8.6 ± 1.4 | 1 |
| H66A | C1 | α0 | 8.0 ± 0.9 | 1 |
| H66N | C1 | α0 | 4.6 ± 0.5 | 0.5 |
| W69L | C1 | α0 | 3.5 ± 0.5 | 0.4 |
| T71A | C1 | α0 | 7.1 ± 1.4 | 0.8 |
| I108A | C1 | α1 | 6.1 ± 0.7 | 0.7 |
| I109W | C1 | α1 | 2.9 ± 0.4 | 0.3 |
| S110A | C1 | α1 | 7.3 ± 1.6 | 0.9 |
| S110N | C1 | α1 | 5.9 ± 1.8 | 0.7 |
| L111A | C1 | α1 | 10.4 ± 1.6 | 1.2 |
| D113A | C1 | α1 | 6.0 ± 1.4 | 0.7 |
| Q114N | C1 | α1 | 14.8 ± 2.1 | 1.7 |
| K117W | C1 | | 6.5 ± 1.1 | 0.8 |
| P212A | C2 | | 10.2 ± 1.5 | 1.2 |
| V255A | C2 | Loop-B | 14.0 ± 1.2 | 1.6 |
| V255G | C2 | Loop-B | 7.9 ± 1.7 | 0.9 |
| V255I | C2 | Loop-B | 5.4 ± 1.3 | 0.6 |
| V255W | C2 | Loop-B | 3.6 ± 1.3 | 0.4 |
| T257A | C2 | Loop-B | 13.2 ± 1.1 | 1.5 |
| T257S | C2 | Loop-B | 6.9 ± 0.8 | 0.8 |
| L260A | C2 | Loop-B | 3.4 ± 1.5 | 0.4 |
| L261A | C2 | β9 | 4.9 ± 1.0 | 0.6 |
| I285A | C2 | β11 | 6.3 ± 1.5 | 0.7 |
| I309A | V3 | | 4.2 ± 1.3 | 0.5 |
| L317A | V3 | | 10.2 ± 1.4 | 1.2 |
| E370A | C3 | α3 | 2.3 ± 0.8 | 0.3 |
| I371A | C3 | α3 | 6.5 ± 0.9 | 0.8 |
| S375A | C3 | β16 | 10.0 ± 1.3 | 1.2 |
| S375W^{d} | C3 | β16 | 14.6 ± 2.8 | 1.7 |
| N377A | C3 | β16 | 3.3 ± 0.6 | 0.4 |
| I420C | C4 | β19 | 2.1 ± 0.4 | 0.2 |
| I420S | C4 | β19 | 4.8 ± 0.8 | 0.6 |
| I423R | C4 | β20 | 2.4 ± 0.4 | 0.3 |
| E429K | C4 | | 9.0 ± 1.4 | 1.1 |
| V430A | C4 | β21 | 5.9 ± 0.6 | 0.7 |
| V430S | C4 | β21 | 2.9 ± 0.3 | 0.3 |
| M475A^{d} | C5 | α5 | 10.2 ± 2.6 | 1.2 |
| W479A | C5 | α5 | 1.3 ± 0.2 | 0.2 |
| D589L | C5 | | 12.1 ± 1.5 | 1.4 |
| W596M | C5 | | 9.4 ± 1.4 | 1.1 |
| YU2 double mutants^{a} | | | | |
| W69L + S375W | C1 + C3 | α0 + β16 | 15.0 ± 2.5 | 1.7 |
| HXBc2-YU2 Chimeras^{a} | | | | |
| (CCR5-tropic) | | | | |
| HXBc2 (YU2 V3) | | | 20.2 ± 2.2 | |
| HXBc2 (YU2 V123) | | | 12.5 ± 1.6 | |

^{a}Recombinant HIV-1 pseudotyped with the indicated Envs was tested for inhibition by 18A; all Envs tested in the virus inhibition assay contain a complete gp41 cytoplasmic tail.
^{b}Inhibition data from 2-5 independent experiments, each performed in triplicate, were averaged. IC₅₀s were calculated by fitting the data to the four-parameter logistic equation.
^{c}Fold change in susceptibility is the ratio of mutant to wild-type IC₅₀ values.
^{d}Changes in these residues are associated with resistance to BMS-806.

Hypersensitivity of several mutants to 18A was observed, with IC₅₀ values decreasing to 5-fold lower than that of the corresponding wild-type Env. Changes associated with hypersensitivity mapped to the α1 and α5 helices of the inner domain, the V2 region, and the β20-β21 element of gp120. Up to 5.2-fold resistance to 18A was also detected and was associated with changes in two regions of gp120: the β20-β21 strands and the V1/V2 variable region. Of interest, the β20-β21 and V1/V2 variable regions are proximal on the available models of the Env trimer (FIG. 4b).

To explore the basis of resistance, the effect of the changes associated with 18A resistance and sensitivity on HIV-1 Env reactivity was explored. Env reactivity describes the propensity of Env to change conformation from the metastable unliganded state to downstream conformations such as the CD4-bound state. HIV-1 variants with high Env reactivity typically exhibit increased sensitivity to inactivation by soluble CD4 (sCD4), antibodies, and incubation in the cold. The susceptibility of the 18A-sensitive and 18A-resistant mutants to soluble CD4 (sCD4), cold and antibodies was examined. Resistance to 18A inhibition correlated with sCD4 reactivity and with cold sensitivity (FIGS. 4e and d). Evaluating the presence of both properties in each mutant suggests that 18A-resistant viruses generally exhibit high Env reactivity, with enhanced sensitivity to sCD4 inhibition and to cold inactivation (FIG. 4e). This implies a preference of 18A for the unliganded state of HIV-1 Env.

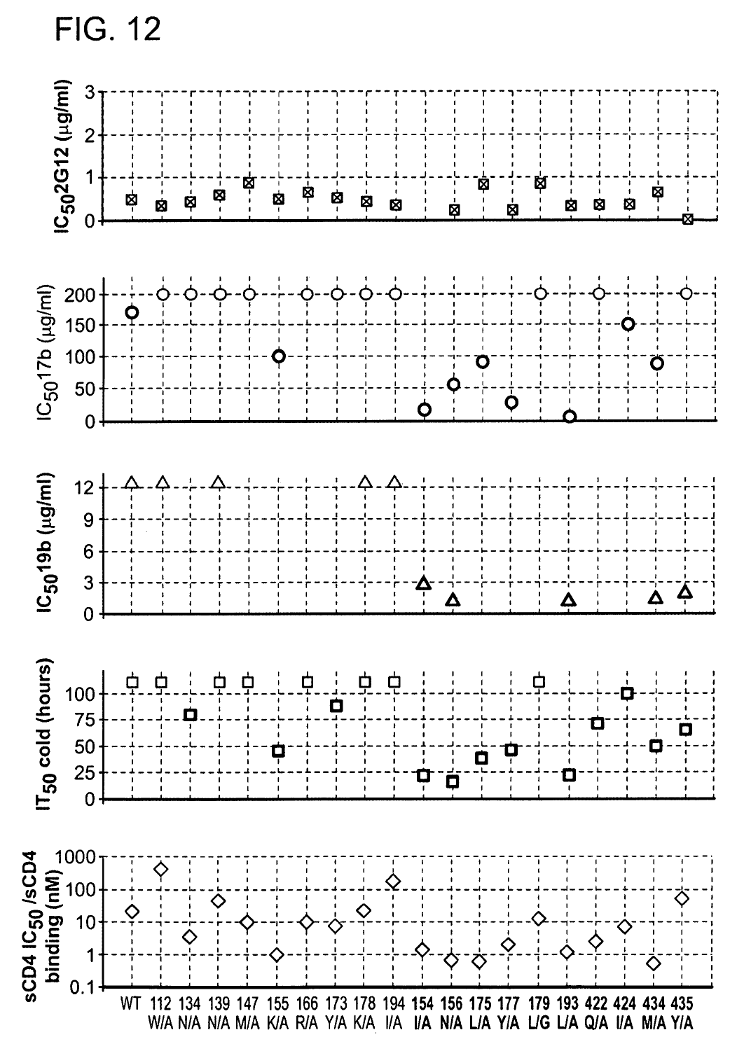

The higher reactivity of 18A-resistant Env mutants predicts that they will more readily assume the CD4-bound conformation. Thus, 18A-resistant viruses should be more sensitive to neutralization by antibodies directed against the CD4-induced (CD4i) and V3 epitopes, which overlap the coreceptor-binding site of gp120 that is induced by CD4 binding. Compared with 18A-sensitive Env variants, the 18A-resistant Env mutants were significantly more sensitive to neutralization by the CD4i antibody, 17b, and the V3-directed antibody, 19b (FIGS. 4f and g). The 2G12 antibody, which is minimally affected by changes in HIV-1 Env reactivity, neutralized both 18A-sensitive and 18A -resistant viruses equivalently (FIG. 4h). The enhanced sensitivity of 18A-resistant mutants to 17b and 19b neutralization supports the hypothesis that the 18A -resistant mutants exhibit higher Env reactivity and are more prone to sample the CD4-bound conformation (FIG. 12).

Some HIV-1_{AD8} Env variants that were previously shown to differ in Env reactivity were nonetheless equally sensitive to 18A inhibition (FIG. 13). Therefore, increases in Env reactivity do not necessarily lead to 18A resistance; the 18A-resistant HIV-1 mutants identified herein thus represent a subset of Env variants with high Env reactivity.

### Example 7

### Effect of 18A on HIV-1 Env Conformation and Receptor-Induced Changes

To gain insight into the mechanism of 18A inhibition of HIV-1 entry, the effect of 18A on the conformation of the HIV-1 Env trimer was studied. The functional, unliganded state of Env is relatively resistant to cold inactivation compared with the CD4-bound Env intermediate. HIV-1_{HXBc2}, a relatively cold-sensitive HIV-1 isolate with a high Env reactivity, displayed decreased sensitivity to cold inactivation in the presence of 18A compared to viruses treated with the controls, DMSO or unrelated compounds (FIG. 4i). These results suggest that 18A can stabilize the unliganded, functional state of Env during a prolonged exposure to cold.

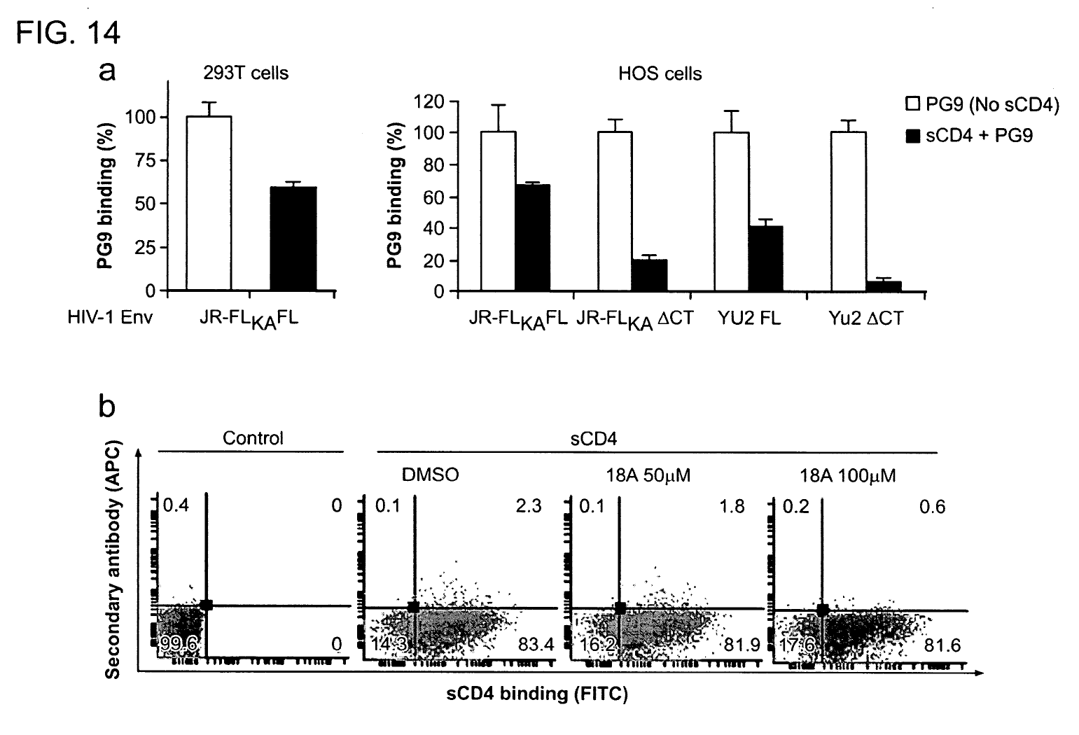

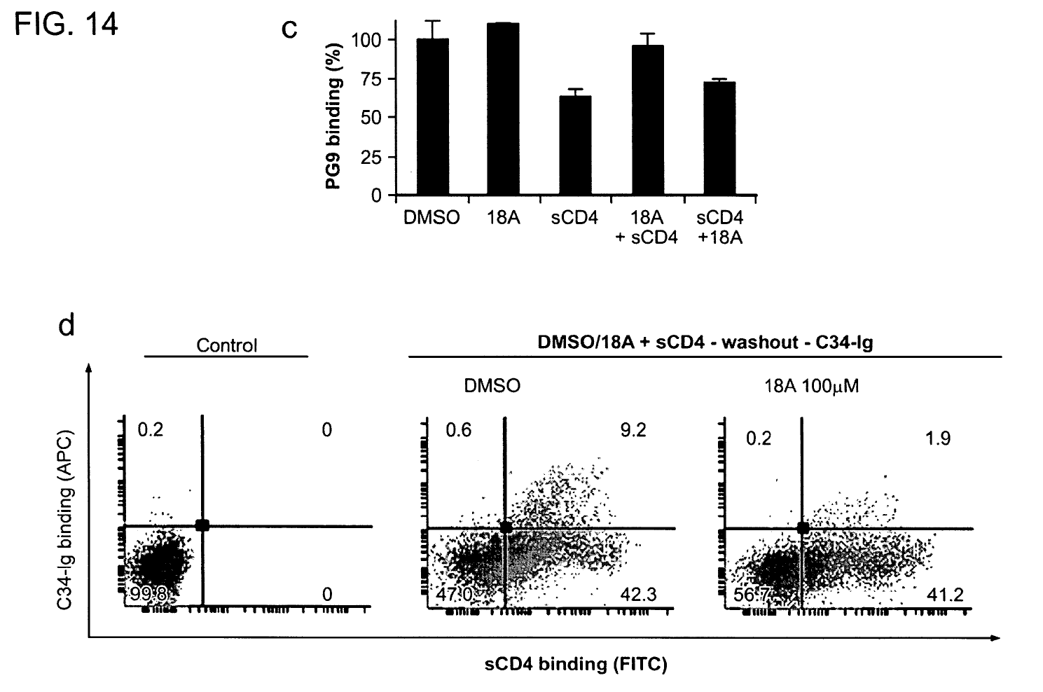

The ability of 18A to interfere with the transition of HIV-1 Env from the unliganded state to the CD4-bound conformation was examined. Binding to CD4 triggers conformational changes in Env that result in an “open” conformation in which the CCR5-binding site on gp120 and the HR1 coiled coil on gp41 are formed and exposed. The CD4-induced opening of the Env spike involves a rearrangement of the membrane-distal trimer association domain of gp120 at the trimer apex; the trimer association domain is composed of the V1/V2 and V3 variable regions of gp120, CD4-induced rearrangement of the V1/V2 region results in a decrease in the binding of the PG9 antibody, which recognizes a V1/V2 epitope that is strongly influenced by quaternary structure. The sCD4-induced reduction in PG9 binding was observed for either full-length or cytoplasmic tail-deleted Env complexes expressed on the surface of 293T or HOS cells; similar results were obtained with Envs derived from wild-type HIV-1_{YU2} or an HIV-1_{JR-FL} variant with E168K+N188A changes in V1/V2, which restores the integrity of the PG9 epitope in that HIV-1 strain (FIG. 14). In the absence of sCD4, the PG9 antibody bound to Env-expressing cells, as shown for the cells expressing the HIV-1_{JR-FL} E168K+N188A variant in FIG. 5a. Treatment with 18A did not affect this basal level of PG9 binding. Prior incubation of Env-expressing cells with sCD4 substantially decreased PG9 binding (from 37.6% to 6.6%). Importantly, addition of 18A prior to sCD4 incubation restored most of the binding signal of PG9 (from 6.6% to 24.1%) without decreasing CD4 binding (FIG. 5a). This effect required incubation with 18A prior to the addition of sCD4 and was consistent over a range of 18A and sCD4 concentrations (FIG. 5b and FIG. 14). These results suggest that 18A inhibits to some extent the CD4-induced conformational rearrangement of the gp120 V1/V2 region.

The effect of 18A on Env recognition by other anti-gp120 antibodies, with and without prior addition of sCD4, was examined. Consistent with the 18A-mediated decrease of the binding of the CD4i antibody 17b to soluble gp120, a reduction in 17b binding to the cell surface-expressed Env trimer was observed in the presence of 18A (FIG. 5c). As expected, incubation with sCD4 resulted in an increase in 17b binding; 18A did not affect this process. We also examined the binding of a V3-directed antibody 19b and two antibodies, 2G12 and PGT121, directed against carbohydrate-dependent gp120 epitopes. As expected, sCD4 reduced the binding of the PGT121 antibody to Env. No significant effect of 18A on the binding of 19b and 2G12, or on the sCD4-induced decrease of PGT121 binding, was observed.

The transition of HIV-1 Env from the unliganded state to the CD4-bound conformation also involves the CD4-induced exposure of the gp41 HR1 region. To examine the effect of 18A on this process, a fusion protein consisting of an immunoglobulin Fc and the gp41 HR2 peptide, which recognizes the HR1 coiled coil, was used. No C34-Ig binding was detected without prior incubation with sCD4 (FIG. 5d). Approximately 37% of the Env-expressing cells bound C34-Ig after preincubation with sCD4. Incubation of the cells with 18A prior to sCD4 addition significantly decreased C34-Ig binding in a dose-dependent manner, with only 4.7% of the cells binding C34-Ig at a 100 μM concentration of 18A. Washing out the compound after sCD4 binding did not reverse the effect, excluding any direct interference of 18A with C34-Ig binding to HR1 (FIG. 14). So, 18A does not interfere with Env binding to CD4 and CCR5, but efficiently blocks two CD4-induced conformational changes in Env: 1) rearrangement of the gp120 V1/V2 region; and 2) formation/exposure of the gp41 HR1 region.

The mechanistic basis of 18A resistance was studied by testing the ability of resistant Env mutants to complete the above conformational rearrangements in the absence and presence of 18A (FIG. 5c-h). As was seen for the wild-type HIV-1_{JR-FL} Env, binding of PG9 to 18A-resistant Env mutants was not significantly affected by 18A (FIG. 5e). Preincubation with sCD4 reduced PG9 binding to most of the Env mutants and this effect was significantly enhanced for the I154A, N156A, L193A and M434A mutants relative to the wild-type Env. 18A-mediated restoration of PG9 binding was very low in these mutants, pointing to a possible pathway to resistance (FIGS. 5e and f). Interestingly, the basal level of PG9 binding to the Y435A mutant was low and insensitive to sCD4 preincubation and to incubation with 18A. The CD4-induced formation exposure of the gp41 HR1 coiled coil on the 18A-resistant mutants, in both the absence and presence of 18A, was also examined. The N156A, L179G and M434A mutants were relatively resistant to the blocking effect of 18A on gp41 HR1 exposure (FIG. 5g). Quantitative analysis demonstrated that the levels of gp120 V1/V2 rearrangement and gp41 HR1 exposure in the presence of 18A both contribute to the 18A-resistant phenotype (FIG. 5h). Thus, the 18A-resistant mutants may use different pathways to resist 18A and are apparently able to undergo rearrangements of the gp120 V1/V2 and gp41 HR1 regions even in the presence of 18A.

### Example 8

### Therapeutic Index

The IC₅₀ of each of the following compounds against JR-FL and A-MLV were measured, and the therapeutic index was calculated. Note: in the table, asymmetrical B rings are drawn in the proper orientation with respect to the structure below.

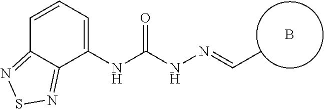

| Compound No. | 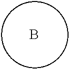 | IC₅₀ (JR-FL) (μM) | IC₅₀ (A-MLV) (μM) | Therapeutic Index (TI) |
|---|---|---|---|---|
| 18A | 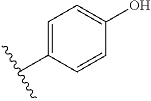 [SMILES: *c1ccc(O)cc1] | 3.6 | 56.5 | 15.9 |
| 18A1 | 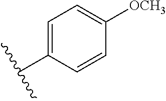 [SMILES: *c1ccc(OC)cc1] | >112 | >112 | NA |
| 18A2 | 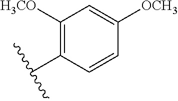 [SMILES: *c1ccc(OC)cc1OC] | 60 | 101 | 1.7 |
| 18A5 | 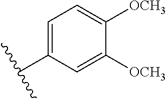 [SMILES: *c1ccc(OC)c(OC)c1] | 5 | 25.9 | 5.1 |
| 18A10 | 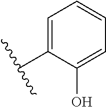 [SMILES: *c1ccccc1O] | ~0.7 | <0.5 | <1 |
| 18A13 | 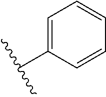 [SMILES: *c1ccccc1] | 16.9 | 75.2 | 4.4 |
| 18A16 | 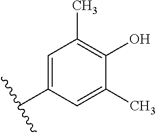 [SMILES: *c1cc(C)c(O)c(C)c1] | 0.2 | 0.1 | 0.5 |
| 18A17 | 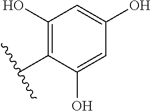 [SMILES: *c1c(O)cc(O)cc1O] | 24.8 | 64 | 2.6 |
| 18A18 | 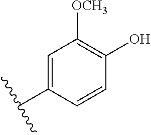 [SMILES: *c1ccc(O)c(OC)c1] | 1.5 | 0.8 | 0.6 |
| 18A19 | 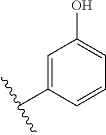 [SMILES: *c1cccc(O)c1] | 6.6 | 46.2 | 7 |
| 18A20 | 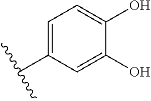 [SMILES: *c1ccc(O)c(O)c1] | 5.2 | 56.3 | 10.8 |
| 18A21 | 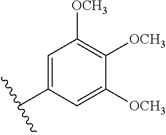 [SMILES: *c1cc(OC)c(OC)c(OC)c1] | >112 | >112 | NA |
| 18A23 (did not reach 100% inhibition) | 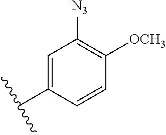 [SMILES: *c1ccc(OC)c(N=[N+]=[N-])c1] | 11 | 77 | 7 |
| 18A25 | 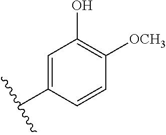 [SMILES: *c1ccc(OC)c(O)c1] | 1.8 | 11.6 | 6.4 |
| 18A30 | 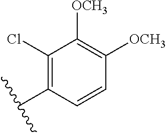 [SMILES: *c1ccc(OC)c(OC)c1Cl] | >112 | >112 | NA |
| 18A32 | 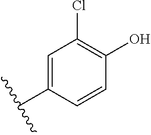 [SMILES: *c1ccc(O)c(Cl)c1] | 4 | 19.2 | 2.4 |
| 18A34 | 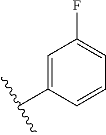 [SMILES: *c1cccc(F)c1] | 10.2 | 69.4 | 6.8 |
| 18A36 | 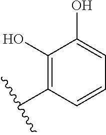 [SMILES: *c1cccc(O)c1O] | 6.2 | 50.5 | 8.1 |
| 18A40 | 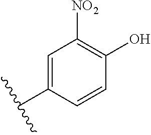 [SMILES: *c1ccc(O)c([N+](=O)[O-])c1] | 7.6 | >112 | >14.7 |
| 18A48 | 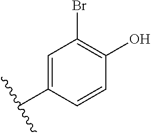 [SMILES: *c1ccc(O)c(Br)c1] | >112 | >112 | NA |
| 18A49 | 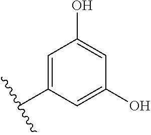 [SMILES: *c1cc(O)cc(O)c1] | 13 | >112 | >8.6 |
| 18A52 | 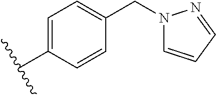 [SMILES: *c1ccc(Cn2cccn2)cc1] | 3.4 | 37.3 | 11 |
| 18A58 | 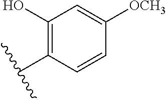 [SMILES: *c1ccc(OC)cc1O] | >112 | >112 | NA |

### INCORPORATION BY REFERENCE

The contents of all references, patent applications, patents, and published patent applications, as well as the Figures and the Sequence Listing, cited throughout this application are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

1. A compound of
(a) Formula I 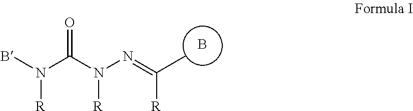
or a pharmaceutically acceptable salt or solvate thereof,
wherein, independently for each occurrence, 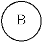
is optionally substituted aryl, optionally substituted heteroaryl, optionally substituted alkenyl, or optionally substituted cycloalkenyl;
R is hydrogen or alkyl;
B′ is optionally substituted aryl, optionally substituted heteroaryl, optionally substituted cycloalkyl, or B′, when taken together with either instance of —NR—, forms a substituted or unsubstituted heterocycloalkyl ring,
provided the compound is not 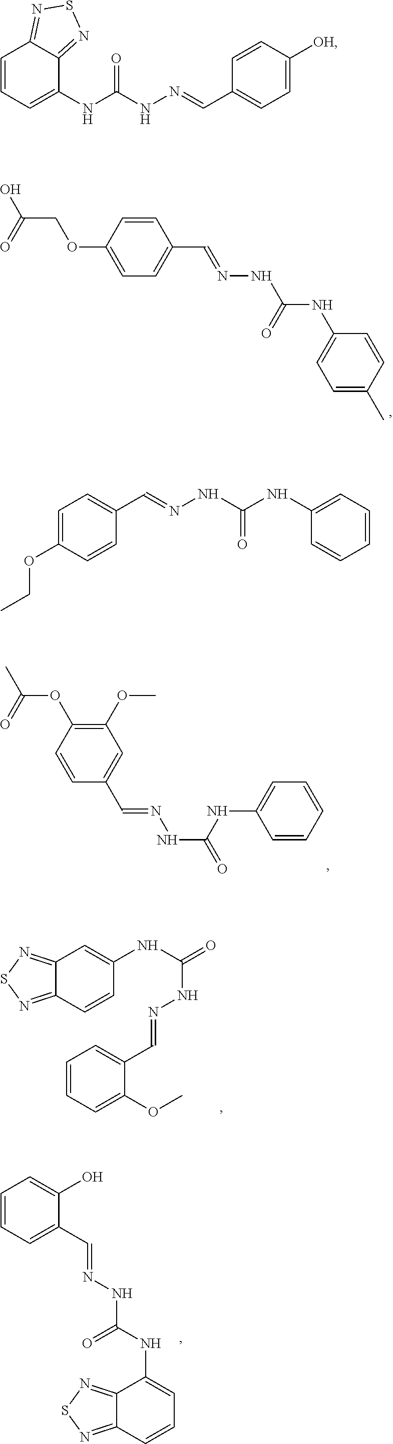 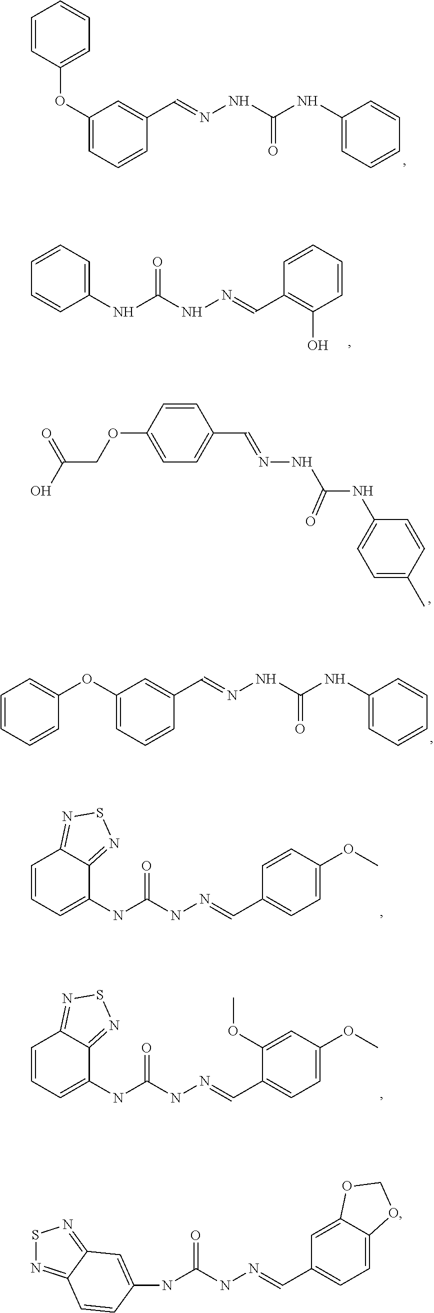 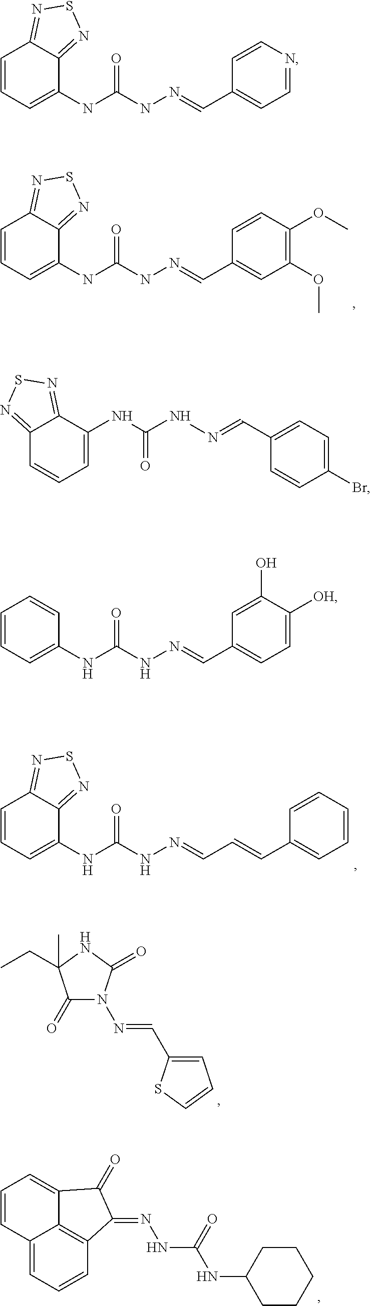 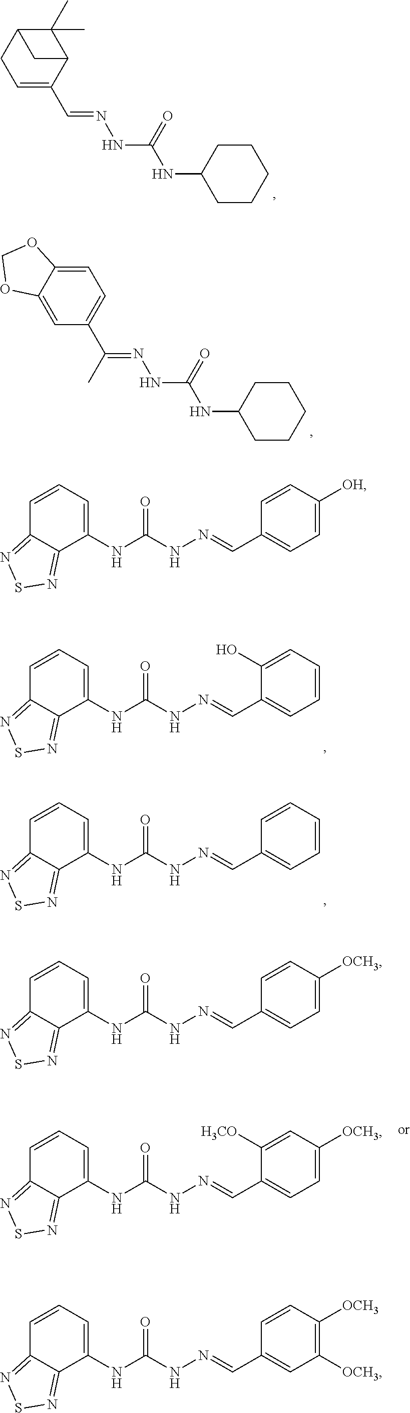
wherein any atoms with an incomplete valence are covalently bonded to one or more hydrogen atoms to complete their valence;
(b) Formula II 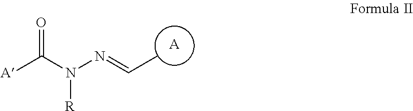
or a pharmaceutically acceptable salt or solvate thereof,
wherein, independently for each occurrence, 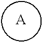
is optionally substituted aryl or optionally substituted heteroaryl, and
A′ is optionally substituted alkyl, optionally substituted cycloalkyl, optionally substituted aryl, or optionally substituted heteroaryl,
provided the compound is not 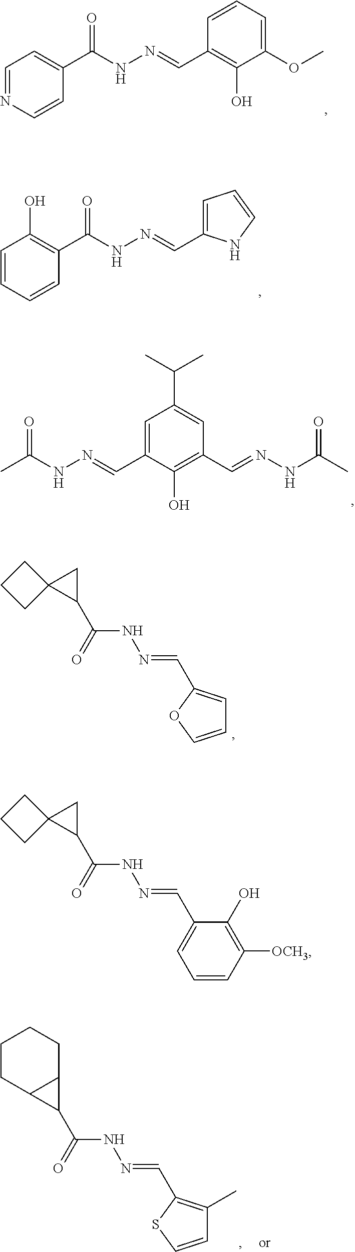 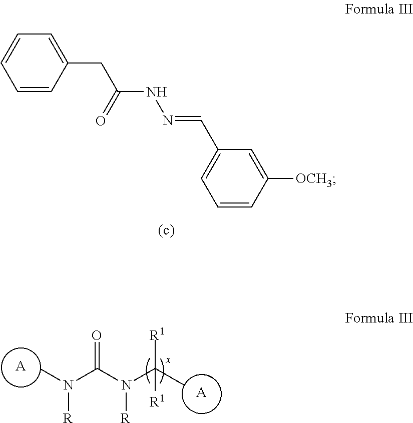
or a pharmaceutically acceptable salt or solvate thereof,
wherein, independently for each occurrence, 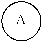
is optionally substituted aryl or optionally substituted heteroaryl;
R is hydrogen or alkyl;
R¹ is hydrogen, hydroxy, alkoxy, or alkyl; and
x is 0, 1, 2, or 3,
provided the compound is not 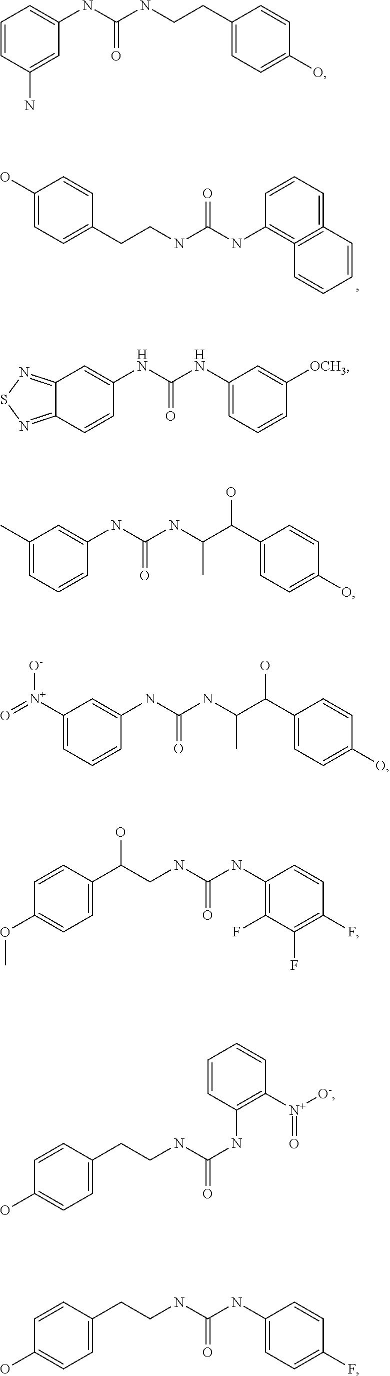 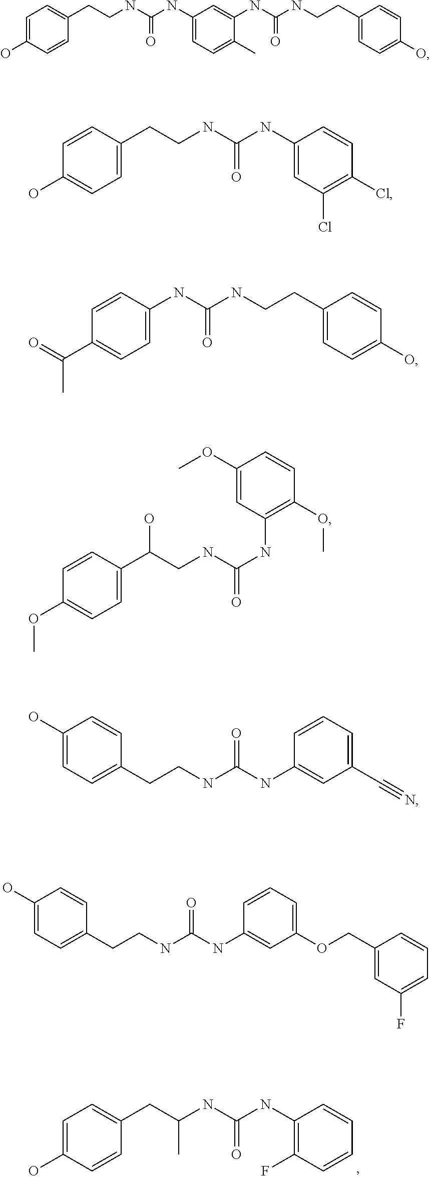 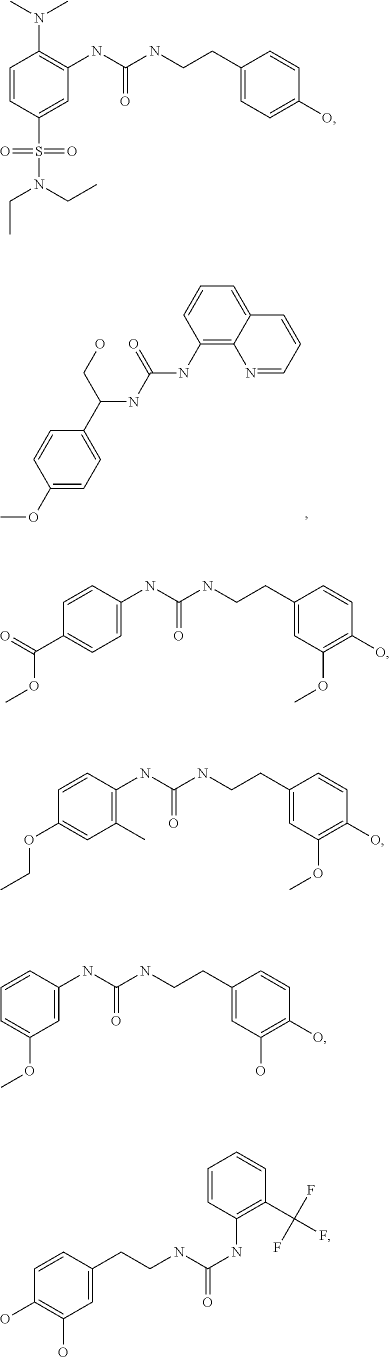 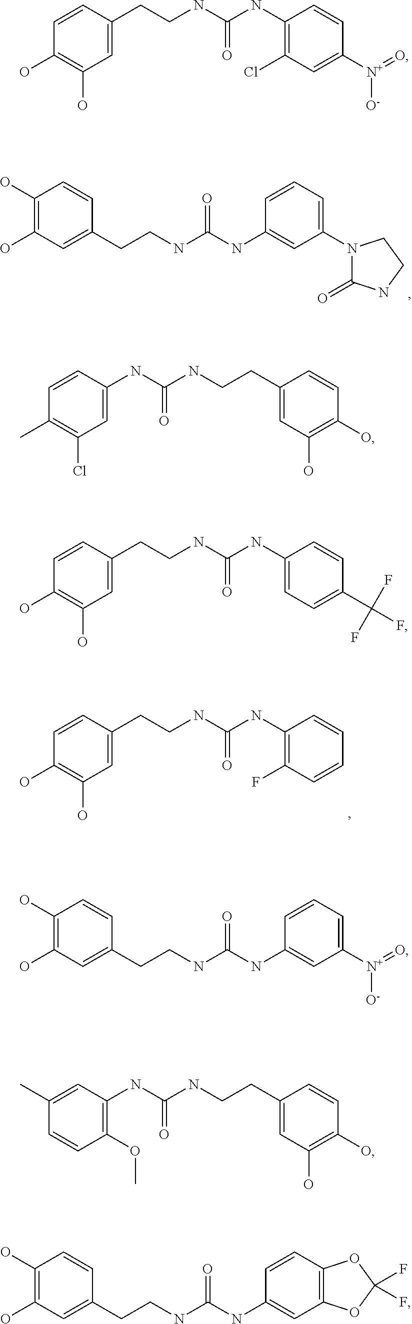 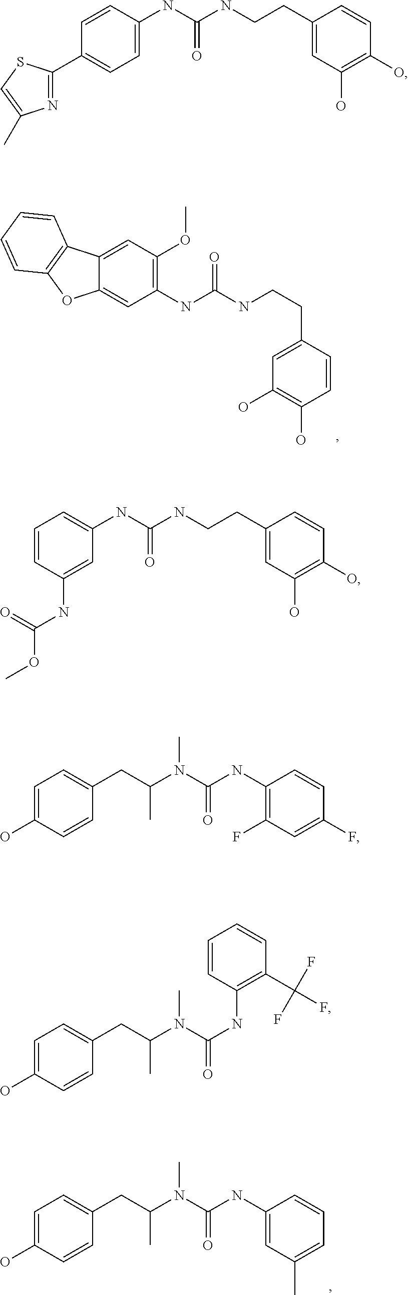 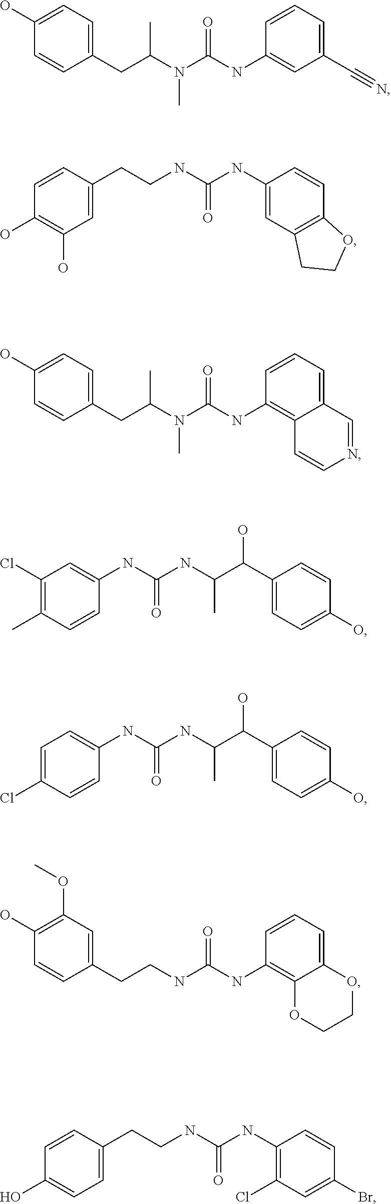 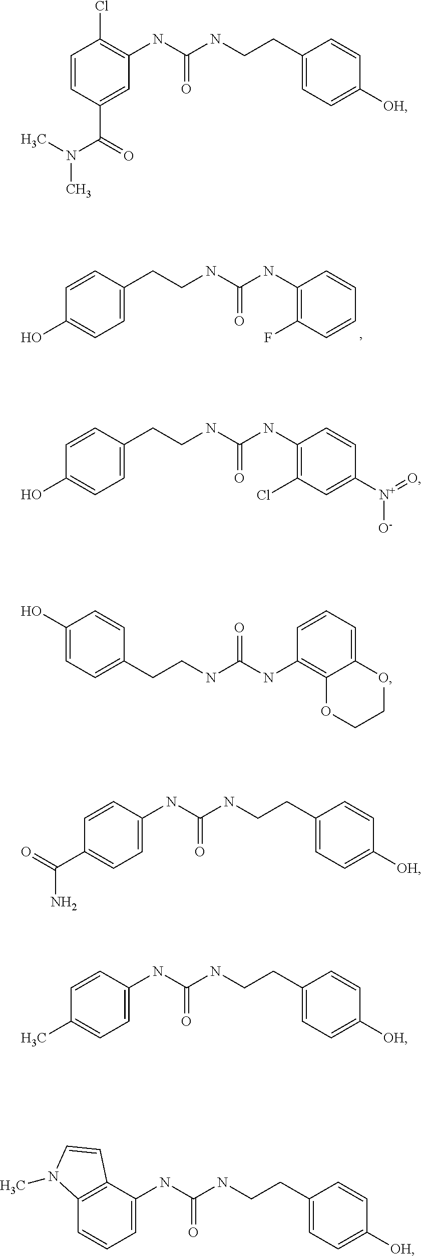 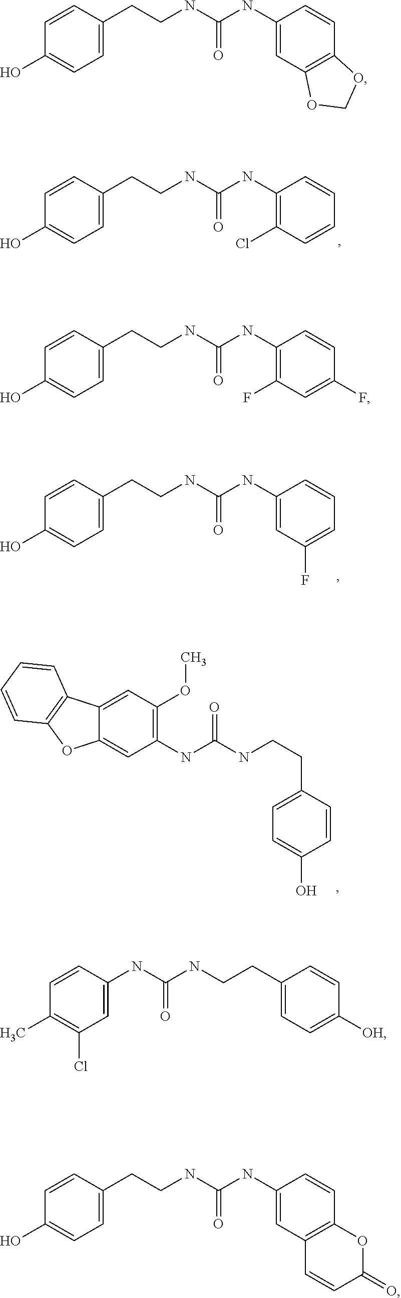 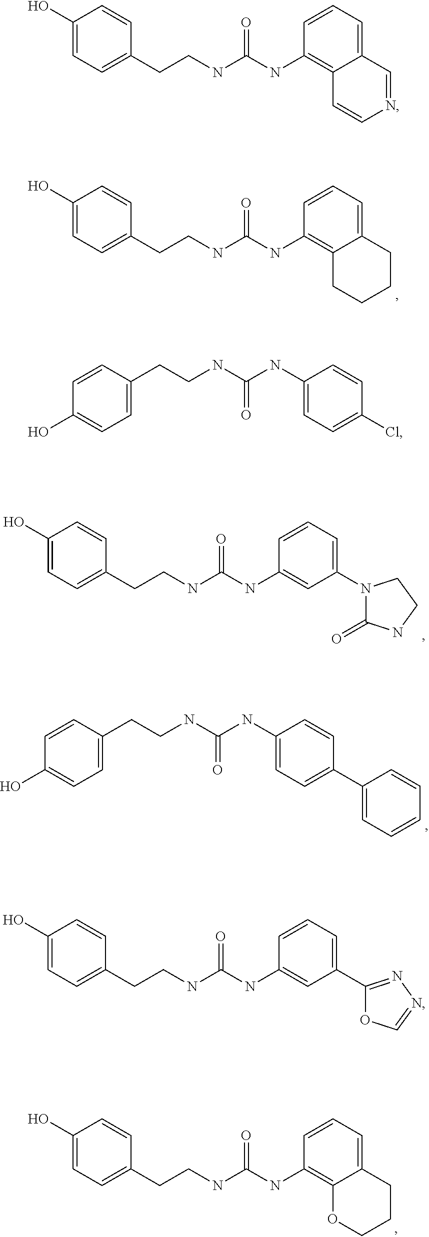 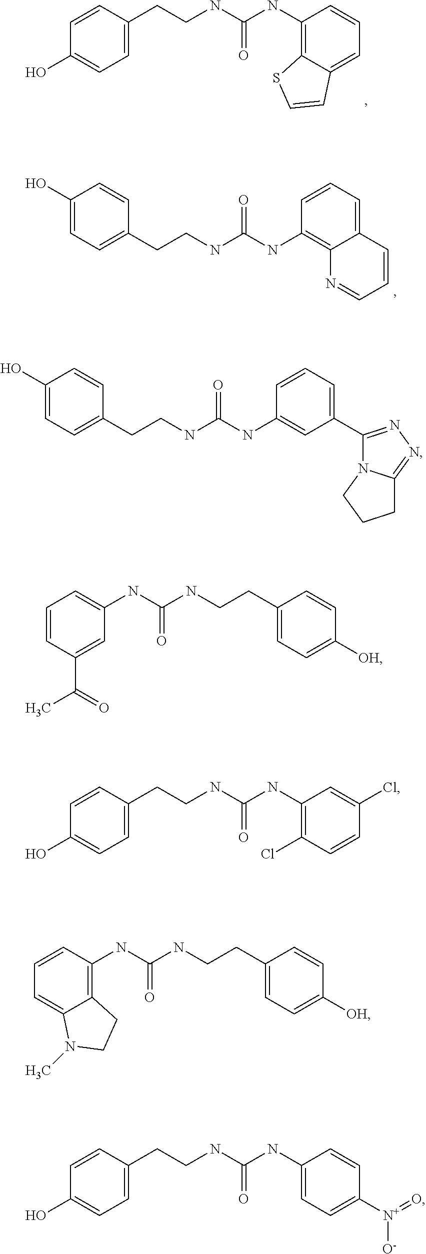 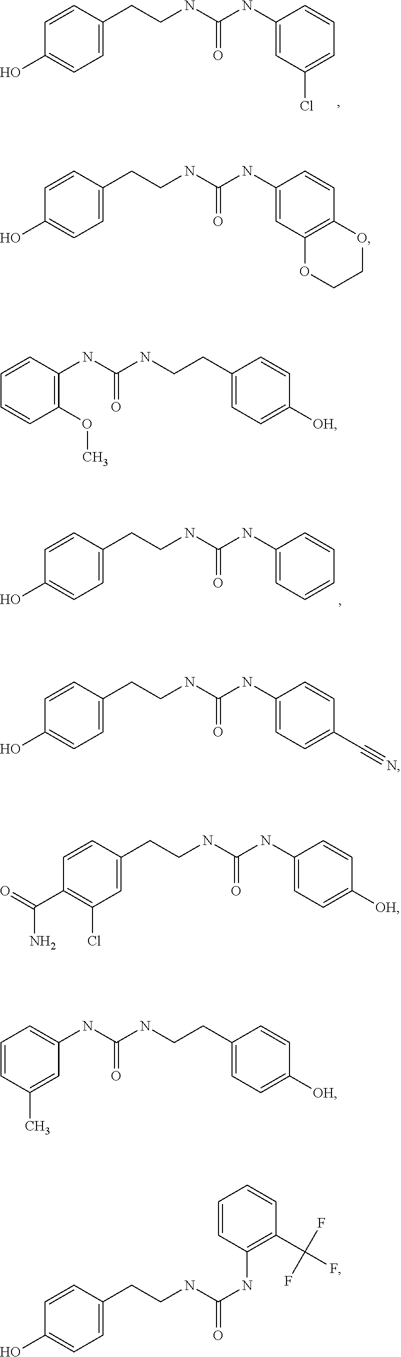 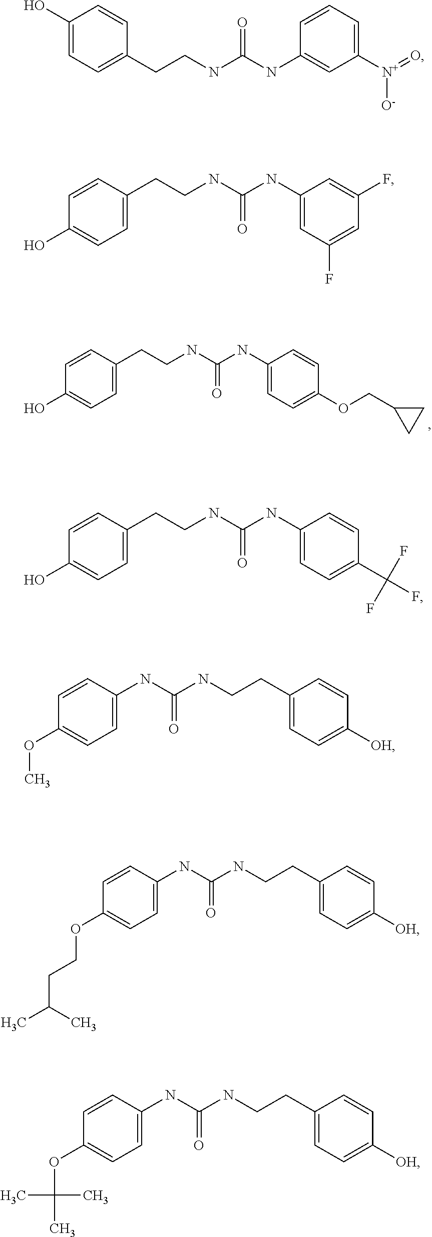 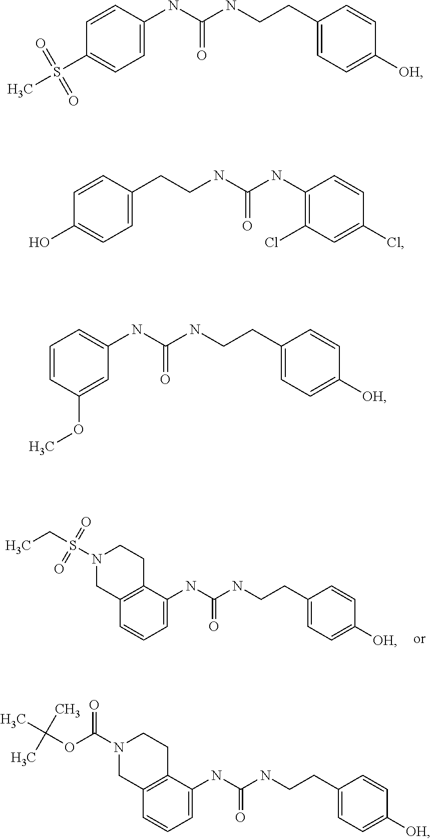 [SMILES: CS(=O)(=O)c1ccc(NC(=O)NCCc2ccc(O)cc2)cc1 ; O=C(NCCc1ccc(O)cc1)Nc1ccc(Cl)cc1Cl ; COc1cccc(NC(=O)NCCc2ccc(O)cc2)c1 ; CCS(=O)(=O)N1CCc2c(cccc2NC(=O)NCCc2ccc(O)cc2)C1 ; "or" ; CC(C)(C)OC(=O)N1CCc2c(cccc2NC(=O)NCCc2ccc(O)cc2)C1]
wherein any atoms with an incomplete valence are covalently bonded to one or more hydrogen atoms to complete their valence;
(d) Formula IV 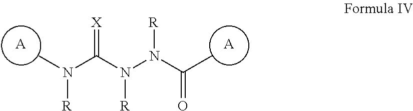
or a pharmaceutically acceptable salt or solvate thereof,
wherein, independently for each occurrence, 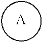
is optionally substituted aryl or optionally substituted heteroaryl;
R is hydrogen or alkyl; and
X is O or S,
provided the compound is not 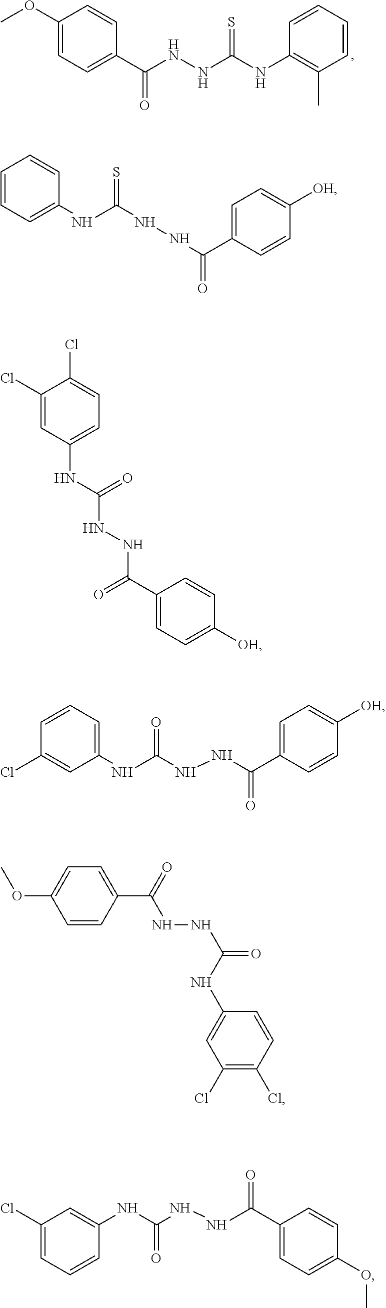 [SMILES: COc1ccc(C(=O)NNC(=S)Nc2ccccc2C)cc1 ; O=C(NNC(=S)Nc1ccccc1)c1ccc(O)cc1 ; O=C(NNC(=O)c1ccc(O)cc1)Nc1ccc(Cl)c(Cl)c1 ; O=C(NNC(=O)c1ccc(O)cc1)Nc1cccc(Cl)c1 ; COc1ccc(C(=O)NNC(=O)Nc2ccc(Cl)c(Cl)c2)cc1 ; COc1ccc(C(=O)NNC(=O)Nc2cccc(Cl)c2)cc1] 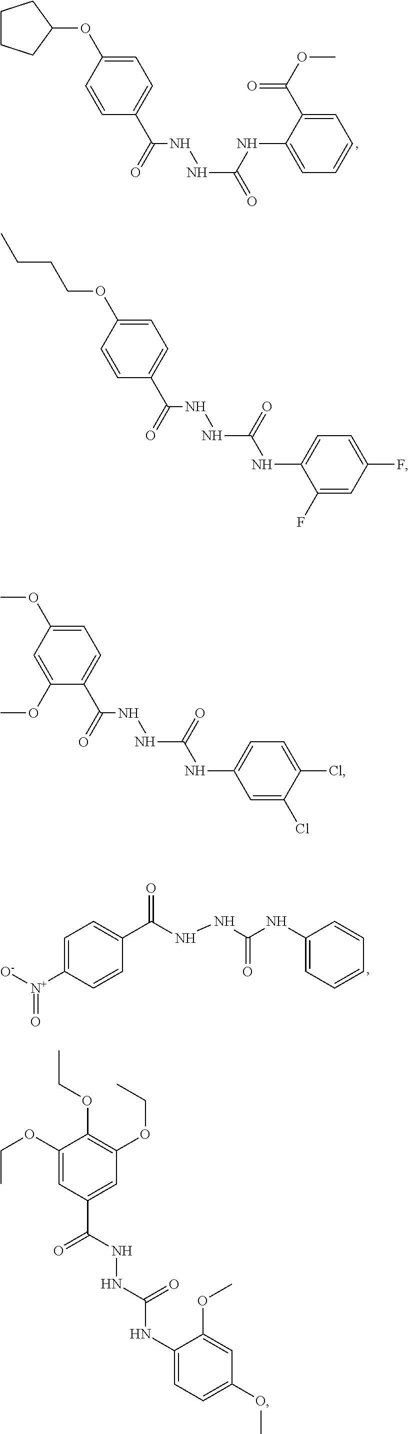 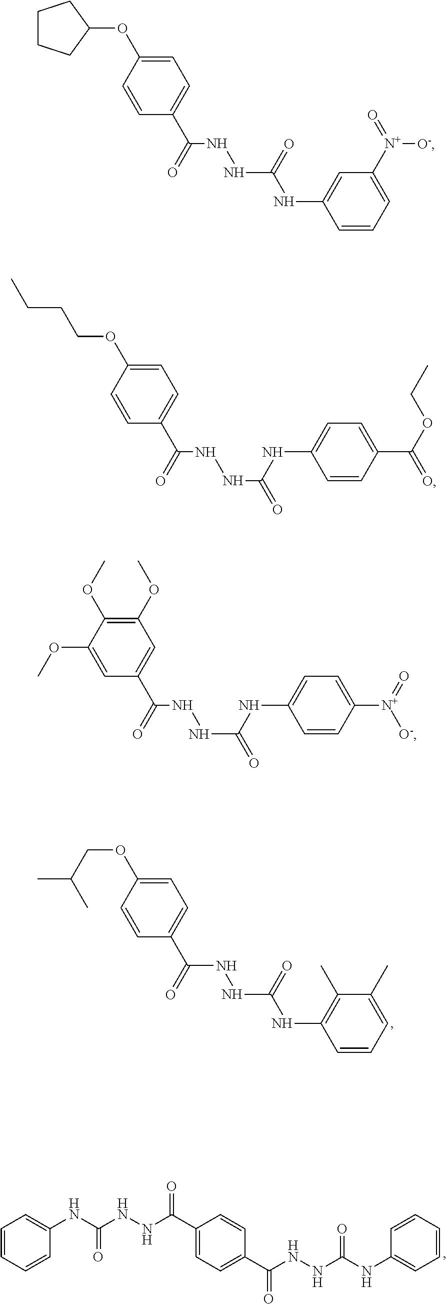 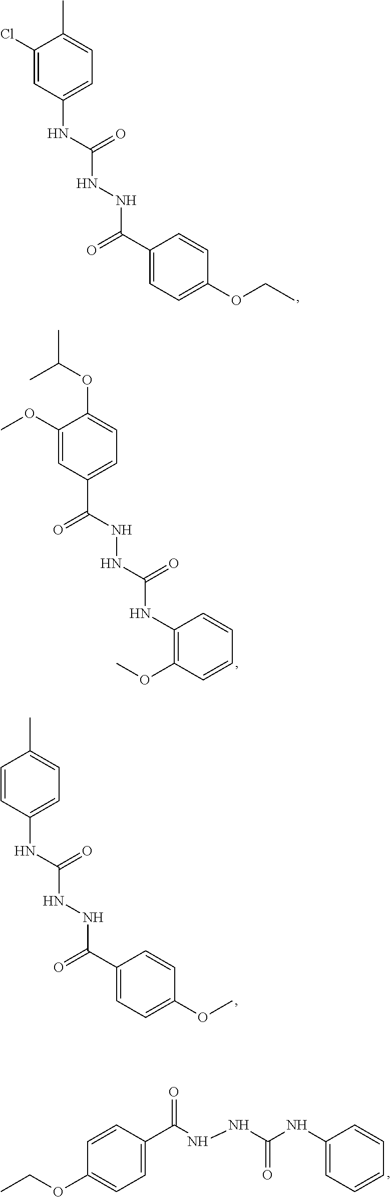 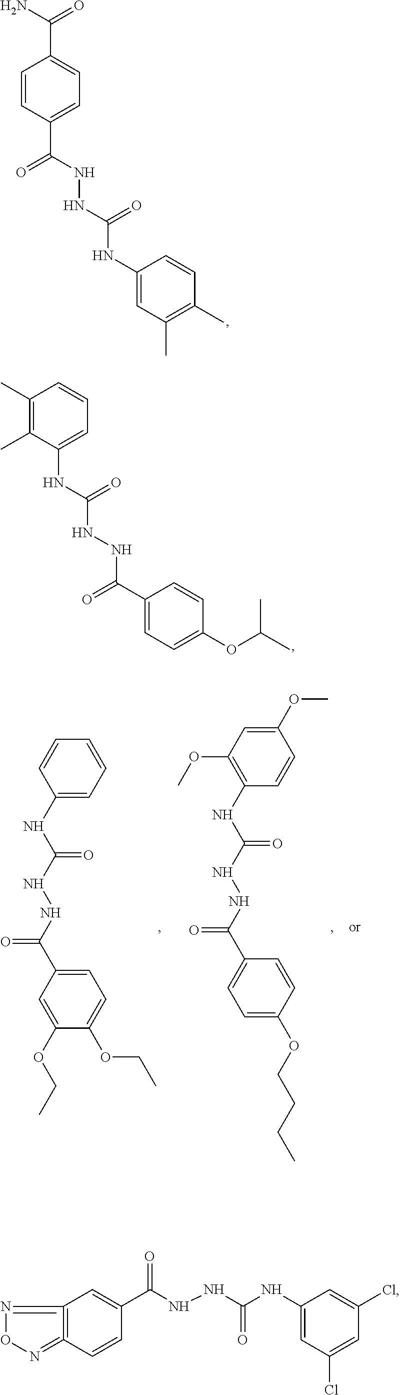
wherein any atoms with an incomplete valence are covalently bonded to one or more hydrogen atoms to complete their valence;
(e) Formula V 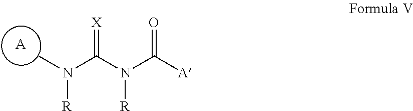
or a pharmaceutically acceptable salt or solvate thereof,
wherein, independently for each occurrence, 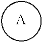
is optionally substituted aryl or optionally substituted heteroaryl;
R is hydrogen or alkyl;
A′ is optionally substituted alkyl, optionally substituted cycloalkyl, optionally substituted aryl, or optionally substituted heteroaryl; and
X is O or S,
provided the compound is not 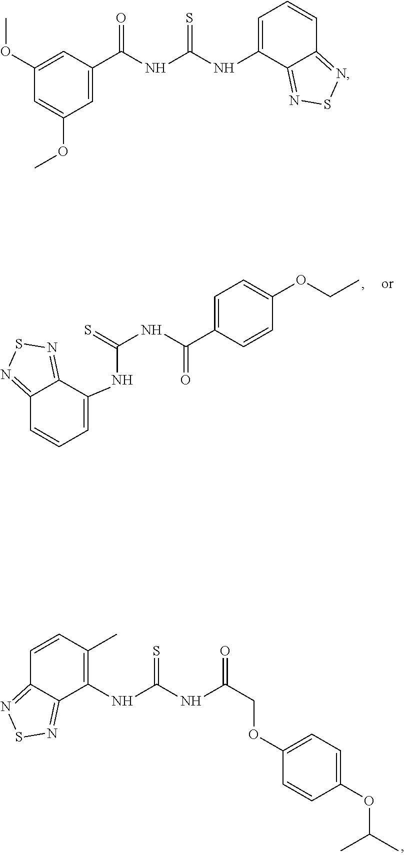 [SMILES: COc1cc(OC)cc(C(=O)NC(=S)Nc2cccc3nsnc23)c1 ; CCOc1ccc(C(=O)NC(=S)Nc2cccc3nsnc23)cc1 ; Cc1ccc2nsnc2c1NC(=S)NC(=O)COc1ccc(OC(C)C)cc1]
wherein any atoms with an incomplete valence are covalently bonded to one or more hydrogen atoms to complete their valence;
(f) Formula VI 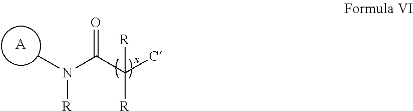
or a pharmaceutically acceptable salt or solvate thereof,
wherein, independently for each occurrence, 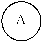
is optionally substituted aryl or optionally substituted heteroaryl;
R is hydrogen or alkyl;
x is 0, 1, 2, or 3; and
C′ is optionally substituted aryl, optionally substituted heteroaryl, optionally substituted cycloalkyl, optionally substituted heterocyclyl, optionally substituted aryloxy, optionally substituted heteroaryloxy, optionally substituted arylthio, or optionally substituted heteroarylthio;
provided the compound is not 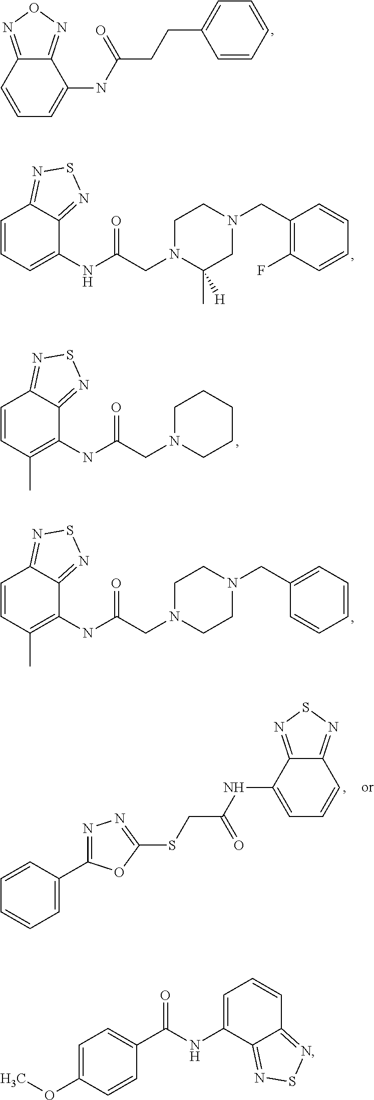
wherein any atoms with an incomplete valence are covalently bonded to one or more hydrogen atoms to complete their valence; or
(g) Formula VII 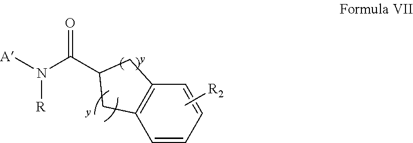
or a pharmaceutically acceptable salt or solvate thereof,
wherein, independently for each occurrence,
A′ is optionally substituted alkyl, optionally substituted cycloalkyl, optionally substituted aryl, or optionally substituted heteroaryl,
R is hydrogen or alkyl;
y is 1 or 2; and
R² is halo, hydroxy, alkoxy, alkylthio, or amino,
provided the compound is not 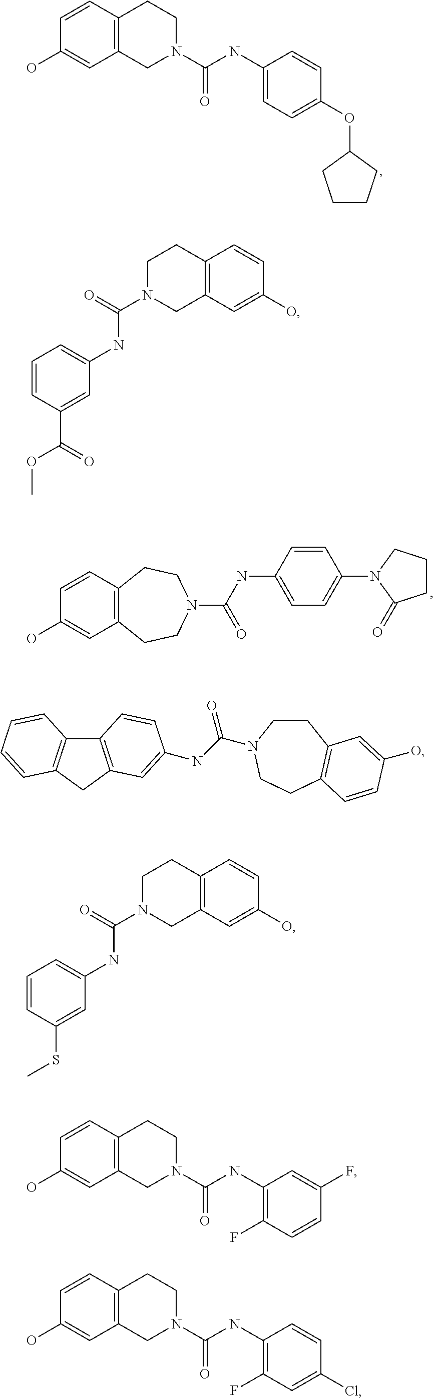 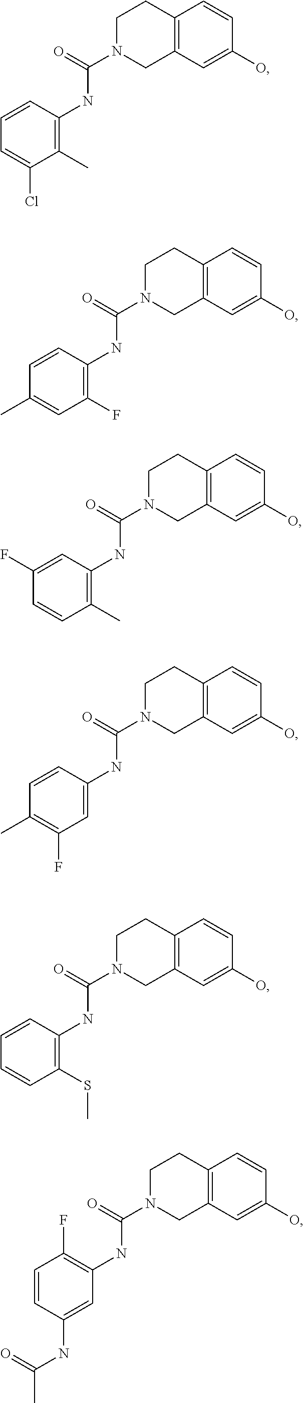 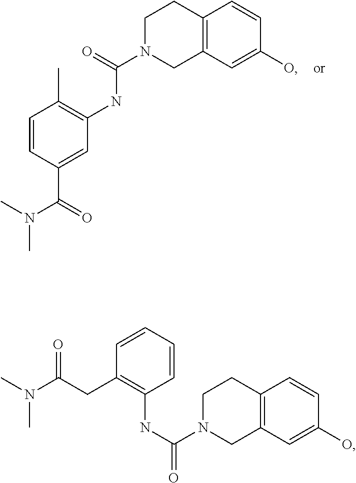
wherein any atoms with an incomplete valence are covalently bonded to one or more hydrogen atoms to complete their valence.

2-7. (canceled)

8. A method of
(a) inhibiting HIV exterior envelope glycoprotein gp120 comprising the step of: contacting HIV with an effective amount of a compound of claim 1;
(b) inhibiting transmission of HIV to a cell comprising the step of: contacting HIV with an effective amount of a compound of claim 1, thereby inhibiting transmission of HIV to said cell; or
(c) inhibiting the progression of HIV infection in a human host comprising the step of: contacting HIV with an effective amount of a compound of claim 1, thereby inhibiting progression of HIV in the human host.

9-10. (canceled)

11. A method of
(a) inhibiting HIV exterior envelope glycoprotein gp120 comprising the step of: contacting HIV with an effective amount of a compound;
(b) inhibiting transmission of HIV to a cell comprising the step of: contacting HIV with an effective amount of a compound, thereby inhibiting transmission of HIV to said cell; or
(c) inhibiting the progression of HIV infection in a human host comprising the step of: contacting HIV with an effective amount of a compound, thereby inhibiting progression of HIV in the human host,
wherein the compound is selected from the group consisting of: 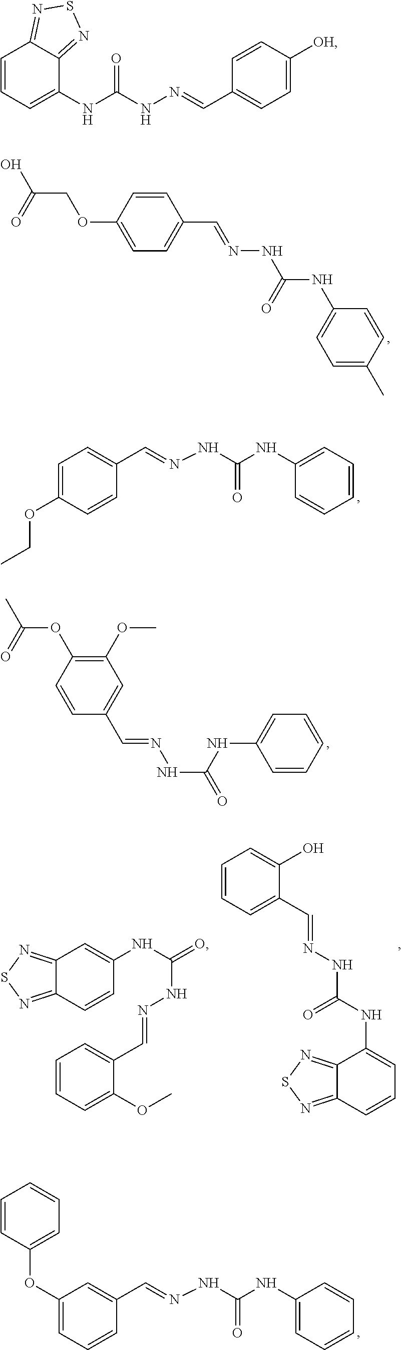 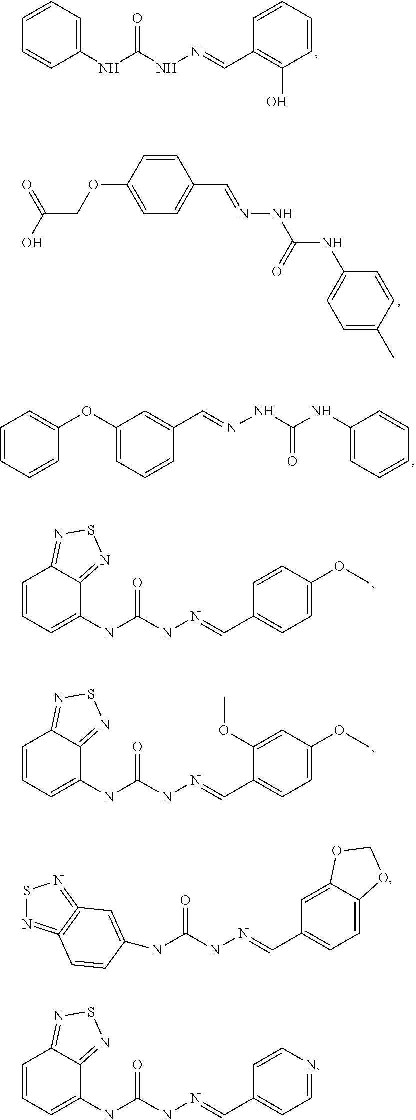 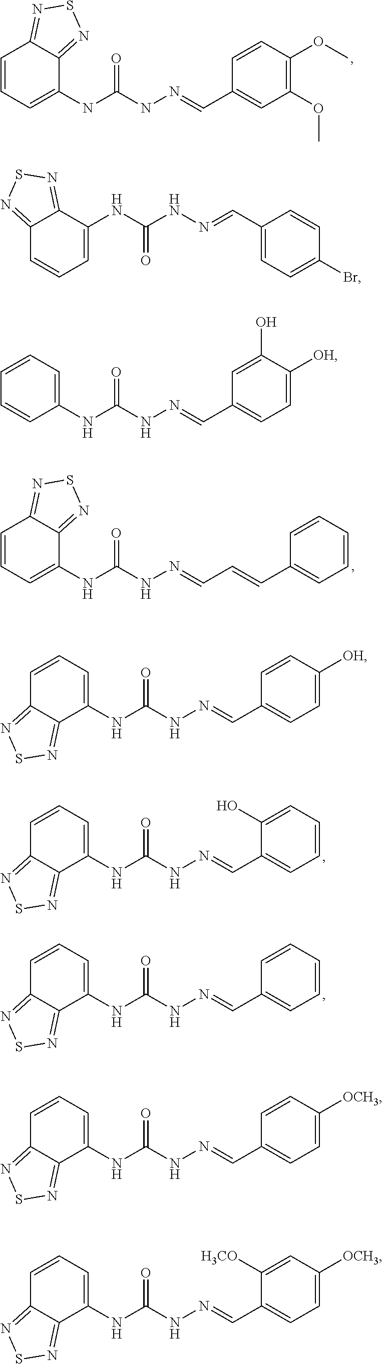 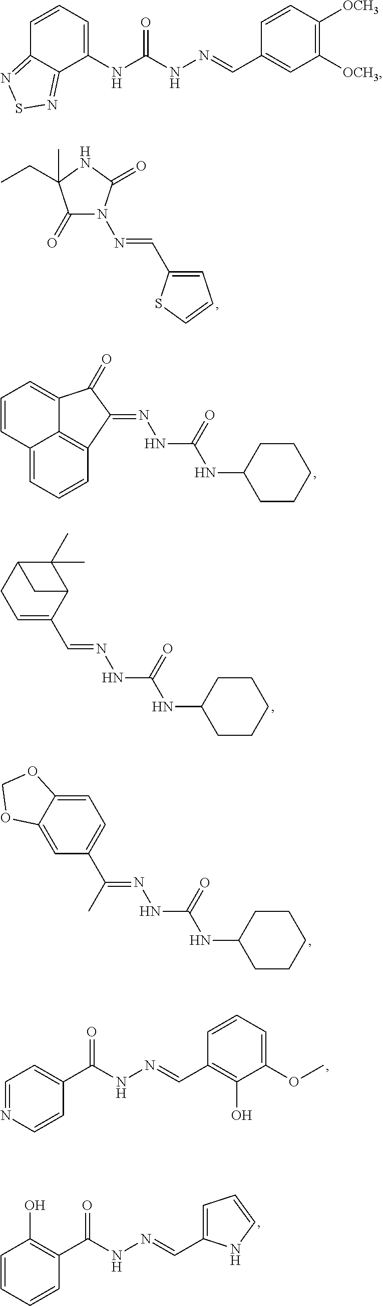 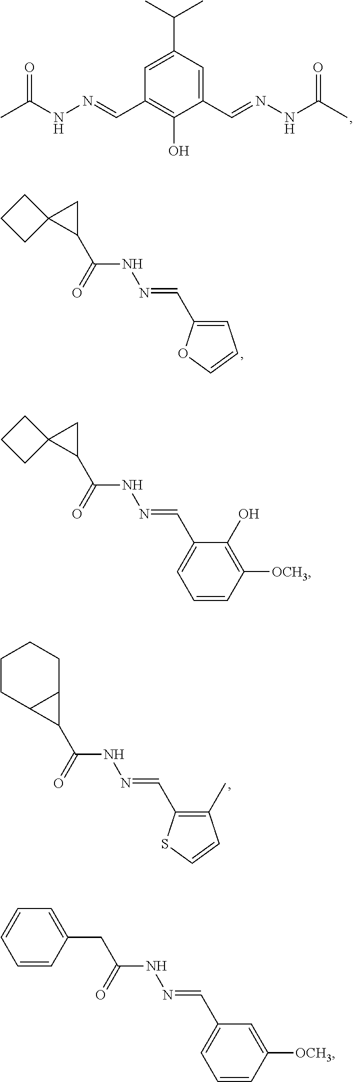 [SMILES: CC(=O)N/N=C/c1cc(C(C)C)cc(/C=N/NC(C)=O)c1O ; O=C(N/N=C/c1ccco1)C1CC12CCC2 ; COc1cccc(/C=N/NC(=O)C2CC23CCC3)c1O ; Cc1ccsc1/C=N/NC(=O)C1C2CCCCC21 ; COc1cccc(/C=N/NC(=O)Cc2ccccc2)c1] 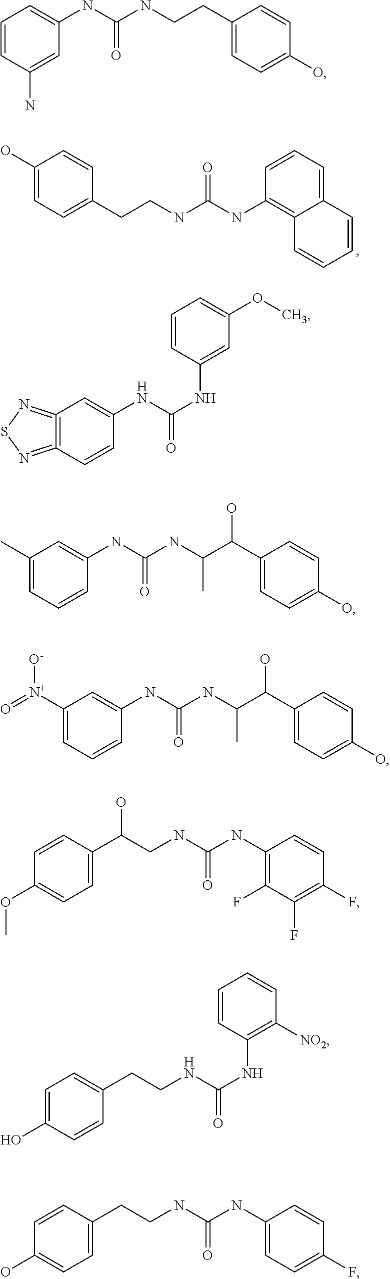 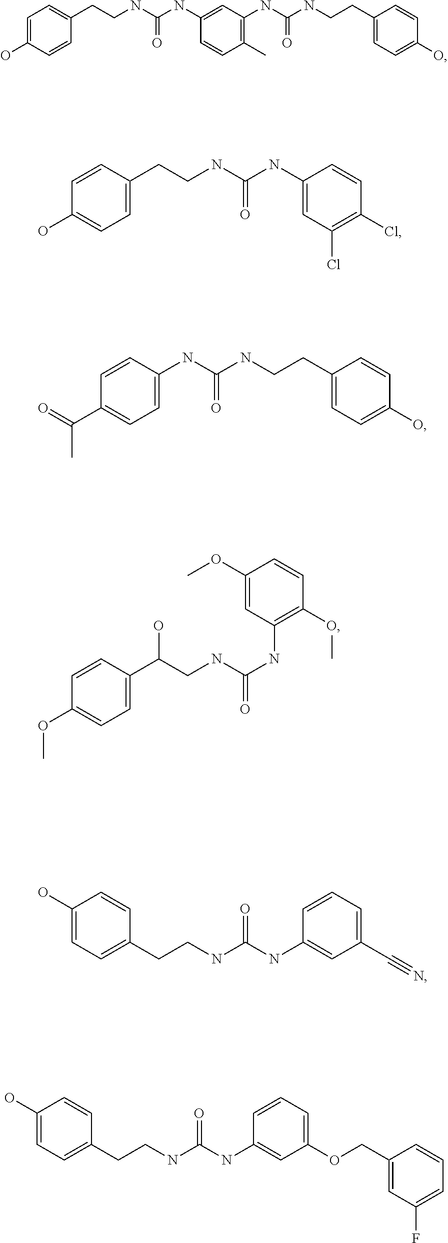 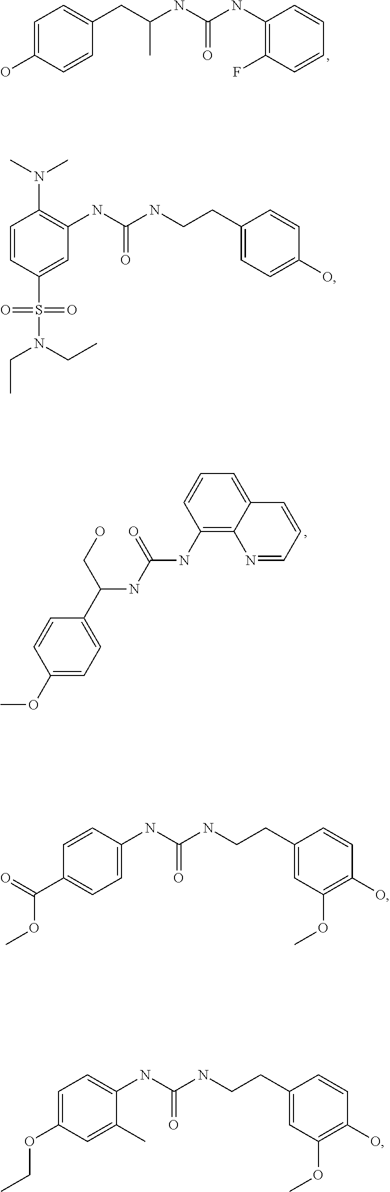 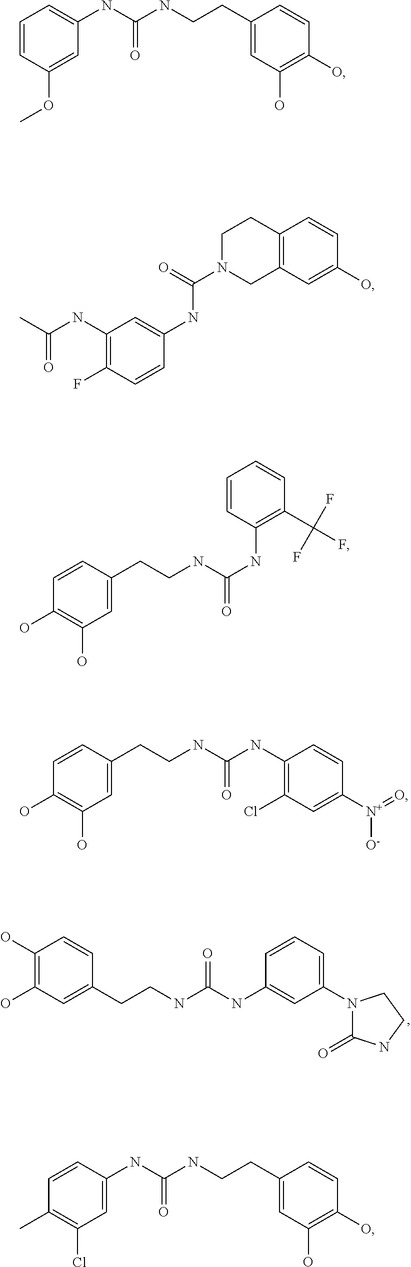 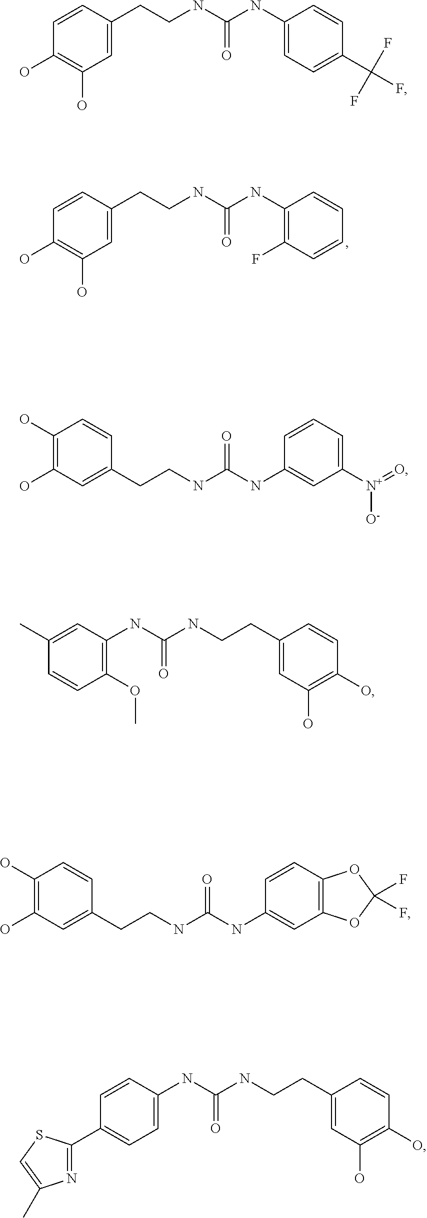 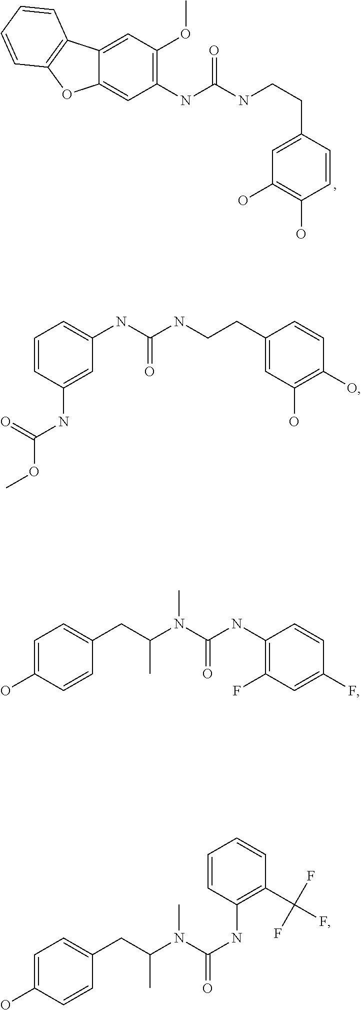 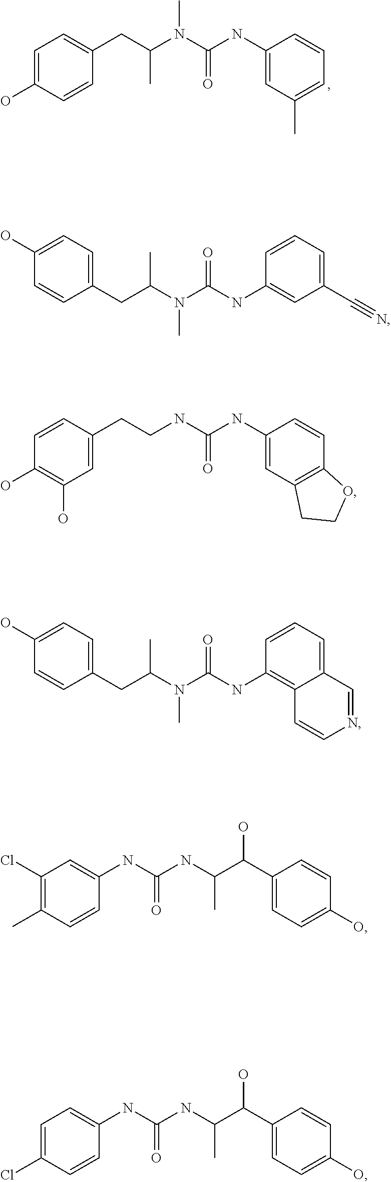 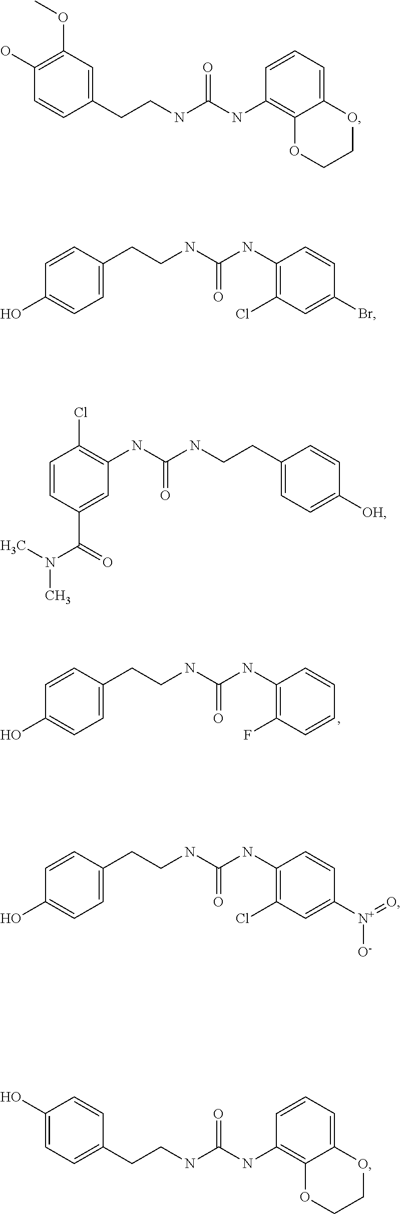 [SMILES: COc1cc(CCNC(=O)Nc2cccc3c2OCCO3)ccc1O ; O=C(NCCc1ccc(O)cc1)Nc1ccc(Br)cc1Cl ; CN(C)C(=O)c1ccc(Cl)c(NC(=O)NCCc2ccc(O)cc2)c1 ; O=C(NCCc1ccc(O)cc1)Nc1ccccc1F ; O=C(NCCc1ccc(O)cc1)Nc1ccc([N+](=O)[O-])cc1Cl ; O=C(NCCc1ccc(O)cc1)Nc1cccc2c1OCCO2] 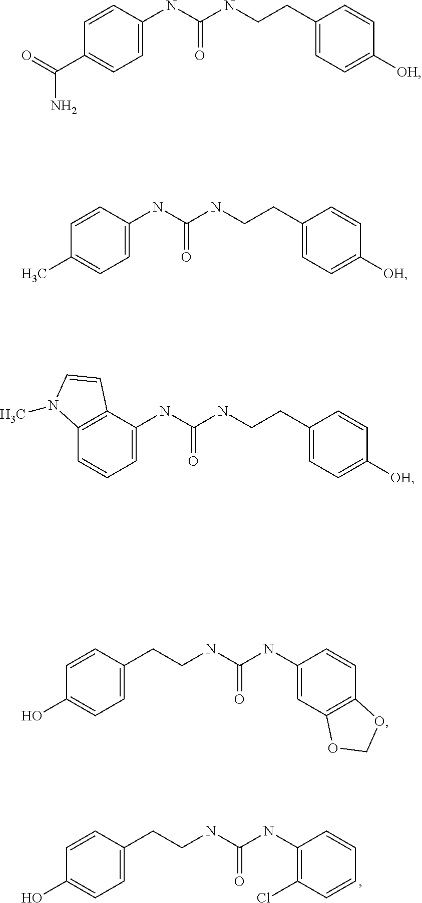 [SMILES: NC(=O)c1ccc(NC(=O)NCCc2ccc(O)cc2)cc1 ; Cc1ccc(NC(=O)NCCc2ccc(O)cc2)cc1 ; Cn1ccc2c(NC(=O)NCCc3ccc(O)cc3)cccc21 ; O=C(NCCc1ccc(O)cc1)Nc1ccc2c(c1)OCO2 ; O=C(NCCc1ccc(O)cc1)Nc1ccccc1Cl] 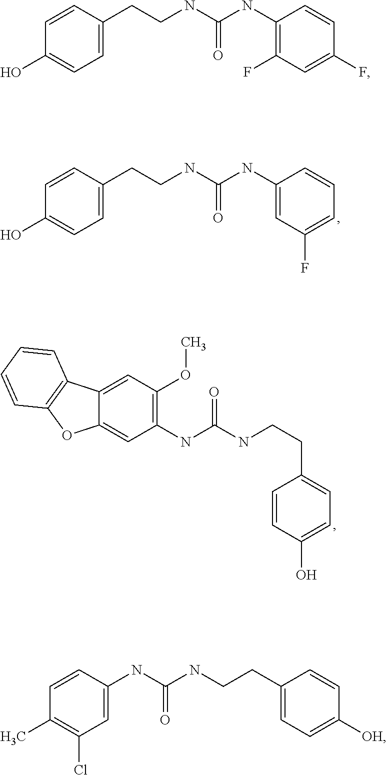 [SMILES: O=C(NCCc1ccc(O)cc1)Nc1ccc(F)cc1F ; O=C(NCCc1ccc(O)cc1)Nc1cccc(F)c1 ; COc1cc2c(cc1NC(=O)NCCc1ccc(O)cc1)oc1ccccc12 ; Cc1ccc(NC(=O)NCCc2ccc(O)cc2)cc1Cl] 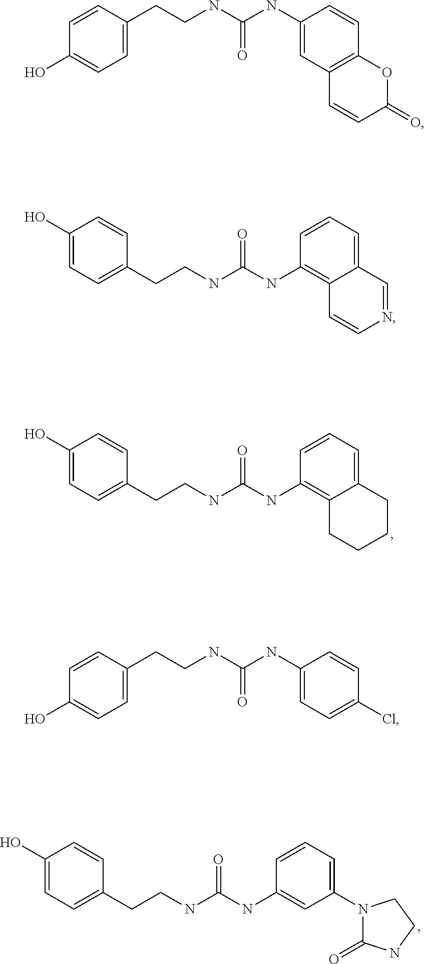 [SMILES: O=C(NCCc1ccc(O)cc1)Nc1ccc2oc(=O)ccc2c1 ; O=C(NCCc1ccc(O)cc1)Nc1cccc2cnccc12 ; O=C(NCCc1ccc(O)cc1)Nc1cccc2c1CCCC2 ; O=C(NCCc1ccc(O)cc1)Nc1ccc(Cl)cc1 ; O=C(NCCc1ccc(O)cc1)Nc1cccc(N2CCNC2=O)c1] 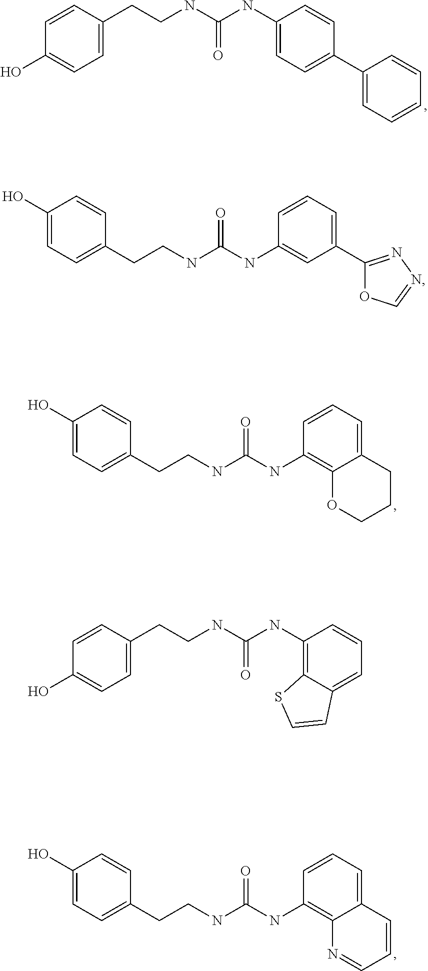 [SMILES: O=C(NCCc1ccc(O)cc1)Nc1ccc(-c2ccccc2)cc1 ; O=C(NCCc1ccc(O)cc1)Nc1cccc(-c2nnco2)c1 ; O=C(NCCc1ccc(O)cc1)Nc1cccc2c1OCCC2 ; O=C(NCCc1ccc(O)cc1)Nc1cccc2ccsc12 ; O=C(NCCc1ccc(O)cc1)Nc1cccc2cccnc12] 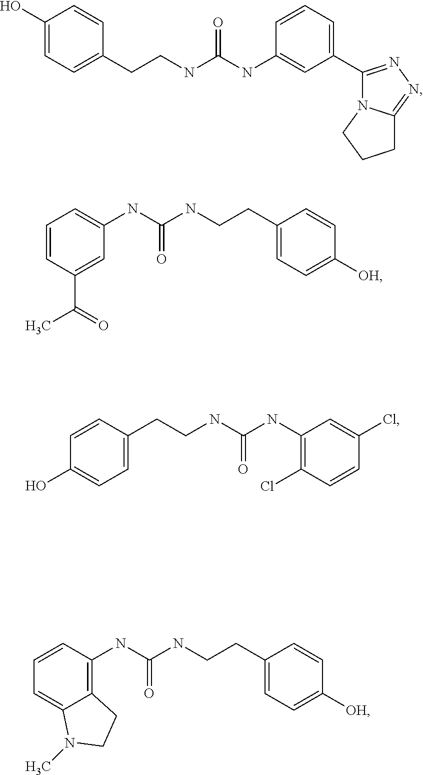 [SMILES: O=C(NCCc1ccc(O)cc1)Nc1cccc(-c2nnc3n2CCC3)c1 ; CC(=O)c1cccc(NC(=O)NCCc2ccc(O)cc2)c1 ; O=C(NCCc1ccc(O)cc1)Nc1cc(Cl)ccc1Cl ; CN1CCc2c(NC(=O)NCCc3ccc(O)cc3)cccc21] 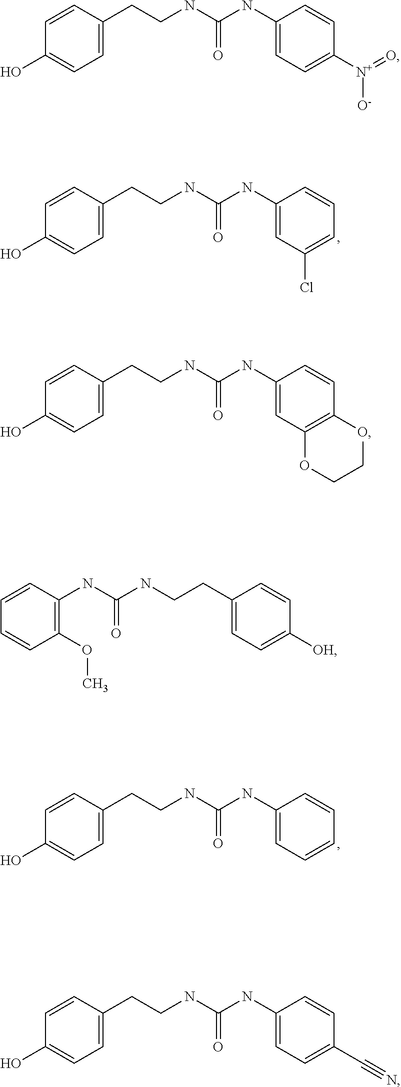 [SMILES: O=C(NCCc1ccc(O)cc1)Nc1ccc([N+](=O)[O-])cc1 ; O=C(NCCc1ccc(O)cc1)Nc1cccc(Cl)c1 ; O=C(NCCc1ccc(O)cc1)Nc1ccc2c(c1)OCCO2 ; COc1ccccc1NC(=O)NCCc1ccc(O)cc1 ; O=C(NCCc1ccc(O)cc1)Nc1ccccc1 ; N#Cc1ccc(NC(=O)NCCc2ccc(O)cc2)cc1] 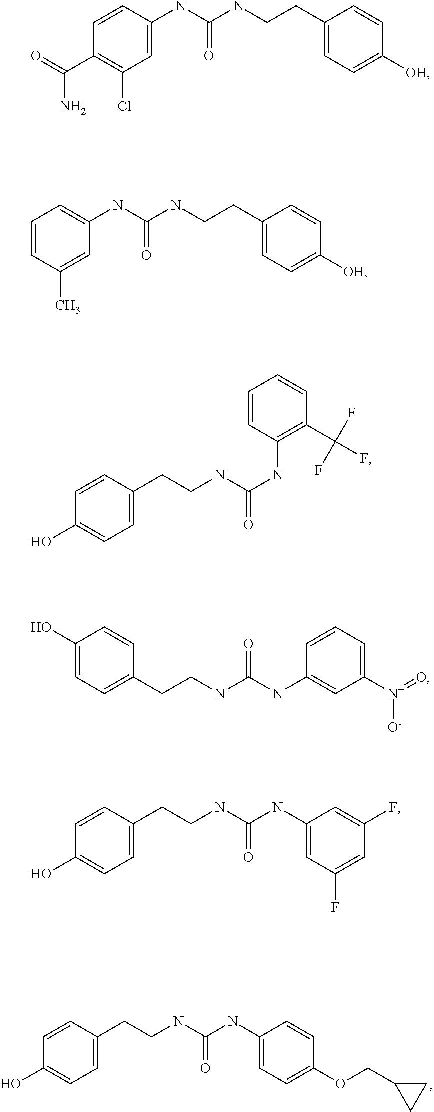 [SMILES: NC(=O)c1ccc(NC(=O)NCCc2ccc(O)cc2)cc1Cl ; Cc1cccc(NC(=O)NCCc2ccc(O)cc2)c1 ; O=C(NCCc1ccc(O)cc1)Nc1ccccc1C(F)(F)F ; O=C(NCCc1ccc(O)cc1)Nc1cccc([N+](=O)[O-])c1 ; O=C(NCCc1ccc(O)cc1)Nc1cc(F)cc(F)c1 ; O=C(NCCc1ccc(O)cc1)Nc1ccc(OCC2CC2)cc1] 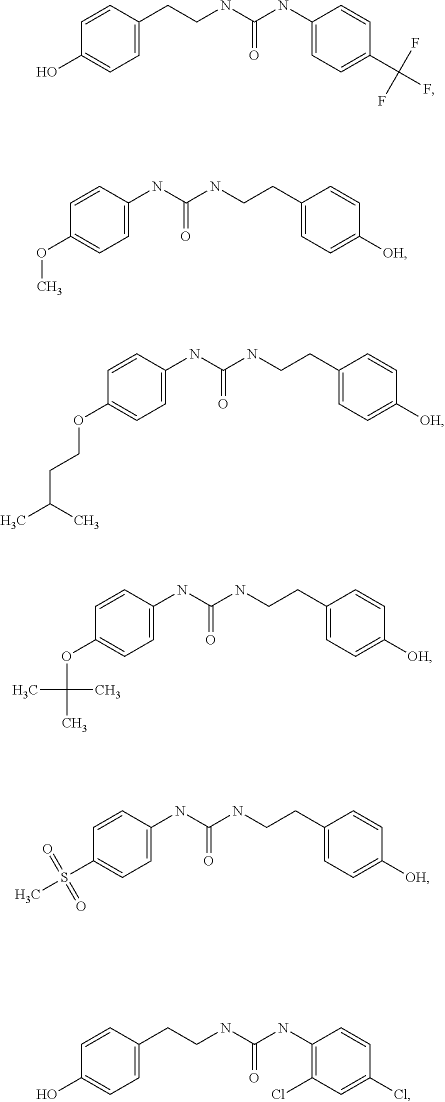 [SMILES: O=C(NCCc1ccc(O)cc1)Nc1ccc(C(F)(F)F)cc1 ; COc1ccc(NC(=O)NCCc2ccc(O)cc2)cc1 ; CC(C)CCOc1ccc(NC(=O)NCCc2ccc(O)cc2)cc1 ; CC(C)(C)Oc1ccc(NC(=O)NCCc2ccc(O)cc2)cc1 ; CS(=O)(=O)c1ccc(NC(=O)NCCc2ccc(O)cc2)cc1 ; O=C(NCCc1ccc(O)cc1)Nc1ccc(Cl)cc1Cl] 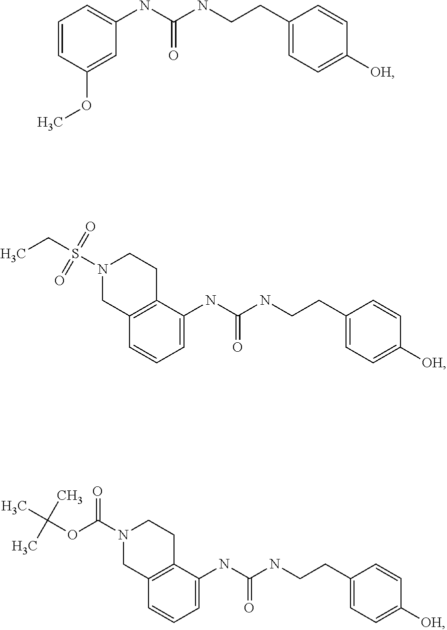 [SMILES: COc1cccc(NC(=O)NCCc2ccc(O)cc2)c1 ; CCS(=O)(=O)N1CCc2c(cccc2NC(=O)NCCc2ccc(O)cc2)C1 ; CC(C)(C)OC(=O)N1CCc2c(cccc2NC(=O)NCCc2ccc(O)cc2)C1] 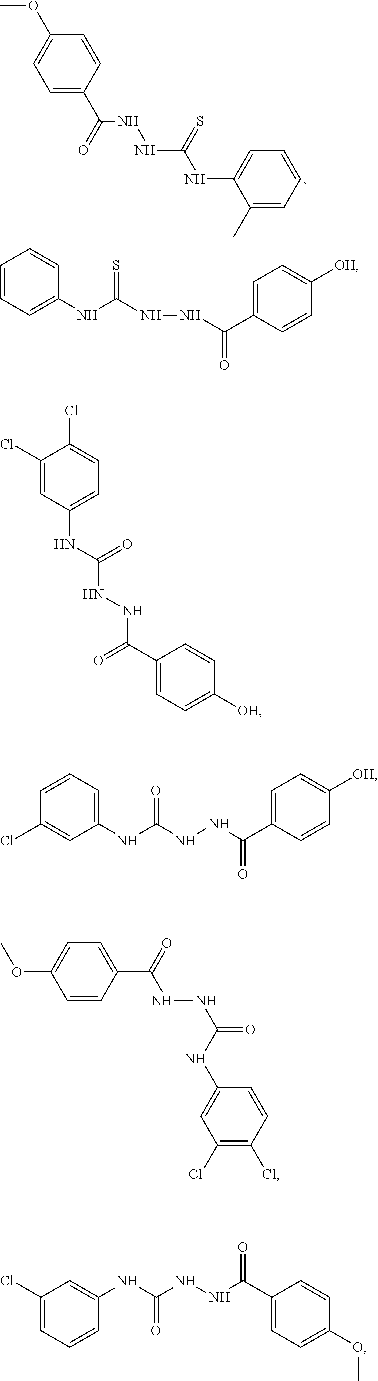 [SMILES: COc1ccc(C(=O)NNC(=S)Nc2ccccc2C)cc1 ; O=C(NNC(=S)Nc1ccccc1)c1ccc(O)cc1 ; O=C(NNC(=O)c1ccc(O)cc1)Nc1ccc(Cl)c(Cl)c1 ; O=C(NNC(=O)c1ccc(O)cc1)Nc1cccc(Cl)c1 ; COc1ccc(C(=O)NNC(=O)Nc2ccc(Cl)c(Cl)c2)cc1 ; COc1ccc(C(=O)NNC(=O)Nc2cccc(Cl)c2)cc1] 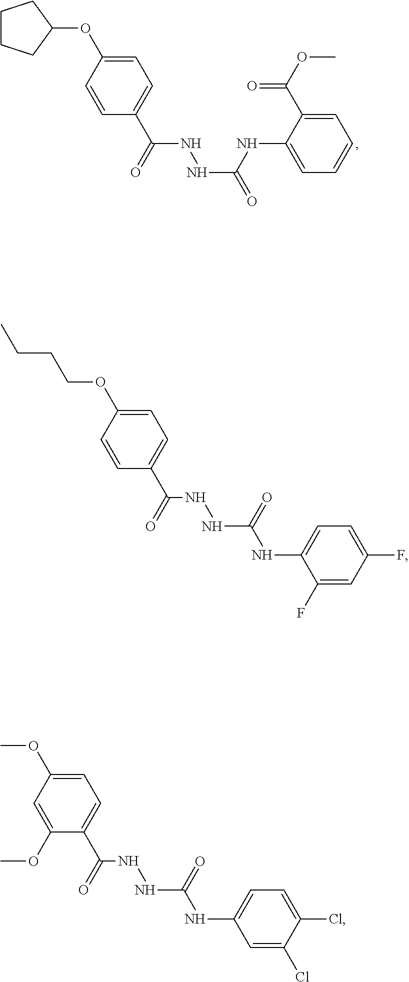 [SMILES: COC(=O)c1ccccc1NC(=O)NNC(=O)c1ccc(OC2CCCC2)cc1 ; CCCCOc1ccc(C(=O)NNC(=O)Nc2ccc(F)cc2F)cc1 ; COc1ccc(C(=O)NNC(=O)Nc2ccc(Cl)c(Cl)c2)c(OC)c1] 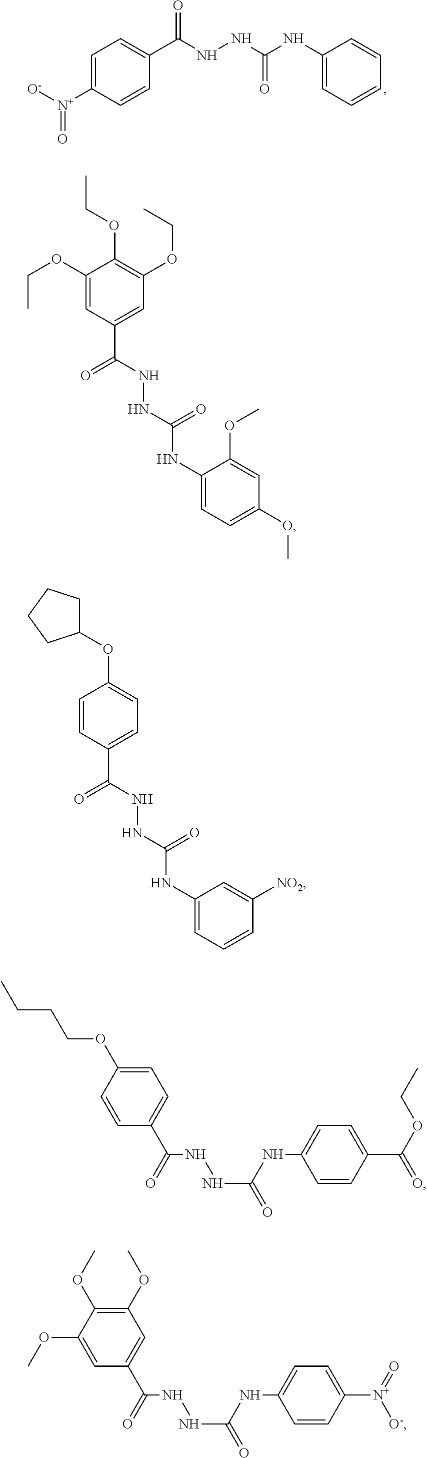 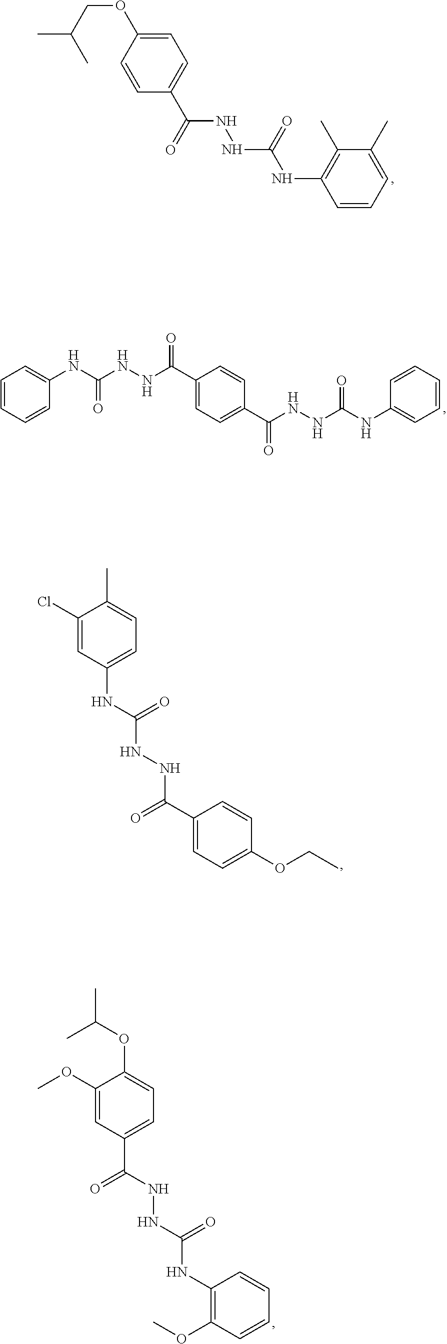 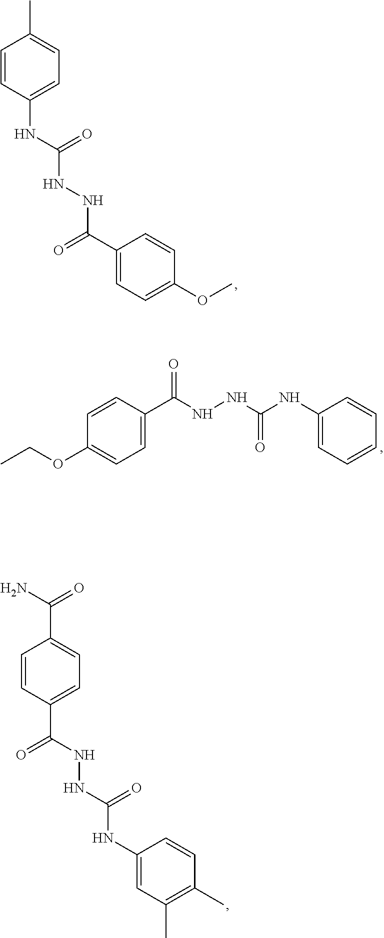 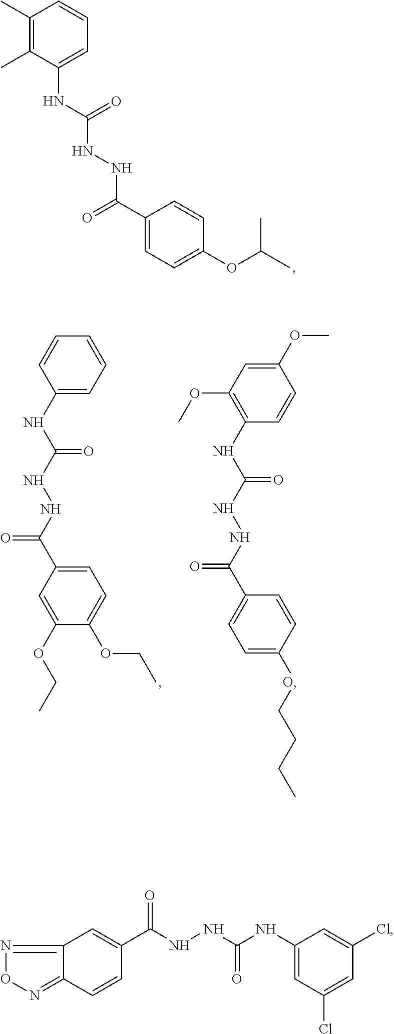 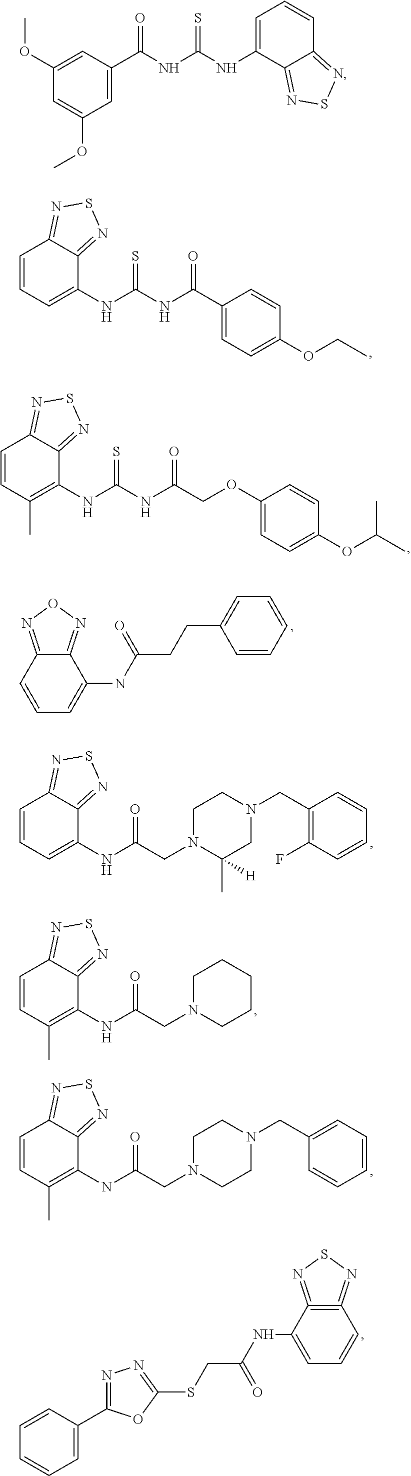 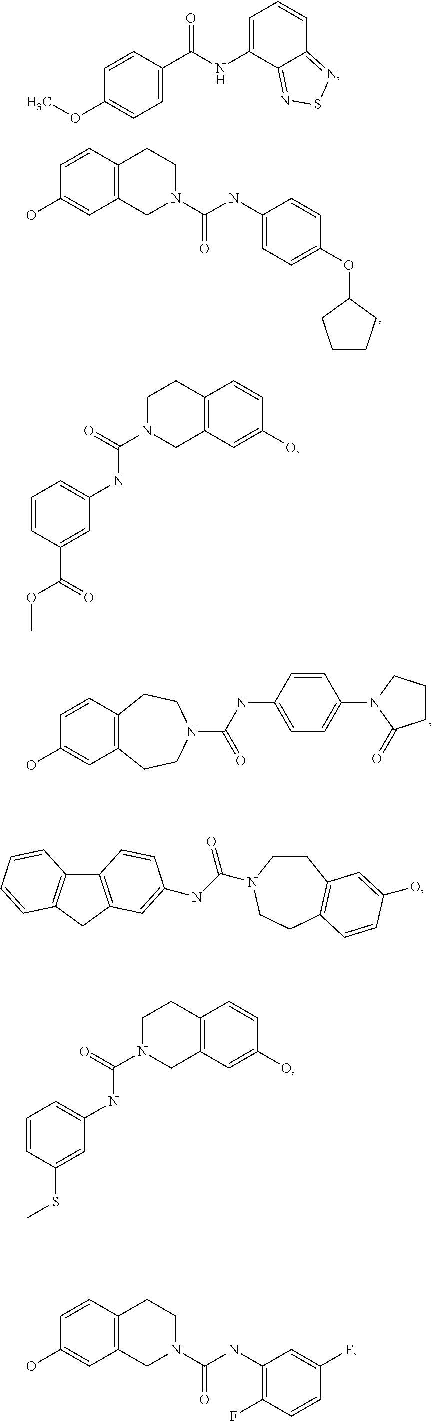 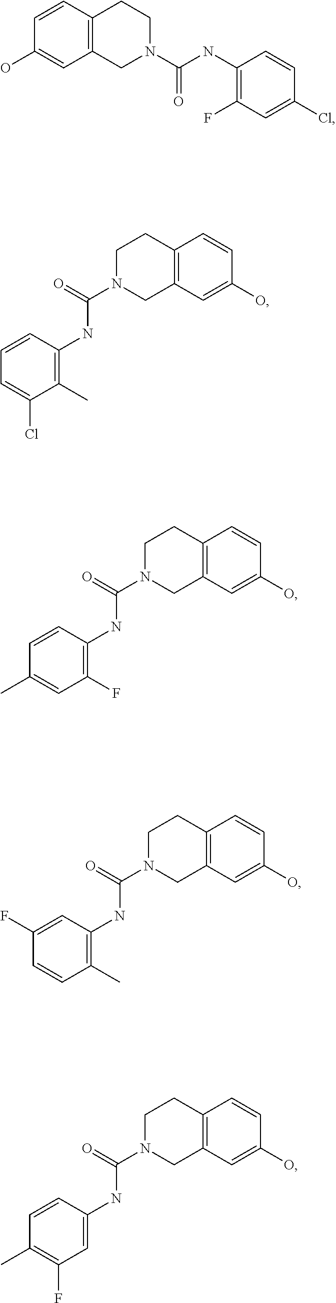 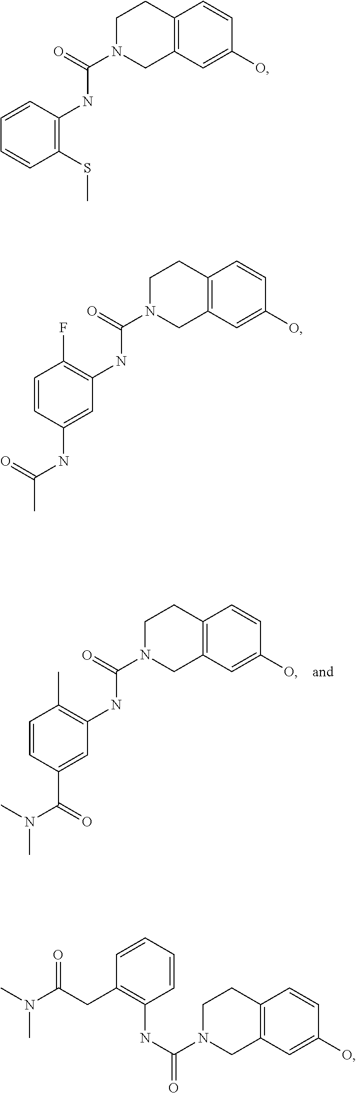
wherein any atoms with an incomplete valence are covalently bonded to one or more hydrogen atoms to complete their valence.

12. The method of claim 11, wherein the compound is

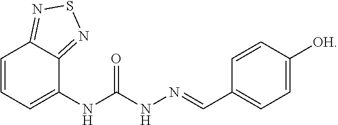 [SMILES: O=C(N/N=C/c1ccc(O)cc1)Nc1cccc2nsnc12]

13. The method of claim 11, wherein the HIV is HIV-1 or HIV-2.

14. The method of claim 8, wherein the HIV is HIV-1 or HIV-2.
